SEQUENCE LISTING (1618 sequences)

SEQ ID NO: 1 - RNA, 20 nt, Synthetic construct
  misc_feature 20: note = invAb
  source 1..20: mol_type = other RNA
ttcttggcaggatggcttct

SEQ ID NO: 2 - RNA, 23 nt, Synthetic construct
  source 1..23: mol_type = other RNA
aagaagccatcctgccaagaatt

SEQ ID NO: 3 - RNA, 20 nt, Synthetic construct
  misc_feature 20: note = invAb
  source 1..20: mol_type = other RNA
tcttggcaggatggcttctc

SEQ ID NO: 4 - RNA, 23 nt, Synthetic construct
  source 1..23: mol_type = other RNA
tgagaagccatcctgccaagatt

SEQ ID NO: 5 - RNA, 20 nt, Synthetic construct
  misc_feature 20: note = invAb
  source 1..20: mol_type = other RNA
cttggcaggatggcttctca

SEQ ID NO: 6 - RNA, 23 nt, Synthetic construct
  source 1..23: mol_type = other RNA
atgagaagccatcctgccaagtt

SEQ ID NO: 7 - RNA, 20 nt, Synthetic construct
  misc_feature 20: note = invAb
  source 1..20: mol_type = other RNA
ttggcaggatggcttctcat

SEQ ID NO: 8 - RNA, 23 nt, Synthetic construct
  source 1..23: mol_type = other RNA
aatgagaagccatcctgccaatt

SEQ ID NO: 9 - RNA, 20 nt, Synthetic construct
  misc_feature 20: note = invAb
  source 1..20: mol_type = other RNA
tggcaggatggcttctcatc

SEQ ID NO: 10 - RNA, 23 nt, Synthetic construct
  source 1..23: mol_type = other RNA
agatgagaagccatcctgccatt

SEQ ID NO: 11 - RNA, 20 nt, Synthetic construct
  misc_feature 20: note = invAb
  source 1..20: mol_type = other RNA
ggcaggatggcttctcatcg

SEQ ID NO: 12 - RNA, 23 nt, Synthetic construct
  source 1..23: mol_type = other RNA
acgatgagaagccatcctgcctt

SEQ ID NO: 13 - RNA, 20 nt, Synthetic construct
  misc_feature 20: note = invAb
  source 1..20: mol_type = other RNA
gcaggatggcttctcatcgt

SEQ ID NO: 14 - RNA, 23 nt, Synthetic construct
  source 1..23: mol_type = other RNA
aacgatgagaagccatcctgctt

SEQ ID NO: 15 - RNA, 20 nt, Synthetic construct
  misc_feature 20: note = invAb
  source 1..20: mol_type = other RNA
caggatggcttctcatcgtc

SEQ ID NO: 16 - RNA, 23 nt, Synthetic construct
  source 1..23: mol_type = other RNA
agacgatgagaagccatcctgtt

SEQ ID NO: 17 - RNA, 20 nt, Synthetic construct
  misc_feature 20: note = invAb
  source 1..20: mol_type = other RNA
aggatggcttctcatcgtct

SEQ ID NO: 18 - RNA, 23 nt, Synthetic construct
  source 1..23: mol_type = other RNA
aagacgatgagaagccatccttt

SEQ ID NO: 19 - RNA, 20 nt, Synthetic construct
  misc_feature 20: note = invAb
  source 1..20: mol_type = other RNA
ggatggcttctcatcgtctg

SEQ ID NO: 20 - RNA, 23 nt, Synthetic construct
  source 1..23: mol_type = other RNA
acagacgatgagaagccatcctt

SEQ ID NO: 21 - RNA, 20 nt, Synthetic construct
  misc_feature 20: note = invAb
  source 1..20: mol_type = other RNA
gatggcttctcatcgtctgc

SEQ ID NO: 22 - RNA, 23 nt, Synthetic construct
  source 1..23: mol_type = other RNA
agcagacgatgagaagccatctt

SEQ ID NO: 23 - RNA, 20 nt, Synthetic construct
  misc_feature 20: note = invAb
  source 1..20: mol_type = other RNA
atggcttctcatcgtctgct

SEQ ID NO: 24 - RNA, 23 nt, Synthetic construct
  source 1..23: mol_type = other RNA
aagcagacgatgagaagccattt

SEQ ID NO: 25 - RNA, 20 nt, Synthetic construct
  misc_feature 20: note = invAb
  source 1..20: mol_type = other RNA
tggcttctcatcgtctgctc

SEQ ID NO: 26 - RNA, 23 nt, Synthetic construct
  source 1..23: mol_type = other RNA
agagcagacgatgagaagccatt

SEQ ID NO: 27 - RNA, 20 nt, Synthetic construct
  misc_feature 20: note = invAb
  source 1..20: mol_type = other RNA
gcttctcatcgtctgctcct

SEQ ID NO: 28 - RNA, 23 nt, Synthetic construct
  source 1..23: mol_type = other RNA
aaggagcagacgatgagaagctt

SEQ ID NO: 29 - RNA, 20 nt, Synthetic construct
  misc_feature 20: note = invAb
  source 1..20: mol_type = other RNA
cttctcatcgtctgctcctc

SEQ ID NO: 30 - RNA, 23 nt, Synthetic construct
  source 1..23: mol_type = other RNA
agaggagcagacgatgagaagtt

SEQ ID NO: 31 - RNA, 20 nt, Synthetic construct
  misc_feature 20: note = invAb
  source 1..20: mol_type = other RNA
ttctcatcgtctgctcctcc

SEQ ID NO: 32 - RNA, 23 nt, Synthetic construct
  source 1..23: mol_type = other RNA
aggaggagcagacgatgagaatt

SEQ ID NO: 33 - RNA, 20 nt, Synthetic construct
  misc_feature 20: note = invAb
  source 1..20: mol_type = other RNA
tctcatcgtctgctcctcct

SEQ ID NO: 34 - RNA, 23 nt, Synthetic construct
  source 1..23: mol_type = other RNA
aaggaggagcagacgatgagatt

SEQ ID NO: 35 - RNA, 20 nt, Synthetic construct
  misc_feature 20: note = invAb
  source 1..20: mol_type = other RNA
ctcatcgtctgctcctcctc

SEQ ID NO: 36 - RNA, 23 nt, Synthetic construct
  source 1..23: mol_type = other RNA
agaggaggagcagacgatgagtt

SEQ ID NO: 37 - RNA, 20 nt, Synthetic construct
  misc_feature 20: note = invAb
  source 1..20: mol_type = other RNA
catcgtctgctcctcctctg

SEQ ID NO: 38 - RNA, 23 nt, Synthetic construct
  source 1..23: mol_type = other RNA
acagaggaggagcagacgatgtt

SEQ ID NO: 39 - RNA, 20 nt, Synthetic construct
  misc_feature 20: note = invAb
  source 1..20: mol_type = other RNA
gctggactggtatttgtgtc

SEQ ID NO: 40 - RNA, 23 nt, Synthetic construct
  source 1..23: mol_type = other RNA
agacacaaataccagtccagctt

SEQ ID NO: 41 - RNA, 20 nt, Synthetic construct
  misc_feature 20: note = invAb
  source 1..20: mol_type = other RNA
ctggactggtatttgtgtct

SEQ ID NO: 42 - RNA, 23 nt, Synthetic construct
  source 1..23: mol_type = other RNA
aagacacaaataccagtccagtt

SEQ ID NO: 43 - RNA, 20 nt, Synthetic construct
  misc_feature 20: note = invAb
  source 1..20: mol_type = other RNA
tggactggtatttgtgtctg

SEQ ID NO: 44 - RNA, 23 nt, Synthetic construct
  source 1..23: mol_type = other RNA
tcagacacaaataccagtccatt

SEQ ID NO: 45 - RNA, 20 nt, Synthetic construct
  misc_feature 20: note = invAb
  source 1..20: mol_type = other RNA
ggactggtatttgtgtctga

SEQ ID NO: 46 - RNA, 23 nt, Synthetic construct
  source 1..23: mol_type = other RNA
atcagacacaaataccagtcctt

SEQ ID NO: 47 - RNA, 20 nt, Synthetic construct
  misc_feature 20: note = invAb
  source 1..20: mol_type = other RNA
gactggtatttgtgtctgag

SEQ ID NO: 48 - RNA, 23 nt, Synthetic construct
  source 1..23: mol_type = other RNA
actcagacacaaataccagtctt

SEQ ID NO: 49 - RNA, 20 nt, Synthetic construct
  misc_feature 20: note = invAb
  source 1..20: mol_type = other RNA
actggtatttgtgtctgagg

SEQ ID NO: 50 - RNA, 23 nt, Synthetic construct
  source 1..23: mol_type = other RNA
acctcagacacaaataccagttt

SEQ ID NO: 51 - RNA, 20 nt, Synthetic construct
  misc_feature 20: note = invAb
  source 1..20: mol_type = other RNA
ctggtatttgtgtctgaggc

SEQ ID NO: 52 - RNA, 23 nt, Synthetic construct
  source 1..23: mol_type = other RNA
agcctcagacacaaataccagtt

SEQ ID NO: 53 - RNA, 20 nt, Synthetic construct
  misc_feature 20: note = invAb
  source 1..20: mol_type = other RNA
tggtatttgtgtctgaggct

SEQ ID NO: 54 - RNA, 23 nt, Synthetic construct
  source 1..23: mol_type = other RNA
aagcctcagacacaaataccatt

SEQ ID NO: 55 - RNA, 20 nt, Synthetic construct
  misc_feature 20: note = invAb
  source 1..20: mol_type = other RNA
ggtatttgtgtctgaggctg

SEQ ID NO: 56 - RNA, 23 nt, Synthetic construct
  source 1..23: mol_type = other RNA
acagcctcagacacaaatacctt

SEQ ID NO: 57 - RNA, 20 nt, Synthetic construct
  misc_feature 20: note = invAb
  source 1..20: mol_type = other RNA
tgaatccaagtgtcctctga

SEQ ID NO: 58 - RNA, 23 nt, Synthetic construct
  source 1..23: mol_type = other RNA
atcagaggacacttggattcatt

SEQ ID NO: 59 - RNA, 20 nt, Synthetic construct
  misc_feature 20: note = invAb
  source 1..20: mol_type = other RNA
gaatccaagtgtcctctgat

SEQ ID NO: 60 - RNA, 23 nt, Synthetic construct
  source 1..23: mol_type = other RNA
aatcagaggacacttggattctt

SEQ ID NO: 61 - RNA, 20 nt, Synthetic construct
  misc_feature 20: note = invAb
  source 1..20: mol_type = other RNA
aatccaagtgtcctctgatg

SEQ ID NO: 62 - RNA, 23 nt, Synthetic construct
  source 1..23: mol_type = other RNA
acatcagaggacacttggatttt

SEQ ID NO: 63 - RNA, 20 nt, Synthetic construct
  misc_feature 20: note = invAb
  source 1..20: mol_type = other RNA
atccaagtgtcctctgatgg

SEQ ID NO: 64 - RNA, 23 nt, Synthetic construct
  source 1..23: mol_type = other RNA
accatcagaggacacttggattt

SEQ ID NO: 65 - RNA, 20 nt, Synthetic construct
  misc_feature 20: note = invAb
  source 1..20: mol_type = other RNA
tccaagtgtcctctgatggt

SEQ ID NO: 66 - RNA, 23 nt, Synthetic construct
  source 1..23: mol_type = other RNA
aaccatcagaggacacttggatt

SEQ ID NO: 67 - RNA, 20 nt, Synthetic construct
  misc_feature 20: note = invAb
  source 1..20: mol_type = other RNA
ccaagtgtcctctgatggtc

SEQ ID NO: 68 - RNA, 23 nt, Synthetic construct
  source 1..23: mol_type = other RNA
tgaccatcagaggacacttggtt

SEQ ID NO: 69 - RNA, 20 nt, Synthetic construct
  misc_feature 20: note = invAb
  source 1..20: mol_type = other RNA
caagtgtcctctgatggtca

SEQ ID NO: 70 - RNA, 23 nt, Synthetic construct
  source 1..23: mol_type = other RNA
ttgaccatcagaggacacttgtt

SEQ ID NO: 71 - RNA, 20 nt, Synthetic construct
  misc_feature 20: note = invAb
  source 1..20: mol_type = other RNA
aagtgtcctctgatggtcaa

SEQ ID NO: 72 - RNA, 23 nt, Synthetic construct
  source 1..23: mol_type = other RNA
tttgaccatcagaggacactttt

SEQ ID NO: 73 - RNA, 20 nt, Synthetic construct
  misc_feature 20: note = invAb
  source 1..20: mol_type = other RNA
agtgtcctctgatggtcaaa

SEQ ID NO: 74 - RNA, 23 nt, Synthetic construct
  source 1..23: mol_type = other RNA
atttgaccatcagaggacacttt

SEQ ID NO: 75 - RNA, 20 nt, Synthetic construct
  misc_feature 20: note = invAb
  source 1..20: mol_type = other RNA
tgtcctctgatggtcaaagt

SEQ ID NO: 76 - RNA, 23 nt, Synthetic construct
  source 1..23: mol_type = other RNA
aactttgaccatcagaggacatt

SEQ ID NO: 77 - RNA, 20 nt, Synthetic construct
  misc_feature 20: note = invAb
  source 1..20: mol_type = other RNA
gtcctctgatggtcaaagtt

SEQ ID NO: 78 - RNA, 23 nt, Synthetic construct
  source 1..23: mol_type = other RNA
aaactttgaccatcagaggactt

SEQ ID NO: 79 - RNA, 20 nt, Synthetic construct
  misc_feature 20: note = invAb
  source 1..20: mol_type = other RNA
tcctctgatggtcaaagttc

SEQ ID NO: 80 - RNA, 23 nt, Synthetic construct
  source 1..23: mol_type = other RNA
agaactttgaccatcagaggatt

SEQ ID NO: 81 - RNA, 20 nt, Synthetic construct
  misc_feature 20: note = invAb
  source 1..20: mol_type = other RNA
cctctgatggtcaaagttct

SEQ ID NO: 82 - RNA, 23 nt, Synthetic construct
  source 1..23: mol_type = other RNA
tagaactttgaccatcagaggtt

SEQ ID NO: 83 - RNA, 20 nt, Synthetic construct
  misc_feature 20: note = invAb
  source 1..20: mol_type = other RNA
ctctgatggtcaaagttcta

SEQ ID NO: 84 - RNA, 23 nt, Synthetic construct
  source 1..23: mol_type = other RNA
atagaactttgaccatcagagtt

SEQ ID NO: 85 - RNA, 20 nt, Synthetic construct
  misc_feature 20: note = invAb
  source 1..20: mol_type = other RNA
tctgatggtcaaagttctag

SEQ ID NO: 86 - RNA, 23 nt, Synthetic construct
  source 1..23: mol_type = other RNA
tctagaactttgaccatcagatt

SEQ ID NO: 87 - RNA, 20 nt, Synthetic construct
  misc_feature 20: note = invAb
  source 1..20: mol_type = other RNA
ctgatggtcaaagttctaga

SEQ ID NO: 88 - RNA, 23 nt, Synthetic construct
  source 1..23: mol_type = other RNA
atctagaactttgaccatcagtt

SEQ ID NO: 89 - RNA, 20 nt, Synthetic construct
  misc_feature 20: note = invAb
  source 1..20: mol_type = other RNA
tgatggtcaaagttctagat

SEQ ID NO: 90 - RNA, 23 nt, Synthetic construct
  source 1..23: mol_type = other RNA
aatctagaactttgaccatcatt

SEQ ID NO: 91 - RNA, 20 nt, Synthetic construct
  misc_feature 20: note = invAb
  source 1..20: mol_type = other RNA
gatggtcaaagttctagatg

SEQ ID NO: 92 - RNA, 23 nt, Synthetic construct
  source 1..23: mol_type = other RNA
acatctagaactttgaccatctt

SEQ ID NO: 93 - RNA, 20 nt, Synthetic construct
  misc_feature 20: note = invAb
  source 1..20: mol_type = other RNA
gccatttgcctctgggaaaa

SEQ ID NO: 94 - RNA, 23 nt, Synthetic construct
  source 1..23: mol_type = other RNA
attttcccagaggcaaatggctt

SEQ ID NO: 95 - RNA, 20 nt, Synthetic construct
  misc_feature 20: note = invAb
  source 1..20: mol_type = other RNA
ccatttgcctctgggaaaac

SEQ ID NO: 96 - RNA, 23 nt, Synthetic construct
  source 1..23: mol_type = other RNA
agttttcccagaggcaaatggtt

SEQ ID NO: 97 - RNA, 20 nt, Synthetic construct
  misc_feature 20: note = invAb
  source 1..20: mol_type = other RNA
catttgcctctgggaaaacc

SEQ ID NO: 98 - RNA, 23 nt, Synthetic construct
  source 1..23: mol_type = other RNA
tggttttcccagaggcaaatgtt

SEQ ID NO: 99 - RNA, 20 nt, Synthetic construct
  misc_feature 20: note = invAb
  source 1..20: mol_type = other RNA
atttgcctctgggaaaacca

SEQ ID NO: 100 - RNA, 23 nt, Synthetic construct
  source 1..23: mol_type = other RNA
atggttttcccagaggcaaattt

SEQ ID NO: 101 - RNA, 20 nt, Synthetic construct
  misc_feature 20: note = invAb
  source 1..20: mol_type = other RNA
tttgcctctgggaaaaccag

SEQ ID NO: 102 - RNA, 23 nt, Synthetic construct
  source 1..23: mol_type = other RNA
actggttttcccagaggcaaatt

SEQ ID NO: 103 - RNA, 20 nt, Synthetic construct
  misc_feature 20: note = invAb
  source 1..20: mol_type = other RNA
ttgcctctgggaaaaccagt

SEQ ID NO: 104 - RNA, 23 nt, Synthetic construct
  source 1..23: mol_type = other RNA
aactggttttcccagaggcaatt

SEQ ID NO: 105 - RNA, 20 nt, Synthetic construct
  misc_feature 20: note = invAb
  source 1..20: mol_type = other RNA
tgcctctgggaaaaccagtg

SEQ ID NO: 106 - RNA, 23 nt, Synthetic construct
  source 1..23: mol_type = other RNA
tcactggttttcccagaggcatt

SEQ ID NO: 107 - RNA, 20 nt, Synthetic construct
  misc_feature 20: note = invAb
  source 1..20: mol_type = other RNA
ctctgggaaaaccagtgagt

SEQ ID NO: 108 - RNA, 23 nt, Synthetic construct
  source 1..23: mol_type = other RNA
aactcactggttttcccagagtt

SEQ ID NO: 109 - RNA, 20 nt, Synthetic construct
  misc_feature 20: note = invAb
  source 1..20: mol_type = other RNA
tctgggaaaaccagtgagtc

SEQ ID NO: 110 - RNA, 23 nt, Synthetic construct
  source 1..23: mol_type = other RNA
agactcactggttttcccagatt

SEQ ID NO: 111 - RNA, 20 nt, Synthetic construct
  misc_feature 20: note = invAb
  source 1..20: mol_type = other RNA
ctgggaaaaccagtgagtct

SEQ ID NO: 112 - RNA, 23 nt, Synthetic construct
  source 1..23: mol_type = other RNA
aagactcactggttttcccagtt

SEQ ID NO: 113 - RNA, 20 nt, Synthetic construct
  misc_feature 20: note = invAb
  source 1..20: mol_type = other RNA
tgggaaaaccagtgagtctg

SEQ ID NO: 114 - RNA, 23 nt, Synthetic construct
  source 1..23: mol_type = other RNA
acagactcactggttttcccatt

SEQ ID NO: 115 - RNA, 20 nt, Synthetic construct
  misc_feature 20: note = invAb
  source 1..20: mol_type = other RNA
gggaaaaccagtgagtctgg

SEQ ID NO: 116 - RNA, 23 nt, Synthetic construct
  source 1..23: mol_type = other RNA
tccagactcactggttttccctt

SEQ ID NO: 117 - RNA, 20 nt, Synthetic construct
  misc_feature 20: note = invAb
  source 1..20: mol_type = other RNA
ggaaaaccagtgagtctgga

SEQ ID NO: 118 - RNA, 23 nt, Synthetic construct
  source 1..23: mol_type = other RNA
atccagactcactggttttcctt

SEQ ID NO: 119 - RNA, 20 nt, Synthetic construct
  misc_feature 20: note = invAb
  source 1..20: mol_type = other RNA
gaaaaccagtgagtctggag

SEQ ID NO: 120 - RNA, 23 nt, Synthetic construct
  source 1..23: mol_type = other RNA
tctccagactcactggttttctt

SEQ ID NO: 121 - RNA, 20 nt, Synthetic construct
  misc_feature 20: note = invAb
  source 1..20: mol_type = other RNA
aaaaccagtgagtctggaga

SEQ ID NO: 122 - RNA, 23 nt, Synthetic construct
  source 1..23: mol_type = other RNA
atctccagactcactggtttttt

SEQ ID NO: 123 - RNA, 20 nt, Synthetic construct
  misc_feature 20: note = invAb
  source 1..20: mol_type = other RNA
aaaccagtgagtctggagag

SEQ ID NO: 124 - RNA, 23 nt, Synthetic construct
  source 1..23: mol_type = other RNA
actctccagactcactggttttt

SEQ ID NO: 125 - RNA, 20 nt, Synthetic construct
  misc_feature 20: note = invAb
  source 1..20: mol_type = other RNA
aaccagtgagtctggagagc

SEQ ID NO: 126 - RNA, 23 nt, Synthetic construct
  source 1..23: mol_type = other RNA
agctctccagactcactggtttt

SEQ ID NO: 127 - RNA, 20 nt, Synthetic construct
  misc_feature 20: note = invAb
  source 1..20: mol_type = other RNA
gtgagtctggagagctgcat

SEQ ID NO: 128 - RNA, 23 nt, Synthetic construct
  source 1..23: mol_type = other RNA
aatgcagctctccagactcactt

SEQ ID NO: 129 - RNA, 20 nt, Synthetic construct
  misc_feature 20: note = invAb
  source 1..20: mol_type = other RNA
tgagtctggagagctgcatg

SEQ ID NO: 130 - RNA, 23 nt, Synthetic construct
  source 1..23: mol_type = other RNA
acatgcagctctccagactcatt

SEQ ID NO: 131 - RNA, 20 nt, Synthetic construct
  misc_feature 20: note = invAb
  source 1..20: mol_type = other RNA
gagtctggagagctgcatgg

SEQ ID NO: 132 - RNA, 23 nt, Synthetic construct
  source 1..23: mol_type = other RNA
accatgcagctctccagactctt

SEQ ID NO: 133 - RNA, 20 nt, Synthetic construct
  misc_feature 20: note = invAb
  source 1..20: mol_type = other RNA
gtctggagagctgcatgggc

SEQ ID NO: 134 - RNA, 23 nt, Synthetic construct
  source 1..23: mol_type = other RNA
agcccatgcagctctccagactt

SEQ ID NO: 135 - RNA, 20 nt, Synthetic construct
  misc_feature 20: note = invAb
  source 1..20: mol_type = other RNA
tctggagagctgcatgggct

SEQ ID NO: 136 - RNA, 23 nt, Synthetic construct
  source 1..23: mol_type = other RNA
aagcccatgcagctctccagatt

SEQ ID NO: 137 - RNA, 20 nt, Synthetic construct
  misc_feature 20: note = invAb
  source 1..20: mol_type = other RNA
ctggagagctgcatgggctc

SEQ ID NO: 138 - RNA, 23 nt, Synthetic construct
  source 1..23: mol_type = other RNA
tgagcccatgcagctctccagtt

SEQ ID NO: 139 - RNA, 20 nt, Synthetic construct
  misc_feature 20: note = invAb
  source 1..20: mol_type = other RNA
ggagagctgcatgggctcac

SEQ ID NO: 140 - RNA, 23 nt, Synthetic construct
  source 1..23: mol_type = other RNA
tgtgagcccatgcagctctcctt

SEQ ID NO: 141 - RNA, 20 nt, Synthetic construct
  misc_feature 20: note = invAb
  source 1..20: mol_type = other RNA
gagagctgcatgggctcaca

SEQ ID NO: 142 - RNA, 23 nt, Synthetic construct
  source 1..23: mol_type = other RNA
ttgtgagcccatgcagctctctt

SEQ ID NO: 143 - RNA, 20 nt, Synthetic construct
  misc_feature 20: note = invAb
  source 1..20: mol_type = other RNA
agagctgcatgggctcacaa

SEQ ID NO: 144 - RNA, 23 nt, Synthetic construct
  source 1..23: mol_type = other RNA
attgtgagcccatgcagctcttt

SEQ ID NO: 145 - RNA, 20 nt, Synthetic construct
  misc_feature 20: note = invAb
  source 1..20: mol_type = other RNA
gagctgcatgggctcacaac

SEQ ID NO: 146 - RNA, 23 nt, Synthetic construct
  source 1..23: mol_type = other RNA
agttgtgagcccatgcagctctt

SEQ ID NO: 147 - RNA, 20 nt, Synthetic construct
  misc_feature 20: note = invAb
  source 1..20: mol_type = other RNA
agctgcatgggctcacaact

SEQ ID NO: 148 - RNA, 23 nt, Synthetic construct
  source 1..23: mol_type = other RNA
aagttgtgagcccatgcagcttt

SEQ ID NO: 149 - RNA, 20 nt, Synthetic construct
  misc_feature 20: note = invAb
  source 1..20: mol_type = other RNA
gctgcatgggctcacaactg

SEQ ID NO: 150 - RNA, 23 nt, Synthetic construct
  source 1..23: mol_type = other RNA
tcagttgtgagcccatgcagctt

SEQ ID NO: 151 - RNA, 20 nt, Synthetic construct
  misc_feature 20: note = invAb
  source 1..20: mol_type = other RNA
ctgcatgggctcacaactga

SEQ ID NO: 152 - RNA, 23 nt, Synthetic construct
  source 1..23: mol_type = other RNA
atcagttgtgagcccatgcagtt

SEQ ID NO: 153 - RNA, 20 nt, Synthetic construct
  misc_feature 20: note = invAb
  source 1..20: mol_type = other RNA
tgcatgggctcacaactgag

SEQ ID NO: 154 - RNA, 23 nt, Synthetic construct
  source 1..23: mol_type = other RNA
actcagttgtgagcccatgcatt

SEQ ID NO: 155 - RNA, 20 nt, Synthetic construct
  misc_feature 20: note = invAb
  source 1..20: mol_type = other RNA
gcatgggctcacaactgagg

SEQ ID NO: 156 - RNA, 23 nt, Synthetic construct
  source 1..23: mol_type = other RNA
tcctcagttgtgagcccatgctt

SEQ ID NO: 157 - RNA, 20 nt, Synthetic construct
  misc_feature 20: note = invAb
  source 1..20: mol_type = other RNA
catgggctcacaactgagga

SEQ ID NO: 158 - RNA, 23 nt, Synthetic construct
  source 1..23: mol_type = other RNA
atcctcagttgtgagcccatgtt

SEQ ID NO: 159 - RNA, 20 nt, Synthetic construct
  misc_feature 20: note = invAb
  source 1..20: mol_type = other RNA
ggctcacaactgaggaggaa

SEQ ID NO: 160 - RNA, 23 nt, Synthetic construct
  source 1..23: mol_type = other RNA
attcctcctcagttgtgagcctt

SEQ ID NO: 161 - RNA, 20 nt, Synthetic construct
  misc_feature 20: note = invAb
  source 1..20: mol_type = other RNA
gctcacaactgaggaggaat

SEQ ID NO: 162 - RNA, 23 nt, Synthetic construct
  source 1..23: mol_type = other RNA
aattcctcctcagttgtgagctt

SEQ ID NO: 163 - RNA, 20 nt, Synthetic construct
  misc_feature 20: note = invAb
  source 1..20: mol_type = other RNA
ctcacaactgaggaggaatt

SEQ ID NO: 164 - RNA, 23 nt, Synthetic construct
  source 1..23: mol_type = other RNA
aaattcctcctcagttgtgagtt

SEQ ID NO: 165 - RNA, 20 nt, Synthetic construct
  misc_feature 20: note = invAb
  source 1..20: mol_type = other RNA
cacaactgaggaggaatttg

SEQ ID NO: 166 - RNA, 23 nt, Synthetic construct
  source 1..23: mol_type = other RNA
acaaattcctcctcagttgtgtt

SEQ ID NO: 167 - RNA, 20 nt, Synthetic construct
  misc_feature 20: note = invAb
  source 1..20: mol_type = other RNA
acaactgaggaggaatttgt

SEQ ID NO: 168 - RNA, 23 nt, Synthetic construct
  source 1..23: mol_type = other RNA
tacaaattcctcctcagttgttt

SEQ ID NO: 169 - RNA, 20 nt, Synthetic construct
  misc_feature 20: note = invAb
  source 1..20: mol_type = other RNA
caactgaggaggaatttgta

SEQ ID NO: 170 - RNA, 23 nt, Synthetic construct
  source 1..23: mol_type = other RNA
atacaaattcctcctcagttgtt

SEQ ID NO: 171 - RNA, 20 nt, Synthetic construct
  misc_feature 20: note = invAb
  source 1..20: mol_type = other RNA
aactgaggaggaatttgtag

SEQ ID NO: 172 - RNA, 23 nt, Synthetic construct
  source 1..23: mol_type = other RNA
tctacaaattcctcctcagtttt

SEQ ID NO: 173 - RNA, 20 nt, Synthetic construct
  misc_feature 20: note = invAb
  source 1..20: mol_type = other RNA
actgaggaggaatttgtaga

SEQ ID NO: 174 - RNA, 23 nt, Synthetic construct
  source 1..23: mol_type = other RNA
ttctacaaattcctcctcagttt

SEQ ID NO: 175 - RNA, 20 nt, Synthetic construct
  misc_feature 20: note = invAb
  source 1..20: mol_type = other RNA
ctgaggaggaatttgtagaa

SEQ ID NO: 176 - RNA, 23 nt, Synthetic construct
  source 1..23: mol_type = other RNA
attctacaaattcctcctcagtt

SEQ ID NO: 177 - RNA, 20 nt, Synthetic construct
  misc_feature 20: note = invAb
  source 1..20: mol_type = other RNA
tgaggaggaatttgtagaag

SEQ ID NO: 178 - RNA, 23 nt, Synthetic construct
  source 1..23: mol_type = other RNA
acttctacaaattcctcctcatt

SEQ ID NO: 179 - RNA, 20 nt, Synthetic construct
  misc_feature 20: note = invAb
  source 1..20: mol_type = other RNA
gaggaggaatttgtagaagg

SEQ ID NO: 180 - RNA, 23 nt, Synthetic construct
  source 1..23: mol_type = other RNA
accttctacaaattcctcctctt

SEQ ID NO: 181 - RNA, 20 nt, Synthetic construct
  misc_feature 20: note = invAb
  source 1..20: mol_type = other RNA
aggaggaatttgtagaaggg

SEQ ID NO: 182 - RNA, 23 nt, Synthetic construct
  source 1..23: mol_type = other RNA
tcccttctacaaattcctccttt

SEQ ID NO: 183 - RNA, 20 nt, Synthetic construct
  misc_feature 20: note = invAb
  source 1..20: mol_type = other RNA
ggaggaatttgtagaaggga

SEQ ID NO: 184 - RNA, 23 nt, Synthetic construct
  source 1..23: mol_type = other RNA
atcccttctacaaattcctcctt

SEQ ID NO: 185 - RNA, 20 nt, Synthetic construct
  misc_feature 20: note = invAb
  source 1..20: mol_type = other RNA
gaggaatttgtagaagggat

SEQ ID NO: 186 - RNA, 23 nt, Synthetic construct
  source 1..23: mol_type = other RNA
tatcccttctacaaattcctctt

SEQ ID NO: 187 - RNA, 20 nt, Synthetic construct
  misc_feature 20: note = invAb
  source 1..20: mol_type = other RNA
ggaatttgtagaagggatat

SEQ ID NO: 188 - RNA, 23 nt, Synthetic construct
  source 1..23: mol_type = other RNA
tatatcccttctacaaattcctt

SEQ ID NO: 189 - RNA, 20 nt, Synthetic construct
  misc_feature 20: note = invAb
  source 1..20: mol_type = other RNA
gaatttgtagaagggatata

SEQ ID NO: 190 - RNA, 23 nt, Synthetic construct
  source 1..23: mol_type = other RNA
atatatcccttctacaaattctt

SEQ ID NO: 191 - RNA, 20 nt, Synthetic construct
  misc_feature 20: note = invAb
  source 1..20: mol_type = other RNA
aatttgtagaagggatatac

SEQ ID NO: 192 - RNA, 23 nt, Synthetic construct
  source 1..23: mol_type = other RNA
tgtatatcccttctacaaatttt

SEQ ID NO: 193 - RNA, 20 nt, Synthetic construct
  misc_feature 20: note = invAb
  source 1..20: mol_type = other RNA
aatttgtagaagggatatac

SEQ ID NO: 194 - RNA, 23 nt, Synthetic construct
  source 1..23: mol_type = other RNA
tgtatatcccttctacaaatttt

SEQ ID NO: 195 - RNA, 20 nt, Synthetic construct
  misc_feature 20: note = invAb
  source 1..20: mol_type = other RNA
atttgtagaagggatataca

SEQ ID NO: 196 - RNA, 23 nt, Synthetic construct
  source 1..23: mol_type = other RNA
ttgtatatcccttctacaaattt

SEQ ID NO: 197 - RNA, 20 nt, Synthetic construct
  misc_feature 20: note = invAb
  source 1..20: mol_type = other RNA
tttgtagaagggatatacaa

SEQ ID NO: 198 - RNA, 23 nt, Synthetic construct
  source 1..23: mol_type = other RNA
tttgtatatcccttctacaaatt

SEQ ID NO: 199 - RNA, 20 nt, Synthetic construct
  misc_feature 20: note = invAb
  source 1..20: mol_type = other RNA
ttgtagaagggatatacaaa

SEQ ID NO: 200 - RNA, 23 nt, Synthetic construct
  source 1..23: mol_type = other RNA
atttgtatatcccttctacaatt

SEQ ID NO: 201 - RNA, 20 nt, Synthetic construct
  misc_feature 20: note = invAb
  source 1..20: mol_type = other RNA
tgtagaagggatatacaaag

SEQ ID NO: 202 - RNA, 23 nt, Synthetic construct
  source 1..23: mol_type = other RNA
actttgtatatcccttctacatt

SEQ ID NO: 203 - RNA, 20 nt, Synthetic construct
  misc_feature 20: note = invAb
  source 1..20: mol_type = other RNA
tgtagaagggatatacaaag

SEQ ID NO: 204 - RNA, 23 nt, Synthetic construct
  source 1..23: mol_type = other RNA
actttgtatatcccttctacatt

SEQ ID NO: 205 - RNA, 20 nt, Synthetic construct
  misc_feature 20: note = invAb
  source 1..20: mol_type = other RNA
gtagaagggatatacaaagt

SEQ ID NO: 206 - RNA, 23 nt, Synthetic construct
  source 1..23: mol_type = other RNA
aactttgtatatcccttctactt

SEQ ID NO: 207 - RNA, 20 nt, Synthetic construct
  misc_feature 20: note = invAb
  source 1..20: mol_type = other RNA
tagaagggatatacaaagtg

SEQ ID NO: 208 - RNA, 23 nt, Synthetic construct
  source 1..23: mol_type = other RNA
acactttgtatatcccttctatt

SEQ ID NO: 209 - RNA, 20 nt, Synthetic construct
  misc_feature 20: note = invAb
  source 1..20: mol_type = other RNA
agaagggatatacaaagtgg

SEQ ID NO: 210 - RNA, 23 nt, Synthetic construct
  source 1..23: mol_type = other RNA
tccactttgtatatcccttcttt

SEQ ID NO: 211 - RNA, 20 nt, Synthetic construct
  misc_feature 20: note = invAb
  source 1..20: mol_type = other RNA
gaagggatatacaaagtgga

SEQ ID NO: 212 - RNA, 23 nt, Synthetic construct
  source 1..23: mol_type = other RNA
ttccactttgtatatcccttctt

SEQ ID NO: 213 - RNA, 20 nt, Synthetic construct
  misc_feature 20: note = invAb
  source 1..20: mol_type = other RNA
gaagggatatacaaagtgga

SEQ ID NO: 214 - RNA, 23 nt, Synthetic construct
  source 1..23: mol_type = other RNA
ttccactttgtatatcccttctt

SEQ ID NO: 215 - RNA, 20 nt, Synthetic construct
  misc_feature 20: note = invAb
  source 1..20: mol_type = other RNA
aagggatatacaaagtggaa

SEQ ID NO: 216 - RNA, 23 nt, Synthetic construct
  source 1..23: mol_type = other RNA
tttccactttgtatatccctttt

SEQ ID NO: 217 - RNA, 20 nt, Synthetic construct
  misc_feature 20: note = invAb
  source 1..20: mol_type = other RNA
agggatatacaaagtggaaa

SEQ ID NO: 218 - RNA, 23 nt, Synthetic construct
  source 1..23: mol_type = other RNA
atttccactttgtatatcccttt

SEQ ID NO: 219 - RNA, 20 nt, Synthetic construct
  misc_feature 20: note = invAb
  source 1..20: mol_type = other RNA
gggatatacaaagtggaaat

SEQ ID NO: 220 - RNA, 23 nt, Synthetic construct
  source 1..23: mol_type = other RNA
tatttccactttgtatatccctt

SEQ ID NO: 221 - RNA, 20 nt, Synthetic construct
  misc_feature 20: note = invAb
  source 1..20: mol_type = other RNA
ggatatacaaagtggaaata

SEQ ID NO: 222 - RNA, 23 nt, Synthetic construct
  source 1..23: mol_type = other RNA
atatttccactttgtatatcctt

SEQ ID NO: 223 - RNA, 20 nt, Synthetic construct
  misc_feature 20: note = invAb
  source 1..20: mol_type = other RNA
gatatacaaagtggaaatag

SEQ ID NO: 224 - RNA, 23 nt, Synthetic construct
  source 1..23: mol_type = other RNA
tctatttccactttgtatatctt

SEQ ID NO: 225 - RNA, 20 nt, Synthetic construct
  misc_feature 20: note = invAb
  source 1..20: mol_type = other RNA
atatacaaagtggaaataga

SEQ ID NO: 226 - RNA, 23 nt, Synthetic construct
  source 1..23: mol_type = other RNA
atctatttccactttgtatattt

SEQ ID NO: 227 - RNA, 20 nt, Synthetic construct
  misc_feature 20: note = invAb
  source 1..20: mol_type = other RNA
tatacaaagtggaaatagac

SEQ ID NO: 228 - RNA, 23 nt, Synthetic construct
  source 1..23: mol_type = other RNA
tgtctatttccactttgtatatt

SEQ ID NO: 229 - RNA, 20 nt, Synthetic construct
  misc_feature 20: note = invAb
  source 1..20: mol_type = other RNA
atacaaagtggaaatagaca

SEQ ID NO: 230 - RNA, 23 nt, Synthetic construct
  source 1..23: mol_type = other RNA
atgtctatttccactttgtattt

SEQ ID NO: 231 - RNA, 20 nt, Synthetic construct
  misc_feature 20: note = invAb
  source 1..20: mol_type = other RNA
atacaaagtggaaatagaca

SEQ ID NO: 232 - RNA, 23 nt, Synthetic construct
  source 1..23: mol_type = other RNA
atgtctatttccactttgtattt

SEQ ID NO: 233 - RNA, 20 nt, Synthetic construct
  misc_feature 20: note = invAb
  source 1..20: mol_type = other RNA
tacaaagtggaaatagacac

SEQ ID NO: 234 - RNA, 23 nt, Synthetic construct
  source 1..23: mol_type = other RNA
agtgtctatttccactttgtatt

SEQ ID NO: 235 - RNA, 20 nt, Synthetic construct
  misc_feature 20: note = invAb
  source 1..20: mol_type = other RNA
acaaagtggaaatagacacc

SEQ ID NO: 236 - RNA, 23 nt, Synthetic construct
  source 1..23: mol_type = other RNA
tggtgtctatttccactttgttt

SEQ ID NO: 237 - RNA, 20 nt, Synthetic construct
  misc_feature 20: note = invAb
  source 1..20: mol_type = other RNA
caaagtggaaatagacacca

SEQ ID NO: 238 - RNA, 23 nt, Synthetic construct
  source 1..23: mol_type = other RNA
ttggtgtctatttccactttgtt

SEQ ID NO: 239 - RNA, 20 nt, Synthetic construct
  misc_feature 20: note = invAb
  source 1..20: mol_type = other RNA
aagtggaaatagacaccaaa

SEQ ID NO: 240 - RNA, 23 nt, Synthetic construct
  source 1..23: mol_type = other RNA
atttggtgtctatttccactttt

SEQ ID NO: 241 - RNA, 20 nt, Synthetic construct
  misc_feature 20: note = invAb
  source 1..20: mol_type = other RNA
aagtggaaatagacaccaaa

SEQ ID NO: 242 - RNA, 23 nt, Synthetic construct
  source 1..23: mol_type = other RNA
atttggtgtctatttccactttt

SEQ ID NO: 243 - RNA, 20 nt, Synthetic construct
  misc_feature 20: note = invAb
  source 1..20: mol_type = other RNA
agtggaaatagacaccaaat

SEQ ID NO: 244 - RNA, 23 nt, Synthetic construct
  source 1..23: mol_type = other RNA
aatttggtgtctatttccacttt

SEQ ID NO: 245 - RNA, 20 nt, Synthetic construct
  misc_feature 20: note = invAb
  source 1..20: mol_type = other RNA
gtggaaatagacaccaaatc

SEQ ID NO: 246 - RNA, 23 nt, Synthetic construct
  source 1..23: mol_type = other RNA
agatttggtgtctatttccactt

SEQ ID NO: 247 - RNA, 20 nt, Synthetic construct
  misc_feature 20: note = invAb
  source 1..20: mol_type = other RNA
tggaaatagacaccaaatct

SEQ ID NO: 248 - RNA, 23 nt, Synthetic construct
  source 1..23: mol_type = other RNA
aagatttggtgtctatttccatt

SEQ ID NO: 249 - RNA, 20 nt, Synthetic construct
  misc_feature 20: note = invAb
  source 1..20: mol_type = other RNA
ggaaatagacaccaaatctt

SEQ ID NO: 250 - RNA, 23 nt, Synthetic construct
  source 1..23: mol_type = other RNA
taagatttggtgtctatttcctt

SEQ ID NO: 251 - RNA, 20 nt, Synthetic construct
  misc_feature 20: note = invAb
  source 1..20: mol_type = other RNA
ggaaatagacaccaaatctt

SEQ ID NO: 252 - RNA, 23 nt, Synthetic construct
  source 1..23: mol_type = other RNA
taagatttggtgtctatttcctt

SEQ ID NO: 253 - RNA, 20 nt, Synthetic construct
  misc_feature 20: note = invAb
  source 1..20: mol_type = other RNA
gaaatagacaccaaatctta

SEQ ID NO: 254 - RNA, 23 nt, Synthetic construct
  source 1..23: mol_type = other RNA
ataagatttggtgtctatttctt

SEQ ID NO: 255 - RNA, 20 nt, Synthetic construct
  misc_feature 20: note = invAb
  source 1..20: mol_type = other RNA
aaatagacaccaaatcttac

SEQ ID NO: 256 - RNA, 23 nt, Synthetic construct
  source 1..23: mol_type = other RNA
agtaagatttggtgtctattttt

SEQ ID NO: 257 - RNA, 20 nt, Synthetic construct
  source 1..20: mol_type = other RNA
aatagacaccaaatcttact

SEQ ID NO: 258 - RNA, 23 nt, Synthetic construct
  source 1..23: mol_type = other RNA
aagtaagatttggtgtctatttt

SEQ ID NO: 259 - RNA, 20 nt, Synthetic construct
  misc_feature 20: note = invAb
  source 1..20: mol_type = other RNA
atagacaccaaatcttactg

SEQ ID NO: 260 - RNA, 23 nt, Synthetic construct
  source 1..23: mol_type = other RNA
acagtaagatttggtgtctattt

SEQ ID NO: 261 - RNA, 20 nt, Synthetic construct
  misc_feature 20: note = invAb
  source 1..20: mol_type = other RNA
atagacaccaaatcttactg

SEQ ID NO: 262 - RNA, 23 nt, Synthetic construct
  source 1..23: mol_type = other RNA
acagtaagatttggtgtctattt

SEQ ID NO: 263 - RNA, 20 nt, Synthetic construct
  misc_feature 20: note = invAb
  source 1..20: mol_type = other RNA
tagacaccaaatcttactgg

SEQ ID NO: 264 - RNA, 23 nt, Synthetic construct
  source 1..23: mol_type = other RNA
tccagtaagatttggtgtctatt

SEQ ID NO: 265 - RNA, 20 nt, Synthetic construct
  misc_feature 20: note = invAb
  source 1..20: mol_type = other RNA
agacaccaaatcttactgga

SEQ ID NO: 266 - RNA, 23 nt, Synthetic construct
  source 1..23: mol_type = other RNA
ttccagtaagatttggtgtcttt

SEQ ID NO: 267 - RNA, 20 nt, Synthetic construct
  misc_feature 20: note = invAb
  source 1..20: mol_type = other RNA
cacttggcatctccccattc

SEQ ID NO: 268 - RNA, 23 nt, Synthetic construct
  source 1..23: mol_type = other RNA
agaatggggagatgccaagtgtt

SEQ ID NO: 269 - RNA, 20 nt, Synthetic construct
  misc_feature 20: note = invAb
  source 1..20: mol_type = other RNA
acttggcatctccccattcc

SEQ ID NO: 270 - RNA, 23 nt, Synthetic construct
  source 1..23: mol_type = other RNA
tggaatggggagatgccaagttt

SEQ ID NO: 271 - RNA, 20 nt, Synthetic construct
  misc_feature 20: note = invAb
  source 1..20: mol_type = other RNA
acttggcatctccccattcc

SEQ ID NO: 272 - RNA, 23 nt, Synthetic construct
  source 1..23: mol_type = other RNA
tggaatggggagatgccaagttt

SEQ ID NO: 273 - RNA, 20 nt, Synthetic construct
  misc_feature 20: note = invAb
  source 1..20: mol_type = other RNA
cttggcatctccccattcca

SEQ ID NO: 274 - RNA, 23 nt, Synthetic construct
  source 1..23: mol_type = other RNA
atggaatggggagatgccaagtt

SEQ ID NO: 275 - RNA, 20 nt, Synthetic construct
  misc_feature 20: note = invAb
  source 1..20: mol_type = other RNA
ttggcatctccccattccat

SEQ ID NO: 276 - RNA, 23 nt, Synthetic construct
  source 1..23: mol_type = other RNA
aatggaatggggagatgccaatt

SEQ ID NO: 277 - RNA, 20 nt, Synthetic construct
  misc_feature 20: note = invAb
  source 1..20: mol_type = other RNA
tggcatctccccattccatg

SEQ ID NO: 278 - RNA, 23 nt, Synthetic construct
  source 1..23: mol_type = other RNA
tcatggaatggggagatgccatt

SEQ ID NO: 279 - RNA, 20 nt, Synthetic construct
  misc_feature 20: note = invAb
  source 1..20: mol_type = other RNA
ggcatctccccattccatga

SEQ ID NO: 280 - RNA, 23 nt, Synthetic construct
  source 1..23: mol_type = other RNA
atcatggaatggggagatgcctt

SEQ ID NO: 281 - RNA, 20 nt, Synthetic construct
  misc_feature 20: note = invAb
  source 1..20: mol_type = other RNA
ggcatctccccattccatga

SEQ ID NO: 282 - RNA, 23 nt, Synthetic construct
  source 1..23: mol_type = other RNA
atcatggaatggggagatgcctt

SEQ ID NO: 283 - RNA, 20 nt, Synthetic construct
  misc_feature 20: note = invAb
  source 1..20: mol_type = other RNA
gcatctccccattccatgag

SEQ ID NO: 284 - RNA, 23 nt, Synthetic construct
  source 1..23: mol_type = other RNA
actcatggaatggggagatgctt

SEQ ID NO: 285 - RNA, 20 nt, Synthetic construct
  misc_feature 20: note = invAb
  source 1..20: mol_type = other RNA
catctccccattccatgagc

SEQ ID NO: 286 - RNA, 23 nt, Synthetic construct
  source 1..23: mol_type = other RNA
tgctcatggaatggggagatgtt

SEQ ID NO: 287 - RNA, 20 nt, Synthetic construct
  misc_feature 20: note = invAb
  source 1..20: mol_type = other RNA
atctccccattccatgagca

SEQ ID NO: 288 - RNA, 23 nt, Synthetic construct
  source 1..23: mol_type = other RNA
atgctcatggaatggggagattt

SEQ ID NO: 289 - RNA, 20 nt, Synthetic construct
  misc_feature 20: note = invAb
  source 1..20: mol_type = other RNA
tctccccattccatgagcat

SEQ ID NO: 290 - RNA, 23 nt, Synthetic construct
  source 1..23: mol_type = other RNA
aatgctcatggaatggggagatt

SEQ ID NO: 291 - RNA, 20 nt, Synthetic construct
  misc_feature 20: note = invAb
  source 1..20: mol_type = other RNA
tctccccattccatgagcat

SEQ ID NO: 292 - RNA, 23 nt, Synthetic construct
  source 1..23: mol_type = other RNA
aatgctcatggaatggggagatt

SEQ ID NO: 293 - RNA, 20 nt, Synthetic construct
  misc_feature 20: note = invAb
  source 1..20: mol_type = other RNA
ctccccattccatgagcatg

SEQ ID NO: 294 - RNA, 23 nt, Synthetic construct
  source 1..23: mol_type = other RNA
acatgctcatggaatggggagtt

SEQ ID NO: 295 - RNA, 20 nt, Synthetic construct
  misc_feature 20: note = invAb
  source 1..20: mol_type = other RNA
tccccattccatgagcatgc

SEQ ID NO: 296 - RNA, 23 nt, Synthetic construct
  source 1..23: mol_type = other RNA
tgcatgctcatggaatggggatt

SEQ ID NO: 297 - RNA, 20 nt, Synthetic construct
  misc_feature 20: note = invAb
  source 1..20: mol_type = other RNA
ccccattccatgagcatgca

SEQ ID NO: 298 - RNA, 23 nt, Synthetic construct
  source 1..23: mol_type = other RNA
atgcatgctcatggaatggggtt

SEQ ID NO: 299 - RNA, 20 nt, Synthetic construct
  source 1..20: mol_type = other RNA
ccattccatgagcatgcaga

SEQ ID NO: 300 - RNA, 23 nt, Synthetic construct
  source 1..23: mol_type = other RNA
atctgcatgctcatggaatggtt

SEQ ID NO: 301 - RNA, 20 nt, Synthetic construct
  misc_feature 20: note = invAb
  source 1..20: mol_type = other RNA
ccattccatgagcatgcaga

SEQ ID NO: 302 - RNA, 23 nt, Synthetic construct
  source 1..23: mol_type = other RNA
atctgcatgctcatggaatggtt

SEQ ID NO: 303 - RNA, 20 nt, Synthetic construct
  misc_feature 20: note = invAb
  source 1..20: mol_type = other RNA
cattccatgagcatgcagag

SEQ ID NO: 304 - RNA, 23 nt, Synthetic construct
  source 1..23: mol_type = other RNA
actctgcatgctcatggaatgtt

SEQ ID NO: 305 - RNA, 20 nt, Synthetic construct
  misc_feature 20: note = invAb
  source 1..20: mol_type = other RNA
attccatgagcatgcagagg

SEQ ID NO: 306 - RNA, 23 nt, Synthetic construct
  source 1..23: mol_type = other RNA
acctctgcatgctcatggaattt

SEQ ID NO: 307 - RNA, 20 nt, Synthetic construct
  misc_feature 20: note = invAb
  source 1..20: mol_type = other RNA
ttccatgagcatgcagaggt

SEQ ID NO: 308 - RNA, 23 nt, Synthetic construct
  source 1..23: mol_type = other RNA
aacctctgcatgctcatggaatt

SEQ ID NO: 309 - RNA, 20 nt, Synthetic construct
  misc_feature 20: note = invAb
  source 1..20: mol_type = other RNA
tccatgagcatgcagaggtg

SEQ ID NO: 310 - RNA, 23 nt, Synthetic construct
  source 1..23: mol_type = other RNA
acacctctgcatgctcatggatt

SEQ ID NO: 311 - RNA, 20 nt, Synthetic construct
  misc_feature 20: note = invAb
  source 1..20: mol_type = other RNA
ccatgagcatgcagaggtgg

SEQ ID NO: 312 - RNA, 23 nt, Synthetic construct
  source 1..23: mol_type = other RNA
accacctctgcatgctcatggtt

SEQ ID NO: 313 - RNA, 20 nt, Synthetic construct
  misc_feature 20: note = invAb
  source 1..20: mol_type = other RNA
catgagcatgcagaggtggt

SEQ ID NO: 314 - RNA, 23 nt, Synthetic construct
  source 1..23: mol_type = other RNA
taccacctctgcatgctcatgtt

SEQ ID NO: 315 - RNA, 20 nt, Synthetic construct
  misc_feature 20: note = invAb
  source 1..20: mol_type = other RNA
atgagcatgcagaggtggta

SEQ ID NO: 316 - RNA, 23 nt, Synthetic construct
  source 1..23: mol_type = other RNA
ataccacctctgcatgctcattt

SEQ ID NO: 317 - RNA, 20 nt, Synthetic construct
  misc_feature 20: note = invAb
  source 1..20: mol_type = other RNA
tgagcatgcagaggtggtat

SEQ ID NO: 318 - RNA, 23 nt, Synthetic construct
  source 1..23: mol_type = other RNA
aataccacctctgcatgctcatt

SEQ ID NO: 319 - RNA, 20 nt, Synthetic construct
  misc_feature 20: note = invAb
  source 1..20: mol_type = other RNA
gagcatgcagaggtggtatt

SEQ ID NO: 320 - RNA, 23 nt, Synthetic construct
  source 1..23: mol_type = other RNA
aaataccacctctgcatgctctt

SEQ ID NO: 321 - RNA, 20 nt, Synthetic construct
  misc_feature 20: note = invAb
  source 1..20: mol_type = other RNA
agcatgcagaggtggtattc

SEQ ID NO: 322 - RNA, 23 nt, Synthetic construct
  source 1..23: mol_type = other RNA
tgaataccacctctgcatgcttt

SEQ ID NO: 323 - RNA, 20 nt, Synthetic construct
  misc_feature 20: note = invAb
  source 1..20: mol_type = other RNA
gcatgcagaggtggtattca

SEQ ID NO: 324 - RNA, 23 nt, Synthetic construct
  source 1..23: mol_type = other RNA
atgaataccacctctgcatgctt

SEQ ID NO: 325 - RNA, 20 nt, Synthetic construct
  misc_feature 20: note = invAb
  source 1..20: mol_type = other RNA
catgcagaggtggtattcac

SEQ ID NO: 326 - RNA, 23 nt, Synthetic construct
  source 1..23: mol_type = other RNA
tgtgaataccacctctgcatgtt

SEQ ID NO: 327 - RNA, 20 nt, Synthetic construct
  misc_feature 20: note = invAb
  source 1..20: mol_type = other RNA
atgcagaggtggtattcaca

SEQ ID NO: 328 - RNA, 23 nt, Synthetic construct
  source 1..23: mol_type = other RNA
atgtgaataccacctctgcattt

SEQ ID NO: 329 - RNA, 20 nt, Synthetic construct
  misc_feature 20: note = invAb
  source 1..20: mol_type = other RNA
agaggtggtattcacagcca

SEQ ID NO: 330 - RNA, 23 nt, Synthetic construct
  source 1..23: mol_type = other RNA
ttggctgtgaataccacctcttt

SEQ ID NO: 331 - RNA, 20 nt, Synthetic construct
  misc_feature 20: note = invAb
  source 1..20: mol_type = other RNA
gaggtggtattcacagccaa

SEQ ID NO: 332 - RNA, 23 nt, Synthetic construct
  source 1..23: mol_type = other RNA
attggctgtgaataccacctctt

SEQ ID NO: 333 - RNA, 20 nt, Synthetic construct
  misc_feature 20: note = invAb
  source 1..20: mol_type = other RNA
aggtggtattcacagccaac

SEQ ID NO: 334 - RNA, 23 nt, Synthetic construct
  source 1..23: mol_type = other RNA
agttggctgtgaataccaccttt

SEQ ID NO: 335 - RNA, 20 nt, Synthetic construct
  misc_feature 20: note = invAb
  source 1..20: mol_type = other RNA
ggtggtattcacagccaacg

SEQ ID NO: 336 - RNA, 23 nt, Synthetic construct
  source 1..23: mol_type = other RNA
tcgttggctgtgaataccacctt

SEQ ID NO: 337 - RNA, 20 nt, Synthetic construct
  misc_feature 20: note = invAb
  source 1..20: mol_type = other RNA
gccgccctgctgagccccta

SEQ ID NO: 338 - RNA, 23 nt, Synthetic construct
  source 1..23: mol_type = other RNA
ataggggctcagcagggcggctt

SEQ ID NO: 339 - RNA, 20 nt, Synthetic construct
  misc_feature 20: note = invAb
  source 1..20: mol_type = other RNA
ccgccctgctgagcccctac

SEQ ID NO: 340 - RNA, 23 nt, Synthetic construct
  source 1..23: mol_type = other RNA
agtaggggctcagcagggcggtt

SEQ ID NO: 341 - RNA, 20 nt, Synthetic construct
  misc_feature 20: note = invAb
  source 1..20: mol_type = other RNA
cgccctgctgagcccctact

SEQ ID NO: 342 - RNA, 23 nt, Synthetic construct
  source 1..23: mol_type = other RNA
aagtaggggctcagcagggcgtt

SEQ ID NO: 343 - RNA, 20 nt, Synthetic construct
  misc_feature 20: note = invAb
  source 1..20: mol_type = other RNA
gccctgctgagcccctactc

SEQ ID NO: 344 - RNA, 23 nt, Synthetic construct
  source 1..23: mol_type = other RNA
agagtaggggctcagcagggctt

SEQ ID NO: 345 - RNA, 20 nt, Synthetic construct
  misc_feature 20: note = invAb
  source 1..20: mol_type = other RNA
ccctgctgagcccctactcc

SEQ ID NO: 346 - RNA, 23 nt, Synthetic construct
  source 1..23: mol_type = other RNA
aggagtaggggctcagcagggtt

SEQ ID NO: 347 - RNA, 20 nt, Synthetic construct
  misc_feature 20: note = invAb
  source 1..20: mol_type = other RNA
cctgctgagcccctactcct

SEQ ID NO: 348 - RNA, 23 nt, Synthetic construct
  source 1..23: mol_type = other RNA
taggagtaggggctcagcaggtt

SEQ ID NO: 349 - RNA, 20 nt, Synthetic construct
  misc_feature 20: note = invAb
  source 1..20: mol_type = other RNA
ctgctgagcccctactccta

SEQ ID NO: 350 - RNA, 23 nt, Synthetic construct
  source 1..23: mol_type = other RNA
ataggagtaggggctcagcagtt

SEQ ID NO: 351 - RNA, 20 nt, Synthetic construct
  misc_feature 20: note = invAb
  source 1..20: mol_type = other RNA
tgctgagcccctactcctat

SEQ ID NO: 352 - RNA, 23 nt, Synthetic construct
  source 1..23: mol_type = other RNA
aataggagtaggggctcagcatt

SEQ ID NO: 353 - RNA, 20 nt, Synthetic construct
  misc_feature 20: note = invAb
  source 1..20: mol_type = other RNA
gctgagcccctactcctatt

SEQ ID NO: 354 - RNA, 23 nt, Synthetic construct
  source 1..23: mol_type = other RNA
aaataggagtaggggctcagctt

SEQ ID NO: 355 - RNA, 20 nt, Synthetic construct
  misc_feature 20: note = invAb
  source 1..20: mol_type = other RNA
ctgagcccctactcctattc

SEQ ID NO: 356 - RNA, 23 nt, Synthetic construct
  source 1..23: mol_type = other RNA
agaataggagtaggggctcagtt

SEQ ID NO: 357 - RNA, 20 nt, Synthetic construct
  misc_feature 20: note = invAb
  source 1..20: mol_type = other RNA
tgagcccctactcctattcc

SEQ ID NO: 358 - RNA, 23 nt, Synthetic construct
  source 1..23: mol_type = other RNA
tggaataggagtaggggctcatt

SEQ ID NO: 359 - RNA, 20 nt, Synthetic construct
  misc_feature 20: note = invAb
  source 1..20: mol_type = other RNA
gagcccctactcctattcca

SEQ ID NO: 360 - RNA, 23 nt, Synthetic construct
  source 1..23: mol_type = other RNA
atggaataggagtaggggctctt

SEQ ID NO: 361 - RNA, 20 nt, Synthetic construct
  misc_feature 20: note = invAb
  source 1..20: mol_type = other RNA
agcccctactcctattccac

SEQ ID NO: 362 - RNA, 23 nt, Synthetic construct
  source 1..23: mol_type = other RNA
agtggaataggagtaggggcttt

SEQ ID NO: 363 - RNA, 20 nt, Synthetic construct
  misc_feature 20: note = invAb
  source 1..20: mol_type = other RNA
gcccctactcctattccacc

SEQ ID NO: 364 - RNA, 23 nt, Synthetic construct
  misc_feature 20: note = invAb
  source 1..23: mol_type = other RNA
tggtggaataggagtaggggctt

SEQ ID NO: 365 - RNA, 20 nt, Synthetic construct
  misc_feature 20: note = invAb
  source 1..20: mol_type = other RNA
cccctactcctattccacca

SEQ ID NO: 366 - RNA, 23 nt, Synthetic construct
  source 1..23: mol_type = other RNA
atggtggaataggagtaggggtt

SEQ ID NO: 367 - RNA, 20 nt, Synthetic construct
  misc_feature 20: note = invAb
  source 1..20: mol_type = other RNA
ccctactcctattccaccac

SEQ ID NO: 368 - RNA, 23 nt, Synthetic construct
  source 1..23: mol_type = other RNA
agtggtggaataggagtagggtt

SEQ ID NO: 369 - RNA, 20 nt, Synthetic construct
  misc_feature 20: note = invAb
  source 1..20: mol_type = other RNA
cctactcctattccaccacg

SEQ ID NO: 370 - RNA, 23 nt, Synthetic construct
  source 1..23: mol_type = other RNA
acgtggtggaataggagtaggtt

SEQ ID NO: 371 - RNA, 20 nt, Synthetic construct
  misc_feature 20: note = invAb
  source 1..20: mol_type = other RNA
ctactcctattccaccacgg

SEQ ID NO: 372 - RNA, 23 nt, Synthetic construct
  source 1..23: mol_type = other RNA
accgtggtggaataggagtagtt

SEQ ID NO: 373 - RNA, 20 nt, Synthetic construct
  misc_feature 20: note = invAb
  source 1..20: mol_type = other RNA
tactcctattccaccacggc

SEQ ID NO: 374 - RNA, 23 nt, Synthetic construct
  source 1..23: mol_type = other RNA
agccgtggtggaataggagtatt

SEQ ID NO: 375 - RNA, 20 nt, Synthetic construct
  misc_feature 20: note = invAb
  source 1..20: mol_type = other RNA
actcctattccaccacggct

SEQ ID NO: 376 - RNA, 23 nt, Synthetic construct
  source 1..23: mol_type = other RNA
aagccgtggtggaataggagttt

SEQ ID NO: 377 - RNA, 20 nt, Synthetic construct
  misc_feature 20: note = invAb
  source 1..20: mol_type = other RNA
ctcctattccaccacggctg

SEQ ID NO: 378 - RNA, 23 nt, Synthetic construct
  source 1..23: mol_type = other RNA
acagccgtggtggaataggagtt

SEQ ID NO: 379 - RNA, 20 nt, Synthetic construct
  misc_feature 20: note = invAb
  source 1..20: mol_type = other RNA
tcctattccaccacggctgt

SEQ ID NO: 380 - RNA, 23 nt, Synthetic construct
  source 1..23: mol_type = other RNA
aacagccgtggtggaataggatt

SEQ ID NO: 381 - RNA, 20 nt, Synthetic construct
  misc_feature 20: note = invAb
  source 1..20: mol_type = other RNA
cctattccaccacggctgtc

SEQ ID NO: 382 - RNA, 23 nt, Synthetic construct
  source 1..23: mol_type = other RNA
agacagccgtggtggaataggtt

SEQ ID NO: 383 - RNA, 20 nt, Synthetic construct
  misc_feature 20: note = invAb
  source 1..20: mol_type = other RNA
ctattccaccacggctgtcg

SEQ ID NO: 384 - RNA, 23 nt, Synthetic construct
  source 1..23: mol_type = other RNA
acgacagccgtggtggaatagtt

SEQ ID NO: 385 - RNA, 20 nt, Synthetic construct
  misc_feature 20: note = invAb
  source 1..20: mol_type = other RNA
tattccaccacggctgtcgt

SEQ ID NO: 386 - RNA, 23 nt, Synthetic construct
  source 1..23: mol_type = other RNA
aacgacagccgtggtggaatatt

SEQ ID NO: 387 - RNA, 20 nt, Synthetic construct
  misc_feature 20: note = invAb
  source 1..20: mol_type = other RNA
attccaccacggctgtcgtc

SEQ ID NO: 388 - RNA, 23 nt, Synthetic construct
  source 1..23: mol_type = other RNA
tgacgacagccgtggtggaattt

SEQ ID NO: 389 - RNA, 20 nt, Synthetic construct
  misc_feature 20: note = invAb
  source 1..20: mol_type = other RNA
ttccaccacggctgtcgtca

SEQ ID NO: 390 - RNA, 23 nt, Synthetic construct
  source 1..23: mol_type = other RNA
atgacgacagccgtggtggaatt

SEQ ID NO: 391 - RNA, 20 nt, Synthetic construct
  misc_feature 20: note = invAb
  source 1..20: mol_type = other RNA
tccaccacggctgtcgtcac

SEQ ID NO: 392 - RNA, 23 nt, Synthetic construct
  source 1..23: mol_type = other RNA
agtgacgacagccgtggtggatt

SEQ ID NO: 393 - RNA, 20 nt, Synthetic construct
  misc_feature 20: note = invAb
  source 1..20: mol_type = other RNA
ccaccacggctgtcgtcacc

SEQ ID NO: 394 - RNA, 23 nt, Synthetic construct
  source 1..23: mol_type = other RNA
tggtgacgacagccgtggtggtt

SEQ ID NO: 395 - RNA, 20 nt, Synthetic construct
  misc_feature 20: note = invAb
  source 1..20: mol_type = other RNA
caccacggctgtcgtcacca

SEQ ID NO: 396 - RNA, 23 nt, Synthetic construct
  source 1..23: mol_type = other RNA
ttggtgacgacagccgtggtgtt

SEQ ID NO: 397 - RNA, 20 nt, Synthetic construct
  misc_feature 20: note = invAb
  source 1..20: mol_type = other RNA
accacggctgtcgtcaccaa

SEQ ID NO: 398 - RNA, 23 nt, Synthetic construct
  source 1..23: mol_type = other RNA
attggtgacgacagccgtggttt

SEQ ID NO: 399 - RNA, 20 nt, Synthetic construct
  misc_feature 20: note = invAb
  source 1..20: mol_type = other RNA
ccacggctgtcgtcaccaat

SEQ ID NO: 400 - RNA, 23 nt, Synthetic construct
  source 1..23: mol_type = other RNA
aattggtgacgacagccgtggtt

SEQ ID NO: 401 - RNA, 20 nt, Synthetic construct
  misc_feature 20: note = invAb
  source 1..20: mol_type = other RNA
cacggctgtcgtcaccaatc

SEQ ID NO: 402 - RNA, 23 nt, Synthetic construct
  source 1..23: mol_type = other RNA
agattggtgacgacagccgtgtt

SEQ ID NO: 403 - RNA, 20 nt, Synthetic construct
  misc_feature 20: note = invAb
  source 1..20: mol_type = other RNA
acggctgtcgtcaccaatcc

SEQ ID NO: 404 - RNA, 23 nt, Synthetic construct
  source 1..23: mol_type = other RNA
aggattggtgacgacagccgttt

SEQ ID NO: 405 - RNA, 20 nt, Synthetic construct
  misc_feature 20: note = invAb
  source 1..20: mol_type = other RNA
cggctgtcgtcaccaatccc

SEQ ID NO: 406 - RNA, 23 nt, Synthetic construct
  source 1..23: mol_type = other RNA
tgggattggtgacgacagccgtt

SEQ ID NO: 407 - RNA, 20 nt, Synthetic construct
  misc_feature 20: note = invAb
  source 1..20: mol_type = other RNA
ggctgtcgtcaccaatccca

SEQ ID NO: 408 - RNA, 23 nt, Synthetic construct
  source 1..23: mol_type = other RNA
ttgggattggtgacgacagcctt

SEQ ID NO: 409 - RNA, 20 nt, Synthetic construct
  misc_feature 20: note = invAb
  source 1..20: mol_type = other RNA
gctgtcgtcaccaatcccaa

SEQ ID NO: 410 - RNA, 23 nt, Synthetic construct
  source 1..23: mol_type = other RNA
attgggattggtgacgacagctt

SEQ ID NO: 411 - RNA, 20 nt, Synthetic construct
  misc_feature 20: note = invAb
  source 1..20: mol_type = other RNA
tgtcgtcaccaatcccaagg

SEQ ID NO: 412 - RNA, 23 nt, Synthetic construct
  source 1..23: mol_type = other RNA
tccttgggattggtgacgacatt

SEQ ID NO: 413 - RNA, 20 nt, Synthetic construct
  misc_feature 20: note = invAb
  source 1..20: mol_type = other RNA
gtcgtcaccaatcccaagga

SEQ ID NO: 414 - RNA, 23 nt, Synthetic construct
  source 1..23: mol_type = other RNA
ttccttgggattggtgacgactt

SEQ ID NO: 415 - RNA, 20 nt, Synthetic construct
  misc_feature 20: note = invAb
  source 1..20: mol_type = other RNA
tcgtcaccaatcccaaggaa

SEQ ID NO: 416 - RNA, 23 nt, Synthetic construct
  source 1..23: mol_type = other RNA
attccttgggattggtgacgatt

SEQ ID NO: 417 - RNA, 20 nt, Synthetic construct
  misc_feature 20: note = invAb
  source 1..20: mol_type = other RNA
cgtcaccaatcccaaggaat

SEQ ID NO: 418 - RNA, 23 nt, Synthetic construct
  source 1..23: mol_type = other RNA
aattccttgggattggtgacgtt

SEQ ID NO: 419 - RNA, 20 nt, Synthetic construct
  misc_feature 20: note = invAb
  source 1..20: mol_type = other RNA
caccaatcccaaggaatgag

SEQ ID NO: 420 - RNA, 23 nt, Synthetic construct
  source 1..23: mol_type = other RNA
actcattccttgggattggtgtt

SEQ ID NO: 421 - RNA, 20 nt, Synthetic construct
  misc_feature 20: note = invAb
  source 1..20: mol_type = other RNA
accaatcccaaggaatgagg

SEQ ID NO: 422 - RNA, 23 nt, Synthetic construct
  source 1..23: mol_type = other RNA
acctcattccttgggattggttt

SEQ ID NO: 423 - RNA, 20 nt, Synthetic construct
  misc_feature 20: note = invAb
  source 1..20: mol_type = other RNA
ccaatcccaaggaatgaggg

SEQ ID NO: 424 - RNA, 23 nt, Synthetic construct
  source 1..23: mol_type = other RNA
tccctcattccttgggattggtt

SEQ ID NO: 425 - RNA, 20 nt, Synthetic construct
  misc_feature 20: note = invAb
  source 1..20: mol_type = other RNA
caatcccaaggaatgaggga

SEQ ID NO: 426 - RNA, 23 nt, Synthetic construct
  source 1..23: mol_type = other RNA
atccctcattccttgggattgtt

SEQ ID NO: 427 - RNA, 20 nt, Synthetic construct
  misc_feature 20: note = invAb
  source 1..20: mol_type = other RNA
aatcccaaggaatgagggac

SEQ ID NO: 428 - RNA, 23 nt, Synthetic construct
  source 1..23: mol_type = other RNA
agtccctcattccttgggatttt

SEQ ID NO: 429 - RNA, 20 nt, Synthetic construct
  misc_feature 20: note = invAb
  source 1..20: mol_type = other RNA
atcccaaggaatgagggact

SEQ ID NO: 430 - RNA, 23 nt, Synthetic construct
  source 1..23: mol_type = other RNA
aagtccctcattccttgggattt

SEQ ID NO: 431 - RNA, 20 nt, Synthetic construct
  misc_feature 20: note = invAb
  source 1..20: mol_type = other RNA
tcccaaggaatgagggactt

SEQ ID NO: 432 - RNA, 23 nt, Synthetic construct
  source 1..23: mol_type = other RNA
aaagtccctcattccttgggatt

SEQ ID NO: 433 - RNA, 20 nt, Synthetic construct
  misc_feature 20: note = invAb
  source 1..20: mol_type = other RNA
cccaaggaatgagggacttc

SEQ ID NO: 434 - RNA, 23 nt, Synthetic construct
  source 1..23: mol_type = other RNA
agaagtccctcattccttgggtt

SEQ ID NO: 435 - RNA, 20 nt, Synthetic construct
  misc_feature 20: note = invAb
  source 1..20: mol_type = other RNA
ccaaggaatgagggacttct

SEQ ID NO: 436 - RNA, 23 nt, Synthetic construct
  source 1..23: mol_type = other RNA
aagaagtccctcattccttggtt

SEQ ID NO: 437 - RNA, 20 nt, Synthetic construct
  misc_feature 20: note = invAb
  source 1..20: mol_type = other RNA
caaggaatgagggacttctc

SEQ ID NO: 438 - RNA, 23 nt, Synthetic construct
  source 1..23: mol_type = other RNA
agagaagtccctcattccttgtt

SEQ ID NO: 439 - RNA, 20 nt, Synthetic construct
  misc_feature 20: note = invAb
  source 1..20: mol_type = other RNA
ctgaaggacgagggatggga

SEQ ID NO: 440 - RNA, 23 nt, Synthetic construct
  source 1..23: mol_type = other RNA
atcccatccctcgtccttcagtt

SEQ ID NO: 441 - RNA, 20 nt, Synthetic construct
  misc_feature 20: note = invAb
  source 1..20: mol_type = other RNA
tgaaggacgagggatgggat

SEQ ID NO: 442 - RNA, 23 nt, Synthetic construct
  source 1..23: mol_type = other RNA
aatcccatccctcgtccttcatt

SEQ ID NO: 443 - RNA, 20 nt, Synthetic construct
  misc_feature 20: note = invAb
  source 1..20: mol_type = other RNA
gaaggacgagggatgggatt

SEQ ID NO: 444 - RNA, 23 nt, Synthetic construct
  source 1..23: mol_type = other RNA
aaatcccatccctcgtccttctt

SEQ ID NO: 445 - RNA, 20 nt, Synthetic construct
  misc_feature 20: note = invAb
  source 1..20: mol_type = other RNA
aaggacgagggatgggattt

SEQ ID NO: 446 - RNA, 23 nt, Synthetic construct
  source 1..23: mol_type = other RNA
aaaatcccatccctcgtcctttt

SEQ ID NO: 447 - RNA, 20 nt, Synthetic construct
  misc_feature 20: note = invAb
  source 1..20: mol_type = other RNA
aggacgagggatgggatttc

SEQ ID NO: 448 - RNA, 23 nt, Synthetic construct
  source 1..23: mol_type = other RNA
tgaaatcccatccctcgtccttt

SEQ ID NO: 449 - RNA, 20 nt, Synthetic construct
  misc_feature 20: note = invAb
  source 1..20: mol_type = other RNA
ggacgagggatgggatttca

SEQ ID NO: 450 - RNA, 23 nt, Synthetic construct
  source 1..23: mol_type = other RNA
atgaaatcccatccctcgtcctt

SEQ ID NO: 451 - RNA, 20 nt, Synthetic construct
  misc_feature 20: note = invAb
  source 1..20: mol_type = other RNA
gacgagggatgggatttcat

SEQ ID NO: 452 - RNA, 23 nt, Synthetic construct
  source 1..23: mol_type = other RNA
aatgaaatcccatccctcgtctt

SEQ ID NO: 453 - RNA, 20 nt, Synthetic construct
  misc_feature 20: note = invAb
  source 1..20: mol_type = other RNA
acgagggatgggatttcatg

SEQ ID NO: 454 - RNA, 23 nt, Synthetic construct
  source 1..23: mol_type = other RNA
acatgaaatcccatccctcgttt

SEQ ID NO: 455 - RNA, 20 nt, Synthetic construct
  misc_feature 20: note = invAb
  source 1..20: mol_type = other RNA
cgagggatgggatttcatgt

SEQ ID NO: 456 - RNA, 23 nt, Synthetic construct
  source 1..23: mol_type = other RNA
tacatgaaatcccatccctcgtt

SEQ ID NO: 457 - RNA, 20 nt, Synthetic construct
  misc_feature 20: note = invAb
  source 1..20: mol_type = other RNA
gagggatgggatttcatgta

SEQ ID NO: 458 - RNA, 23 nt, Synthetic construct
  source 1..23: mol_type = other RNA
ttacatgaaatcccatccctctt

SEQ ID NO: 459 - RNA, 20 nt, Synthetic construct
  misc_feature 20: note = invAb
  source 1..20: mol_type = other RNA
agggatgggatttcatgtaa

SEQ ID NO: 460 - RNA, 23 nt, Synthetic construct
  source 1..23: mol_type = other RNA
attacatgaaatcccatcccttt

SEQ ID NO: 461 - RNA, 20 nt, Synthetic construct
  misc_feature 20: note = invAb
  source 1..20: mol_type = other RNA
gggatgggatttcatgtaac

SEQ ID NO: 462 - RNA, 23 nt, Synthetic construct
  source 1..23: mol_type = other RNA
agttacatgaaatcccatccctt

SEQ ID NO: 463 - RNA, 20 nt, Synthetic construct
  misc_feature 20: note = invAb
  source 1..20: mol_type = other RNA
ggatgggatttcatgtaacc

SEQ ID NO: 464 - RNA, 23 nt, Synthetic construct
  source 1..23: mol_type = other RNA
tggttacatgaaatcccatcctt

SEQ ID NO: 465 - RNA, 20 nt, Synthetic construct
  misc_feature 20: note = invAb
  source 1..20: mol_type = other RNA
gatgggatttcatgtaacca

SEQ ID NO: 466 - RNA, 23 nt, Synthetic construct
  source 1..23: mol_type = other RNA
ttggttacatgaaatcccatctt

SEQ ID NO: 467 - RNA, 20 nt, Synthetic construct
  misc_feature 20: note = invAb
  source 1..20: mol_type = other RNA
atgggatttcatgtaaccaa

SEQ ID NO: 468 - RNA, 23 nt, Synthetic construct
  source 1..23: mol_type = other RNA
attggttacatgaaatcccattt

SEQ ID NO: 469 - RNA, 20 nt, Synthetic construct
  misc_feature 20: note = invAb
  source 1..20: mol_type = other RNA
tgggatttcatgtaaccaag

SEQ ID NO: 470 - RNA, 23 nt, Synthetic construct
  source 1..23: mol_type = other RNA
tcttggttacatgaaatcccatt

SEQ ID NO: 471 - RNA, 20 nt, Synthetic construct
  misc_feature 20: note = invAb
  source 1..20: mol_type = other RNA
gggatttcatgtaaccaaga

SEQ ID NO: 472 - RNA, 23 nt, Synthetic construct
  source 1..23: mol_type = other RNA
atcttggttacatgaaatccctt

SEQ ID NO: 473 - RNA, 20 nt, Synthetic construct
  misc_feature 20: note = invAb
  source 1..20: mol_type = other RNA
ggatttcatgtaaccaagag

SEQ ID NO: 474 - RNA, 23 nt, Synthetic construct
  source 1..23: mol_type = other RNA
actcttggttacatgaaatcctt

SEQ ID NO: 475 - RNA, 20 nt, Synthetic construct
  misc_feature 20: note = invAb
  source 1..20: mol_type = other RNA
gatttcatgtaaccaagagt

SEQ ID NO: 476 - RNA, 23 nt, Synthetic construct
  source 1..23: mol_type = other RNA
tactcttggttacatgaaatctt

SEQ ID NO: 477 - RNA, 20 nt, Synthetic construct
  misc_feature 20: note = invAb
  source 1..20: mol_type = other RNA
atttcatgtaaccaagagta

SEQ ID NO: 478 - RNA, 23 nt, Synthetic construct
  source 1..23: mol_type = other RNA
atactcttggttacatgaaattt

SEQ ID NO: 479 - RNA, 20 nt, Synthetic construct
  misc_feature 20: note = invAb
  source 1..20: mol_type = other RNA
tttcatgtaaccaagagtat

SEQ ID NO: 480 - RNA, 23 nt, Synthetic construct
  source 1..23: mol_type = other RNA
aatactcttggttacatgaaatt

SEQ ID NO: 481 - RNA, 20 nt, Synthetic construct
  misc_feature 20: note = invAb
  source 1..20: mol_type = other RNA
ttcatgtaaccaagagtatt

SEQ ID NO: 482 - RNA, 23 nt, Synthetic construct
  source 1..23: mol_type = other RNA
aaatactcttggttacatgaatt

SEQ ID NO: 483 - RNA, 20 nt, Synthetic construct
  misc_feature 20: note = invAb
  source 1..20: mol_type = other RNA
tcatgtaaccaagagtattc

SEQ ID NO: 484 - RNA, 23 nt, Synthetic construct
  source 1..23: mol_type = other RNA
agaatactcttggttacatgatt

SEQ ID NO: 485 - RNA, 20 nt, Synthetic construct
  misc_feature 20: note = invAb
  source 1..20: mol_type = other RNA
catgtaaccaagagtattcc

SEQ ID NO: 486 - RNA, 23 nt, Synthetic construct
  source 1..23: mol_type = other RNA
tggaatactcttggttacatgtt

SEQ ID NO: 487 - RNA, 20 nt, Synthetic construct
  misc_feature 20: note = invAb
  source 1..20: mol_type = other RNA
atgtaaccaagagtattcca

SEQ ID NO: 488 - RNA, 23 nt, Synthetic construct
  source 1..23: mol_type = other RNA
atggaatactcttggttacattt

SEQ ID NO: 489 - RNA, 20 nt, Synthetic construct
  misc_feature 20: note = invAb
  source 1..20: mol_type = other RNA
tgtaaccaagagtattccat

SEQ ID NO: 490 - RNA, 23 nt, Synthetic construct
  source 1..23: mol_type = other RNA
aatggaatactcttggttacatt

SEQ ID NO: 491 - RNA, 20 nt, Synthetic construct
  misc_feature 20: note = invAb
  source 1..20: mol_type = other RNA
gtaaccaagagtattccatt

SEQ ID NO: 492 - RNA, 23 nt, Synthetic construct
  source 1..23: mol_type = other RNA
aaatggaatactcttggttactt

SEQ ID NO: 493 - RNA, 20 nt, Synthetic construct
  misc_feature 20: note = invAb
  source 1..20: mol_type = other RNA
taaccaagagtattccattt

SEQ ID NO: 494 - RNA, 23 nt, Synthetic construct
  source 1..23: mol_type = other RNA
aaaatggaatactcttggttatt

SEQ ID NO: 495 - RNA, 20 nt, Synthetic construct
  misc_feature 20: note = invAb
  source 1..20: mol_type = other RNA
aaccaagagtattccatttt

SEQ ID NO: 496 - RNA, 23 nt, Synthetic construct
  source 1..23: mol_type = other RNA
aaaaatggaatactcttggtttt

SEQ ID NO: 497 - RNA, 20 nt, Synthetic construct
  misc_feature 20: note = invAb
  source 1..20: mol_type = other RNA
accaagagtattccattttt

SEQ ID NO: 498 - RNA, 23 nt, Synthetic construct
  source 1..23: mol_type = other RNA
taaaaatggaatactcttggttt

SEQ ID NO: 499 - RNA, 20 nt, Synthetic construct
  misc_feature 20: note = invAb
  source 1..20: mol_type = other RNA
ccaagagtattccattttta

SEQ ID NO: 500 - RNA, 23 nt, Synthetic construct
  source 1..23: mol_type = other RNA
ataaaaatggaatactcttggtt

SEQ ID NO: 501 - RNA, 20 nt, Synthetic construct
  misc_feature 20: note = invAb
  source 1..20: mol_type = other RNA
caagagtattccatttttac

SEQ ID NO: 502 - RNA, 23 nt, Synthetic construct
  source 1..23: mol_type = other RNA
agtaaaaatggaatactcttgtt

SEQ ID NO: 503 - RNA, 20 nt, Synthetic construct
  misc_feature 20: note = invAb
  source 1..20: mol_type = other RNA
aagagtattccatttttact

SEQ ID NO: 504 - RNA, 23 nt, Synthetic construct
  source 1..23: mol_type = other RNA
tagtaaaaatggaatactctttt

SEQ ID NO: 505 - RNA, 20 nt, Synthetic construct
  source 1..20: mol_type = other RNA
agagtattccatttttacta

SEQ ID NO: 506 - RNA, 23 nt, Synthetic construct
  source 1..23: mol_type = other RNA
ttagtaaaaatggaatactcttt

SEQ ID NO: 507 - RNA, 20 nt, Synthetic construct
  misc_feature 20: note = invAb
  source 1..20: mol_type = other RNA
gagtattccatttttactaa

SEQ ID NO: 508 - RNA, 23 nt, Synthetic construct
  source 1..23: mol_type = other RNA
tttagtaaaaatggaatactctt

SEQ ID NO: 509 - RNA, 20 nt, Synthetic construct
  misc_feature 20: note = invAb
  source 1..20: mol_type = other RNA
agtattccatttttactaaa

SEQ ID NO: 510 - RNA, 23 nt, Synthetic construct
  source 1..23: mol_type = other RNA
atttagtaaaaatggaatacttt

SEQ ID NO: 511 - RNA, 20 nt, Synthetic construct
  misc_feature 20: note = invAb
  source 1..20: mol_type = other RNA
tccatttttactaaagcagt

SEQ ID NO: 512 - RNA, 23 nt, Synthetic construct
  source 1..23: mol_type = other RNA
aactgctttagtaaaaatggatt

SEQ ID NO: 513 - RNA, 20 nt, Synthetic construct
  misc_feature 20: note = invAb
  source 1..20: mol_type = other RNA
catttttactaaagcagtgt

SEQ ID NO: 514 - RNA, 23 nt, Synthetic construct
  source 1..23: mol_type = other RNA
aacactgctttagtaaaaatgtt

SEQ ID NO: 515 - RNA, 20 nt, Synthetic construct
  misc_feature 20: note = invAb
  source 1..20: mol_type = other RNA
atttttactaaagcagtgtt

SEQ ID NO: 516 - RNA, 23 nt, Synthetic construct
  source 1..23: mol_type = other RNA
aaacactgctttagtaaaaattt

SEQ ID NO: 517 - RNA, 20 nt, Synthetic construct
  misc_feature 20: note = invAb
  source 1..20: mol_type = other RNA
tttttactaaagcagtgttt

SEQ ID NO: 518 - RNA, 23 nt, Synthetic construct
  source 1..23: mol_type = other RNA
aaaacactgctttagtaaaaatt

SEQ ID NO: 519 - RNA, 20 nt, Synthetic construct
  misc_feature 20: note = invAb
  source 1..20: mol_type = other RNA
ttttactaaagcagtgtttt

SEQ ID NO: 520 - RNA, 23 nt, Synthetic construct
  source 1..23: mol_type = other RNA
aaaaacactgctttagtaaaatt

SEQ ID NO: 521 - RNA, 20 nt, Synthetic construct
  misc_feature 20: note = invAb
  source 1..20: mol_type = other RNA
ttactaaagcagtgttttca

SEQ ID NO: 522 - RNA, 23 nt, Synthetic construct
  source 1..23: mol_type = other RNA
atgaaaacactgctttagtaatt

SEQ ID NO: 523 - RNA, 20 nt, Synthetic construct
  misc_feature 20: note = invAb
  source 1..20: mol_type = other RNA
tactaaagcagtgttttcac

SEQ ID NO: 524 - RNA, 23 nt, Synthetic construct
  source 1..23: mol_type = other RNA
agtgaaaacactgctttagtatt

SEQ ID NO: 525 - RNA, 20 nt, Synthetic construct
  misc_feature 20: note = invAb
  source 1..20: mol_type = other RNA
actaaagcagtgttttcacc

SEQ ID NO: 526 - RNA, 23 nt, Synthetic construct
  source 1..23: mol_type = other RNA
aggtgaaaacactgctttagttt

SEQ ID NO: 527 - RNA, 20 nt, Synthetic construct
  misc_feature 20: note = invAb
  source 1..20: mol_type = other RNA
ctaaagcagtgttttcacct

SEQ ID NO: 528 - RNA, 23 nt, Synthetic construct
  source 1..23: mol_type = other RNA
aaggtgaaaacactgctttagtt

SEQ ID NO: 529 - RNA, 20 nt, Synthetic construct
  misc_feature 20: note = invAb
  source 1..20: mol_type = other RNA
taaagcagtgttttcacctc

SEQ ID NO: 530 - RNA, 23 nt, Synthetic construct
  source 1..23: mol_type = other RNA
tgaggtgaaaacactgctttatt

SEQ ID NO: 531 - RNA, 20 nt, Synthetic construct
  misc_feature 20: note = invAb
  source 1..20: mol_type = other RNA
aaagcagtgttttcacctca

SEQ ID NO: 532 - RNA, 23 nt, Synthetic construct
  source 1..23: mol_type = other RNA
atgaggtgaaaacactgcttttt

SEQ ID NO: 533 - RNA, 20 nt, Synthetic construct
  misc_feature 20: note = invAb
  source 1..20: mol_type = other RNA
aagcagtgttttcacctcat

SEQ ID NO: 534 - RNA, 23 nt, Synthetic construct
  source 1..23: mol_type = other RNA
tatgaggtgaaaacactgctttt

SEQ ID NO: 535 - RNA, 20 nt, Synthetic construct
  misc_feature 20: note = invAb
  source 1..20: mol_type = other RNA
agtccaggcagagacaataa

SEQ ID NO: 536 - RNA, 23 nt, Synthetic construct
  source 1..23: mol_type = other RNA
tttattgtctctgcctggacttt

SEQ ID NO: 537 - RNA, 20 nt, Synthetic construct
  misc_feature 20: note = invAb
  source 1..20: mol_type = other RNA
gtccaggcagagacaataaa

SEQ ID NO: 538 - RNA, 23 nt, Synthetic construct
  source 1..23: mol_type = other RNA
ttttattgtctctgcctggactt

SEQ ID NO: 539 - RNA, 20 nt, Synthetic construct
  misc_feature 20: note = invAb
  source 1..20: mol_type = other RNA
tccaggcagagacaataaaa

SEQ ID NO: 540 - RNA, 23 nt, Synthetic construct
  source 1..23: mol_type = other RNA
attttattgtctctgcctggatt

SEQ ID NO: 541 - RNA, 20 nt, Synthetic construct
  misc_feature 20: note = invAb
  source 1..20: mol_type = other RNA
ccaggcagagacaataaaac

SEQ ID NO: 542 - RNA, 23 nt, Synthetic construct
  source 1..23: mol_type = other RNA
tgttttattgtctctgcctggtt

SEQ ID NO: 543 - RNA, 20 nt, Synthetic construct
  misc_feature 20: note = invAb
  source 1..20: mol_type = other RNA
caggcagagacaataaaaca

SEQ ID NO: 544 - RNA, 23 nt, Synthetic construct
  source 1..23: mol_type = other RNA
atgttttattgtctctgcctgtt

SEQ ID NO: 545 - RNA, 20 nt, Synthetic construct
  misc_feature 20: note = invAb
  source 1..20: mol_type = other RNA
aggcagagacaataaaacat

SEQ ID NO: 546 - RNA, 23 nt, Synthetic construct
  source 1..23: mol_type = other RNA
aatgttttattgtctctgccttt

SEQ ID NO: 547 - RNA, 20 nt, Synthetic construct
  misc_feature 20: note = invAb
  source 1..20: mol_type = other RNA
ggcagagacaataaaacatt

SEQ ID NO: 548 - RNA, 23 nt, Synthetic construct
  source 1..23: mol_type = other RNA
aaatgttttattgtctctgcctt

SEQ ID NO: 549 - RNA, 20 nt, Synthetic construct
  misc_feature 20: note = invAb
  source 1..20: mol_type = other RNA
agacaataaaacattcctgt

SEQ ID NO: 550 - RNA, 23 nt, Synthetic construct
  source 1..23: mol_type = other RNA
aacaggaatgttttattgtcttt

SEQ ID NO: 551 - RNA, 20 nt, Synthetic construct
  misc_feature 20: note = invAb
  source 1..20: mol_type = other RNA
gacaataaaacattcctgtg

SEQ ID NO: 552 - RNA, 23 nt, Synthetic construct
  source 1..23: mol_type = other RNA
tcacaggaatgttttattgtctt

SEQ ID NO: 553 - RNA, 20 nt, Synthetic construct
  misc_feature 20: note = invAb
  source 1..20: mol_type = other RNA
acaataaaacattcctgtga

SEQ ID NO: 554 - RNA, 23 nt, Synthetic construct
  source 1..23: mol_type = other RNA
ttcacaggaatgttttattgttt

SEQ ID NO: 555 - RNA, 20 nt, Synthetic construct
  misc_feature 20: note = invAb
  source 1..20: mol_type = other RNA
caataaaacattcctgtgaa

SEQ ID NO: 556 - RNA, 23 nt, Synthetic construct
  source 1..23: mol_type = other RNA
tttcacaggaatgttttattgtt

SEQ ID NO: 557 - RNA, 20 nt, Synthetic construct
  misc_feature 20: note = invAb
  source 1..20: mol_type = other RNA
aataaaacattcctgtgaaa

SEQ ID NO: 558 - RNA, 23 nt, Synthetic construct
  source 1..23: mol_type = other RNA
atttcacaggaatgttttatttt

SEQ ID NO: 559 - RNA, 20 nt, Synthetic construct
  misc_feature 20: note = invAb
  source 1..20: mol_type = other RNA
ataaaacattcctgtgaaag

SEQ ID NO: 560 - RNA, 23 nt, Synthetic construct
  source 1..23: mol_type = other RNA
actttcacaggaatgttttattt

SEQ ID NO: 561 - RNA, 20 nt, Synthetic construct
  misc_feature 20: note = invAb
  source 1..20: mol_type = other RNA
taaaacattcctgtgaaagg

SEQ ID NO: 562 - RNA, 23 nt, Synthetic construct
  source 1..23: mol_type = other RNA
acctttcacaggaatgttttatt

SEQ ID NO: 563 - RNA, 20 nt, Synthetic construct
  misc_feature 20: note = invAb
  source 1..20: mol_type = other RNA
aaaacattcctgtgaaaggc

SEQ ID NO: 564 - RNA, 23 nt, Synthetic construct
  source 1..23: mol_type = other RNA
tgcctttcacaggaatgtttttt

SEQ ID NO: 565 - RNA, 20 nt, Synthetic construct
  misc_feature 20: note = invAb
  source 1..20: mol_type = other RNA
aaacattcctgtgaaaggca

SEQ ID NO: 566 - RNA, 23 nt, Synthetic construct
  source 1..23: mol_type = other RNA
atgcctttcacaggaatgttttt

SEQ ID NO: 567 - RNA, 20 nt, Synthetic construct
  misc_feature 20: note = invAb
  source 1..20: mol_type = other RNA
ttcttggcaggatggcttct

SEQ ID NO: 568 - RNA, 23 nt, Synthetic construct
  source 1..23: mol_type = other RNA
aagaagccatcctgccaagaatt

SEQ ID NO: 569 - RNA, 20 nt, Synthetic construct
  misc_feature 20: note = invAb
  source 1..20: mol_type = other RNA
tcttggcaggatggcttctc

SEQ ID NO: 570 - RNA, 23 nt, Synthetic construct
  source 1..23: mol_type = other RNA
tgagaagccatcctgccaagatt

SEQ ID NO: 571 - RNA, 20 nt, Synthetic construct
  misc_feature 20: note = invAb
  source 1..20: mol_type = other RNA
cttggcaggatggcttctca

SEQ ID NO: 572 - RNA, 23 nt, Synthetic construct
  source 1..23: mol_type = other RNA
atgagaagccatcctgccaagtt

SEQ ID NO: 573 - RNA, 20 nt, Synthetic construct
  misc_feature 20: note = invAb
  source 1..20: mol_type = other RNA
ttggcaggatggcttctcat

SEQ ID NO: 574 - RNA, 23 nt, Synthetic construct
  source 1..23: mol_type = other RNA
aatgagaagccatcctgccaatt

SEQ ID NO: 575 - RNA, 20 nt, Synthetic construct
  misc_feature 20: note = invAb
  source 1..20: mol_type = other RNA
tggcaggatggcttctcatc

SEQ ID NO: 576 - RNA, 23 nt, Synthetic construct
  source 1..23: mol_type = other RNA
agatgagaagccatcctgccatt

SEQ ID NO: 577 - RNA, 20 nt, Synthetic construct
  misc_feature 20: note = invAb
  source 1..20: mol_type = other RNA
ggcaggatggcttctcatcg

SEQ ID NO: 578 - RNA, 23 nt, Synthetic construct
  source 1..23: mol_type = other RNA
acgatgagaagccatcctgcctt

SEQ ID NO: 579 - RNA, 20 nt, Synthetic construct
  misc_feature 20: note = invAb
  source 1..20: mol_type = other RNA
gcaggatggcttctcatcgt

SEQ ID NO: 580 - RNA, 23 nt, Synthetic construct
  source 1..23: mol_type = other RNA
aacgatgagaagccatcctgctt

SEQ ID NO: 581 - RNA, 20 nt, Synthetic construct
  misc_feature 20: note = invAb
  source 1..20: mol_type = other RNA
caggatggcttctcatcgtc

SEQ ID NO: 582 - RNA, 23 nt, Synthetic construct
  source 1..23: mol_type = other RNA
agacgatgagaagccatcctgtt

SEQ ID NO: 583 - RNA, 20 nt, Synthetic construct
  misc_feature 20: note = invAb
  source 1..20: mol_type = other RNA
aggatggcttctcatcgtct

SEQ ID NO: 584 - RNA, 23 nt, Synthetic construct
  source 1..23: mol_type = other RNA
aagacgatgagaagccatccttt

SEQ ID NO: 585 - RNA, 20 nt, Synthetic construct
  misc_feature 20: note = invAb
  source 1..20: mol_type = other RNA
ggatggcttctcatcgtctg

SEQ ID NO: 586 - RNA, 23 nt, Synthetic construct
  source 1..23: mol_type = other RNA
acagacgatgagaagccatcctt

SEQ ID NO: 587 - RNA, 20 nt, Synthetic construct
  misc_feature 20: note = invAb
  source 1..20: mol_type = other RNA
gatggcttctcatcgtctgc

SEQ ID NO: 588 - RNA, 23 nt, Synthetic construct
  source 1..23: mol_type = other RNA
agcagacgatgagaagccatctt

SEQ ID NO: 589 - RNA, 20 nt, Synthetic construct
  misc_feature 20: note = invAb
  source 1..20: mol_type = other RNA
atggcttctcatcgtctgct

SEQ ID NO: 590 - RNA, 23 nt, Synthetic construct
  source 1..23: mol_type = other RNA
aagcagacgatgagaagccattt

SEQ ID NO: 591 - RNA, 20 nt, Synthetic construct
  misc_feature 20: note = invAb
  source 1..20: mol_type = other RNA
tggcttctcatcgtctgctc

SEQ ID NO: 592 - RNA, 23 nt, Synthetic construct
  source 1..23: mol_type = other RNA
agagcagacgatgagaagccatt

SEQ ID NO: 593 - RNA, 20 nt, Synthetic construct
  misc_feature 20: note = invAb
  source 1..20: mol_type = other RNA
gcttctcatcgtctgctcct

SEQ ID NO: 594 - RNA, 23 nt, Synthetic construct
  source 1..23: mol_type = other RNA
aaggagcagacgatgagaagctt

SEQ ID NO: 595 - RNA, 20 nt, Synthetic construct
  misc_feature 20: note = invAb
  source 1..20: mol_type = other RNA
cttctcatcgtctgctcctc

SEQ ID NO: 596 - RNA, 23 nt, Synthetic construct
  source 1..23: mol_type = other RNA
agaggagcagacgatgagaagtt

SEQ ID NO: 597 - RNA, 20 nt, Synthetic construct
  misc_feature 20: note = invAb
  source 1..20: mol_type = other RNA
ttctcatcgtctgctcctcc

SEQ ID NO: 598 - RNA, 23 nt, Synthetic construct
  source 1..23: mol_type = other RNA
aggaggagcagacgatgagaatt

SEQ ID NO: 599 - RNA, 20 nt, Synthetic construct
  misc_feature 20: note = invAb
  source 1..20: mol_type = other RNA
tctcatcgtctgctcctcct

SEQ ID NO: 600 - RNA, 23 nt, Synthetic construct
  source 1..23: mol_type = other RNA
aaggaggagcagacgatgagatt

SEQ ID NO: 601 - RNA, 20 nt, Synthetic construct
  misc_feature 20: note = invAb
  source 1..20: mol_type = other RNA
ctcatcgtctgctcctcctc

SEQ ID NO: 602 - RNA, 23 nt, Synthetic construct
  source 1..23: mol_type = other RNA
agaggaggagcagacgatgagtt

SEQ ID NO: 603 - RNA, 20 nt, Synthetic construct
  misc_feature 20: note = invAb
  source 1..20: mol_type = other RNA
catcgtctgctcctcctctg

SEQ ID NO: 604 - RNA, 23 nt, Synthetic construct
  source 1..23: mol_type = other RNA
acagaggaggagcagacgatgtt

SEQ ID NO: 605 - RNA, 20 nt, Synthetic construct
  misc_feature 20: note = invAb
  source 1..20: mol_type = other RNA
gctggactggtatttgtgtc

SEQ ID NO: 606 - RNA, 23 nt, Synthetic construct
  source 1..23: mol_type = other RNA
agacacaaataccagtccagctt

SEQ ID NO: 607 - RNA, 20 nt, Synthetic construct
  misc_feature 20: note = invAb
  source 1..20: mol_type = other RNA
ctggactggtatttgtgtct

SEQ ID NO: 608 - RNA, 23 nt, Synthetic construct
  source 1..23: mol_type = other RNA
aagacacaaataccagtccagtt

SEQ ID NO: 609 - RNA, 20 nt, Synthetic construct
  misc_feature 20: note = invAb
  source 1..20: mol_type = other RNA
tggactggtatttgtgtctg

SEQ ID NO: 610 - RNA, 23 nt, Synthetic construct
  source 1..23: mol_type = other RNA
tcagacacaaataccagtccatt

SEQ ID NO: 611 - RNA, 20 nt, Synthetic construct
  misc_feature 20: note = invAb
  source 1..20: mol_type = other RNA
ggactggtatttgtgtctga

SEQ ID NO: 612 - RNA, 23 nt, Synthetic construct
  source 1..23: mol_type = other RNA
atcagacacaaataccagtcctt

SEQ ID NO: 613 - RNA, 20 nt, Synthetic construct
  misc_feature 20: note = invAb
  source 1..20: mol_type = other RNA
gactggtatttgtgtctgag

SEQ ID NO: 614 - RNA, 23 nt, Synthetic construct
  source 1..23: mol_type = other RNA
actcagacacaaataccagtctt

SEQ ID NO: 615 - RNA, 20 nt, Synthetic construct
  misc_feature 20: note = invAb
  source 1..20: mol_type = other RNA
actggtatttgtgtctgagg

SEQ ID NO: 616 - RNA, 23 nt, Synthetic construct
  source 1..23: mol_type = other RNA
acctcagacacaaataccagttt

SEQ ID NO: 617 - RNA, 20 nt, Synthetic construct
  misc_feature 20: note = invAb
  source 1..20: mol_type = other RNA
ctggtatttgtgtctgaggc

SEQ ID NO: 618 - RNA, 23 nt, Synthetic construct
  source 1..23: mol_type = other RNA
agcctcagacacaaataccagtt

SEQ ID NO: 619 - RNA, 20 nt, Synthetic construct
  misc_feature 20: note = invAb
  source 1..20: mol_type = other RNA
tggtatttgtgtctgaggct

SEQ ID NO: 620 - RNA, 23 nt, Synthetic construct
  source 1..23: mol_type = other RNA
aagcctcagacacaaataccatt

SEQ ID NO: 621 - RNA, 20 nt, Synthetic construct
  misc_feature 20: note = invAb
  source 1..20: mol_type = other RNA
ggtatttgtgtctgaggctg

SEQ ID NO: 622 - RNA, 23 nt, Synthetic construct
  source 1..23: mol_type = other RNA
acagcctcagacacaaatacctt

SEQ ID NO: 623 - RNA, 20 nt, Synthetic construct
  misc_feature 20: note = invAb
  source 1..20: mol_type = other RNA
tgaatccaagtgtcctctga

SEQ ID NO: 624 - RNA, 23 nt, Synthetic construct
  source 1..23: mol_type = other RNA
atcagaggacacttggattcatt

SEQ ID NO: 625 - RNA, 20 nt, Synthetic construct
  misc_feature 20: note = invAb
  source 1..20: mol_type = other RNA
gaatccaagtgtcctctgat

SEQ ID NO: 626 - RNA, 23 nt, Synthetic construct
  source 1..23: mol_type = other RNA
aatcagaggacacttggattctt

SEQ ID NO: 627 - RNA, 20 nt, Synthetic construct
  misc_feature 20: note = invAb
  source 1..20: mol_type = other RNA
aatccaagtgtcctctgatg

SEQ ID NO: 628 - RNA, 23 nt, Synthetic construct
  source 1..23: mol_type = other RNA
acatcagaggacacttggatttt

SEQ ID NO: 629 - RNA, 20 nt, Synthetic construct
  source 1..20: mol_type = other RNA
atccaagtgtcctctgatgg

SEQ ID NO: 630 - RNA, 23 nt, Synthetic construct
  source 1..23: mol_type = other RNA
accatcagaggacacttggattt

SEQ ID NO: 631 - RNA, 20 nt, Synthetic construct
  misc_feature 20: note = invAb
  source 1..20: mol_type = other RNA
tccaagtgtcctctgatggt

SEQ ID NO: 632 - RNA, 23 nt, Synthetic construct
  source 1..23: mol_type = other RNA
aaccatcagaggacacttggatt

SEQ ID NO: 633 - RNA, 20 nt, Synthetic construct
  misc_feature 20: note = invAb
  source 1..20: mol_type = other RNA
ccaagtgtcctctgatggtc

SEQ ID NO: 634 - RNA, 23 nt, Synthetic construct
  source 1..23: mol_type = other RNA
tgaccatcagaggacacttggtt

SEQ ID NO: 635 - RNA, 20 nt, Synthetic construct
  misc_feature 20: note = invAb
  source 1..20: mol_type = other RNA
caagtgtcctctgatggtca

SEQ ID NO: 636 - RNA, 23 nt, Synthetic construct
  source 1..23: mol_type = other RNA
ttgaccatcagaggacacttgtt

SEQ ID NO: 637 - RNA, 20 nt, Synthetic construct
  misc_feature 20: note = invAb
  source 1..20: mol_type = other RNA
aagtgtcctctgatggtcaa

SEQ ID NO: 638 - RNA, 23 nt, Synthetic construct
  source 1..23: mol_type = other RNA
tttgaccatcagaggacactttt

SEQ ID NO: 639 - RNA, 20 nt, Synthetic construct
  misc_feature 20: note = invAb
  source 1..20: mol_type = other RNA
agtgtcctctgatggtcaaa

SEQ ID NO: 640 - RNA, 23 nt, Synthetic construct
  source 1..23: mol_type = other RNA
atttgaccatcagaggacacttt

SEQ ID NO: 641 - RNA, 20 nt, Synthetic construct
  misc_feature 20: note = invAb
  source 1..20: mol_type = other RNA
tgtcctctgatggtcaaagt

SEQ ID NO: 642 - RNA, 23 nt, Synthetic construct
  source 1..23: mol_type = other RNA
aactttgaccatcagaggacatt

SEQ ID NO: 643 - RNA, 20 nt, Synthetic construct
  misc_feature 20: note = invAb
  source 1..20: mol_type = other RNA
gtcctctgatggtcaaagtt

SEQ ID NO: 644 - RNA, 23 nt, Synthetic construct
  source 1..23: mol_type = other RNA
aaactttgaccatcagaggactt

SEQ ID NO: 645 - RNA, 20 nt, Synthetic construct
  misc_feature 20: note = invAb
  source 1..20: mol_type = other RNA
tcctctgatggtcaaagttc

SEQ ID NO: 646 - RNA, 23 nt, Synthetic construct
  source 1..23: mol_type = other RNA
agaactttgaccatcagaggatt

SEQ ID NO: 647 - RNA, 20 nt, Synthetic construct
  misc_feature 20: note = invAb
  source 1..20: mol_type = other RNA
cctctgatggtcaaagttct

SEQ ID NO: 648 - RNA, 23 nt, Synthetic construct
  source 1..23: mol_type = other RNA
tagaactttgaccatcagaggtt

SEQ ID NO: 649 - RNA, 20 nt, Synthetic construct
  misc_feature 20: note = invAb
  source 1..20: mol_type = other RNA
ctctgatggtcaaagttcta

SEQ ID NO: 650 - RNA, 23 nt, Synthetic construct
  source 1..23: mol_type = other RNA
atagaactttgaccatcagagtt

SEQ ID NO: 651 - RNA, 20 nt, Synthetic construct
  misc_feature 20: note = invAb
  source 1..20: mol_type = other RNA
tctgatggtcaaagttctag

SEQ ID NO: 652 - RNA, 23 nt, Synthetic construct
  source 1..23: mol_type = other RNA
tctagaactttgaccatcagatt

SEQ ID NO: 653 - RNA, 20 nt, Synthetic construct
  misc_feature 20: note = invAb
  source 1..20: mol_type = other RNA
ctgatggtcaaagttctaga

SEQ ID NO: 654 - RNA, 23 nt, Synthetic construct
  source 1..23: mol_type = other RNA
atctagaactttgaccatcagtt

SEQ ID NO: 655 - RNA, 20 nt, Synthetic construct
  misc_feature 20: note = invAb
  source 1..20: mol_type = other RNA
tgatggtcaaagttctagat

SEQ ID NO: 656 - RNA, 23 nt, Synthetic construct
  source 1..23: mol_type = other RNA
aatctagaactttgaccatcatt

SEQ ID NO: 657 - RNA, 20 nt, Synthetic construct
  misc_feature 20: note = invAb
  source 1..20: mol_type = other RNA
gatggtcaaagttctagatg

SEQ ID NO: 658 - RNA, 23 nt, Synthetic construct
  source 1..23: mol_type = other RNA
acatctagaactttgaccatctt

SEQ ID NO: 659 - RNA, 20 nt, Synthetic construct
  misc_feature 20: note = invAb
  source 1..20: mol_type = other RNA
gccatttgcctctgggaaaa

SEQ ID NO: 660 - RNA, 23 nt, Synthetic construct
  source 1..23: mol_type = other RNA
attttcccagaggcaaatggctt

SEQ ID NO: 661 - RNA, 20 nt, Synthetic construct
  misc_feature 20: note = invAb
  source 1..20: mol_type = other RNA
ccatttgcctctgggaaaac

SEQ ID NO: 662 - RNA, 23 nt, Synthetic construct
  source 1..23: mol_type = other RNA
agttttcccagaggcaaatggtt

SEQ ID NO: 663 - RNA, 20 nt, Synthetic construct
  misc_feature 20: note = invAb
  source 1..20: mol_type = other RNA
catttgcctctgggaaaacc

SEQ ID NO: 664 - RNA, 23 nt, Synthetic construct
  source 1..23: mol_type = other RNA
tggttttcccagaggcaaatgtt

SEQ ID NO: 665 - RNA, 20 nt, Synthetic construct
  misc_feature 20: note = invAb
  source 1..20: mol_type = other RNA
atttgcctctgggaaaacca

SEQ ID NO: 666 - RNA, 23 nt, Synthetic construct
  source 1..23: mol_type = other RNA
atggttttcccagaggcaaattt

SEQ ID NO: 667 - RNA, 20 nt, Synthetic construct
  misc_feature 20: note = invAb
  source 1..20: mol_type = other RNA
tttgcctctgggaaaaccag

SEQ ID NO: 668 - RNA, 23 nt, Synthetic construct
  source 1..23: mol_type = other RNA
actggttttcccagaggcaaatt

SEQ ID NO: 669 - RNA, 20 nt, Synthetic construct
  misc_feature 20: note = invAb
  source 1..20: mol_type = other RNA
ttgcctctgggaaaaccagt

SEQ ID NO: 670 - RNA, 23 nt, Synthetic construct
  source 1..23: mol_type = other RNA
aactggttttcccagaggcaatt

SEQ ID NO: 671 - RNA, 20 nt, Synthetic construct
  misc_feature 20: note = invAb
  source 1..20: mol_type = other RNA
tgcctctgggaaaaccagtg

SEQ ID NO: 672 - RNA, 23 nt, Synthetic construct
  source 1..23: mol_type = other RNA
tcactggttttcccagaggcatt

SEQ ID NO: 673 - RNA, 20 nt, Synthetic construct
  misc_feature 20: note = invAb
  source 1..20: mol_type = other RNA
ctctgggaaaaccagtgagt

SEQ ID NO: 674 - RNA, 23 nt, Synthetic construct
  source 1..23: mol_type = other RNA
aactcactggttttcccagagtt

SEQ ID NO: 675 - RNA, 20 nt, Synthetic construct
  misc_feature 20: note = invAb
  source 1..20: mol_type = other RNA
tctgggaaaaccagtgagtc

SEQ ID NO: 676 - RNA, 23 nt, Synthetic construct
  source 1..23: mol_type = other RNA
agactcactggttttcccagatt

SEQ ID NO: 677 - RNA, 20 nt, Synthetic construct
  misc_feature 20: note = invAb
  source 1..20: mol_type = other RNA
ctgggaaaaccagtgagtct

SEQ ID NO: 678 - RNA, 23 nt, Synthetic construct
  source 1..23: mol_type = other RNA
aagactcactggttttcccagtt

SEQ ID NO: 679 - RNA, 20 nt, Synthetic construct
  misc_feature 20: note = invAb
  source 1..20: mol_type = other RNA
tgggaaaaccagtgagtctg

SEQ ID NO: 680 - RNA, 23 nt, Synthetic construct
  source 1..23: mol_type = other RNA
acagactcactggttttcccatt

SEQ ID NO: 681 - RNA, 20 nt, Synthetic construct
  misc_feature 20: note = invAb
  source 1..20: mol_type = other RNA
gggaaaaccagtgagtctgg

SEQ ID NO: 682 - RNA, 23 nt, Synthetic construct
  source 1..23: mol_type = other RNA
tccagactcactggttttccctt

SEQ ID NO: 683 - RNA, 20 nt, Synthetic construct
  misc_feature 20: note = invAb
  source 1..20: mol_type = other RNA
ggaaaaccagtgagtctgga

SEQ ID NO: 684 - RNA, 23 nt, Synthetic construct
  source 1..23: mol_type = other RNA
atccagactcactggttttcctt

SEQ ID NO: 685 - RNA, 20 nt, Synthetic construct
  misc_feature 20: note = invAb
  source 1..20: mol_type = other RNA
gaaaaccagtgagtctggag

SEQ ID NO: 686 - RNA, 23 nt, Synthetic construct
  source 1..23: mol_type = other RNA
tctccagactcactggttttctt

SEQ ID NO: 687 - RNA, 20 nt, Synthetic construct
  misc_feature 20: note = invAb
  source 1..20: mol_type = other RNA
aaaaccagtgagtctggaga

SEQ ID NO: 688 - RNA, 23 nt, Synthetic construct
  source 1..23: mol_type = other RNA
atctccagactcactggtttttt

SEQ ID NO: 689 - RNA, 20 nt, Synthetic construct
  misc_feature 20: note = invAb
  source 1..20: mol_type = other RNA
aaaccagtgagtctggagag

SEQ ID NO: 690 - RNA, 23 nt, Synthetic construct
  source 1..23: mol_type = other RNA
actctccagactcactggttttt

SEQ ID NO: 691 - RNA, 20 nt, Synthetic construct
  misc_feature 20: note = invAb
  source 1..20: mol_type = other RNA
aaccagtgagtctggagagc

SEQ ID NO: 692 - RNA, 23 nt, Synthetic construct
  source 1..23: mol_type = other RNA
agctctccagactcactggtttt

SEQ ID NO: 693 - RNA, 20 nt, Synthetic construct
  misc_feature 20: note = invAb
  source 1..20: mol_type = other RNA
gtgagtctggagagctgcat

SEQ ID NO: 694 - RNA, 23 nt, Synthetic construct
  source 1..23: mol_type = other RNA
aatgcagctctccagactcactt

SEQ ID NO: 695 - RNA, 20 nt, Synthetic construct
  misc_feature 20: note = invAb
  source 1..20: mol_type = other RNA
tgagtctggagagctgcatg

SEQ ID NO: 696 - RNA, 23 nt, Synthetic construct
  source 1..23: mol_type = other RNA
acatgcagctctccagactcatt

SEQ ID NO: 697 - RNA, 20 nt, Synthetic construct
  misc_feature 20: note = invAb
  source 1..20: mol_type = other RNA
gagtctggagagctgcatgg

SEQ ID NO: 698 - RNA, 23 nt, Synthetic construct
  source 1..23: mol_type = other RNA
accatgcagctctccagactctt

SEQ ID NO: 699 - RNA, 20 nt, Synthetic construct
  misc_feature 20: note = invAb
  source 1..20: mol_type = other RNA
gtctggagagctgcatgggc

SEQ ID NO: 700 - RNA, 23 nt, Synthetic construct
  source 1..23: mol_type = other RNA
agcccatgcagctctccagactt

SEQ ID NO: 701 - RNA, 20 nt, Synthetic construct
  misc_feature 20: note = invAb
  source 1..20: mol_type = other RNA
tctggagagctgcatgggct

SEQ ID NO: 702 - RNA, 23 nt, Synthetic construct
  source 1..23: mol_type = other RNA
aagcccatgcagctctccagatt

SEQ ID NO: 703 - RNA, 20 nt, Synthetic construct
  misc_feature 20: note = invAb
  source 1..20: mol_type = other RNA
ctggagagctgcatgggctc

SEQ ID NO: 704 - RNA, 23 nt, Synthetic construct
  source 1..23: mol_type = other RNA
tgagcccatgcagctctccagtt

SEQ ID NO: 705 - RNA, 20 nt, Synthetic construct
  misc_feature 20: note = invAb
  source 1..20: mol_type = other RNA
ggagagctgcatgggctcac

SEQ ID NO: 706 - RNA, 23 nt, Synthetic construct
  source 1..23: mol_type = other RNA
tgtgagcccatgcagctctcctt

SEQ ID NO: 707 - RNA, 20 nt, Synthetic construct
  misc_feature 20: note = invAb
  source 1..20: mol_type = other RNA
gagagctgcatgggctcaca

SEQ ID NO: 708 - RNA, 23 nt, Synthetic construct
  source 1..23: mol_type = other RNA
ttgtgagcccatgcagctctctt

SEQ ID NO: 709 - RNA, 20 nt, Synthetic construct
  misc_feature 20: note = invAb
  source 1..20: mol_type = other RNA
agagctgcatgggctcacaa

SEQ ID NO: 710 - RNA, 23 nt, Synthetic construct
  source 1..23: mol_type = other RNA
attgtgagcccatgcagctcttt

SEQ ID NO: 711 - RNA, 20 nt, Synthetic construct
  misc_feature 20: note = invAb
  source 1..20: mol_type = other RNA
gagctgcatgggctcacaac

SEQ ID NO: 712 - RNA, 23 nt, Synthetic construct
  source 1..23: mol_type = other RNA
agttgtgagcccatgcagctctt

SEQ ID NO: 713 - RNA, 20 nt, Synthetic construct
  misc_feature 20: note = invAb
  source 1..20: mol_type = other RNA
agctgcatgggctcacaact

SEQ ID NO: 714 - RNA, 23 nt, Synthetic construct
  source 1..23: mol_type = other RNA
aagttgtgagcccatgcagcttt

SEQ ID NO: 715 - RNA, 20 nt, Synthetic construct
  misc_feature 20: note = invAb
  source 1..20: mol_type = other RNA
gctgcatgggctcacaactg

SEQ ID NO: 716 - RNA, 23 nt, Synthetic construct
  source 1..23: mol_type = other RNA
tcagttgtgagcccatgcagctt

SEQ ID NO: 717 - RNA, 20 nt, Synthetic construct
  misc_feature 20: note = invAb
  source 1..20: mol_type = other RNA
ctgcatgggctcacaactga

SEQ ID NO: 718 - RNA, 23 nt, Synthetic construct
  source 1..23: mol_type = other RNA
atcagttgtgagcccatgcagtt

SEQ ID NO: 719 - RNA, 20 nt, Synthetic construct
  misc_feature 20: note = invAb
  source 1..20: mol_type = other RNA
tgcatgggctcacaactgag

SEQ ID NO: 720 - RNA, 23 nt, Synthetic construct
  source 1..23: mol_type = other RNA
actcagttgtgagcccatgcatt

SEQ ID NO: 721 - RNA, 20 nt, Synthetic construct
  misc_feature 20: note = invAb
  source 1..20: mol_type = other RNA
gcatgggctcacaactgagg

SEQ ID NO: 722 - RNA, 23 nt, Synthetic construct
  source 1..23: mol_type = other RNA
tcctcagttgtgagcccatgctt

SEQ ID NO: 723 - RNA, 20 nt, Synthetic construct
  misc_feature 20: note = invAb
  source 1..20: mol_type = other RNA
catgggctcacaactgagga

SEQ ID NO: 724 - RNA, 23 nt, Synthetic construct
  source 1..23: mol_type = other RNA
atcctcagttgtgagcccatgtt

SEQ ID NO: 725 - RNA, 20 nt, Synthetic construct
  misc_feature 20: note = invAb
  source 1..20: mol_type = other RNA
ggctcacaactgaggaggaa

SEQ ID NO: 726 - RNA, 23 nt, Synthetic construct
  source 1..23: mol_type = other RNA
attcctcctcagttgtgagcctt

SEQ ID NO: 727 - RNA, 20 nt, Synthetic construct
  misc_feature 20: note = invAb
  source 1..20: mol_type = other RNA
gctcacaactgaggaggaat

SEQ ID NO: 728 - RNA, 23 nt, Synthetic construct
  source 1..23: mol_type = other RNA
aattcctcctcagttgtgagctt

SEQ ID NO: 729 - RNA, 20 nt, Synthetic construct
  misc_feature 20: note = invAb
  source 1..20: mol_type = other RNA
ctcacaactgaggaggaatt

SEQ ID NO: 730 - RNA, 23 nt, Synthetic construct
  source 1..23: mol_type = other RNA
aaattcctcctcagttgtgagtt

SEQ ID NO: 731 - RNA, 20 nt, Synthetic construct
  misc_feature 20: note = invAb
  source 1..20: mol_type = other RNA
cacaactgaggaggaatttg

SEQ ID NO: 732 - RNA, 23 nt, Synthetic construct
  source 1..23: mol_type = other RNA
acaaattcctcctcagttgtgtt

SEQ ID NO: 733 - RNA, 20 nt, Synthetic construct
  misc_feature 20: note = invAb
  source 1..20: mol_type = other RNA
acaactgaggaggaatttgt

SEQ ID NO: 734 - RNA, 23 nt, Synthetic construct
  source 1..23: mol_type = other RNA
tacaaattcctcctcagttgttt

SEQ ID NO: 735 - RNA, 20 nt, Synthetic construct
  misc_feature 20: note = invAb
  source 1..20: mol_type = other RNA
caactgaggaggaatttgta

SEQ ID NO: 736 - RNA, 23 nt, Synthetic construct
  source 1..23: mol_type = other RNA
atacaaattcctcctcagttgtt

SEQ ID NO: 737 - RNA, 20 nt, Synthetic construct
  misc_feature 20: note = invAb
  source 1..20: mol_type = other RNA
aactgaggaggaatttgtag

SEQ ID NO: 738 - RNA, 23 nt, Synthetic construct
  source 1..23: mol_type = other RNA
tctacaaattcctcctcagtttt

SEQ ID NO: 739 - RNA, 20 nt, Synthetic construct
  misc_feature 20: note = invAb
  source 1..20: mol_type = other RNA
actgaggaggaatttgtaga

SEQ ID NO: 740 - RNA, 23 nt, Synthetic construct
  source 1..23: mol_type = other RNA
ttctacaaattcctcctcagttt

SEQ ID NO: 741 - RNA, 20 nt, Synthetic construct
  misc_feature 20: note = invAb
  source 1..20: mol_type = other RNA
ctgaggaggaatttgtagaa

SEQ ID NO: 742 - RNA, 23 nt, Synthetic construct
  source 1..23: mol_type = other RNA
attctacaaattcctcctcagtt

SEQ ID NO: 743 - RNA, 20 nt, Synthetic construct
  misc_feature 20: note = invAb
  source 1..20: mol_type = other RNA
tgaggaggaatttgtagaag

SEQ ID NO: 744 - RNA, 23 nt, Synthetic construct
  source 1..23: mol_type = other RNA
acttctacaaattcctcctcatt

SEQ ID NO: 745 - RNA, 20 nt, Synthetic construct
  misc_feature 20: note = invAb
  source 1..20: mol_type = other RNA
gaggaggaatttgtagaagg

SEQ ID NO: 746 - RNA, 23 nt, Synthetic construct
  source 1..23: mol_type = other RNA
accttctacaaattcctcctctt

SEQ ID NO: 747 - RNA, 20 nt, Synthetic construct
  misc_feature 20: note = invAb
  source 1..20: mol_type = other RNA
aggaggaatttgtagaaggg

SEQ ID NO: 748 - RNA, 23 nt, Synthetic construct
  source 1..23: mol_type = other RNA
tcccttctacaaattcctccttt

SEQ ID NO: 749 - RNA, 20 nt, Synthetic construct
  misc_feature 20: note = invAb
  source 1..20: mol_type = other RNA
ggaggaatttgtagaaggga

SEQ ID NO: 750 - RNA, 23 nt, Synthetic construct
  source 1..23: mol_type = other RNA
atcccttctacaaattcctcctt

SEQ ID NO: 751 - RNA, 20 nt, Synthetic construct
  misc_feature 20: note = invAb
  source 1..20: mol_type = other RNA
gaggaatttgtagaagggat

SEQ ID NO: 752 - RNA, 23 nt, Synthetic construct
  source 1..23: mol_type = other RNA
tatcccttctacaaattcctctt

SEQ ID NO: 753 - RNA, 20 nt, Synthetic construct
  misc_feature 20: note = invAb
  source 1..20: mol_type = other RNA
ggaatttgtagaagggatat

SEQ ID NO: 754 - RNA, 23 nt, Synthetic construct
  source 1..23: mol_type = other RNA
tatatcccttctacaaattcctt

SEQ ID NO: 755 - RNA, 20 nt, Synthetic construct
  misc_feature 20: note = invAb
  source 1..20: mol_type = other RNA
gaatttgtagaagggatata

SEQ ID NO: 756 - RNA, 23 nt, Synthetic construct
  source 1..23: mol_type = other RNA
atatatcccttctacaaattctt

SEQ ID NO: 757 - RNA, 20 nt, Synthetic construct
  misc_feature 20: note = invAb
  source 1..20: mol_type = other RNA
aatttgtagaagggatatac

SEQ ID NO: 758 - RNA, 23 nt, Synthetic construct
  source 1..23: mol_type = other RNA
tgtatatcccttctacaaatttt

SEQ ID NO: 759 - RNA, 20 nt, Synthetic construct
  misc_feature 20: note = invAb
  source 1..20: mol_type = other RNA
aatttgtagaagggatatac

SEQ ID NO: 760 - RNA, 23 nt, Synthetic construct
  source 1..23: mol_type = other RNA
tgtatatcccttctacaaatttt

SEQ ID NO: 761 - RNA, 20 nt, Synthetic construct
  misc_feature 20: note = invAb
  source 1..20: mol_type = other RNA
atttgtagaagggatataca

SEQ ID NO: 762 - RNA, 23 nt, Synthetic construct
  source 1..23: mol_type = other RNA
ttgtatatcccttctacaaattt

SEQ ID NO: 763 - RNA, 20 nt, Synthetic construct
  misc_feature 20: note = invAb
  source 1..20: mol_type = other RNA
tttgtagaagggatatacaa

SEQ ID NO: 764 - RNA, 23 nt, Synthetic construct
  source 1..23: mol_type = other RNA
tttgtatatcccttctacaaatt

SEQ ID NO: 765 - RNA, 20 nt, Synthetic construct
  misc_feature 20: note = invAb
  source 1..20: mol_type = other RNA
ttgtagaagggatatacaaa

SEQ ID NO: 766 - RNA, 23 nt, Synthetic construct
  source 1..23: mol_type = other RNA
atttgtatatcccttctacaatt

SEQ ID NO: 767 - RNA, 20 nt, Synthetic construct
  misc_feature 20: note = invAb
  source 1..20: mol_type = other RNA
tgtagaagggatatacaaag

SEQ ID NO: 768 - RNA, 23 nt, Synthetic construct
  source 1..23: mol_type = other RNA
actttgtatatcccttctacatt

SEQ ID NO: 769 - RNA, 20 nt, Synthetic construct
  misc_feature 20: note = invAb
  source 1..20: mol_type = other RNA
tgtagaagggatatacaaag

SEQ ID NO: 770 - RNA, 23 nt, Synthetic construct
  source 1..23: mol_type = other RNA
actttgtatatcccttctacatt

SEQ ID NO: 771 - RNA, 20 nt, Synthetic construct
  misc_feature 20: note = invAb
  source 1..20: mol_type = other RNA
gtagaagggatatacaaagt

SEQ ID NO: 772 - RNA, 23 nt, Synthetic construct
  source 1..23: mol_type = other RNA
aactttgtatatcccttctactt

SEQ ID NO: 773 - RNA, 20 nt, Synthetic construct
  misc_feature 20: note = invAb
  source 1..20: mol_type = other RNA
tagaagggatatacaaagtg

SEQ ID NO: 774 - RNA, 23 nt, Synthetic construct
  source 1..23: mol_type = other RNA
acactttgtatatcccttctatt

SEQ ID NO: 775 - RNA, 20 nt, Synthetic construct
  misc_feature 20: note = invAb
  source 1..20: mol_type = other RNA
agaagggatatacaaagtgg

SEQ ID NO: 776 - RNA, 23 nt, Synthetic construct
  source 1..23: mol_type = other RNA
tccactttgtatatcccttcttt

SEQ ID NO: 777 - RNA, 20 nt, Synthetic construct
  misc_feature 20: note = invAb
  source 1..20: mol_type = other RNA
gaagggatatacaaagtgga

SEQ ID NO: 778 - RNA, 23 nt, Synthetic construct
  source 1..23: mol_type = other RNA
ttccactttgtatatcccttctt

SEQ ID NO: 779 - RNA, 20 nt, Synthetic construct
  misc_feature 20: note = invAb
  source 1..20: mol_type = other RNA
gaagggatatacaaagtgga

SEQ ID NO: 780 - RNA, 23 nt, Synthetic construct
  source 1..23: mol_type = other RNA
ttccactttgtatatcccttctt

SEQ ID NO: 781 - RNA, 20 nt, Synthetic construct
  misc_feature 20: note = invAb
  source 1..20: mol_type = other RNA
aagggatatacaaagtggaa

SEQ ID NO: 782 - RNA, 23 nt, Synthetic construct
  source 1..23: mol_type = other RNA
tttccactttgtatatccctttt

SEQ ID NO: 783 - RNA, 20 nt, Synthetic construct
  misc_feature 20: note = invAb
  source 1..20: mol_type = other RNA
agggatatacaaagtggaaa

SEQ ID NO: 784 - RNA, 23 nt, Synthetic construct
  source 1..23: mol_type = other RNA
atttccactttgtatatcccttt

SEQ ID NO: 785 - RNA, 20 nt, Synthetic construct
  misc_feature 20: note = invAb
  source 1..20: mol_type = other RNA
gggatatacaaagtggaaat

SEQ ID NO: 786 - RNA, 23 nt, Synthetic construct
  source 1..23: mol_type = other RNA
tatttccactttgtatatccctt

SEQ ID NO: 787 - RNA, 20 nt, Synthetic construct
  misc_feature 20: note = invAb
  source 1..20: mol_type = other RNA
ggatatacaaagtggaaata

SEQ ID NO: 788 - RNA, 23 nt, Synthetic construct
  source 1..23: mol_type = other RNA
atatttccactttgtatatcctt

SEQ ID NO: 789 - RNA, 20 nt, Synthetic construct
  misc_feature 20: note = invAb
  source 1..20: mol_type = other RNA
gatatacaaagtggaaatag

SEQ ID NO: 790 - RNA, 23 nt, Synthetic construct
  source 1..23: mol_type = other RNA
tctatttccactttgtatatctt

SEQ ID NO: 791 - RNA, 20 nt, Synthetic construct
  misc_feature 20: note = invAb
  source 1..20: mol_type = other RNA
atatacaaagtggaaataga

SEQ ID NO: 792 - RNA, 23 nt, Synthetic construct
  source 1..23: mol_type = other RNA
atctatttccactttgtatattt

SEQ ID NO: 793 - RNA, 20 nt, Synthetic construct
  misc_feature 20: note = invAb
  source 1..20: mol_type = other RNA
tatacaaagtggaaatagac

SEQ ID NO: 794 - RNA, 23 nt, Synthetic construct
  source 1..23: mol_type = other RNA
tgtctatttccactttgtatatt

SEQ ID NO: 795 - RNA, 20 nt, Synthetic construct
  misc_feature 20: note = invAb
  source 1..20: mol_type = other RNA
atacaaagtggaaatagaca

SEQ ID NO: 796 - RNA, 23 nt, Synthetic construct
  source 1..23: mol_type = other RNA
atgtctatttccactttgtattt

SEQ ID NO: 797 - RNA, 20 nt, Synthetic construct
  misc_feature 20: note = invAb
  source 1..20: mol_type = other RNA
atacaaagtggaaatagaca

SEQ ID NO: 798 - RNA, 23 nt, Synthetic construct
  source 1..23: mol_type = other RNA
atgtctatttccactttgtattt

SEQ ID NO: 799 - RNA, 20 nt, Synthetic construct
  misc_feature 20: note = invAb
  source 1..20: mol_type = other RNA
tacaaagtggaaatagacac

SEQ ID NO: 800 - RNA, 23 nt, Synthetic construct
  source 1..23: mol_type = other RNA
agtgtctatttccactttgtatt

SEQ ID NO: 801 - RNA, 20 nt, Synthetic construct
  misc_feature 20: note = invAb
  source 1..20: mol_type = other RNA
acaaagtggaaatagacacc

SEQ ID NO: 802 - RNA, 23 nt, Synthetic construct
  source 1..23: mol_type = other RNA
tggtgtctatttccactttgttt

SEQ ID NO: 803 - RNA, 20 nt, Synthetic construct
  misc_feature 20: note = invAb
  source 1..20: mol_type = other RNA
caaagtggaaatagacacca

SEQ ID NO: 804 - RNA, 23 nt, Synthetic construct
  source 1..23: mol_type = other RNA
ttggtgtctatttccactttgtt

SEQ ID NO: 805 - RNA, 20 nt, Synthetic construct
  misc_feature 20: note = invAb
  source 1..20: mol_type = other RNA
aagtggaaatagacaccaaa

SEQ ID NO: 806 - RNA, 23 nt, Synthetic construct
  source 1..23: mol_type = other RNA
atttggtgtctatttccactttt

SEQ ID NO: 807 - RNA, 20 nt, Synthetic construct
  misc_feature 20: note = invAb
  source 1..20: mol_type = other RNA
aagtggaaatagacaccaaa

SEQ ID NO: 808 - RNA, 23 nt, Synthetic construct
  source 1..23: mol_type = other RNA
atttggtgtctatttccactttt

SEQ ID NO: 809 - RNA, 20 nt, Synthetic construct
  misc_feature 20: note = invAb
  source 1..20: mol_type = other RNA
agtggaaatagacaccaaat

SEQ ID NO: 810 - RNA, 23 nt, Synthetic construct
  source 1..23: mol_type = other RNA
aatttggtgtctatttccacttt

SEQ ID NO: 811 - RNA, 20 nt, Synthetic construct
  misc_feature 20: note = invAb
  source 1..20: mol_type = other RNA
gtggaaatagacaccaaatc

SEQ ID NO: 812 - RNA, 23 nt, Synthetic construct
  source 1..23: mol_type = other RNA
agatttggtgtctatttccactt

SEQ ID NO: 813 - RNA, 20 nt, Synthetic construct
  misc_feature 20: note = invAb
  source 1..20: mol_type = other RNA
tggaaatagacaccaaatct

SEQ ID NO: 814 - RNA, 23 nt, Synthetic construct
  source 1..23: mol_type = other RNA
aagatttggtgtctatttccatt

SEQ ID NO: 815 - RNA, 20 nt, Synthetic construct
  misc_feature 20: note = invAb
  source 1..20: mol_type = other RNA
ggaaatagacaccaaatctt

SEQ ID NO: 816 - RNA, 23 nt, Synthetic construct
  source 1..23: mol_type = other RNA
taagatttggtgtctatttcctt

SEQ ID NO: 817 - RNA, 20 nt, Synthetic construct
  misc_feature 20: note = invAb
  source 1..20: mol_type = other RNA
ggaaatagacaccaaatctt

SEQ ID NO: 818 - RNA, 23 nt, Synthetic construct
  source 1..23: mol_type = other RNA
taagatttggtgtctatttcctt

SEQ ID NO: 819 - RNA, 20 nt, Synthetic construct
  misc_feature 20: note = invAb
  source 1..20: mol_type = other RNA
gaaatagacaccaaatctta

SEQ ID NO: 820 - RNA, 23 nt, Synthetic construct
  source 1..23: mol_type = other RNA
ataagatttggtgtctatttctt

SEQ ID NO: 821 - RNA, 20 nt, Synthetic construct
  misc_feature 20: note = invAb
  source 1..20: mol_type = other RNA
aaatagacaccaaatcttac

SEQ ID NO: 822 - RNA, 23 nt, Synthetic construct
  source 1..23: mol_type = other RNA
agtaagatttggtgtctattttt

SEQ ID NO: 823 - RNA, 20 nt, Synthetic construct
  misc_feature 20: note = invAb
  source 1..20: mol_type = other RNA
aatagacaccaaatcttact

SEQ ID NO: 824 - RNA, 23 nt, Synthetic construct
  source 1..23: mol_type = other RNA
aagtaagatttggtgtctatttt

SEQ ID NO: 825 - RNA, 20 nt, Synthetic construct
  misc_feature 20: note = invAb
  source 1..20: mol_type = other RNA
atagacaccaaatcttactg

SEQ ID NO: 826 - RNA, 23 nt, Synthetic construct
  source 1..23: mol_type = other RNA
acagtaagatttggtgtctattt

SEQ ID NO: 827 - RNA, 20 nt, Synthetic construct
  misc_feature 20: note = invAb
  source 1..20: mol_type = other RNA
atagacaccaaatcttactg

SEQ ID NO: 828 - RNA, 23 nt, Synthetic construct
  source 1..23: mol_type = other RNA
acagtaagatttggtgtctattt

SEQ ID NO: 829 - RNA, 20 nt, Synthetic construct
  misc_feature 20: note = invAb
  source 1..20: mol_type = other RNA
tagacaccaaatcttactgg

SEQ ID NO: 830 - RNA, 23 nt, Synthetic construct
  source 1..23: mol_type = other RNA
tccagtaagatttggtgtctatt

SEQ ID NO: 831 - RNA, 20 nt, Synthetic construct
  misc_feature 20: note = invAb
  source 1..20: mol_type = other RNA
agacaccaaatcttactgga

SEQ ID NO: 832 - RNA, 23 nt, Synthetic construct
  source 1..23: mol_type = other RNA
ttccagtaagatttggtgtcttt

SEQ ID NO: 833 - RNA, 20 nt, Synthetic construct
  misc_feature 20: note = invAb
  source 1..20: mol_type = other RNA
cacttggcatctccccattc

SEQ ID NO: 834 - RNA, 23 nt, Synthetic construct
  source 1..23: mol_type = other RNA
agaatggggagatgccaagtgtt

SEQ ID NO: 835 - RNA, 20 nt, Synthetic construct
  misc_feature 20: note = invAb
  source 1..20: mol_type = other RNA
acttggcatctccccattcc

SEQ ID NO: 836 - RNA, 23 nt, Synthetic construct
  source 1..23: mol_type = other RNA
tggaatggggagatgccaagttt

SEQ ID NO: 837 - RNA, 20 nt, Synthetic construct
  misc_feature 20: note = invAb
  source 1..20: mol_type = other RNA
acttggcatctccccattcc

SEQ ID NO: 838 - RNA, 23 nt, Synthetic construct
  source 1..23: mol_type = other RNA
tggaatggggagatgccaagttt

SEQ ID NO: 839 - RNA, 20 nt, Synthetic construct
  misc_feature 20: note = invAb
  source 1..20: mol_type = other RNA
cttggcatctccccattcca

SEQ ID NO: 840 - RNA, 23 nt, Synthetic construct
  source 1..23: mol_type = other RNA
atggaatggggagatgccaagtt

SEQ ID NO: 841 - RNA, 20 nt, Synthetic construct
  misc_feature 20: note = invAb
  source 1..20: mol_type = other RNA
ttggcatctccccattccat

SEQ ID NO: 842 - RNA, 23 nt, Synthetic construct
  source 1..23: mol_type = other RNA
aatggaatggggagatgccaatt

SEQ ID NO: 843 - RNA, 20 nt, Synthetic construct
  misc_feature 20: note = invAb
  source 1..20: mol_type = other RNA
tggcatctccccattccatg

SEQ ID NO: 844 - RNA, 23 nt, Synthetic construct
  source 1..23: mol_type = other RNA
tcatggaatggggagatgccatt

SEQ ID NO: 845 - RNA, 20 nt, Synthetic construct
  misc_feature 20: note = invAb
  source 1..20: mol_type = other RNA
ggcatctccccattccatga

SEQ ID NO: 846 - RNA, 23 nt, Synthetic construct
  source 1..23: mol_type = other RNA
atcatggaatggggagatgcctt

SEQ ID NO: 847 - RNA, 20 nt, Synthetic construct
  misc_feature 20: note = invAb
  source 1..20: mol_type = other RNA
ggcatctccccattccatga

SEQ ID NO: 848 - RNA, 23 nt, Synthetic construct
  source 1..23: mol_type = other RNA
atcatggaatggggagatgcctt

SEQ ID NO: 849 - RNA, 20 nt, Synthetic construct
  misc_feature 20: note = invAb
  source 1..20: mol_type = other RNA
gcatctccccattccatgag

SEQ ID NO: 850 - RNA, 23 nt, Synthetic construct
  source 1..23: mol_type = other RNA
actcatggaatggggagatgctt

SEQ ID NO: 851 - RNA, 20 nt, Synthetic construct
  misc_feature 20: note = invAb
  source 1..20: mol_type = other RNA
catctccccattccatgagc

SEQ ID NO: 852 - RNA, 23 nt, Synthetic construct
  source 1..23: mol_type = other RNA
tgctcatggaatggggagatgtt

SEQ ID NO: 853 - RNA, 20 nt, Synthetic construct
  misc_feature 20: note = invAb
  source 1..20: mol_type = other RNA
atctccccattccatgagca

SEQ ID NO: 854 - RNA, 23 nt, Synthetic construct
  source 1..23: mol_type = other RNA
atgctcatggaatggggagattt

SEQ ID NO: 855 - RNA, 20 nt, Synthetic construct
  misc_feature 20: note = invAb
  source 1..20: mol_type = other RNA
tctccccattccatgagcat

SEQ ID NO: 856 - RNA, 23 nt, Synthetic construct
  source 1..23: mol_type = other RNA
aatgctcatggaatggggagatt

SEQ ID NO: 857 - RNA, 20 nt, Synthetic construct
  misc_feature 20: note = invAb
  source 1..20: mol_type = other RNA
tctccccattccatgagcat

SEQ ID NO: 858 - RNA, 23 nt, Synthetic construct
  source 1..23: mol_type = other RNA
aatgctcatggaatggggagatt

SEQ ID NO: 859 - RNA, 20 nt, Synthetic construct
  misc_feature 20: note = invAb
  source 1..20: mol_type = other RNA
ctccccattccatgagcatg

SEQ ID NO: 860 - RNA, 23 nt, Synthetic construct
  source 1..23: mol_type = other RNA
acatgctcatggaatggggagtt

SEQ ID NO: 861 - RNA, 20 nt, Synthetic construct
  misc_feature 20: note = invAb
  source 1..20: mol_type = other RNA
tccccattccatgagcatgc

SEQ ID NO: 862 - RNA, 23 nt, Synthetic construct
  source 1..23: mol_type = other RNA
tgcatgctcatggaatggggatt

SEQ ID NO: 863 - RNA, 20 nt, Synthetic construct
  misc_feature 20: note = invAb
  source 1..20: mol_type = other RNA
ccccattccatgagcatgca

SEQ ID NO: 864 - RNA, 23 nt, Synthetic construct
  source 1..23: mol_type = other RNA
atgcatgctcatggaatggggtt

SEQ ID NO: 865 - RNA, 20 nt, Synthetic construct
  misc_feature 20: note = invAb
  source 1..20: mol_type = other RNA
ccattccatgagcatgcaga

SEQ ID NO: 866 - RNA, 23 nt, Synthetic construct
  source 1..23: mol_type = other RNA
atctgcatgctcatggaatggtt

SEQ ID NO: 867 - RNA, 20 nt, Synthetic construct
  misc_feature 20: note = invAb
  source 1..20: mol_type = other RNA
ccattccatgagcatgcaga

SEQ ID NO: 868 - RNA, 23 nt, Synthetic construct
  source 1..23: mol_type = other RNA
atctgcatgctcatggaatggtt

SEQ ID NO: 869 - RNA, 20 nt, Synthetic construct
  misc_feature 20: note = invAb
  source 1..20: mol_type = other RNA
cattccatgagcatgcagag

SEQ ID NO: 870 - RNA, 23 nt, Synthetic construct
  source 1..23: mol_type = other RNA
actctgcatgctcatggaatgtt

SEQ ID NO: 871 - RNA, 20 nt, Synthetic construct
  misc_feature 20: note = invAb
  source 1..20: mol_type = other RNA
attccatgagcatgcagagg

SEQ ID NO: 872 - RNA, 23 nt, Synthetic construct
  source 1..23: mol_type = other RNA
acctctgcatgctcatggaattt

SEQ ID NO: 873 - RNA, 20 nt, Synthetic construct
  source 1..20: mol_type = other RNA
ttccatgagcatgcagaggt

SEQ ID NO: 874 - RNA, 23 nt, Synthetic construct
  source 1..23: mol_type = other RNA
aacctctgcatgctcatggaatt

SEQ ID NO: 875 - RNA, 20 nt, Synthetic construct
  misc_feature 20: note = invAb
  source 1..20: mol_type = other RNA
tccatgagcatgcagaggtg

SEQ ID NO: 876 - RNA, 23 nt, Synthetic construct
  source 1..23: mol_type = other RNA
acacctctgcatgctcatggatt

SEQ ID NO: 877 - RNA, 20 nt, Synthetic construct
  misc_feature 20: note = invAb
  source 1..20: mol_type = other RNA
ccatgagcatgcagaggtgg

SEQ ID NO: 878 - RNA, 23 nt, Synthetic construct
  source 1..23: mol_type = other RNA
accacctctgcatgctcatggtt

SEQ ID NO: 879 - RNA, 20 nt, Synthetic construct
  misc_feature 20: note = invAb
  source 1..20: mol_type = other RNA
catgagcatgcagaggtggt

SEQ ID NO: 880 - RNA, 23 nt, Synthetic construct
  source 1..23: mol_type = other RNA
taccacctctgcatgctcatgtt

SEQ ID NO: 881 - RNA, 20 nt, Synthetic construct
  misc_feature 20: note = invAb
  source 1..20: mol_type = other RNA
atgagcatgcagaggtggta

SEQ ID NO: 882 - RNA, 23 nt, Synthetic construct
  source 1..23: mol_type = other RNA
ataccacctctgcatgctcattt

SEQ ID NO: 883 - RNA, 20 nt, Synthetic construct
  misc_feature 20: note = invAb
  source 1..20: mol_type = other RNA
tgagcatgcagaggtggtat

SEQ ID NO: 884 - RNA, 23 nt, Synthetic construct
  source 1..23: mol_type = other RNA
aataccacctctgcatgctcatt

SEQ ID NO: 885 - RNA, 20 nt, Synthetic construct
  misc_feature 20: note = invAb
  source 1..20: mol_type = other RNA
gagcatgcagaggtggtatt

SEQ ID NO: 886 - RNA, 23 nt, Synthetic construct
  source 1..23: mol_type = other RNA
aaataccacctctgcatgctctt

SEQ ID NO: 887 - RNA, 20 nt, Synthetic construct
  misc_feature 20: note = invAb
  source 1..20: mol_type = other RNA
agcatgcagaggtggtattc

SEQ ID NO: 888 - RNA, 23 nt, Synthetic construct
  source 1..23: mol_type = other RNA
tgaataccacctctgcatgcttt

SEQ ID NO: 889 - RNA, 20 nt, Synthetic construct
  misc_feature 20: note = invAb
  source 1..20: mol_type = other RNA
gcatgcagaggtggtattca

SEQ ID NO: 890 - RNA, 23 nt, Synthetic construct
  source 1..23: mol_type = other RNA
atgaataccacctctgcatgctt

SEQ ID NO: 891 - RNA, 20 nt, Synthetic construct
  misc_feature 20: note = invAb
  source 1..20: mol_type = other RNA
catgcagaggtggtattcac

SEQ ID NO: 892 - RNA, 23 nt, Synthetic construct
  source 1..23: mol_type = other RNA
tgtgaataccacctctgcatgtt

SEQ ID NO: 893 - RNA, 20 nt, Synthetic construct
  misc_feature 20: note = invAb
  source 1..20: mol_type = other RNA
atgcagaggtggtattcaca

SEQ ID NO: 894 - RNA, 23 nt, Synthetic construct
  source 1..23: mol_type = other RNA
atgtgaataccacctctgcattt

SEQ ID NO: 895 - RNA, 20 nt, Synthetic construct
  misc_feature 20: note = invAb
  source 1..20: mol_type = other RNA
agaggtggtattcacagcca

SEQ ID NO: 896 - RNA, 23 nt, Synthetic construct
  source 1..23: mol_type = other RNA
ttggctgtgaataccacctcttt

SEQ ID NO: 897 - RNA, 20 nt, Synthetic construct
  misc_feature 20: note = invAb
  source 1..20: mol_type = other RNA
gaggtggtattcacagccaa

SEQ ID NO: 898 - RNA, 23 nt, Synthetic construct
  source 1..23: mol_type = other RNA
attggctgtgaataccacctctt

SEQ ID NO: 899 - RNA, 20 nt, Synthetic construct
  misc_feature 20: note = invAb
  source 1..20: mol_type = other RNA
aggtggtattcacagccaac

SEQ ID NO: 900 - RNA, 23 nt, Synthetic construct
  source 1..23: mol_type = other RNA
agttggctgtgaataccaccttt

SEQ ID NO: 901 - RNA, 20 nt, Synthetic construct
  misc_feature 20: note = invAb
  source 1..20: mol_type = other RNA
ggtggtattcacagccaacg

SEQ ID NO: 902 - RNA, 23 nt, Synthetic construct
  source 1..23: mol_type = other RNA
tcgttggctgtgaataccacctt

SEQ ID NO: 903 - RNA, 20 nt, Synthetic construct
  misc_feature 20: note = invAb
  source 1..20: mol_type = other RNA
gccgccctgctgagccccta

SEQ ID NO: 904 - RNA, 23 nt, Synthetic construct
  source 1..23: mol_type = other RNA
ataggggctcagcagggcggctt

SEQ ID NO: 905 - RNA, 20 nt, Synthetic construct
  misc_feature 20: note = invAb
  source 1..20: mol_type = other RNA
ccgccctgctgagcccctac

SEQ ID NO: 906 - RNA, 23 nt, Synthetic construct
  source 1..23: mol_type = other RNA
agtaggggctcagcagggcggtt

SEQ ID NO: 907 - RNA, 20 nt, Synthetic construct
  misc_feature 20: note = invAb
  source 1..20: mol_type = other RNA
cgccctgctgagcccctact

SEQ ID NO: 908 - RNA, 23 nt, Synthetic construct
  source 1..23: mol_type = other RNA
aagtaggggctcagcagggcgtt

SEQ ID NO: 909 - RNA, 20 nt, Synthetic construct
  misc_feature 20: note = invAb
  source 1..20: mol_type = other RNA
gccctgctgagcccctactc

SEQ ID NO: 910 - RNA, 23 nt, Synthetic construct
  source 1..23: mol_type = other RNA
agagtaggggctcagcagggctt

SEQ ID NO: 911 - RNA, 20 nt, Synthetic construct
  misc_feature 20: note = invAb
  source 1..20: mol_type = other RNA
ccctgctgagcccctactcc

SEQ ID NO: 912 - RNA, 23 nt, Synthetic construct
  source 1..23: mol_type = other RNA
aggagtaggggctcagcagggtt

SEQ ID NO: 913 - RNA, 20 nt, Synthetic construct
  misc_feature 20: note = invAb
  source 1..20: mol_type = other RNA
cctgctgagcccctactcct

SEQ ID NO: 914 - RNA, 23 nt, Synthetic construct
  source 1..23: mol_type = other RNA
taggagtaggggctcagcaggtt

SEQ ID NO: 915 - RNA, 20 nt, Synthetic construct
  misc_feature 20: note = invAb
  source 1..20: mol_type = other RNA
ctgctgagcccctactccta

SEQ ID NO: 916 - RNA, 23 nt, Synthetic construct
  source 1..23: mol_type = other RNA
ataggagtaggggctcagcagtt

SEQ ID NO: 917 - RNA, 20 nt, Synthetic construct
  misc_feature 20: note = invAb
  source 1..20: mol_type = other RNA
tgctgagcccctactcctat

SEQ ID NO: 918 - RNA, 23 nt, Synthetic construct
  source 1..23: mol_type = other RNA
aataggagtaggggctcagcatt

SEQ ID NO: 919 - RNA, 20 nt, Synthetic construct
  misc_feature 20: note = invAb
  source 1..20: mol_type = other RNA
gctgagcccctactcctatt

SEQ ID NO: 920 - RNA, 23 nt, Synthetic construct
  source 1..23: mol_type = other RNA
aaataggagtaggggctcagctt

SEQ ID NO: 921 - RNA, 20 nt, Synthetic construct
  misc_feature 20: note = invAb
  source 1..20: mol_type = other RNA
ctgagcccctactcctattc

SEQ ID NO: 922 - RNA, 23 nt, Synthetic construct
  source 1..23: mol_type = other RNA
agaataggagtaggggctcagtt

SEQ ID NO: 923 - RNA, 20 nt, Synthetic construct
  misc_feature 20: note = invAb
  source 1..20: mol_type = other RNA
tgagcccctactcctattcc

SEQ ID NO: 924 - RNA, 23 nt, Synthetic construct
  source 1..23: mol_type = other RNA
tggaataggagtaggggctcatt

SEQ ID NO: 925 - RNA, 20 nt, Synthetic construct
  misc_feature 20: note = invAb
  source 1..20: mol_type = other RNA
gagcccctactcctattcca

SEQ ID NO: 926 - RNA, 23 nt, Synthetic construct
  source 1..23: mol_type = other RNA
atggaataggagtaggggctctt

SEQ ID NO: 927 - RNA, 20 nt, Synthetic construct
  misc_feature 20: note = invAb
  source 1..20: mol_type = other RNA
agcccctactcctattccac

SEQ ID NO: 928 - RNA, 23 nt, Synthetic construct
  source 1..23: mol_type = other RNA
agtggaataggagtaggggcttt

SEQ ID NO: 929 - RNA, 20 nt, Synthetic construct
  misc_feature 20: note = invAb
  source 1..20: mol_type = other RNA
gcccctactcctattccacc

SEQ ID NO: 930 - RNA, 23 nt, Synthetic construct
  source 1..23: mol_type = other RNA
tggtggaataggagtaggggctt

SEQ ID NO: 931 - RNA, 20 nt, Synthetic construct
  misc_feature 20: note = invAb
  source 1..20: mol_type = other RNA
cccctactcctattccacca

SEQ ID NO: 932 - RNA, 23 nt, Synthetic construct
  source 1..23: mol_type = other RNA
atggtggaataggagtaggggtt

SEQ ID NO: 933 - RNA, 20 nt, Synthetic construct
  misc_feature 20: note = invAb
  source 1..20: mol_type = other RNA
ccctactcctattccaccac

SEQ ID NO: 934 - RNA, 23 nt, Synthetic construct
  source 1..23: mol_type = other RNA
agtggtggaataggagtagggtt

SEQ ID NO: 935 - RNA, 20 nt, Synthetic construct
  misc_feature 20: note = invAb
  source 1..20: mol_type = other RNA
cctactcctattccaccacg

SEQ ID NO: 936 - RNA, 23 nt, Synthetic construct
  source 1..23: mol_type = other RNA
acgtggtggaataggagtaggtt

SEQ ID NO: 937 - RNA, 20 nt, Synthetic construct
  misc_feature 20: note = invAb
  source 1..20: mol_type = other RNA
ctactcctattccaccacgg

SEQ ID NO: 938 - RNA, 23 nt, Synthetic construct
  source 1..23: mol_type = other RNA
accgtggtggaataggagtagtt

SEQ ID NO: 939 - RNA, 20 nt, Synthetic construct
  misc_feature 20: note = invAb
  source 1..20: mol_type = other RNA
tactcctattccaccacggc

SEQ ID NO: 940 - RNA, 23 nt, Synthetic construct
  source 1..23: mol_type = other RNA
agccgtggtggaataggagtatt

SEQ ID NO: 941 - RNA, 20 nt, Synthetic construct
  misc_feature 20: note = invAb
  source 1..20: mol_type = other RNA
actcctattccaccacggct

SEQ ID NO: 942 - RNA, 23 nt, Synthetic construct
  source 1..23: mol_type = other RNA
aagccgtggtggaataggagttt

SEQ ID NO: 943 - RNA, 20 nt, Synthetic construct
  misc_feature 20: note = invAb
  source 1..20: mol_type = other RNA
ctcctattccaccacggctg

SEQ ID NO: 944 - RNA, 23 nt, Synthetic construct
  source 1..23: mol_type = other RNA
acagccgtggtggaataggagtt

SEQ ID NO: 945 - RNA, 20 nt, Synthetic construct
  misc_feature 20: note = invAb
  source 1..20: mol_type = other RNA
tcctattccaccacggctgt

SEQ ID NO: 946 - RNA, 23 nt, Synthetic construct
  source 1..23: mol_type = other RNA
aacagccgtggtggaataggatt

SEQ ID NO: 947 - RNA, 20 nt, Synthetic construct
  misc_feature 20: note = invAb
  source 1..20: mol_type = other RNA
cctattccaccacggctgtc

SEQ ID NO: 948 - RNA, 23 nt, Synthetic construct
  source 1..23: mol_type = other RNA
agacagccgtggtggaataggtt

SEQ ID NO: 949 - RNA, 20 nt, Synthetic construct
  misc_feature 20: note = invAb
  source 1..20: mol_type = other RNA
ctattccaccacggctgtcg

SEQ ID NO: 950 - RNA, 23 nt, Synthetic construct
  source 1..23: mol_type = other RNA
acgacagccgtggtggaatagtt

SEQ ID NO: 951 - RNA, 20 nt, Synthetic construct
  misc_feature 20: note = invAb
  source 1..20: mol_type = other RNA
tattccaccacggctgtcgt

SEQ ID NO: 952 - RNA, 23 nt, Synthetic construct
  source 1..23: mol_type = other RNA
aacgacagccgtggtggaatatt

SEQ ID NO: 953 - RNA, 20 nt, Synthetic construct
  misc_feature 20: note = invAb
  source 1..20: mol_type = other RNA
attccaccacggctgtcgtc

SEQ ID NO: 954 - RNA, 23 nt, Synthetic construct
  source 1..23: mol_type = other RNA
tgacgacagccgtggtggaattt

SEQ ID NO: 955 - RNA, 20 nt, Synthetic construct
  misc_feature 20: note = invAb
  source 1..20: mol_type = other RNA
ttccaccacggctgtcgtca

SEQ ID NO: 956 - RNA, 23 nt, Synthetic construct
  source 1..23: mol_type = other RNA
atgacgacagccgtggtggaatt

SEQ ID NO: 957 - RNA, 20 nt, Synthetic construct
  misc_feature 20: note = invAb
  source 1..20: mol_type = other RNA
tccaccacggctgtcgtcac

SEQ ID NO: 958 - RNA, 23 nt, Synthetic construct
  source 1..23: mol_type = other RNA
agtgacgacagccgtggtggatt

SEQ ID NO: 959 - RNA, 20 nt, Synthetic construct
  misc_feature 20: note = invAb
  source 1..20: mol_type = other RNA
ccaccacggctgtcgtcacc

SEQ ID NO: 960 - RNA, 23 nt, Synthetic construct
  source 1..23: mol_type = other RNA
tggtgacgacagccgtggtggtt

SEQ ID NO: 961 - RNA, 20 nt, Synthetic construct
  misc_feature 20: note = invAb
  source 1..20: mol_type = other RNA
caccacggctgtcgtcacca

SEQ ID NO: 962 - RNA, 23 nt, Synthetic construct
  source 1..23: mol_type = other RNA
ttggtgacgacagccgtggtgtt

SEQ ID NO: 963 - RNA, 20 nt, Synthetic construct
  misc_feature 20: note = invAb
  source 1..20: mol_type = other RNA
accacggctgtcgtcaccaa

SEQ ID NO: 964 - RNA, 23 nt, Synthetic construct
  source 1..23: mol_type = other RNA
attggtgacgacagccgtggttt

SEQ ID NO: 965 - RNA, 20 nt, Synthetic construct
  misc_feature 20: note = invAb
  source 1..20: mol_type = other RNA
ccacggctgtcgtcaccaat

SEQ ID NO: 966 - RNA, 23 nt, Synthetic construct
  source 1..23: mol_type = other RNA
aattggtgacgacagccgtggtt

SEQ ID NO: 967 - RNA, 20 nt, Synthetic construct
  misc_feature 20: note = invAb
  source 1..20: mol_type = other RNA
cacggctgtcgtcaccaatc

SEQ ID NO: 968 - RNA, 23 nt, Synthetic construct
  source 1..23: mol_type = other RNA
agattggtgacgacagccgtgtt

SEQ ID NO: 969 - RNA, 20 nt, Synthetic construct
  misc_feature 20: note = invAb
  source 1..20: mol_type = other RNA
acggctgtcgtcaccaatcc

SEQ ID NO: 970 - RNA, 23 nt, Synthetic construct
  source 1..23: mol_type = other RNA
aggattggtgacgacagccgttt

SEQ ID NO: 971 - RNA, 20 nt, Synthetic construct
  misc_feature 20: note = invAb
  source 1..20: mol_type = other RNA
cggctgtcgtcaccaatccc

SEQ ID NO: 972 - RNA, 23 nt, Synthetic construct
  source 1..23: mol_type = other RNA
tgggattggtgacgacagccgtt

SEQ ID NO: 973 - RNA, 20 nt, Synthetic construct
  misc_feature 20: note = invAb
  source 1..20: mol_type = other RNA
ggctgtcgtcaccaatccca

SEQ ID NO: 974 - RNA, 23 nt, Synthetic construct
  source 1..23: mol_type = other RNA
ttgggattggtgacgacagcctt

SEQ ID NO: 975 - RNA, 20 nt, Synthetic construct
  misc_feature 20: note = invAb
  source 1..20: mol_type = other RNA
gctgtcgtcaccaatcccaa

SEQ ID NO: 976 - RNA, 23 nt, Synthetic construct
  source 1..23: mol_type = other RNA
attgggattggtgacgacagctt

SEQ ID NO: 977 - RNA, 20 nt, Synthetic construct
  misc_feature 20: note = invAb
  source 1..20: mol_type = other RNA
tgtcgtcaccaatcccaagg

SEQ ID NO: 978 - RNA, 23 nt, Synthetic construct
  source 1..23: mol_type = other RNA
tccttgggattggtgacgacatt

SEQ ID NO: 979 - RNA, 20 nt, Synthetic construct
  misc_feature 20: note = invAb
  source 1..20: mol_type = other RNA
gtcgtcaccaatcccaagga

SEQ ID NO: 980 - RNA, 23 nt, Synthetic construct
  source 1..23: mol_type = other RNA
ttccttgggattggtgacgactt

SEQ ID NO: 981 - RNA, 20 nt, Synthetic construct
  misc_feature 20: note = invAb
  source 1..20: mol_type = other RNA
tcgtcaccaatcccaaggaa

SEQ ID NO: 982 - RNA, 23 nt, Synthetic construct
  source 1..23: mol_type = other RNA
attccttgggattggtgacgatt

SEQ ID NO: 983 - RNA, 20 nt, Synthetic construct
  misc_feature 20: note = invAb
  source 1..20: mol_type = other RNA
cgtcaccaatcccaaggaat

SEQ ID NO: 984 - RNA, 23 nt, Synthetic construct
  source 1..23: mol_type = other RNA
aattccttgggattggtgacgtt

SEQ ID NO: 985 - RNA, 20 nt, Synthetic construct
  misc_feature 20: note = invAb
  source 1..20: mol_type = other RNA
caccaatcccaaggaatgag

SEQ ID NO: 986 - RNA, 23 nt, Synthetic construct
  source 1..23: mol_type = other RNA
actcattccttgggattggtgtt

SEQ ID NO: 987 - RNA, 20 nt, Synthetic construct
  misc_feature 20: note = invAb
  source 1..20: mol_type = other RNA
accaatcccaaggaatgagg

SEQ ID NO: 988 - RNA, 23 nt, Synthetic construct
  source 1..23: mol_type = other RNA
acctcattccttgggattggttt

SEQ ID NO: 989 - RNA, 20 nt, Synthetic construct
  misc_feature 20: note = invAb
  source 1..20: mol_type = other RNA
ccaatcccaaggaatgaggg

SEQ ID NO: 990 - RNA, 23 nt, Synthetic construct
  source 1..23: mol_type = other RNA
tccctcattccttgggattggtt

SEQ ID NO: 991 - RNA, 20 nt, Synthetic construct
  misc_feature 20: note = invAb
  source 1..20: mol_type = other RNA
caatcccaaggaatgaggga

SEQ ID NO: 992 - RNA, 23 nt, Synthetic construct
  source 1..23: mol_type = other RNA
atccctcattccttgggattgtt

SEQ ID NO: 993 - RNA, 20 nt, Synthetic construct
  misc_feature 20: note = invAb
  source 1..20: mol_type = other RNA
aatcccaaggaatgagggac

SEQ ID NO: 994 - RNA, 23 nt, Synthetic construct
  source 1..23: mol_type = other RNA
agtccctcattccttgggatttt

SEQ ID NO: 995 - RNA, 20 nt, Synthetic construct
  misc_feature 20: note = invAb
  source 1..20: mol_type = other RNA
atcccaaggaatgagggact

SEQ ID NO: 996 - RNA, 23 nt, Synthetic construct
  source 1..23: mol_type = other RNA
aagtccctcattccttgggattt

SEQ ID NO: 997 - RNA, 20 nt, Synthetic construct
  misc_feature 20: note = invAb
  source 1..20: mol_type = other RNA
tcccaaggaatgagggactt

SEQ ID NO: 998 - RNA, 23 nt, Synthetic construct
  source 1..23: mol_type = other RNA
aaagtccctcattccttgggatt

SEQ ID NO: 999 - RNA, 20 nt, Synthetic construct
  misc_feature 20: note = invAb
  source 1..20: mol_type = other RNA
cccaaggaatgagggacttc

SEQ ID NO: 1000 - RNA, 23 nt, Synthetic construct
  source 1..23: mol_type = other RNA
agaagtccctcattccttgggtt

SEQ ID NO: 1001 - RNA, 20 nt, Synthetic construct
  misc_feature 20: note = invAb
  source 1..20: mol_type = other RNA
ccaaggaatgagggacttct

SEQ ID NO: 1002 - RNA, 23 nt, Synthetic construct
  source 1..23: mol_type = other RNA
aagaagtccctcattccttggtt

SEQ ID NO: 1003 - RNA, 20 nt, Synthetic construct
  misc_feature 20: note = invAb
  source 1..20: mol_type = other RNA
caaggaatgagggacttctc

SEQ ID NO: 1004 - RNA, 23 nt, Synthetic construct
  source 1..23: mol_type = other RNA
agagaagtccctcattccttgtt

SEQ ID NO: 1005 - RNA, 20 nt, Synthetic construct
  misc_feature 20: note = invAb
  source 1..20: mol_type = other RNA
ctgaaggacgagggatggga

SEQ ID NO: 1006 - RNA, 23 nt, Synthetic construct
  source 1..23: mol_type = other RNA
atcccatccctcgtccttcagtt

SEQ ID NO: 1007 - RNA, 20 nt, Synthetic construct
  misc_feature 20: note = invAb
  source 1..20: mol_type = other RNA
tgaaggacgagggatgggat

SEQ ID NO: 1008 - RNA, 23 nt, Synthetic construct
  source 1..23: mol_type = other RNA
aatcccatccctcgtccttcatt

SEQ ID NO: 1009 - RNA, 20 nt, Synthetic construct
  misc_feature 20: note = invAb
  source 1..20: mol_type = other RNA
gaaggacgagggatgggatt

SEQ ID NO: 1010 - RNA, 23 nt, Synthetic construct
  source 1..23: mol_type = other RNA
aaatcccatccctcgtccttctt

SEQ ID NO: 1011 - RNA, 20 nt, Synthetic construct
  misc_feature 20: note = invAb
  source 1..20: mol_type = other RNA
aaggacgagggatgggattt

SEQ ID NO: 1012 - RNA, 23 nt, Synthetic construct
  source 1..23: mol_type = other RNA
aaaatcccatccctcgtcctttt

SEQ ID NO: 1013 - RNA, 20 nt, Synthetic construct
  misc_feature 20: note = invAb
  source 1..20: mol_type = other RNA
aggacgagggatgggatttc

SEQ ID NO: 1014 - RNA, 23 nt, Synthetic construct
  source 1..23: mol_type = other RNA
tgaaatcccatccctcgtccttt

SEQ ID NO: 1015 - RNA, 20 nt, Synthetic construct
  misc_feature 20: note = invAb
  source 1..20: mol_type = other RNA
ggacgagggatgggatttca

SEQ ID NO: 1016 - RNA, 23 nt, Synthetic construct
  source 1..23: mol_type = other RNA
atgaaatcccatccctcgtcctt

SEQ ID NO: 1017 - RNA, 20 nt, Synthetic construct
  misc_feature 20: note = invAb
  source 1..20: mol_type = other RNA
gacgagggatgggatttcat

SEQ ID NO: 1018 - RNA, 23 nt, Synthetic construct
  source 1..23: mol_type = other RNA
aatgaaatcccatccctcgtctt

SEQ ID NO: 1019 - RNA, 20 nt, Synthetic construct
  misc_feature 20: note = invAb
  source 1..20: mol_type = other RNA
acgagggatgggatttcatg

SEQ ID NO: 1020 - RNA, 23 nt, Synthetic construct
  source 1..23: mol_type = other RNA
acatgaaatcccatccctcgttt

SEQ ID NO: 1021 - RNA, 20 nt, Synthetic construct
  misc_feature 20: note = invAb
  source 1..20: mol_type = other RNA
cgagggatgggatttcatgt

SEQ ID NO: 1022 - RNA, 23 nt, Synthetic construct
  source 1..23: mol_type = other RNA
tacatgaaatcccatccctcgtt

SEQ ID NO: 1023 - RNA, 20 nt, Synthetic construct
  misc_feature 20: note = invAb
  source 1..20: mol_type = other RNA
gagggatgggatttcatgta

SEQ ID NO: 1024 - RNA, 23 nt, Synthetic construct
  source 1..23: mol_type = other RNA
ttacatgaaatcccatccctctt

SEQ ID NO: 1025 - RNA, 20 nt, Synthetic construct
  misc_feature 20: note = invAb
  source 1..20: mol_type = other RNA
agggatgggatttcatgtaa

SEQ ID NO: 1026 - RNA, 23 nt, Synthetic construct
  source 1..23: mol_type = other RNA
attacatgaaatcccatcccttt

SEQ ID NO: 1027 - RNA, 20 nt, Synthetic construct
  misc_feature 20: note = invAb
  source 1..20: mol_type = other RNA
gggatgggatttcatgtaac

SEQ ID NO: 1028 - RNA, 23 nt, Synthetic construct
  source 1..23: mol_type = other RNA
agttacatgaaatcccatccctt

SEQ ID NO: 1029 - RNA, 20 nt, Synthetic construct
  misc_feature 20: note = invAb
  source 1..20: mol_type = other RNA
ggatgggatttcatgtaacc

SEQ ID NO: 1030 - RNA, 23 nt, Synthetic construct
  source 1..23: mol_type = other RNA
tggttacatgaaatcccatcctt

SEQ ID NO: 1031 - RNA, 20 nt, Synthetic construct
  misc_feature 20: note = invAb
  source 1..20: mol_type = other RNA
gatgggatttcatgtaacca

SEQ ID NO: 1032 - RNA, 23 nt, Synthetic construct
  source 1..23: mol_type = other RNA
ttggttacatgaaatcccatctt

SEQ ID NO: 1033 - RNA, 20 nt, Synthetic construct
  misc_feature 20: note = invAb
  source 1..20: mol_type = other RNA
atgggatttcatgtaaccaa

SEQ ID NO: 1034 - RNA, 23 nt, Synthetic construct
  source 1..23: mol_type = other RNA
attggttacatgaaatcccattt

SEQ ID NO: 1035 - RNA, 20 nt, Synthetic construct
  misc_feature 20: note = invAb
  source 1..20: mol_type = other RNA
tgggatttcatgtaaccaag

SEQ ID NO: 1036 - RNA, 23 nt, Synthetic construct
  source 1..23: mol_type = other RNA
tcttggttacatgaaatcccatt

SEQ ID NO: 1037 - RNA, 20 nt, Synthetic construct
  misc_feature 20: note = invAb
  source 1..20: mol_type = other RNA
gggatttcatgtaaccaaga

SEQ ID NO: 1038 - RNA, 23 nt, Synthetic construct
  source 1..23: mol_type = other RNA
atcttggttacatgaaatccctt

SEQ ID NO: 1039 - RNA, 20 nt, Synthetic construct
  misc_feature 20: note = invAb
  source 1..20: mol_type = other RNA
ggatttcatgtaaccaagag

SEQ ID NO: 1040 - RNA, 23 nt, Synthetic construct
  source 1..23: mol_type = other RNA
actcttggttacatgaaatcctt

SEQ ID NO: 1041 - RNA, 20 nt, Synthetic construct
  misc_feature 20: note = invAb
  source 1..20: mol_type = other RNA
gatttcatgtaaccaagagt

SEQ ID NO: 1042 - RNA, 23 nt, Synthetic construct
  source 1..23: mol_type = other RNA
tactcttggttacatgaaatctt

SEQ ID NO: 1043 - RNA, 20 nt, Synthetic construct
  misc_feature 20: note = invAb
  source 1..20: mol_type = other RNA
atttcatgtaaccaagagta

SEQ ID NO: 1044 - RNA, 23 nt, Synthetic construct
  source 1..23: mol_type = other RNA
atactcttggttacatgaaattt

SEQ ID NO: 1045 - RNA, 20 nt, Synthetic construct
  misc_feature 20: note = invAb
  source 1..20: mol_type = other RNA
tttcatgtaaccaagagtat

SEQ ID NO: 1046 - RNA, 23 nt, Synthetic construct
  source 1..23: mol_type = other RNA
aatactcttggttacatgaaatt

SEQ ID NO: 1047 - RNA, 20 nt, Synthetic construct
  misc_feature 20: note = invAb
  source 1..20: mol_type = other RNA
ttcatgtaaccaagagtatt

SEQ ID NO: 1048 - RNA, 23 nt, Synthetic construct
  source 1..23: mol_type = other RNA
aaatactcttggttacatgaatt

SEQ ID NO: 1049 - RNA, 20 nt, Synthetic construct
  misc_feature 20: note = invAb
  source 1..20: mol_type = other RNA
tcatgtaaccaagagtattc

SEQ ID NO: 1050 - RNA, 23 nt, Synthetic construct
  source 1..23: mol_type = other RNA
agaatactcttggttacatgatt

SEQ ID NO: 1051 - RNA, 20 nt, Synthetic construct
  misc_feature 20: note = invAb
  source 1..20: mol_type = other RNA
catgtaaccaagagtattcc

SEQ ID NO: 1052 - RNA, 23 nt, Synthetic construct
  source 1..23: mol_type = other RNA
tggaatactcttggttacatgtt

SEQ ID NO: 1053 - RNA, 20 nt, Synthetic construct
  misc_feature 20: note = invAb
  source 1..20: mol_type = other RNA
atgtaaccaagagtattcca

SEQ ID NO: 1054 - RNA, 23 nt, Synthetic construct
  source 1..23: mol_type = other RNA
atggaatactcttggttacattt

SEQ ID NO: 1055 - RNA, 20 nt, Synthetic construct
  misc_feature 20: note = invAb
  source 1..20: mol_type = other RNA
tgtaaccaagagtattccat

SEQ ID NO: 1056 - RNA, 23 nt, Synthetic construct
  source 1..23: mol_type = other RNA
aatggaatactcttggttacatt

SEQ ID NO: 1057 - RNA, 20 nt, Synthetic construct
  misc_feature 20: note = invAb
  source 1..20: mol_type = other RNA
gtaaccaagagtattccatt

SEQ ID NO: 1058 - RNA, 23 nt, Synthetic construct
  source 1..23: mol_type = other RNA
aaatggaatactcttggttactt

SEQ ID NO: 1059 - RNA, 20 nt, Synthetic construct
  misc_feature 20: note = invAb
  source 1..20: mol_type = other RNA
taaccaagagtattccattt

SEQ ID NO: 1060 - RNA, 23 nt, Synthetic construct
  source 1..23: mol_type = other RNA
aaaatggaatactcttggttatt

SEQ ID NO: 1061 - RNA, 20 nt, Synthetic construct
  misc_feature 20: note = invAb
  source 1..20: mol_type = other RNA
aaccaagagtattccatttt

SEQ ID NO: 1062 - RNA, 23 nt, Synthetic construct
  source 1..23: mol_type = other RNA
aaaaatggaatactcttggtttt

SEQ ID NO: 1063 - RNA, 20 nt, Synthetic construct
  misc_feature 20: note = invAb
  source 1..20: mol_type = other RNA
accaagagtattccattttt

SEQ ID NO: 1064 - RNA, 23 nt, Synthetic construct
  source 1..23: mol_type = other RNA
taaaaatggaatactcttggttt

SEQ ID NO: 1065 - RNA, 20 nt, Synthetic construct
  misc_feature 20: note = invAb
  source 1..20: mol_type = other RNA
ccaagagtattccattttta

SEQ ID NO: 1066 - RNA, 23 nt, Synthetic construct
  source 1..23: mol_type = other RNA
ataaaaatggaatactcttggtt

SEQ ID NO: 1067 - RNA, 20 nt, Synthetic construct
  misc_feature 20: note = invAb
  source 1..20: mol_type = other RNA
caagagtattccatttttac

SEQ ID NO: 1068 - RNA, 23 nt, Synthetic construct
  source 1..23: mol_type = other RNA
agtaaaaatggaatactcttgtt

SEQ ID NO: 1069 - RNA, 20 nt, Synthetic construct
  misc_feature 20: note = invAb
  source 1..20: mol_type = other RNA
aagagtattccatttttact

SEQ ID NO: 1070 - RNA, 23 nt, Synthetic construct
  source 1..23: mol_type = other RNA
tagtaaaaatggaatactctttt

SEQ ID NO: 1071 - RNA, 20 nt, Synthetic construct
  misc_feature 20: note = invAb
  source 1..20: mol_type = other RNA
agagtattccatttttacta

SEQ ID NO: 1072 - RNA, 23 nt, Synthetic construct
  source 1..23: mol_type = other RNA
ttagtaaaaatggaatactcttt

SEQ ID NO: 1073 - RNA, 20 nt, Synthetic construct
  misc_feature 20: note = invAb
  source 1..20: mol_type = other RNA
gagtattccatttttactaa

SEQ ID NO: 1074 - RNA, 23 nt, Synthetic construct
  source 1..23: mol_type = other RNA
tttagtaaaaatggaatactctt

SEQ ID NO: 1075 - RNA, 20 nt, Synthetic construct
  misc_feature 20: note = invAb
  source 1..20: mol_type = other RNA
agtattccatttttactaaa

SEQ ID NO: 1076 - RNA, 23 nt, Synthetic construct
  source 1..23: mol_type = other RNA
atttagtaaaaatggaatacttt

SEQ ID NO: 1077 - RNA, 20 nt, Synthetic construct
  misc_feature 20: note = invAb
  source 1..20: mol_type = other RNA
tccatttttactaaagcagt

SEQ ID NO: 1078 - RNA, 23 nt, Synthetic construct
  source 1..23: mol_type = other RNA
aactgctttagtaaaaatggatt

SEQ ID NO: 1079 - RNA, 20 nt, Synthetic construct
  misc_feature 20: note = invAb
  source 1..20: mol_type = other RNA
catttttactaaagcagtgt

SEQ ID NO: 1080 - RNA, 23 nt, Synthetic construct
  source 1..23: mol_type = other RNA
aacactgctttagtaaaaatgtt

SEQ ID NO: 1081 - RNA, 20 nt, Synthetic construct
  misc_feature 20: note = invAb
  source 1..20: mol_type = other RNA
atttttactaaagcagtgtt

SEQ ID NO: 1082 - RNA, 23 nt, Synthetic construct
  source 1..23: mol_type = other RNA
aaacactgctttagtaaaaattt

SEQ ID NO: 1083 - RNA, 20 nt, Synthetic construct
  misc_feature 20: note = invAb
  source 1..20: mol_type = other RNA
tttttactaaagcagtgttt

SEQ ID NO: 1084 - RNA, 23 nt, Synthetic construct
  source 1..23: mol_type = other RNA
aaaacactgctttagtaaaaatt

SEQ ID NO: 1085 - RNA, 20 nt, Synthetic construct
  misc_feature 20: note = invAb
  source 1..20: mol_type = other RNA
ttttactaaagcagtgtttt

SEQ ID NO: 1086 - RNA, 23 nt, Synthetic construct
  source 1..23: mol_type = other RNA
aaaaacactgctttagtaaaatt

SEQ ID NO: 1087 - RNA, 20 nt, Synthetic construct
  misc_feature 20: note = invAb
  source 1..20: mol_type = other RNA
ttactaaagcagtgttttca

SEQ ID NO: 1088 - RNA, 23 nt, Synthetic construct
  source 1..23: mol_type = other RNA
atgaaaacactgctttagtaatt

SEQ ID NO: 1089 - RNA, 20 nt, Synthetic construct
  misc_feature 20: note = invAb
  source 1..20: mol_type = other RNA
tactaaagcagtgttttcac

SEQ ID NO: 1090 - RNA, 23 nt, Synthetic construct
  source 1..23: mol_type = other RNA
agtgaaaacactgctttagtatt

SEQ ID NO: 1091 - RNA, 20 nt, Synthetic construct
  misc_feature 20: note = invAb
  source 1..20: mol_type = other RNA
actaaagcagtgttttcacc

SEQ ID NO: 1092 - RNA, 23 nt, Synthetic construct
  source 1..23: mol_type = other RNA
aggtgaaaacactgctttagttt

SEQ ID NO: 1093 - RNA, 20 nt, Synthetic construct
  misc_feature 20: note = invAb
  source 1..20: mol_type = other RNA
ctaaagcagtgttttcacct

SEQ ID NO: 1094 - RNA, 23 nt, Synthetic construct
  source 1..23: mol_type = other RNA
aaggtgaaaacactgctttagtt

SEQ ID NO: 1095 - RNA, 20 nt, Synthetic construct
  misc_feature 20: note = invAb
  source 1..20: mol_type = other RNA
taaagcagtgttttcacctc

SEQ ID NO: 1096 - RNA, 23 nt, Synthetic construct
  source 1..23: mol_type = other RNA
tgaggtgaaaacactgctttatt

SEQ ID NO: 1097 - RNA, 20 nt, Synthetic construct
  misc_feature 20: note = invAb
  source 1..20: mol_type = other RNA
aaagcagtgttttcacctca

SEQ ID NO: 1098 - RNA, 23 nt, Synthetic construct
  source 1..23: mol_type = other RNA
atgaggtgaaaacactgcttttt

SEQ ID NO: 1099 - RNA, 20 nt, Synthetic construct
  misc_feature 20: note = invAb
  source 1..20: mol_type = other RNA
aagcagtgttttcacctcat

SEQ ID NO: 1100 - RNA, 23 nt, Synthetic construct
  source 1..23: mol_type = other RNA
tatgaggtgaaaacactgctttt

SEQ ID NO: 1101 - RNA, 20 nt, Synthetic construct
  misc_feature 20: note = invAb
  source 1..20: mol_type = other RNA
agtccaggcagagacaataa

SEQ ID NO: 1102 - RNA, 23 nt, Synthetic construct
  source 1..23: mol_type = other RNA
tttattgtctctgcctggacttt

SEQ ID NO: 1103 - RNA, 20 nt, Synthetic construct
  misc_feature 20: note = invAb
  source 1..20: mol_type = other RNA
gtccaggcagagacaataaa

SEQ ID NO: 1104 - RNA, 23 nt, Synthetic construct
  source 1..23: mol_type = other RNA
ttttattgtctctgcctggactt

SEQ ID NO: 1105 - RNA, 20 nt, Synthetic construct
  misc_feature 20: note = invAb
  source 1..20: mol_type = other RNA
tccaggcagagacaataaaa

SEQ ID NO: 1106 - RNA, 23 nt, Synthetic construct
  source 1..23: mol_type = other RNA
attttattgtctctgcctggatt

SEQ ID NO: 1107 - RNA, 20 nt, Synthetic construct
  misc_feature 20: note = invAb
  source 1..20: mol_type = other RNA
ccaggcagagacaataaaac

SEQ ID NO: 1108 - RNA, 23 nt, Synthetic construct
  source 1..23: mol_type = other RNA
tgttttattgtctctgcctggtt

SEQ ID NO: 1109 - RNA, 20 nt, Synthetic construct
  misc_feature 20: note = invAb
  source 1..20: mol_type = other RNA
caggcagagacaataaaaca

SEQ ID NO: 1110 - RNA, 23 nt, Synthetic construct
  source 1..23: mol_type = other RNA
atgttttattgtctctgcctgtt

SEQ ID NO: 1111 - RNA, 20 nt, Synthetic construct
  misc_feature 20: note = invAb
  source 1..20: mol_type = other RNA
aggcagagacaataaaacat

SEQ ID NO: 1112 - RNA, 23 nt, Synthetic construct
  source 1..23: mol_type = other RNA
aatgttttattgtctctgccttt

SEQ ID NO: 1113 - RNA, 20 nt, Synthetic construct
  misc_feature 20: note = invAb
  source 1..20: mol_type = other RNA
ggcagagacaataaaacatt

SEQ ID NO: 1114 - RNA, 23 nt, Synthetic construct
  source 1..23: mol_type = other RNA
aaatgttttattgtctctgcctt

SEQ ID NO: 1115 - RNA, 20 nt, Synthetic construct
  misc_feature 20: note = invAb
  source 1..20: mol_type = other RNA
agacaataaaacattcctgt

SEQ ID NO: 1116 - RNA, 23 nt, Synthetic construct
  source 1..23: mol_type = other RNA
aacaggaatgttttattgtcttt

SEQ ID NO: 1117 - RNA, 20 nt, Synthetic construct
  misc_feature 20: note = invAb
  source 1..20: mol_type = other RNA
gacaataaaacattcctgtg

SEQ ID NO: 1118 - RNA, 23 nt, Synthetic construct
  source 1..23: mol_type = other RNA
tcacaggaatgttttattgtctt

SEQ ID NO: 1119 - RNA, 20 nt, Synthetic construct
  misc_feature 20: note = invAb
  source 1..20: mol_type = other RNA
acaataaaacattcctgtga

SEQ ID NO: 1120 - RNA, 23 nt, Synthetic construct
  source 1..23: mol_type = other RNA
ttcacaggaatgttttattgttt

SEQ ID NO: 1121 - RNA, 20 nt, Synthetic construct
  misc_feature 20: note = invAb
  source 1..20: mol_type = other RNA
caataaaacattcctgtgaa

SEQ ID NO: 1122 - RNA, 23 nt, Synthetic construct
  source 1..23: mol_type = other RNA
tttcacaggaatgttttattgtt

SEQ ID NO: 1123 - RNA, 20 nt, Synthetic construct
  misc_feature 20: note = invAb
  source 1..20: mol_type = other RNA
aataaaacattcctgtgaaa

SEQ ID NO: 1124 - RNA, 23 nt, Synthetic construct
  source 1..23: mol_type = other RNA
atttcacaggaatgttttatttt

SEQ ID NO: 1125 - RNA, 20 nt, Synthetic construct
  misc_feature 20: note = invAb
  source 1..20: mol_type = other RNA
ataaaacattcctgtgaaag

SEQ ID NO: 1126 - RNA, 23 nt, Synthetic construct
  source 1..23: mol_type = other RNA
actttcacaggaatgttttattt

SEQ ID NO: 1127 - RNA, 20 nt, Synthetic construct
  misc_feature 20: note = invAb
  source 1..20: mol_type = other RNA
taaaacattcctgtgaaagg

SEQ ID NO: 1128 - RNA, 23 nt, Synthetic construct
  source 1..23: mol_type = other RNA
acctttcacaggaatgttttatt

SEQ ID NO: 1129 - RNA, 20 nt, Synthetic construct
  misc_feature 20: note = invAb
  source 1..20: mol_type = other RNA
aaaacattcctgtgaaaggc

SEQ ID NO: 1130 - RNA, 23 nt, Synthetic construct
  source 1..23: mol_type = other RNA
tgcctttcacaggaatgtttttt

SEQ ID NO: 1131 - RNA, 20 nt, Synthetic construct
  misc_feature 20: note = invAb
  source 1..20: mol_type = other RNA
aaacattcctgtgaaaggca

SEQ ID NO: 1132 - RNA, 23 nt, Synthetic construct
  source 1..23: mol_type = other RNA
atgcctttcacaggaatgttttt

SEQ ID NO: 1133 - RNA, 20 nt, Synthetic construct
  misc_feature 1: note = sGalNAc3
  misc_feature 20: note = invAb
  source 1..20: mol_type = other RNA
tcttggcaggatggcttctc

SEQ ID NO: 1134 - RNA, 23 nt, Synthetic construct
  source 1..23: mol_type = other RNA
tgagaagccatcctgccaagatt

SEQ ID NO: 1135 - RNA, 20 nt, Synthetic construct
  misc_feature 1: note = sGalNAc3
  misc_feature 20: note = invAb
  source 1..20: mol_type = other RNA
tcttggcaggatggcatctc

SEQ ID NO: 1136 - RNA, 23 nt, Synthetic construct
  source 1..23: mol_type = other RNA
tgagatgccatcctgccaagatt

SEQ ID NO: 1137 - RNA, 20 nt, Synthetic construct
  misc_feature 1: note = sGalNAc3
  misc_feature 20: note = invAb
  source 1..20: mol_type = other RNA
tcttggcaggatgggttctc

SEQ ID NO: 1138 - RNA, 23 nt, Synthetic construct
  source 1..23: mol_type = other RNA
tgagaacccatcctgccaagatt

SEQ ID NO: 1139 - RNA, 20 nt, Synthetic construct
  misc_feature 1: note = sGalNAc3
  misc_feature 20: note = invAb
  source 1..20: mol_type = other RNA
tcttggcaggatgccttctc

SEQ ID NO: 1140 - RNA, 23 nt, Synthetic construct
  source 1..23: mol_type = other RNA
tgagaaggcatcctgccaagatt

SEQ ID NO: 1141 - RNA, 20 nt, Synthetic construct
  misc_feature 1: note = sGalNAc3
  misc_feature 20: note = invAb
  source 1..20: mol_type = other RNA
cttggcaggatggcttctca

SEQ ID NO: 1142 - RNA, 23 nt, Synthetic construct
  source 1..23: mol_type = other RNA
atgagaagccatcctgccaagtt

SEQ ID NO: 1143 - RNA, 20 nt, Synthetic construct
  misc_feature 1: note = sGalNAc3
  misc_feature 20: note = invAb
  source 1..20: mol_type = other RNA
tggcaggatggcttctcatc

SEQ ID NO: 1144 - RNA, 23 nt, Synthetic construct
  source 1..23: mol_type = other RNA
agatgagaagccatcctgccatt

SEQ ID NO: 1145 - RNA, 20 nt, Synthetic construct
  misc_feature 1: note = sGalNAc3
  misc_feature 20: note = invAb
  source 1..20: mol_type = other RNA
gcaggatggcttctcatcgt

SEQ ID NO: 1146 - RNA, 23 nt, Synthetic construct
  source 1..23: mol_type = other RNA
aacgatgagaagccatcctgctt

SEQ ID NO: 1147 - RNA, 20 nt, Synthetic construct
  misc_feature 1: note = sGalNAc3
  misc_feature 20: note = invAb
  source 1..20: mol_type = other RNA
caggatggcttctcatcgtc

SEQ ID NO: 1148 - RNA, 23 nt, Synthetic construct
  source 1..23: mol_type = other RNA
agacgatgagaagccatcctgtt

SEQ ID NO: 1149 - RNA, 20 nt, Synthetic construct
  misc_feature 1: note = sGalNAc3
  misc_feature 20: note = invAb
  source 1..20: mol_type = other RNA
caggatggcttctcttcgtc

SEQ ID NO: 1150 - RNA, 23 nt, Synthetic construct
  source 1..23: mol_type = other RNA
agacgaagagaagccatcctgtt

SEQ ID NO: 1151 - RNA, 20 nt, Synthetic construct
  misc_feature 1: note = sGalNAc3
  misc_feature 20: note = invAb
  source 1..20: mol_type = other RNA
aggatggcttctcatcgtct

SEQ ID NO: 1152 - RNA, 23 nt, Synthetic construct
  source 1..23: mol_type = other RNA
aagacgatgagaagccatccttt

SEQ ID NO: 1153 - RNA, 20 nt, Synthetic construct
  misc_feature 1: note = sGalNAc3
  misc_feature 20: note = invAb
  source 1..20: mol_type = other RNA
gatggcttctcatcgtctgc

SEQ ID NO: 1154 - RNA, 23 nt, Synthetic construct
  source 1..23: mol_type = other RNA
agcagacgatgagaagccatctt

SEQ ID NO: 1155 - RNA, 18 nt, Synthetic construct
  misc_feature 1: note = sGalNAc3
  misc_feature 18: note = invAb
  source 1..18: mol_type = other RNA
ggcttctcatcgtctgct

SEQ ID NO: 1156 - RNA, 21 nt, Synthetic construct
  source 1..21: mol_type = other RNA
gagcagacgatgagaagcctt

SEQ ID NO: 1157 - RNA, 18 nt, Synthetic construct
  misc_feature 1: note = sGalNAc3
  misc_feature 18: note = invAb
  source 1..18: mol_type = other RNA
ggcttctcatcgtctgct

SEQ ID NO: 1158 - RNA, 21 nt, Synthetic construct
  source 1..21: mol_type = other RNA
gagcagacgatgagaagcctt

SEQ ID NO: 1159 - RNA, 20 nt, Synthetic construct
  misc_feature 1: note = sGalNAc3
  misc_feature 20: note = invAb
  source 1..20: mol_type = other RNA
atggcttctcatcgtctgct

SEQ ID NO: 1160 - RNA, 23 nt, Synthetic construct
  source 1..23: mol_type = other RNA
aagcagacgatgagaagccattt

SEQ ID NO: 1161 - RNA, 20 nt, Synthetic construct
  misc_feature 1: note = sGalNAc3
  misc_feature 20: note = invAb
  source 1..20: mol_type = other RNA
atggcttctcatcgactgct

SEQ ID NO: 1162 - RNA, 23 nt, Synthetic construct
  source 1..23: mol_type = other RNA
aagcagtcgatgagaagccattt

SEQ ID NO: 1163 - RNA, 20 nt, Synthetic construct
  misc_feature 1: note = sGalNAc3
  misc_feature 20: note = invAb
  source 1..20: mol_type = other RNA
atggcttctcatcctctgct

SEQ ID NO: 1164 - RNA, 23 nt, Synthetic construct
  source 1..23: mol_type = other RNA
aagcagaggatgagaagccattt

SEQ ID NO: 1165 - RNA, 20 nt, Synthetic construct
  misc_feature 1: note = sGalNAc3
  misc_feature 20: note = invAb
  source 1..20: mol_type = other RNA
atggcttctcatcgtctgct

SEQ ID NO: 1166 - RNA, 23 nt, Synthetic construct
  source 1..23: mol_type = other RNA
aagcagacgatgagaagccattt

SEQ ID NO: 1167 - RNA, 20 nt, Synthetic construct
  misc_feature 1: note = sGalNAc3
  misc_feature 20: note = invAb
  source 1..20: mol_type = other RNA
atggcttctcatcctctgct

SEQ ID NO: 1168 - RNA, 23 nt, Synthetic construct
  source 1..23: mol_type = other RNA
aagcagaggatgagaagccattt

SEQ ID NO: 1169 - RNA, 20 nt, Synthetic construct
  misc_feature 1: note = sGalNAc3
  misc_feature 20: note = invAb
  source 1..20: mol_type = other RNA
atggcttctcatcgtctgct

SEQ ID NO: 1170 - RNA, 23 nt, Synthetic construct
  misc_feature 1: note = sGalNAc3
  source 1..23: mol_type = other RNA
aagcagacgatgagaagccattt

SEQ ID NO: 1171 - RNA, 20 nt, Synthetic construct
  misc_feature 20: note = invAb
  source 1..20: mol_type = other RNA
atggcttctcatcctctgct

SEQ ID NO: 1172 - RNA, 23 nt, Synthetic construct
  source 1..23: mol_type = other RNA
aagcagaggatgagaagccattt

SEQ ID NO: 1173 - RNA, 20 nt, Synthetic construct
  misc_feature 1: note = sGalNAc3
  misc_feature 20: note = invAb
  source 1..20: mol_type = other RNA
atggcttctcatcgtctgct

SEQ ID NO: 1174 - RNA, 23 nt, Synthetic construct
  source 1..23: mol_type = other RNA
aagcagacgatgagaagccattt

SEQ ID NO: 1175 - RNA, 18 nt, Synthetic construct
  misc_feature 1: note = sGalNAc3
  misc_feature 18: note = invAb
  source 1..18: mol_type = other RNA
ggcttctcatcctctgct

SEQ ID NO: 1176 - RNA, 21 nt, Synthetic construct
  source 1..21: mol_type = other RNA
aagcagaggatgagaagcctt

SEQ ID NO: 1177 - RNA, 20 nt, Synthetic construct
  misc_feature 1: note = sGalNAc3
  misc_feature 20: note = invAb
  source 1..20: mol_type = other RNA
atggcttctcatcctctgct

SEQ ID NO: 1178 - RNA, 23 nt, Synthetic construct
  source 1..23: mol_type = other RNA
aagcagaggatgagaagccatcc

SEQ ID NO: 1179 - RNA, 20 nt, Synthetic construct
  misc_feature 1: note = sGalNAc3
  misc_feature 20: note = invAb
  source 1..20: mol_type = other RNA
atggcttctcatcctctgct

SEQ ID NO: 1180 - RNA, 23 nt, Synthetic construct
  source 1..23: mol_type = other RNA
aagcagaggatgagaagccatcc

SEQ ID NO: 1181 - RNA, 20 nt, Synthetic construct
  misc_feature 20: note = invAb
  misc_feature 1: note = sGalNAc3
  source 1..20: mol_type = other RNA
atggcttctcatcctctgct

SEQ ID NO: 1182 - RNA, 23 nt, Synthetic construct
  source 1..23: mol_type = other RNA
aagcagaggatgagaagccatcc

SEQ ID NO: 1183 - RNA, 20 nt, Synthetic construct
  misc_feature 1: note = sGalNAc3
  misc_feature 20: note = invAb
  source 1..20: mol_type = other RNA
atggcttctcatcctctgct

SEQ ID NO: 1184 - RNA, 23 nt, Synthetic construct
  source 1..23: mol_type = other RNA
aagcagaggatgagaagccatcc

SEQ ID NO: 1185 - RNA, 20 nt, Synthetic construct
  misc_feature 1: note = sGalNAc3
  misc_feature 20: note = invAb
  source 1..20: mol_type = other RNA
atggcttctcatcgtctgct

SEQ ID NO: 1186 - RNA, 21 nt, Synthetic construct
  source 1..21: mol_type = other RNA
aagcagacgatgagaagccat

SEQ ID NO: 1187 - RNA, 20 nt, Synthetic construct
  misc_feature 1: note = sGalNAc3
  misc_feature 20: note = invAb
  source 1..20: mol_type = other RNA
atggcttctcatcgactgct

SEQ ID NO: 1188 - RNA, 21 nt, Synthetic construct
  source 1..21: mol_type = other RNA
aagcagtcgatgagaagccat

SEQ ID NO: 1189 - RNA, 20 nt, Synthetic construct
  misc_feature 1: note = sGalNAc3
  misc_feature 20: note = invAb
  source 1..20: mol_type = other RNA
atggcttctcatcctctgct

SEQ ID NO: 1190 - RNA, 21 nt, Synthetic construct
  source 1..21: mol_type = other RNA
aagcagaggatgagaagccat

SEQ ID NO: 1191 - RNA, 18 nt, Synthetic construct
  misc_feature 1: note = sGalNAc3
  misc_feature 18: note = invAb
  source 1..18: mol_type = other RNA
ggcttctcatcgtctgct

SEQ ID NO: 1192 - RNA, 21 nt, Synthetic construct
  source 1..21: mol_type = other RNA
aagcagacgatgagaagcctt

SEQ ID NO: 1193 - RNA, 18 nt, Synthetic construct
  misc_feature 1: note = sGalNAc3
  misc_feature 18: note = invAb
  source 1..18: mol_type = other RNA
ggcttctcatcctctgct

SEQ ID NO: 1194 - RNA, 21 nt, Synthetic construct
  source 1..21: mol_type = other RNA
aagcagaggatgagaagcctt

SEQ ID NO: 1195 - RNA, 20 nt, Synthetic construct
  misc_feature 1: note = sGalNAc3
  misc_feature 20: note = invAb
  source 1..20: mol_type = other RNA
atggcttctcatcgtctgct

SEQ ID NO: 1196 - RNA, 23 nt, Synthetic construct
  source 1..23: mol_type = other RNA
aagcagacgatgagaagccattt

SEQ ID NO: 1197 - RNA, 20 nt, Synthetic construct
  misc_feature 1: note = sGalNAc3
  misc_feature 20: note = invAb
  source 1..20: mol_type = other RNA
atggcttctcatcctctgct

SEQ ID NO: 1198 - RNA, 23 nt, Synthetic construct
  source 1..23: mol_type = other RNA
aagcagaggatgagaagccattt

SEQ ID NO: 1199 - RNA, 20 nt, Synthetic construct
  misc_feature 1: note = sGalNAc3
  misc_feature 20: note = invAb
  source 1..20: mol_type = other RNA
atggcttctcatcgtctgct

SEQ ID NO: 1200 - RNA, 23 nt, Synthetic construct
  source 1..23: mol_type = other RNA
aagcagacgatgagaagccattt

SEQ ID NO: 1201 - RNA, 20 nt, Synthetic construct
  misc_feature 1: note = sGalNAc3
  misc_feature 20: note = invAb
  source 1..20: mol_type = other RNA
atggcttctcatcctctgct

SEQ ID NO: 1202 - RNA, 23 nt, Synthetic construct
  source 1..23: mol_type = other RNA
aagcagaggatgagaagccattt

SEQ ID NO: 1203 - RNA, 18 nt, Synthetic construct
  misc_feature 1: note = sGalNAc3
  misc_feature 18: note = invAb
  source 1..18: mol_type = other RNA
ggcttctcatcctctgct

SEQ ID NO: 1204 - RNA, 21 nt, Synthetic construct
  source 1..21: mol_type = other RNA
aagcagaggatgagaagcctt

SEQ ID NO: 1205 - RNA, 18 nt, Synthetic construct
  misc_feature 1: note = sGalNAc3
  misc_feature 18: note = invAb
  source 1..18: mol_type = other RNA
ggcttctcatcctctgct

SEQ ID NO: 1206 - RNA, 21 nt, Synthetic construct
  source 1..21: mol_type = other RNA
aagcagaggatgagaagcctt

SEQ ID NO: 1207 - RNA, 18 nt, Synthetic construct
  misc_feature 1: note = sGalNAc3
  misc_feature 18: note = invAb
  source 1..18: mol_type = other RNA
ggcttctcatcgtctgct

SEQ ID NO: 1208 - RNA, 21 nt, Synthetic construct
  source 1..21: mol_type = other RNA
aagcagacgatgagaagcctt

SEQ ID NO: 1209 - RNA, 18 nt, Synthetic construct
  misc_feature 1: note = sGalNAc3
  misc_feature 18: note = invAb
  source 1..18: mol_type = other RNA
ggcttctcatcctctgct

SEQ ID NO: 1210 - RNA, 21 nt, Synthetic construct
  source 1..21: mol_type = other RNA
aagcagaggatgagaagcctt

SEQ ID NO: 1211 - RNA, 20 nt, Synthetic construct
  misc_feature 1: note = sGalNAc3
  misc_feature 20: note = invAb
  source 1..20: mol_type = other RNA
gcttctcatcgtctgctcct

SEQ ID NO: 1212 - RNA, 23 nt, Synthetic construct
  source 1..23: mol_type = other RNA
aaggagcagacgatgagaagctt

SEQ ID NO: 1213 - RNA, 20 nt, Synthetic construct
  misc_feature 1: note = sGalNAc3
  misc_feature 20: note = invAb
  source 1..20: mol_type = other RNA
gcttctcatcgtctggtcct

SEQ ID NO: 1214 - RNA, 23 nt, Synthetic construct
  source 1..23: mol_type = other RNA
aaggaccagacgatgagaagctt

SEQ ID NO: 1215 - RNA, 20 nt, Synthetic construct
  misc_feature 1: note = sGalNAc3
  misc_feature 20: note = invAb
  source 1..20: mol_type = other RNA
cttctcatcgtctgctcctc

SEQ ID NO: 1216 - RNA, 23 nt, Synthetic construct
  source 1..23: mol_type = other RNA
agaggagcagacgatgagaagtt

SEQ ID NO: 1217 - RNA, 20 nt, Synthetic construct
  misc_feature 1: note = sGalNAc3
  misc_feature 20: note = invAb
  source 1..20: mol_type = other RNA
ttctcatcgtctgctcctcc

SEQ ID NO: 1218 - RNA, 23 nt, Synthetic construct
  source 1..23: mol_type = other RNA
aggaggagcagacgatgagaatt

SEQ ID NO: 1219 - RNA, 20 nt, Synthetic construct
  misc_feature 1: note = sGalNAc3
  misc_feature 20: note = invAb
  source 1..20: mol_type = other RNA
tctcatcgtctgctcctcct

SEQ ID NO: 1220 - RNA, 23 nt, Synthetic construct
  source 1..23: mol_type = other RNA
aaggaggagcagacgatgagatt

SEQ ID NO: 1221 - RNA, 20 nt, Synthetic construct
  misc_feature 1: note = sGalNAc3
  misc_feature 20: note = invAb
  source 1..20: mol_type = other RNA
tctcatcgtctgctcgtcct

SEQ ID NO: 1222 - RNA, 23 nt, Synthetic construct
  source 1..23: mol_type = other RNA
aaggacgagcagacgatgagatt

SEQ ID NO: 1223 - RNA, 20 nt, Synthetic construct
  misc_feature 1: note = sGalNAc3
  misc_feature 20: note = invAb
  source 1..20: mol_type = other RNA
ctcatcgtctgctcctcctc

SEQ ID NO: 1224 - RNA, 23 nt, Synthetic construct
  source 1..23: mol_type = other RNA
agaggaggagcagacgatgagtt

SEQ ID NO: 1225 - RNA, 18 nt, Synthetic construct
  misc_feature 1: note = sGalNAc3
  misc_feature 18: note = invAb
  source 1..18: mol_type = other RNA
tcgtctgctcctcctctg

SEQ ID NO: 1226 - RNA, 21 nt, Synthetic construct
  source 1..21: mol_type = other RNA
gcagaggaggagcagacgatt

SEQ ID NO: 1227 - RNA, 18 nt, Synthetic construct
  misc_feature 1: note = sGalNAc3
  misc_feature 18: note = invAb
  source 1..18: mol_type = other RNA
tcgtctgctcctcctctg

SEQ ID NO: 1228 - RNA, 21 nt, Synthetic construct
  source 1..21: mol_type = other RNA
gcagaggaggagcagacgatt

SEQ ID NO: 1229 - RNA, 20 nt, Synthetic construct
  misc_feature 1: note = sGalNAc3
  misc_feature 20: note = invAb
  source 1..20: mol_type = other RNA
catcgtctgctcctgctctg

SEQ ID NO: 1230 - RNA, 23 nt, Synthetic construct
  source 1..23: mol_type = other RNA
acagagcaggagcagacgatgtt

SEQ ID NO: 1231 - RNA, 20 nt, Synthetic construct
  misc_feature 1: note = sGalNAc3
  misc_feature 20: note = invAb
  source 1..20: mol_type = other RNA
catcgtctgctcctgctctg

SEQ ID NO: 1232 - RNA, 21 nt, Synthetic construct
  source 1..21: mol_type = other RNA
acagagcaggagcagacgatg

SEQ ID NO: 1233 - RNA, 20 nt, Synthetic construct
  misc_feature 1: note = sGalNAc3
  misc_feature 20: note = invAb
  source 1..20: mol_type = other RNA
catcgtctgctcctcctctg

SEQ ID NO: 1234 - RNA, 23 nt, Synthetic construct
  source 1..23: mol_type = other RNA
acagaggaggagcagacgatgtt

SEQ ID NO: 1235 - RNA, 20 nt, Synthetic construct
  misc_feature 1: note = sGalNAc3
  misc_feature 20: note = invAb
  source 1..20: mol_type = other RNA
catcgtctgctccacctctg

SEQ ID NO: 1236 - RNA, 23 nt, Synthetic construct
  source 1..23: mol_type = other RNA
acagaggtggagcagacgatgtt

SEQ ID NO: 1237 - RNA, 18 nt, Synthetic construct
  misc_feature 1: note = sGalNAc3
  misc_feature 18: note = invAb
  source 1..18: mol_type = other RNA
tgccttgctggactggta

SEQ ID NO: 1238 - RNA, 21 nt, Synthetic construct
  source 1..21: mol_type = other RNA
ataccagtccagcaaggcatt

SEQ ID NO: 1239 - RNA, 18 nt, Synthetic construct
  misc_feature 1: note = sGalNAc3
  misc_feature 18: note = invAb
  source 1..18: mol_type = other RNA
tgccttgctggactggta

SEQ ID NO: 1240 - RNA, 21 nt, Synthetic construct
  source 1..21: mol_type = other RNA
ataccagtccagcaaggcatt

SEQ ID NO: 1241 - RNA, 20 nt, Synthetic construct
  misc_feature 1: note = sGalNAc3
  misc_feature 20: note = invAb
  source 1..20: mol_type = other RNA
gctggactggtatttgtgtc

SEQ ID NO: 1242 - RNA, 23 nt, Synthetic construct
  source 1..23: mol_type = other RNA
agacacaaataccagtccagctt

SEQ ID NO: 1243 - RNA, 20 nt, Synthetic construct
  misc_feature 1: note = sGalNAc3
  misc_feature 20: note = invAb
  source 1..20: mol_type = other RNA
gctggactggtatttctgtc

SEQ ID NO: 1244 - RNA, 23 nt, Synthetic construct
  source 1..23: mol_type = other RNA
agacagaaataccagtccagctt

SEQ ID NO: 1245 - RNA, 20 nt, Synthetic construct
  misc_feature 1: note = sGalNAc3
  misc_feature 20: note = invAb
  source 1..20: mol_type = other RNA
ctggactggtatttgtgtct

SEQ ID NO: 1246 - RNA, 23 nt, Synthetic construct
  source 1..23: mol_type = other RNA
aagacacaaataccagtccagtt

SEQ ID NO: 1247 - RNA, 20 nt, Synthetic construct
  misc_feature 1: note = sGalNAc3
  misc_feature 20: note = invAb
  source 1..20: mol_type = other RNA
tggactggtatttctgtctg

SEQ ID NO: 1248 - RNA, 23 nt, Synthetic construct
  source 1..23: mol_type = other RNA
tcagacagaaataccagtccatt

SEQ ID NO: 1249 - RNA, 20 nt, Synthetic construct
  misc_feature 1: note = sGalNAc3
  misc_feature 20: note = invAb
  source 1..20: mol_type = other RNA
tggactggtatttctgtctg

SEQ ID NO: 1250 - RNA, 21 nt, Synthetic construct
  source 1..21: mol_type = other RNA
tcagacagaaataccagtcca

SEQ ID NO: 1251 - RNA, 20 nt, Synthetic construct
  misc_feature 1: note = sGalNAc3
  misc_feature 20: note = invAb
  source 1..20: mol_type = other RNA
tggactggtatttgtgtctg

SEQ ID NO: 1252 - RNA, 23 nt, Synthetic construct
  source 1..23: mol_type = other RNA
tcagacacaaataccagtccatt

SEQ ID NO: 1253 - RNA, 20 nt, Synthetic construct
  misc_feature 1: note = sGalNAc3
  misc_feature 20: note = invAb
  source 1..20: mol_type = other RNA
ggactggtatttgtgtctga

SEQ ID NO: 1254 - RNA, 23 nt, Synthetic construct
  source 1..23: mol_type = other RNA
atcagacacaaataccagtcctt

SEQ ID NO: 1255 - RNA, 20 nt, Synthetic construct
  misc_feature 1: note = sGalNAc3
  misc_feature 20: note = invAb
  source 1..20: mol_type = other RNA
actggtatttgtgtctgagg

SEQ ID NO: 1256 - RNA, 23 nt, Synthetic construct
  source 1..23: mol_type = other RNA
acctcagacacaaataccagttt

SEQ ID NO: 1257 - RNA, 20 nt, Synthetic construct
  misc_feature 1: note = sGalNAc3
  misc_feature 20: note = invAb
  source 1..20: mol_type = other RNA
ctggtatttgtgtctgaggc

SEQ ID NO: 1258 - RNA, 23 nt, Synthetic construct
  source 1..23: mol_type = other RNA
agcctcagacacaaataccagtt

SEQ ID NO: 1259 - RNA, 20 nt, Synthetic construct
  misc_feature 1: note = sGalNAc3
  misc_feature 20: note = invAb
  source 1..20: mol_type = other RNA
ggtatttgtgtctgacgctg

SEQ ID NO: 1260 - RNA, 23 nt, Synthetic construct
  source 1..23: mol_type = other RNA
acagcgtcagacacaaataccag

SEQ ID NO: 1261 - RNA, 20 nt, Synthetic construct
  misc_feature 1: note = sGalNAc3
  misc_feature 20: note = invAb
  source 1..20: mol_type = other RNA
ggtatttgtgtctgacgctg

SEQ ID NO: 1262 - RNA, 23 nt, Synthetic construct
  source 1..23: mol_type = other RNA
acagcgtcagacacaaataccag

SEQ ID NO: 1263 - RNA, 20 nt, Synthetic construct
  misc_feature 1: note = sGalNAc3
  misc_feature 20: note = invAb
  source 1..20: mol_type = other RNA
ggtatttgtgtctgacgctg

SEQ ID NO: 1264 - RNA, 23 nt, Synthetic construct
  source 1..23: mol_type = other RNA
acagcgtcagacacaaataccag

SEQ ID NO: 1265 - RNA, 20 nt, Synthetic construct
  misc_feature 1: note = sGalNAc3
  misc_feature 20: note = invAb
  source 1..20: mol_type = other RNA
ggtatttgtgtctgacgctg

SEQ ID NO: 1266 - RNA, 23 nt, Synthetic construct
  source 1..23: mol_type = other RNA
acagcgtcagacacaaataccag

SEQ ID NO: 1267 - RNA, 20 nt, Synthetic construct
  misc_feature 1: note = sGalNAc3
  misc_feature 20: note = invAb
  source 1..20: mol_type = other RNA
ggtatttgtgtctgaggctg

SEQ ID NO: 1268 - RNA, 23 nt, Synthetic construct
  source 1..23: mol_type = other RNA
acagcctcagacacaaatacctt

SEQ ID NO: 1269 - RNA, 20 nt, Synthetic construct
  misc_feature 1: note = sGalNAc3
  misc_feature 20: note = invAb
  source 1..20: mol_type = other RNA
ggtatttgtgtctgacgctg

SEQ ID NO: 1270 - RNA, 23 nt, Synthetic construct
  source 1..23: mol_type = other RNA
acagcgtcagacacaaatacctt

SEQ ID NO: 1271 - RNA, 20 nt, Synthetic construct
  misc_feature 1: note = sGalNAc3
  misc_feature 20: note = invAb
  source 1..20: mol_type = other RNA
tgaatccaagtgtcctctga

SEQ ID NO: 1272 - RNA, 23 nt, Synthetic construct
  source 1..23: mol_type = other RNA
atcagaggacacttggattcatt

SEQ ID NO: 1273 - RNA, 20 nt, Synthetic construct
  misc_feature 1: note = sGalNAc3
  misc_feature 20: note = invAb
  source 1..20: mol_type = other RNA
tgaatccaagtgtgctctga

SEQ ID NO: 1274 - RNA, 23 nt, Synthetic construct
  source 1..23: mol_type = other RNA
atcagagcacacttggattcatt

SEQ ID NO: 1275 - RNA, 20 nt, Synthetic construct
  misc_feature 1: note = sGalNAc3
  misc_feature 20: note = invAb
  source 1..20: mol_type = other RNA
aatccaagtgtcctctgatg

SEQ ID NO: 1276 - RNA, 23 nt, Synthetic construct
  source 1..23: mol_type = other RNA
acatcagaggacacttggatttt

SEQ ID NO: 1277 - RNA, 20 nt, Synthetic construct
  misc_feature 1: note = sGalNAc3
  misc_feature 20: note = invAb
  source 1..20: mol_type = other RNA
atccaagtgtcctctgatgg

SEQ ID NO: 1278 - RNA, 23 nt, Synthetic construct
  source 1..23: mol_type = other RNA
accatcagaggacacttggattt

SEQ ID NO: 1279 - RNA, 20 nt, Synthetic construct
  misc_feature 1: note = sGalNAc3
  misc_feature 20: note = invAb
  source 1..20: mol_type = other RNA
atccaagtgtcctctcatgg

SEQ ID NO: 1280 - RNA, 23 nt, Synthetic construct
  source 1..23: mol_type = other RNA
accatgagaggacacttggattt

SEQ ID NO: 1281 - RNA, 20 nt, Synthetic construct
  misc_feature 1: note = sGalNAc3
  misc_feature 20: note = invAb
  source 1..20: mol_type = other RNA
atccaagtgtcctcagatgg

SEQ ID NO: 1282 - RNA, 23 nt, Synthetic construct
  source 1..23: mol_type = other RNA
accatctgaggacacttggattt

SEQ ID NO: 1283 - RNA, 20 nt, Synthetic construct
  misc_feature 1: note = sGalNAc3
  misc_feature 20: note = invAb
  source 1..20: mol_type = other RNA
tccaagtgtcctctcatggt

SEQ ID NO: 1284 - RNA, 23 nt, Synthetic construct
  source 1..23: mol_type = other RNA
aaccatgagaggacacttggatt

SEQ ID NO: 1285 - RNA, 20 nt, Synthetic construct
  misc_feature 1: note = sGalNAc3
  misc_feature 20: note = invAb
  source 1..20: mol_type = other RNA
tccaagtgtcctctcatggt

SEQ ID NO: 1286 - RNA, 21 nt, Synthetic construct
  source 1..21: mol_type = other RNA
aaccatgagaggacacttgga

SEQ ID NO: 1287 - RNA, 18 nt, Synthetic construct
  misc_feature 1: note = sGalNAc3
  misc_feature 18: note = invAb
  source 1..18: mol_type = other RNA
caagtgtcctctcatggt

SEQ ID NO: 1288 - RNA, 21 nt, Synthetic construct
  source 1..21: mol_type = other RNA
aaccatgagaggacacttgtt

SEQ ID NO: 1289 - RNA, 20 nt, Synthetic construct
  misc_feature 1: note = sGalNAc3
  misc_feature 20: note = invAb
  source 1..20: mol_type = other RNA
tccaagtgtcctctgatggt

SEQ ID NO: 1290 - RNA, 23 nt, Synthetic construct
  source 1..23: mol_type = other RNA
aaccatcagaggacacttggatt

SEQ ID NO: 1291 - RNA, 20 nt, Synthetic construct
  misc_feature 1: note = sGalNAc3
  misc_feature 20: note = invAb
  source 1..20: mol_type = other RNA
tccaagtgtcctctgttggt

SEQ ID NO: 1292 - RNA, 23 nt, Synthetic construct
  source 1..23: mol_type = other RNA
aaccaacagaggacacttggatt

SEQ ID NO: 1293 - RNA, 20 nt, Synthetic construct
  misc_feature 1: note = sGalNAc3
  misc_feature 20: note = invAb
  source 1..20: mol_type = other RNA
tccaagtgtcctctcatggt

SEQ ID NO: 1294 - RNA, 23 nt, Synthetic construct
  source 1..23: mol_type = other RNA
aaccatgagaggacacttggatt

SEQ ID NO: 1295 - RNA, 18 nt, Synthetic construct
  misc_feature 1: note = sGalNAc3
  misc_feature 18: note = invAb
  source 1..18: mol_type = other RNA
caagtgtcctctcatggt

SEQ ID NO: 1296 - RNA, 21 nt, Synthetic construct
  source 1..21: mol_type = other RNA
aaccatgagaggacacttgtt

SEQ ID NO: 1297 - RNA, 20 nt, Synthetic construct
  misc_feature 1: note = sGalNAc3
  misc_feature 20: note = invAb
  source 1..20: mol_type = other RNA
ccaagtgtcctctgatggtc

SEQ ID NO: 1298 - RNA, 23 nt, Synthetic construct
  source 1..23: mol_type = other RNA
tgaccatcagaggacacttggtt

SEQ ID NO: 1299 - RNA, 20 nt, Synthetic construct
  misc_feature 1: note = sGalNAc3
  misc_feature 20: note = invAb
  source 1..20: mol_type = other RNA
ccaagtgtcctctgttggtc

SEQ ID NO: 1300 - RNA, 23 nt, Synthetic construct
  source 1..23: mol_type = other RNA
tgaccaacagaggacacttggtt

SEQ ID NO: 1301 - RNA, 20 nt, Synthetic construct
  misc_feature 1: note = sGalNAc3
  misc_feature 20: note = invAb
  source 1..20: mol_type = other RNA
ccaagtgtcctctgatggtc

SEQ ID NO: 1302 - RNA, 23 nt, Synthetic construct
  source 1..23: mol_type = other RNA
tgaccatcagaggacacttggtt

SEQ ID NO: 1303 - RNA, 20 nt, Synthetic construct
  misc_feature 1: note = sGalNAc3
  misc_feature 20: note = invAb
  source 1..20: mol_type = other RNA
ccaagtgtcctctgatggtc

SEQ ID NO: 1304 - RNA, 23 nt, Synthetic construct
  source 1..23: mol_type = other RNA
tgaccatcagaggacacttggtt

SEQ ID NO: 1305 - RNA, 20 nt, Synthetic construct
  misc_feature 1: note = sGalNAc3
  misc_feature 20: note = invAb
  source 1..20: mol_type = other RNA
ccaagtgtcctctgttggtc

SEQ ID NO: 1306 - RNA, 21 nt, Synthetic construct
  source 1..21: mol_type = other RNA
tgaccaacagaggacacttgg

SEQ ID NO: 1307 - RNA, 20 nt, Synthetic construct
  misc_feature 1: note = sGalNAc3
  misc_feature 20: note = invAb
  source 1..20: mol_type = other RNA
ccaagtgtcctctgttggtc

SEQ ID NO: 1308 - RNA, 21 nt, Synthetic construct
  source 1..21: mol_type = other RNA
tgaccaacagaggacacttgg

SEQ ID NO: 1309 - RNA, 20 nt, Synthetic construct
  misc_feature 1: note = sGalNAc3
  misc_feature 20: note = invAb
  source 1..20: mol_type = other RNA
ccaagtgtcctctgttggtc

SEQ ID NO: 1310 - RNA, 21 nt, Synthetic construct
  source 1..21: mol_type = other RNA
tgaccaacagaggacacttgg

SEQ ID NO: 1311 - RNA, 20 nt, Synthetic construct
  misc_feature 1: note = sGalNAc3
  misc_feature 20: note = invAb
  source 1..20: mol_type = other RNA
ccaagtgtcctctgttggtc

SEQ ID NO: 1312 - RNA, 23 nt, Synthetic construct
  source 1..23: mol_type = other RNA
tgaccaacagaggacacttggat

SEQ ID NO: 1313 - RNA, 20 nt, Synthetic construct
  misc_feature 1: note = sGalNAc3
  misc_feature 20: note = invAb
  source 1..20: mol_type = other RNA
ccaagtgtcctctgttggtc

SEQ ID NO: 1314 - RNA, 23 nt, Synthetic construct
  source 1..23: mol_type = other RNA
tgaccaacagaggacacttggat

SEQ ID NO: 1315 - RNA, 20 nt, Synthetic construct
  misc_feature 1: note = sGalNAc3
  misc_feature 20: note = invAb
  source 1..20: mol_type = other RNA
ccaagtgtcctctgttggtc

SEQ ID NO: 1316 - RNA, 23 nt, Synthetic construct
  source 1..23: mol_type = other RNA
tgaccaacagaggacacttggat

SEQ ID NO: 1317 - RNA, 20 nt, Synthetic construct
  misc_feature 1: note = sGalNAc3
  misc_feature 20: note = invAb
  source 1..20: mol_type = other RNA
ccaagtgtcctctgttggtc

SEQ ID NO: 1318 - RNA, 23 nt, Synthetic construct
  source 1..23: mol_type = other RNA
tgaccaacagaggacacttggat

SEQ ID NO: 1319 - RNA, 20 nt, Synthetic construct
  misc_feature 1: note = sGalNAc3
  misc_feature 20: note = invAb
  source 1..20: mol_type = other RNA
ccaagtgtcctctgatggtc

SEQ ID NO: 1320 - RNA, 21 nt, Synthetic construct
  source 1..21: mol_type = other RNA
tgaccatcagaggacacttgg

SEQ ID NO: 1321 - RNA, 20 nt, Synthetic construct
  misc_feature 1: note = sGalNAc3
  misc_feature 20: note = invAb
  source 1..20: mol_type = other RNA
ccaagtgtcctctgttggtc

SEQ ID NO: 1322 - RNA, 21 nt, Synthetic construct
  source 1..21: mol_type = other RNA
tgaccaacagaggacacttgg

SEQ ID NO: 1323 - RNA, 18 nt, Synthetic construct
  misc_feature 1: note = sGalNAc3
  misc_feature 18: note = invAb
  source 1..18: mol_type = other RNA
aagtgtcctctgatggtc

SEQ ID NO: 1324 - RNA, 21 nt, Synthetic construct
  source 1..21: mol_type = other RNA
tgaccatcagaggacactttt

SEQ ID NO: 1325 - RNA, 18 nt, Synthetic construct
  misc_feature 1: note = sGalNAc3
  misc_feature 18: note = invAb
  source 1..18: mol_type = other RNA
aagtgtcctctgttggtc

SEQ ID NO: 1326 - RNA, 21 nt, Synthetic construct
  source 1..21: mol_type = other RNA
tgaccaacagaggacactttt

SEQ ID NO: 1327 - RNA, 20 nt, Synthetic construct
  misc_feature 1: note = sGalNAc3
  misc_feature 20: note = invAb
  source 1..20: mol_type = other RNA
ccaagtgtcctctgatggtc

SEQ ID NO: 1328 - RNA, 23 nt, Synthetic construct
  source 1..23: mol_type = other RNA
tgaccatcagaggacacttggtt

SEQ ID NO: 1329 - RNA, 20 nt, Synthetic construct
  misc_feature 1: note = sGalNAc3
  misc_feature 20: note = invAb
  source 1..20: mol_type = other RNA
ccaagtgtcctctgatggtc

SEQ ID NO: 1330 - RNA, 23 nt, Synthetic construct
  source 1..23: mol_type = other RNA
tgaccatcagaggacacttggtt

SEQ ID NO: 1331 - RNA, 20 nt, Synthetic construct
  misc_feature 1: note = sGalNAc3
  misc_feature 20: note = invAb
  source 1..20: mol_type = other RNA
ccaagtgtcctctgaaggtc

SEQ ID NO: 1332 - RNA, 23 nt, Synthetic construct
  source 1..23: mol_type = other RNA
tgaccttcagaggacacttggtt

SEQ ID NO: 1333 - RNA, 20 nt, Synthetic construct
  misc_feature 1: note = sGalNAc3
  misc_feature 20: note = invAb
  source 1..20: mol_type = other RNA
ccaagtgtcctctgttggtc

SEQ ID NO: 1334 - RNA, 23 nt, Synthetic construct
  source 1..23: mol_type = other RNA
tgaccaacagaggacacttggtt

SEQ ID NO: 1335 - RNA, 20 nt, Synthetic construct
  misc_feature 1: note = sGalNAc3
  misc_feature 20: note = invAb
  source 1..20: mol_type = other RNA
ccaagtgtcctctcatggtc

SEQ ID NO: 1336 - RNA, 23 nt, Synthetic construct
  source 1..23: mol_type = other RNA
tgaccatgagaggacacttggtt

SEQ ID NO: 1337 - RNA, 18 nt, Synthetic construct
  misc_feature 1: note = sGalNAc3
  misc_feature 18: note = invAb
  source 1..18: mol_type = other RNA
aagtgtcctctgatggtc

SEQ ID NO: 1338 - RNA, 21 nt, Synthetic construct
  source 1..21: mol_type = other RNA
tgaccatcagaggacactttt

SEQ ID NO: 1339 - RNA, 18 nt, Synthetic construct
  misc_feature 1: note = sGalNAc3
  misc_feature 18: note = invAb
  source 1..18: mol_type = other RNA
aagtgtcctctgttggtc

SEQ ID NO: 1340 - RNA, 21 nt, Synthetic construct
  source 1..21: mol_type = other RNA
tgaccaacagaggacactttt

SEQ ID NO: 1341 - RNA, 20 nt, Synthetic construct
  misc_feature 1: note = sGalNAc3
  misc_feature 20: note = invAb
  source 1..20: mol_type = other RNA
caagtgtcctctgatggtca

SEQ ID NO: 1342 - RNA, 23 nt, Synthetic construct
  source 1..23: mol_type = other RNA
ttgaccatcagaggacacttgtt

SEQ ID NO: 1343 - RNA, 20 nt, Synthetic construct
  misc_feature 1: note = sGalNAc3
  misc_feature 20: note = invAb
  source 1..20: mol_type = other RNA
caagtgtcctctgatcgtca

SEQ ID NO: 1344 - RNA, 23 nt, Synthetic construct
  source 1..23: mol_type = other RNA
ttgacgatcagaggacacttgtt

SEQ ID NO: 1345 - RNA, 20 nt, Synthetic construct
  misc_feature 1: note = sGalNAc3
  misc_feature 20: note = invAb
  source 1..20: mol_type = other RNA
caagtgtcctctgaaggtca

SEQ ID NO: 1346 - RNA, 23 nt, Synthetic construct
  source 1..23: mol_type = other RNA
ttgaccttcagaggacacttgtt

SEQ ID NO: 1347 - RNA, 20 nt, Synthetic construct
  misc_feature 1: note = sGalNAc3
  misc_feature 20: note = invAb
  source 1..20: mol_type = other RNA
caagtgtcctctgttggtca

SEQ ID NO: 1348 - RNA, 23 nt, Synthetic construct
  source 1..23: mol_type = other RNA
ttgaccaacagaggacacttgtt

SEQ ID NO: 1349 - RNA, 20 nt, Synthetic construct
  misc_feature 1: note = sGalNAc3
  misc_feature 20: note = invAb
  source 1..20: mol_type = other RNA
tcctctgatggtcaaagttc

SEQ ID NO: 1350 - RNA, 23 nt, Synthetic construct
  misc_feature 20: note = invAb
  source 1..23: mol_type = other RNA
agaactttgaccatcagaggatt

SEQ ID NO: 1351 - RNA, 20 nt, Synthetic construct
  misc_feature 1: note = sGalNAc3
  misc_feature 20: note = invAb
  source 1..20: mol_type = other RNA
tcctctgatggtcatagttc

SEQ ID NO: 1352 - RNA, 23 nt, Synthetic construct
  source 1..23: mol_type = other RNA
agaactatgaccatcagaggatt

SEQ ID NO: 1353 - RNA, 20 nt, Synthetic construct
  misc_feature 1: note = sGalNAc3
  misc_feature 20: note = invAb
  source 1..20: mol_type = other RNA
cctctgatggtcaaagttct

SEQ ID NO: 1354 - RNA, 23 nt, Synthetic construct
  source 1..23: mol_type = other RNA
tagaactttgaccatcagaggtt

SEQ ID NO: 1355 - RNA, 20 nt, Synthetic construct
  misc_feature 1: note = sGalNAc3
  misc_feature 20: note = invAb
  source 1..20: mol_type = other RNA
cctctgatggtcaaacttct

SEQ ID NO: 1356 - RNA, 23 nt, Synthetic construct
  source 1..23: mol_type = other RNA
tagaagtttgaccatcagaggtt

SEQ ID NO: 1357 - RNA, 20 nt, Synthetic construct
  misc_feature 1: note = sGalNAc3
  misc_feature 20: note = invAb
  source 1..20: mol_type = other RNA
ctctgatggtcaaagttcta

SEQ ID NO: 1358 - RNA, 23 nt, Synthetic construct
  source 1..23: mol_type = other RNA
atagaactttgaccatcagagtt

SEQ ID NO: 1359 - RNA, 20 nt, Synthetic construct
  misc_feature 1: note = sGalNAc3
  misc_feature 20: note = invAb
  source 1..20: mol_type = other RNA
ctctgatggtcaaagttcta

SEQ ID NO: 1360 - RNA, 21 nt, Synthetic construct
  source 1..21: mol_type = other RNA
atagaactttgaccatcagag

SEQ ID NO: 1361 - RNA, 18 nt, Synthetic construct
  misc_feature 1: note = sGalNAc3
  misc_feature 18: note = invAb
  source 1..18: mol_type = other RNA
ctgatggtcaaagttcta

SEQ ID NO: 1362 - RNA, 21 nt, Synthetic construct
  source 1..21: mol_type = other RNA
atagaactttgaccatcagtt

SEQ ID NO: 1363 - RNA, 20 nt, Synthetic construct
  misc_feature 1: note = sGalNAc3
  misc_feature 20: note = invAb
  source 1..20: mol_type = other RNA
ctctgatggtcaaagttcta

SEQ ID NO: 1364 - RNA, 23 nt, Synthetic construct
  source 1..23: mol_type = other RNA
atagaactttgaccatcagagtt

SEQ ID NO: 1365 - RNA, 18 nt, Synthetic construct
  misc_feature 1: note = sGalNAc3
  misc_feature 18: note = invAb
  source 1..18: mol_type = other RNA
ctgatggtcaaagttcta

SEQ ID NO: 1366 - RNA, 21 nt, Synthetic construct
  source 1..21: mol_type = other RNA
atagaactttgaccatcagtt

SEQ ID NO: 1367 - RNA, 20 nt, Synthetic construct
  misc_feature 1: note = sGalNAc3
  misc_feature 20: note = invAb
  source 1..20: mol_type = other RNA
tctgatggtcaaacttctag

SEQ ID NO: 1368 - RNA, 23 nt, Synthetic construct
  source 1..23: mol_type = other RNA
tctagaagtttgaccatcagatt

SEQ ID NO: 1369 - RNA, 20 nt, Synthetic construct
  misc_feature 1: note = sGalNAc3
  misc_feature 20: note = invAb
  source 1..20: mol_type = other RNA
tctgatggtcaaacttctag

SEQ ID NO: 1370 - RNA, 21 nt, Synthetic construct
  source 1..21: mol_type = other RNA
tctagaagtttgaccatcaga

SEQ ID NO: 1371 - RNA, 20 nt, Synthetic construct
  misc_feature 1: note = sGalNAc3
  misc_feature 20: note = invAb
  source 1..20: mol_type = other RNA
tctgatggtcaaacttctag

SEQ ID NO: 1372 - RNA, 21 nt, Synthetic construct
  source 1..21: mol_type = other RNA
tctagaagtttgaccatcaga

SEQ ID NO: 1373 - RNA, 20 nt, Synthetic construct
  misc_feature 1: note = sGalNAc3
  misc_feature 20: note = invAb
  source 1..20: mol_type = other RNA
tctgatggtcaaacttctag

SEQ ID NO: 1374 - RNA, 21 nt, Synthetic construct
  source 1..21: mol_type = other RNA
tctagaagtttgaccatcaga

SEQ ID NO: 1375 - RNA, 20 nt, Synthetic construct
  misc_feature 1: note = sGalNAc3
  misc_feature 20: note = invAb
  source 1..20: mol_type = other RNA
tctgatggtcaaacttctag

SEQ ID NO: 1376 - RNA, 23 nt, Synthetic construct
  source 1..23: mol_type = other RNA
tctagaagtttgaccatcagagg

SEQ ID NO: 1377 - RNA, 20 nt, Synthetic construct
  misc_feature 1: note = sGalNAc3
  misc_feature 20: note = invAb
  source 1..20: mol_type = other RNA
tctgatggtcaaacttctag

SEQ ID NO: 1378 - RNA, 23 nt, Synthetic construct
  source 1..23: mol_type = other RNA
tctagaagtttgaccatcagagg

SEQ ID NO: 1379 - RNA, 20 nt, Synthetic construct
  misc_feature 1: note = sGalNAc3
  misc_feature 20: note = invAb
  source 1..20: mol_type = other RNA
tctgatggtcaaacttctag

SEQ ID NO: 1380 - RNA, 23 nt, Synthetic construct
  source 1..23: mol_type = other RNA
tctagaagtttgaccatcagagg

SEQ ID NO: 1381 - RNA, 20 nt, Synthetic construct
  misc_feature 1: note = sGalNAc3
  misc_feature 20: note = invAb
  source 1..20: mol_type = other RNA
tctgatggtcaaacttctag

SEQ ID NO: 1382 - RNA, 23 nt, Synthetic construct
  source 1..23: mol_type = other RNA
tctagaagtttgaccatcagagg

SEQ ID NO: 1383 - RNA, 20 nt, Synthetic construct
  misc_feature 1: note = sGalNAc3
  misc_feature 20: note = invAb
  source 1..20: mol_type = other RNA
tctgatggtcaaacttctag

SEQ ID NO: 1384 - RNA, 21 nt, Synthetic construct
  source 1..21: mol_type = other RNA
tctagaagtttgaccatcaga

SEQ ID NO: 1385 - RNA, 18 nt, Synthetic construct
  misc_feature 1: note = sGalNAc3
  misc_feature 18: note = invAb
  source 1..18: mol_type = other RNA
tgatggtcaaacttctag

SEQ ID NO: 1386 - RNA, 21 nt, Synthetic construct
  source 1..21: mol_type = other RNA
tctagaagtttgaccatcatt

SEQ ID NO: 1387 - RNA, 20 nt, Synthetic construct
  misc_feature 1: note = sGalNAc3
  misc_feature 18: note = invAb
  source 1..20: mol_type = other RNA
tctgatggtcaaagttctag

SEQ ID NO: 1388 - RNA, 23 nt, Synthetic construct
  source 1..23: mol_type = other RNA
tctagaactttgaccatcagatt

SEQ ID NO: 1389 - RNA, 20 nt, Synthetic construct
  misc_feature 1: note = sGalNAc3
  misc_feature 20: note = invAb
  source 1..20: mol_type = other RNA
tctgatggtcaaagtactag

SEQ ID NO: 1390 - RNA, 23 nt, Synthetic construct
  source 1..23: mol_type = other RNA
tctagtactttgaccatcagatt

SEQ ID NO: 1391 - RNA, 20 nt, Synthetic construct
  misc_feature 1: note = sGalNAc3
  misc_feature 20: note = invAb
  source 1..20: mol_type = other RNA
tctgatggtcaaacttctag

SEQ ID NO: 1392 - RNA, 23 nt, Synthetic construct
  source 1..23: mol_type = other RNA
tctagaagtttgaccatcagatt

SEQ ID NO: 1393 - RNA, 18 nt, Synthetic construct
  misc_feature 1: note = sGalNAc3
  misc_feature 18: note = invAb
  source 1..18: mol_type = other RNA
tgatggtcaaacttctag

SEQ ID NO: 1394 - RNA, 21 nt, Synthetic construct
  source 1..21: mol_type = other RNA
tctagaagtttgaccatcatt

SEQ ID NO: 1395 - RNA, 20 nt, Synthetic construct
  misc_feature 1: note = sGalNAc3
  misc_feature 20: note = invAb
  source 1..20: mol_type = other RNA
ctgatggtcaaagttctaga

SEQ ID NO: 1396 - RNA, 23 nt, Synthetic construct
  source 1..23: mol_type = other RNA
atctagaactttgaccatcagtt

SEQ ID NO: 1397 - RNA, 20 nt, Synthetic construct
  misc_feature 1: note = sGalNAc3
  misc_feature 20: note = invAb
  source 1..20: mol_type = other RNA
ctgatggtcaaagtactaga

SEQ ID NO: 1398 - RNA, 23 nt, Synthetic construct
  source 1..23: mol_type = other RNA
atctagtactttgaccatcagtt

SEQ ID NO: 1399 - RNA, 20 nt, Synthetic construct
  misc_feature 1: note = sGalNAc3
  misc_feature 20: note = invAb
  source 1..20: mol_type = other RNA
ctgatggtcaaagatctaga

SEQ ID NO: 1400 - RNA, 23 nt, Synthetic construct
  source 1..23: mol_type = other RNA
atctagatctttgaccatcagtt

SEQ ID NO: 1401 - RNA, 20 nt, Synthetic construct
  misc_feature 1: note = sGalNAc3
  misc_feature 20: note = invAb
  source 1..20: mol_type = other RNA
ctgatggtcaaagttctaga

SEQ ID NO: 1402 - RNA, 21 nt, Synthetic construct
  source 1..21: mol_type = other RNA
atctagaactttgaccatcag

SEQ ID NO: 1403 - RNA, 20 nt, Synthetic construct
  misc_feature 1: note = sGalNAc3
  misc_feature 20: note = invAb
  source 1..20: mol_type = other RNA
ctgatggtcaaagtactaga

SEQ ID NO: 1404 - RNA, 21 nt, Synthetic construct
  source 1..21: mol_type = other RNA
atctagtactttgaccatcag

SEQ ID NO: 1405 - RNA, 20 nt, Synthetic construct
  misc_feature 1: note = sGalNAc3
  misc_feature 20: note = invAb
  source 1..20: mol_type = other RNA
ctgatggtcaaagatctaga

SEQ ID NO: 1406 - RNA, 21 nt, Synthetic construct
  source 1..21: mol_type = other RNA
atctagatctttgaccatcag

SEQ ID NO: 1407 - RNA, 18 nt, Synthetic construct
  misc_feature 1: note = sGalNAc3
  misc_feature 18: note = invAb
  source 1..18: mol_type = other RNA
gatggtcaaagttctaga

SEQ ID NO: 1408 - RNA, 21 nt, Synthetic construct
  source 1..21: mol_type = other RNA
atctagaactttgaccatctt

SEQ ID NO: 1409 - RNA, 18 nt, Synthetic construct
  misc_feature 1: note = sGalNAc3
  misc_feature 18: note = invAb
  source 1..18: mol_type = other RNA
gatggtcaaagttctaga

SEQ ID NO: 1410 - RNA, 21 nt, Synthetic construct
  source 1..21: mol_type = other RNA
atctagaactttgaccatctt

SEQ ID NO: 1411 - RNA, 18 nt, Synthetic construct
  misc_feature 1: note = sGalNAc3
  misc_feature 18: note = invAb
  source 1..18: mol_type = other RNA
gatggtcaaagatctaga

SEQ ID NO: 1412 - RNA, 21 nt, Synthetic construct
  source 1..21: mol_type = other RNA
atctagatctttgaccatctt

SEQ ID NO: 1413 - RNA, 20 nt, Synthetic construct
  misc_feature 1: note = sGalNAc3
  misc_feature 20: note = invAb
  source 1..20: mol_type = other RNA
ctgatggtcaaagttctaga

SEQ ID NO: 1414 - RNA, 23 nt, Synthetic construct
  source 1..23: mol_type = other RNA
atctagaactttgaccatcagtt

SEQ ID NO: 1415 - RNA, 20 nt, Synthetic construct
  misc_feature 1: note = sGalNAc3
  misc_feature 20: note = invAb
  source 1..20: mol_type = other RNA
ctgatggtcaaagatctaga

SEQ ID NO: 1416 - RNA, 23 nt, Synthetic construct
  source 1..23: mol_type = other RNA
atctagatctttgaccatcagtt

SEQ ID NO: 1417 - RNA, 18 nt, Synthetic construct
  misc_feature 1: note = sGalNAc3
  misc_feature 18: note = invAb
  source 1..18: mol_type = other RNA
gatggtcaaagttctaga

SEQ ID NO: 1418 - RNA, 21 nt, Synthetic construct
  source 1..21: mol_type = other RNA
atctagaactttgaccatctt

SEQ ID NO: 1419 - RNA, 18 nt, Synthetic construct
  misc_feature 1: note = sGalNAc3
  misc_feature 18: note = invAb
  source 1..18: mol_type = other RNA
gatggtcaaagttctaga

SEQ ID NO: 1420 - RNA, 21 nt, Synthetic construct
  source 1..21: mol_type = other RNA
atctagaactttgaccatctt

SEQ ID NO: 1421 - RNA, 18 nt, Synthetic construct
  misc_feature 1: note = sGalNAc3
  misc_feature 18: note = invAb
  source 1..18: mol_type = other RNA
gatggtcaaagatctaga

SEQ ID NO: 1422 - RNA, 21 nt, Synthetic construct
  source 1..21: mol_type = other RNA
atctagatctttgaccatctt

SEQ ID NO: 1423 - RNA, 20 nt, Synthetic construct
  misc_feature 1: note = sGalNAc3
  misc_feature 20: note = invAb
  source 1..20: mol_type = other RNA
gatggtcaaagttcttgatg

SEQ ID NO: 1424 - RNA, 23 nt, Synthetic construct
  source 1..23: mol_type = other RNA
acatcaagaactttgaccatctt

SEQ ID NO: 1425 - RNA, 20 nt, Synthetic construct
  misc_feature 1: note = sGalNAc3
  misc_feature 20: note = invAb
  source 1..20: mol_type = other RNA
gatggtcaaagttcttgatg

SEQ ID NO: 1426 - RNA, 23 nt, Synthetic construct
  source 1..23: mol_type = other RNA
acatcaagaactttgaccatcag

SEQ ID NO: 1427 - RNA, 20 nt, Synthetic construct
  misc_feature 1: note = sGalNAc3
  misc_feature 20: note = invAb
  source 1..20: mol_type = other RNA
gatggtcaaagttgtagatg

SEQ ID NO: 1428 - RNA, 23 nt, Synthetic construct
  source 1..23: mol_type = other RNA
acatctacaactttgaccatcag

SEQ ID NO: 1429 - RNA, 20 nt, Synthetic construct
  misc_feature 1: note = sGalNAc3
  misc_feature 20: note = invAb
  source 1..20: mol_type = other RNA
gatggtcaaagttcttgatg

SEQ ID NO: 1430 - RNA, 23 nt, Synthetic construct
  source 1..23: mol_type = other RNA
acatcaagaactttgaccatcag

SEQ ID NO: 1431 - RNA, 20 nt, Synthetic construct
  misc_feature 1: note = sGalNAc3
  misc_feature 20: note = invAb
  source 1..20: mol_type = other RNA
gatggtcaaagttgtagatg

SEQ ID NO: 1432 - RNA, 23 nt, Synthetic construct
  source 1..23: mol_type = other RNA
acatctacaactttgaccatcag

SEQ ID NO: 1433 - RNA, 20 nt, Synthetic construct
  misc_feature 1: note = sGalNAc3
  misc_feature 20: note = invAb
  source 1..20: mol_type = other RNA
gatggtcaaagttcttgatg

SEQ ID NO: 1434 - RNA, 23 nt, Synthetic construct
  source 1..23: mol_type = other RNA
acatcaagaactttgaccatcag

SEQ ID NO: 1435 - RNA, 20 nt, Synthetic construct
  misc_feature 1: note = sGalNAc3
  misc_feature 20: note = invAb
  source 1..20: mol_type = other RNA
gatggtcaaagttgtagatg

SEQ ID NO: 1436 - RNA, 23 nt, Synthetic construct
  source 1..23: mol_type = other RNA
acatctacaactttgaccatcag

SEQ ID NO: 1437 - RNA, 20 nt, Synthetic construct
  misc_feature 1: note = sGalNAc3
  misc_feature 20: note = invAb
  source 1..20: mol_type = other RNA
gatggtcaaagttcttgatg

SEQ ID NO: 1438 - RNA, 23 nt, Synthetic construct
  source 1..23: mol_type = other RNA
acatcaagaactttgaccatcag

SEQ ID NO: 1439 - RNA, 20 nt, Synthetic construct
  misc_feature 1: note = sGalNAc3
  misc_feature 20: note = invAb
  source 1..20: mol_type = other RNA
gatggtcaaagttgtagatg

SEQ ID NO: 1440 - RNA, 23 nt, Synthetic construct
  source 1..23: mol_type = other RNA
acatctacaactttgaccatcag

SEQ ID NO: 1441 - RNA, 20 nt, Synthetic construct
  misc_feature 1: note = sGalNAc3
  misc_feature 20: note = invAb
  source 1..20: mol_type = other RNA
gatggtcaaagttcttgatg

SEQ ID NO: 1442 - RNA, 21 nt, Synthetic construct
  source 1..21: mol_type = other RNA
acatcaagaactttgaccatc

SEQ ID NO: 1443 - RNA, 18 nt, Synthetic construct
  misc_feature 1: note = sGalNAc3
  misc_feature 18: note = invAb
  source 1..18: mol_type = other RNA
tggtcaaagttcttgatg

SEQ ID NO: 1444 - RNA, 21 nt, Synthetic construct
  source 1..21: mol_type = other RNA
acatcaagaactttgaccatt

SEQ ID NO: 1445 - RNA, 20 nt, Synthetic construct
  misc_feature 1: note = sGalNAc3
  misc_feature 20: note = invAb
  source 1..20: mol_type = other RNA
gatggtcaaagttctagatg

SEQ ID NO: 1446 - RNA, 23 nt, Synthetic construct
  source 1..23: mol_type = other RNA
acatctagaactttgaccatctt

SEQ ID NO: 1447 - RNA, 20 nt, Synthetic construct
  misc_feature 1: note = sGalNAc3
  misc_feature 20: note = invAb
  source 1..20: mol_type = other RNA
gatggtcaaagttcttgatg

SEQ ID NO: 1448 - RNA, 23 nt, Synthetic construct
  source 1..23: mol_type = other RNA
acatcaagaactttgaccatctt

SEQ ID NO: 1449 - RNA, 20 nt, Synthetic construct
  misc_feature 1: note = sGalNAc3
  misc_feature 20: note = invAb
  source 1..20: mol_type = other RNA
gatggtcaaagttgtagatg

SEQ ID NO: 1450 - RNA, 23 nt, Synthetic construct
  source 1..23: mol_type = other RNA
acatctacaactttgaccatctt

SEQ ID NO: 1451 - RNA, 18 nt, Synthetic construct
  misc_feature 1: note = sGalNAc3
  misc_feature 18: note = invAb
  source 1..18: mol_type = other RNA
tggtcaaagttcttgatg

SEQ ID NO: 1452 - RNA, 21 nt, Synthetic construct
  source 1..21: mol_type = other RNA
acatcaagaactttgaccatt

SEQ ID NO: 1453 - RNA, 20 nt, Synthetic construct
  misc_feature 1: note = sGalNAc3
  misc_feature 20: note = invAb
  source 1..20: mol_type = other RNA
tgggaaaaccagtgagtctg

SEQ ID NO: 1454 - RNA, 23 nt, Synthetic construct
  source 1..23: mol_type = other RNA
acagactcactggttttcccatt

SEQ ID NO: 1455 - RNA, 20 nt, Synthetic construct
  misc_feature 1: note = sGalNAc3
  misc_feature 20: note = invAb
  source 1..20: mol_type = other RNA
aatttgtagaagggatatac

SEQ ID NO: 1456 - RNA, 23 nt, Synthetic construct
  source 1..23: mol_type = other RNA
tgtatatcccttctacaaatttt

SEQ ID NO: 1457 - RNA, 20 nt, Synthetic construct
  misc_feature 1: note = sGalNAc3
  misc_feature 20: note = invAb
  source 1..20: mol_type = other RNA
aatttgtagaaggcatatac

SEQ ID NO: 1458 - RNA, 23 nt, Synthetic construct
  source 1..23: mol_type = other RNA
tgtatatgccttctacaaatttt

SEQ ID NO: 1459 - RNA, 20 nt, Synthetic construct
  misc_feature 1: note = sGalNAc3
  misc_feature 20: note = invAb
  source 1..20: mol_type = other RNA
ttgtagaagggatatacaaa

SEQ ID NO: 1460 - RNA, 23 nt, Synthetic construct
  source 1..23: mol_type = other RNA
atttgtatatcccttctacaatt

SEQ ID NO: 1461 - RNA, 20 nt, Synthetic construct
  misc_feature 1: note = sGalNAc3
  misc_feature 20: note = invAb
  source 1..20: mol_type = other RNA
ttgtagaagggatatacaaa

SEQ ID NO: 1462 - RNA, 21 nt, Synthetic construct
  source 1..21: mol_type = other RNA
atttgtatatcccttctacaa

SEQ ID NO: 1463 - RNA, 18 nt, Synthetic construct
  misc_feature 1: note = sGalNAc3
  misc_feature 18: note = invAb
  source 1..18: mol_type = other RNA
gtagaagggatatacaaa

SEQ ID NO: 1464 - RNA, 21 nt, Synthetic construct
  source 1..21: mol_type = other RNA
atttgtatatcccttctactt

SEQ ID NO: 1465 - RNA, 20 nt, Synthetic construct
  misc_feature 1: note = sGalNAc3
  misc_feature 20: note = invAb
  source 1..20: mol_type = other RNA
ttgtagaagggatatacaaa

SEQ ID NO: 1466 - RNA, 23 nt, Synthetic construct
  source 1..23: mol_type = other RNA
atttgtatatcccttctacaatt

SEQ ID NO: 1467 - RNA, 18 nt, Synthetic construct
  misc_feature 1: note = sGalNAc3
  misc_feature 18: note = invAb
  source 1..18: mol_type = other RNA
gtagaagggatatacaaa

SEQ ID NO: 1468 - RNA, 21 nt, Synthetic construct
  source 1..21: mol_type = other RNA
atttgtatatcccttctactt

SEQ ID NO: 1469 - RNA, 18 nt, Synthetic construct
  misc_feature 1: note = sGalNAc3
  misc_feature 18: note = invAb
  source 1..18: mol_type = other RNA
gaagggatatacaaagtg

SEQ ID NO: 1470 - RNA, 21 nt, Synthetic construct
  source 1..21: mol_type = other RNA
ccactttgtatatcccttctt

SEQ ID NO: 1471 - RNA, 18 nt, Synthetic construct
  misc_feature 1: note = sGalNAc3
  misc_feature 18: note = invAb
  source 1..18: mol_type = other RNA
gaagggatatacaaagtg

SEQ ID NO: 1472 - RNA, 21 nt, Synthetic construct
  source 1..21: mol_type = other RNA
ccactttgtatatcccttctt

SEQ ID NO: 1473 - RNA, 20 nt, Synthetic construct
  misc_feature 1: note = sGalNAc3
  misc_feature 20: note = invAb
  source 1..20: mol_type = other RNA
tagaagggatatacaaagtg

SEQ ID NO: 1474 - RNA, 23 nt, Synthetic construct
  source 1..23: mol_type = other RNA
acactttgtatatcccttctatt

SEQ ID NO: 1475 - RNA, 20 nt, Synthetic construct
  misc_feature 1: note = sGalNAc3
  misc_feature 20: note = invAb
  source 1..20: mol_type = other RNA
tagaagggatatagaaagtg

SEQ ID NO: 1476 - RNA, 23 nt, Synthetic construct
  source 1..23: mol_type = other RNA
acactttctatatcccttctatt

SEQ ID NO: 1477 - RNA, 20 nt, Synthetic construct
  misc_feature 1: note = sGalNAc3
  misc_feature 20: note = invAb
  source 1..20: mol_type = other RNA
tagaagggatatagaaagtg

SEQ ID NO: 1478 - RNA, 23 nt, Synthetic construct
  source 1..23: mol_type = other RNA
acactttctatatcccttctatt

SEQ ID NO: 1479 - RNA, 20 nt, Synthetic construct
  misc_feature 1: note = sGalNAc3
  misc_feature 20: note = invAb
  source 1..20: mol_type = other RNA
tagaagggatatagaaagtg

SEQ ID NO: 1480 - RNA, 23 nt, Synthetic construct
  source 1..23: mol_type = other RNA
acactttctatatcccttctatt

SEQ ID NO: 1481 - RNA, 20 nt, Synthetic construct
  misc_feature 1: note = sGalNAc3
  misc_feature 20: note = invAb
  source 1..20: mol_type = other RNA
tagaagggatatagaaagtg

SEQ ID NO: 1482 - RNA, 23 nt, Synthetic construct
  source 1..23: mol_type = other RNA
acactttctatatcccttctatt

SEQ ID NO: 1483 - RNA, 18 nt, Synthetic construct
  misc_feature 1: note = sGalNAc3
  misc_feature 18: note = invAb
  source 1..18: mol_type = other RNA
gaagggatatacaaagtg

SEQ ID NO: 1484 - RNA, 21 nt, Synthetic construct
  source 1..21: mol_type = other RNA
acactttgtatatcccttctt

SEQ ID NO: 1485 - RNA, 20 nt, Synthetic construct
  misc_feature 1: note = sGalNAc3
  misc_feature 20: note = invAb
  source 1..20: mol_type = other RNA
tagaagggatatacatagtg

SEQ ID NO: 1486 - RNA, 23 nt, Synthetic construct
  source 1..23: mol_type = other RNA
acactatgtatatcccttctaca

SEQ ID NO: 1487 - RNA, 20 nt, Synthetic construct
  misc_feature 1: note = sGalNAc3
  misc_feature 20: note = invAb
  source 1..20: mol_type = other RNA
tagaagggatatactaagtg

SEQ ID NO: 1488 - RNA, 23 nt, Synthetic construct
  source 1..23: mol_type = other RNA
acacttagtatatcccttctaca

SEQ ID NO: 1489 - RNA, 20 nt, Synthetic construct
  misc_feature 1: note = sGalNAc3
  misc_feature 20: note = invAb
  source 1..20: mol_type = other RNA
tagaagggatatagaaagtg

SEQ ID NO: 1490 - RNA, 23 nt, Synthetic construct
  source 1..23: mol_type = other RNA
acactttctatatcccttctaca

SEQ ID NO: 1491 - RNA, 20 nt, Synthetic construct
  misc_feature 1: note = sGalNAc3
  misc_feature 20: note = invAb
  source 1..20: mol_type = other RNA
tagaagggatatacatagtg

SEQ ID NO: 1492 - RNA, 23 nt, Synthetic construct
  source 1..23: mol_type = other RNA
acactatgtatatcccttctaca

SEQ ID NO: 1493 - RNA, 20 nt, Synthetic construct
  misc_feature 1: note = sGalNAc3
  misc_feature 20: note = invAb
  source 1..20: mol_type = other RNA
tagaagggatatactaagtg

SEQ ID NO: 1494 - RNA, 23 nt, Synthetic construct
  source 1..23: mol_type = other RNA
acacttagtatatcccttctaca

SEQ ID NO: 1495 - RNA, 20 nt, Synthetic construct
  misc_feature 1: note = sGalNAc3
  misc_feature 20: note = invAb
  source 1..20: mol_type = other RNA
tagaagggatatagaaagtg

SEQ ID NO: 1496 - RNA, 23 nt, Synthetic construct
  source 1..23: mol_type = other RNA
acactttctatatcccttctaca

SEQ ID NO: 1497 - RNA, 20 nt, Synthetic construct
  misc_feature 1: note = sGalNAc3
  misc_feature 20: note = invAb
  source 1..20: mol_type = other RNA
tagaagggatatacatagtg

SEQ ID NO: 1498 - RNA, 23 nt, Synthetic construct
  source 1..23: mol_type = other RNA
acactatgtatatcccttctaca

SEQ ID NO: 1499 - RNA, 20 nt, Synthetic construct
  misc_feature 1: note = sGalNAc3
  misc_feature 20: note = invAb
  source 1..20: mol_type = other RNA
tagaagggatatactaagtg

SEQ ID NO: 1500 - RNA, 23 nt, Synthetic construct
  source 1..23: mol_type = other RNA
acacttagtatatcccttctaca

SEQ ID NO: 1501 - RNA, 20 nt, Synthetic construct
  misc_feature 1: note = sGalNAc3
  misc_feature 20: note = invAb
  source 1..20: mol_type = other RNA
tagaagggatatagaaagtg

SEQ ID NO: 1502 - RNA, 23 nt, Synthetic construct
  source 1..23: mol_type = other RNA
acactttctatatcccttctaca

SEQ ID NO: 1503 - RNA, 20 nt, Synthetic construct
  misc_feature 1: note = sGalNAc3
  misc_feature 20: note = invAb
  source 1..20: mol_type = other RNA
tagaagggatatacatagtg

SEQ ID NO: 1504 - RNA, 23 nt, Synthetic construct
  source 1..23: mol_type = other RNA
acactatgtatatcccttctaca

SEQ ID NO: 1505 - RNA, 20 nt, Synthetic construct
  misc_feature 1: note = sGalNAc3
  misc_feature 20: note = invAb
  source 1..20: mol_type = other RNA
tagaagggatatactaagtg

SEQ ID NO: 1506 - RNA, 23 nt, Synthetic construct
  source 1..23: mol_type = other RNA
acacttagtatatcccttctaca

SEQ ID NO: 1507 - RNA, 20 nt, Synthetic construct
  misc_feature 20: note = invAb
  source 1..20: mol_type = other RNA
tagaagggatatagaaagtg

SEQ ID NO: 1508 - RNA, 23 nt, Synthetic construct
  source 1..23: mol_type = other RNA
acactttctatatcccttctaca

SEQ ID NO: 1509 - RNA, 20 nt, Synthetic construct
  misc_feature 1: note = sGalNAc3
  misc_feature 20: note = invAb
  source 1..20: mol_type = other RNA
tagaagggatatacaaagtg

SEQ ID NO: 1510 - RNA, 21 nt, Synthetic construct
  source 1..21: mol_type = other RNA
acactttgtatatcccttcta

SEQ ID NO: 1511 - RNA, 20 nt, Synthetic construct
  misc_feature 1: note = sGalNAc3
  misc_feature 20: note = invAb
  source 1..20: mol_type = other RNA
tagaagggatatagaaagtg

SEQ ID NO: 1512 - RNA, 21 nt, Synthetic construct
  source 1..21: mol_type = other RNA
acactttctatatcccttcta

SEQ ID NO: 1513 - RNA, 18 nt, Synthetic construct
  misc_feature 1: note = sGalNAc3
  misc_feature 18: note = invAb
  source 1..18: mol_type = other RNA
gaagggatatagaaagtg

SEQ ID NO: 1514 - RNA, 21 nt, Synthetic construct
  source 1..21: mol_type = other RNA
acactttctatatcccttctt

SEQ ID NO: 1515 - RNA, 20 nt, Synthetic construct
  misc_feature 1: note = sGalNAc3
  misc_feature 20: note = invAb
  source 1..20: mol_type = other RNA
tagaagggatatagaaagtg

SEQ ID NO: 1516 - RNA, 23 nt, Synthetic construct
  source 1..23: mol_type = other RNA
acactttctatatcccttctatt

SEQ ID NO: 1517 - RNA, 20 nt, Synthetic construct
  misc_feature 1: note = sGalNAc3
  misc_feature 20: note = invAb
  source 1..20: mol_type = other RNA
tagaagggatatacaaagtg

SEQ ID NO: 1518 - RNA, 23 nt, Synthetic construct
  source 1..23: mol_type = other RNA
acactttgtatatcccttctatt

SEQ ID NO: 1519 - RNA, 20 nt, Synthetic construct
  misc_feature 1: note = sGalNAc3
  misc_feature 20: note = invAb
  source 1..20: mol_type = other RNA
tagaagggatatacatagtg

SEQ ID NO: 1520 - RNA, 23 nt, Synthetic construct
  source 1..23: mol_type = other RNA
acactatgtatatcccttctatt

SEQ ID NO: 1521 - RNA, 20 nt, Synthetic construct
  misc_feature 1: note = sGalNAc3
  misc_feature 20: note = invAb
  source 1..20: mol_type = other RNA
tagaagggatatactaagtg

SEQ ID NO: 1522 - RNA, 23 nt, Synthetic construct
  source 1..23: mol_type = other RNA
acacttagtatatcccttctatt

SEQ ID NO: 1523 - RNA, 20 nt, Synthetic construct
  misc_feature 1: note = sGalNAc3
  misc_feature 20: note = invAb
  source 1..20: mol_type = other RNA
tagaagggatatagaaagtg

SEQ ID NO: 1524 - RNA, 23 nt, Synthetic construct
  source 1..23: mol_type = other RNA
acactttctatatcccttctatt

SEQ ID NO: 1525 - RNA, 18 nt, Synthetic construct
  misc_feature 1: note = sGalNAc3
  misc_feature 18: note = invAb
  source 1..18: mol_type = other RNA
gaagggatatacaaagtg

SEQ ID NO: 1526 - RNA, 21 nt, Synthetic construct
  source 1..21: mol_type = other RNA
acactttgtatatcccttctt

SEQ ID NO: 1527 - RNA, 18 nt, Synthetic construct
  misc_feature 1: note = sGalNAc3
  misc_feature 18: note = invAb
  source 1..18: mol_type = other RNA
gaagggatatacaaagtg

SEQ ID NO: 1528 - RNA, 21 nt, Synthetic construct
  source 1..21: mol_type = other RNA
acactttgtatatcccttctt

SEQ ID NO: 1529 - RNA, 18 nt, Synthetic construct
  misc_feature 1: note = sGalNAc3
  misc_feature 18: note = invAb
  source 1..18: mol_type = other RNA
gaagggatatagaaagtg

SEQ ID NO: 1530 - RNA, 21 nt, Synthetic construct
  source 1..21: mol_type = other RNA
acactttctatatcccttctt

SEQ ID NO: 1531 - RNA, 20 nt, Synthetic construct
  misc_feature 1: note = sGalNAc3
  misc_feature 20: note = invAb
  source 1..20: mol_type = other RNA
tacaaagtggaaatagacac

SEQ ID NO: 1532 - RNA, 23 nt, Synthetic construct
  source 1..23: mol_type = other RNA
agtgtctatttccactttgtatt

SEQ ID NO: 1533 - RNA, 20 nt, Synthetic construct
  misc_feature 1: note = sGalNAc3
  misc_feature 20: note = invAb
  source 1..20: mol_type = other RNA
tacaaagtggaaattgacac

SEQ ID NO: 1534 - RNA, 23 nt, Synthetic construct
  source 1..23: mol_type = other RNA
agtgtcaatttccactttgtatt

SEQ ID NO: 1535 - RNA, 20 nt, Synthetic construct
  misc_feature 1: note = sGalNAc3
  misc_feature 20: note = invAb
  source 1..20: mol_type = other RNA
caaagtggaaatagacacca

SEQ ID NO: 1536 - RNA, 23 nt, Synthetic construct
  source 1..23: mol_type = other RNA
ttggtgtctatttccactttgtt

SEQ ID NO: 1537 - RNA, 20 nt, Synthetic construct
  misc_feature 1: note = sGalNAc3
  misc_feature 20: note = invAb
  source 1..20: mol_type = other RNA
gtggaaatagacaccaaatc

SEQ ID NO: 1538 - RNA, 23 nt, Synthetic construct
  source 1..23: mol_type = other RNA
agatttggtgtctatttccactt

SEQ ID NO: 1539 - RNA, 20 nt, Synthetic construct
  misc_feature 1: note = sGalNAc3
  misc_feature 20: note = invAb
  source 1..20: mol_type = other RNA
gtggaaatagacagcaaatc

SEQ ID NO: 1540 - RNA, 23 nt, Synthetic construct
  source 1..23: mol_type = other RNA
agatttgctgtctatttccactt

SEQ ID NO: 1541 - RNA, 20 nt, Synthetic construct
  misc_feature 1: note = sGalNAc3
  misc_feature 20: note = invAb
  source 1..20: mol_type = other RNA
ggaaatagacaccaaatctt

SEQ ID NO: 1542 - RNA, 23 nt, Synthetic construct
  source 1..23: mol_type = other RNA
taagatttggtgtctatttcctt

SEQ ID NO: 1543 - RNA, 20 nt, Synthetic construct
  misc_feature 1: note = sGalNAc3
  misc_feature 20: note = invAb
  source 1..20: mol_type = other RNA
ggaaatagacaccatatctt

SEQ ID NO: 1544 - RNA, 23 nt, Synthetic construct
  source 1..23: mol_type = other RNA
taagatatggtgtctatttcctt

SEQ ID NO: 1545 - RNA, 20 nt, Synthetic construct
  misc_feature 1: note = sGalNAc3
  misc_feature 20: note = invAb
  source 1..20: mol_type = other RNA
gaaatagacaccaaatctta

SEQ ID NO: 1546 - RNA, 23 nt, Synthetic construct
  source 1..23: mol_type = other RNA
ataagatttggtgtctatttctt

SEQ ID NO: 1547 - RNA, 20 nt, Synthetic construct
  misc_feature 1: note = sGalNAc3
  misc_feature 20: note = invAb
  source 1..20: mol_type = other RNA
gaaatagacaccaaaactta

SEQ ID NO: 1548 - RNA, 23 nt, Synthetic construct
  source 1..23: mol_type = other RNA
ataagttttggtgtctatttctt

SEQ ID NO: 1549 - RNA, 20 nt, Synthetic construct
  misc_feature 1: note = sGalNAc3
  misc_feature 20: note = invAb
  source 1..20: mol_type = other RNA
gaaatagacaccaattctta

SEQ ID NO: 1550 - RNA, 23 nt, Synthetic construct
  source 1..23: mol_type = other RNA
ataagaattggtgtctatttctt

SEQ ID NO: 1551 - RNA, 20 nt, Synthetic construct
  misc_feature 1: note = sGalNAc3
  misc_feature 20: note = invAb
  source 1..20: mol_type = other RNA
aaatagacaccaaatcttac

SEQ ID NO: 1552 - RNA, 23 nt, Synthetic construct
  source 1..23: mol_type = other RNA
agtaagatttggtgtctattttt

SEQ ID NO: 1553 - RNA, 20 nt, Synthetic construct
  misc_feature 1: note = sGalNAc3
  misc_feature 20: note = invAb
  source 1..20: mol_type = other RNA
aatagacaccaaatcttact

SEQ ID NO: 1554 - RNA, 23 nt, Synthetic construct
  source 1..23: mol_type = other RNA
aagtaagatttggtgtctatttt

SEQ ID NO: 1555 - RNA, 20 nt, Synthetic construct
  misc_feature 1: note = sGalNAc3
  misc_feature 20: note = invAb
  source 1..20: mol_type = other RNA
aatagacaccaaatcatact

SEQ ID NO: 1556 - RNA, 23 nt, Synthetic construct
  source 1..23: mol_type = other RNA
aagtatgatttggtgtctatttt

SEQ ID NO: 1557 - RNA, 20 nt, Synthetic construct
  misc_feature 1: note = sGalNAc3
  misc_feature 20: note = invAb
  source 1..20: mol_type = other RNA
aatagacaccaaaacttact

SEQ ID NO: 1558 - RNA, 23 nt, Synthetic construct
  source 1..23: mol_type = other RNA
aagtaagttttggtgtctatttt

SEQ ID NO: 1559 - RNA, 20 nt, Synthetic construct
  misc_feature 1: note = sGalNAc3
  misc_feature 20: note = invAb
  source 1..20: mol_type = other RNA
atagacaccaaatcttactg

SEQ ID NO: 1560 - RNA, 23 nt, Synthetic construct
  source 1..23: mol_type = other RNA
acagtaagatttggtgtctattt

SEQ ID NO: 1561 - RNA, 20 nt, Synthetic construct
  misc_feature 1: note = sGalNAc3
  misc_feature 20: note = invAb
  source 1..20: mol_type = other RNA
atagacaccaaatcatactg

SEQ ID NO: 1562 - RNA, 23 nt, Synthetic construct
  source 1..23: mol_type = other RNA
acagtatgatttggtgtctattt

SEQ ID NO: 1563 - RNA, 20 nt, Synthetic construct
  misc_feature 1: note = sGalNAc3
  misc_feature 20: note = invAb
  source 1..20: mol_type = other RNA
tagacaccaaatcttactgg

SEQ ID NO: 1564 - RNA, 23 nt, Synthetic construct
  source 1..23: mol_type = other RNA
tccagtaagatttggtgtctatt

SEQ ID NO: 1565 - RNA, 20 nt, Synthetic construct
  misc_feature 1: note = sGalNAc3
  misc_feature 20: note = invAb
  source 1..20: mol_type = other RNA
tagacaccaaatcttactgg

SEQ ID NO: 1566 - RNA, 21 nt, Synthetic construct
  source 1..21: mol_type = other RNA
tccagtaagatttggtgtcta

SEQ ID NO: 1567 - RNA, 18 nt, Synthetic construct
  misc_feature 1: note = sGalNAc3
  misc_feature 18: note = invAb
  source 1..18: mol_type = other RNA
gacaccaaatcttactgg

SEQ ID NO: 1568 - RNA, 21 nt, Synthetic construct
  source 1..21: mol_type = other RNA
tccagtaagatttggtgtctt

SEQ ID NO: 1569 - RNA, 20 nt, Synthetic construct
  misc_feature 1: note = sGalNAc3
  misc_feature 20: note = invAb
  source 1..20: mol_type = other RNA
tagacaccaaatcttactgg

SEQ ID NO: 1570 - RNA, 23 nt, Synthetic construct
  source 1..23: mol_type = other RNA
tccagtaagatttggtgtctatt

SEQ ID NO: 1571 - RNA, 20 nt, Synthetic construct
  misc_feature 1: note = sGalNAc3
  misc_feature 20: note = invAb
  source 1..20: mol_type = other RNA
tagacaccaaatctttctgg

SEQ ID NO: 1572 - RNA, 23 nt, Synthetic construct
  source 1..23: mol_type = other RNA
tccagaaagatttggtgtctatt

SEQ ID NO: 1573 - RNA, 18 nt, Synthetic construct
  misc_feature 1: note = sGalNAc3
  misc_feature 18: note = invAb
  source 1..18: mol_type = other RNA
gacaccaaatcttactgg

SEQ ID NO: 1574 - RNA, 21 nt, Synthetic construct
  source 1..21: mol_type = other RNA
tccagtaagatttggtgtctt

SEQ ID NO: 1575 - RNA, 20 nt, Synthetic construct
  misc_feature 1: note = sGalNAc3
  misc_feature 20: note = invAb
  source 1..20: mol_type = other RNA
agacaccaaatcttactgga

SEQ ID NO: 1576 - RNA, 23 nt, Synthetic construct
  source 1..23: mol_type = other RNA
ttccagtaagatttggtgtcttt

SEQ ID NO: 1577 - RNA, 20 nt, Synthetic construct
  misc_feature 1: note = sGalNAc3
  misc_feature 20: note = invAb
  source 1..20: mol_type = other RNA
agacaccaaatctttctgga

SEQ ID NO: 1578 - RNA, 23 nt, Synthetic construct
  source 1..23: mol_type = other RNA
ttccagaaagatttggtgtcttt

SEQ ID NO: 1579 - RNA, 20 nt, Synthetic construct
  misc_feature 1: note = sGalNAc3
  misc_feature 20: note = invAb
  source 1..20: mol_type = other RNA
agacaccaaatctaactgga

SEQ ID NO: 1580 - RNA, 23 nt, Synthetic construct
  source 1..23: mol_type = other RNA
ttccagttagatttggtgtcttt

SEQ ID NO: 1581 - RNA, 20 nt, Synthetic construct
  misc_feature 1: note = sGalNAc3
  misc_feature 20: note = invAb
  source 1..20: mol_type = other RNA
acttggcatctccccattcc

SEQ ID NO: 1582 - RNA, 23 nt, Synthetic construct
  source 1..23: mol_type = other RNA
tggaatggggagatgccaagttt

SEQ ID NO: 1583 - RNA, 20 nt, Synthetic construct
  misc_feature 1: note = sGalNAc3
  misc_feature 20: note = invAb
  source 1..20: mol_type = other RNA
catctccccattccatgagc

SEQ ID NO: 1584 - RNA, 23 nt, Synthetic construct
  source 1..23: mol_type = other RNA
tgctcatggaatggggagatgtt

SEQ ID NO: 1585 - RNA, 20 nt, Synthetic construct
  misc_feature 1: note = sGalNAc3
  misc_feature 20: note = invAb
  source 1..20: mol_type = other RNA
catctccccattccttgagc

SEQ ID NO: 1586 - RNA, 23 nt, Synthetic construct
  source 1..23: mol_type = other RNA
tgctcaaggaatggggagatgtt

SEQ ID NO: 1587 - RNA, 20 nt, Synthetic construct
  misc_feature 1: note = sGalNAc3
  misc_feature 20: note = invAb
  source 1..20: mol_type = other RNA
catctccccattcgatgagc

SEQ ID NO: 1588 - RNA, 23 nt, Synthetic construct
  source 1..23: mol_type = other RNA
tgctcatcgaatggggagatgtt

SEQ ID NO: 1589 - RNA, 20 nt, Synthetic construct
  misc_feature 1: note = sGalNAc3
  misc_feature 20: note = invAb
  source 1..20: mol_type = other RNA
tccccattccatgtgcatgc

SEQ ID NO: 1590 - RNA, 23 nt, Synthetic construct
  source 1..23: mol_type = other RNA
tgcatgcacatggaatggggatt

SEQ ID NO: 1591 - RNA, 20 nt, Synthetic construct
  misc_feature 1: note = sGalNAc3
  misc_feature 20: note = invAb
  source 1..20: mol_type = other RNA
tccccattccatgtgcatgc

SEQ ID NO: 1592 - RNA, 23 nt, Synthetic construct
  source 1..23: mol_type = other RNA
tgcatgcacatggaatggggaga

SEQ ID NO: 1593 - RNA, 20 nt, Synthetic construct
  misc_feature 1: note = sGalNAc3
  misc_feature 20: note = invAb
  source 1..20: mol_type = other RNA
tccccattccatgtgcatgc

SEQ ID NO: 1594 - RNA, 23 nt, Synthetic construct
  source 1..23: mol_type = other RNA
tgcatgcacatggaatggggaga

SEQ ID NO: 1595 - RNA, 20 nt, Synthetic construct
  misc_feature 1: note = sGalNAc3
  misc_feature 20: note = invAb
  source 1..20: mol_type = other RNA
tccccattccatgtgcatgc

SEQ ID NO: 1596 - RNA, 23 nt, Synthetic construct
  source 1..23: mol_type = other RNA
tgcatgcacatggaatggggaga

SEQ ID NO: 1597 - RNA, 20 nt, Synthetic construct
  misc_feature 1: note = sGalNAc3
  misc_feature 20: note = invAb
  source 1..20: mol_type = other RNA
tccccattccatgtgcatgc

SEQ ID NO: 1598 - RNA, 23 nt, Synthetic construct
  source 1..23: mol_type = other RNA
tgcatgcacatggaatggggaga

SEQ ID NO: 1599 - RNA, 20 nt, Synthetic construct
  misc_feature 1: note = sGalNAc3
  misc_feature 20: note = invAb
  source 1..20: mol_type = other RNA
tccccattccatgtgcatgc

SEQ ID NO: 1600 - RNA, 21 nt, Synthetic construct
  source 1..21: mol_type = other RNA
tgcatgcacatggaatgggga

SEQ ID NO: 1601 - RNA, 18 nt, Synthetic construct
  misc_feature 1: note = sGalNAc3
  misc_feature 18: note = invAb
  source 1..18: mol_type = other RNA
cccattccatgtgcatgc

SEQ ID NO: 1602 - RNA, 21 nt, Synthetic construct
  source 1..21: mol_type = other RNA
tgcatgcacatggaatgggtt

SEQ ID NO: 1603 - RNA, 20 nt, Synthetic construct
  misc_feature 1: note = sGalNAc3
  misc_feature 20: note = invAb
  source 1..20: mol_type = other RNA
tccccattccatgagcatgc

SEQ ID NO: 1604 - RNA, 23 nt, Synthetic construct
  source 1..23: mol_type = other RNA
tgcatgctcatggaatggggatt

SEQ ID NO: 1605 - RNA, 20 nt, Synthetic construct
  misc_feature 1: note = sGalNAc3
  misc_feature 20: note = invAb
  source 1..20: mol_type = other RNA
tccccattccatgtgcatgc

SEQ ID NO: 1606 - RNA, 23 nt, Synthetic construct
  source 1..23: mol_type = other RNA
tgcatgcacatggaatggggatt

SEQ ID NO: 1607 - RNA, 18 nt, Synthetic construct
  misc_feature 1: note = sGalNAc3
  misc_feature 18: note = invAb
  source 1..18: mol_type = other RNA
cccattccatgtgcatgc

SEQ ID NO: 1608 - RNA, 21 nt, Synthetic construct
  source 1..21: mol_type = other RNA
tgcatgcacatggaatgggtt

SEQ ID NO: 1609 - RNA, 20 nt, Synthetic construct
  misc_feature 1: note = sGalNAc3
  misc_feature 20: note = invAb
  source 1..20: mol_type = other RNA
ccccattccatgagcatgca

SEQ ID NO: 1610 - RNA, 23 nt, Synthetic construct
  source 1..23: mol_type = other RNA
atgcatgctcatggaatggggtt

SEQ ID NO: 1611 - RNA, 20 nt, Synthetic construct
  misc_feature 1: note = sGalNAc3
  misc_feature 20: note = invAb
  source 1..20: mol_type = other RNA
ccccattccatgagcatgca

SEQ ID NO: 1612 - RNA, 21 nt, Synthetic construct
  source 1..21: mol_type = other RNA
atgcatgctcatggaatgggg

SEQ ID NO: 1613 - RNA, 18 nt, Synthetic construct
  misc_feature 1: note = sGalNAc3
  misc_feature 18: note = invAb
  source 1..18: mol_type = other RNA
ccattccatgagcatgca

SEQ ID NO: 1614 - RNA, 21 nt, Synthetic construct
  source 1..21: mol_type = other RNA
atgcatgctcatggaatggtt

SEQ ID NO: 1615 - RNA, 20 nt, Synthetic construct
  misc_feature 1: note = sGalNAc3
  misc_feature 20: note = invAb
  source 1..20: mol_type = other RNA
ccccattccatgagcatgca

SEQ ID NO: 1616 - RNA, 23 nt, Synthetic construct
  source 1..23: mol_type = other RNA
atgcatgctcatggaatggggtt

SEQ ID NO: 1617 - RNA, 18 nt, Synthetic construct
  misc_feature 1: note = sGalNAc3
  misc_feature 18: note = invAb
  source 1..18: mol_type = other RNA
ccattccatgagcatgca

SEQ ID NO: 1618 - RNA, 21 nt, Synthetic construct
  source 1..21: mol_type = other RNA
atgcatgctcatggaatggtt

(19) United States
(12) Patent Application Publication
(10) Pub. No.: US 2026/0209759 A1
(43) Pub. Date: Jul. 23, 2026

(54) RNAI CONSTRUCTS FOR INHIBITING TTR EXPRESSION AND METHODS OF USE THEREOF

(71) Applicant: AMGEN INC., Thousand Oaks, CA (US)

(72) Inventors: Ingrid RULIFSON, Palo Alto, CA (US); Bryan MEADE, Westlake Village, CA (US); Jason C. LONG, Newbury Park, CA (US); Justin K. MURRAY, Moorpark, CA (US)

(73) Assignee: AMGEN INC., Thousand Oaks, CA (US)

(21) Appl. No.: 19/139,853

(22) PCT Filed: Dec. 15, 2023
(86) PCT No.: PCT/US2023/084321

§ 371 Date: Jun. 16, 2025

Related U.S. Application Data
(60) Provisional application No. 63/433,263, filed on Dec. 16, 2022.

(51) Int. Cl. C12N 15/113; A61K 31/712; A61K 31/7125; A61K 31/713; A61P 7/00; C12N 15/86

(52) U.S. Cl. CPC C12N 15/113; A61K 31/712; A61K 31/7125; A61K 31/713; A61P 7/00; C12N 15/86; C12N 2310/11; C12N 2310/14; C12N 2310/315; C12N 2310/321; C12N 2310/322; C12N 2750/14143

(57) ABSTRACT

The disclosure relates to RNAi constructs, such as siRNA, for reducing expression of the TTR gene. Methods of using such RNAi constructs to treat or prevent cardiac disease, such as transthyretin-associated cardiomyopathy (ATTR-CM), are also described.

### CROSS-REFERENCE TO RELATED APPLICATIONS

This application claims the benefit of priority to U.S. Provisional Application No. 63/433,263, filed Dec. 16, 2022, which is hereby incorporated by reference in its entirety for all purposes.

### FIELD OF THE INVENTION

The present invention relates to compositions and methods for modulating liver expression of Transthyretin (TTR). In particular, the present invention relates to nucleic acid-based therapeutics for reducing TTR expression via RNA interference and methods of using such nucleic acid-based therapeutics to treat or prevent cardiac disease, such as transthyretin-associated cardiomyopathy (ATTR-CM).

### BACKGROUND OF THE INVENTION

Transthyretin-associated cardiomyopathy (ATTR-CM) is a chronic, progressive disorder subclassified into two types by the sequence of the transthyretin (TTR) gene: hereditary transthyretin amyloid CM (hATTR-CM) and wild type transthyretin amyloid CM (wtATTR-CM). Both TTR mRNA and TTR protein are predominantly made in the liver; secreted TTR circulates as a tetramer functioning as a carrier protein. It is speculated that triggers such as genetic mutations or increased expression in TTR or effects of aging can lead to instability of the secreted tetrameric structure leading to protein misfolding, aggregation and amyloid plaque deposition, resulting in cardiac tissue dysfunction and heart failure. A GalNac-conjugated small interfering RNA (siRNA) that silences TTR mRNA, and thus knocks down TTR protein production, is expected to have positive outcomes in patients with ATTR-CM, including reduced cardiovascular-related mortality and cardiovascular-related hospitalization.

Amyloidosis is a disorder characterized by misfolded precursor proteins that form depositions of fibrillar aggregates, known as amyloid, in the extracellular space of tissues including the heart, kidney, brain, pancreas, and liver. Cardiac amyloidosis (CA) is a complex form of restrictive cardiomyopathy with high morbidity and mortality. CA typically arises from either misfolded transthyretin (TTR), described as ATTR amyloidosis, or immunoglobulin light-chain aggregation, described as AL amyloidosis.

ATTR-cardiomyopathy (CM) is a chronic, progressive disorder subclassified by the sequence of the TTR gene: hereditary transthyretin amyloid CM (hATTR-CM), a rare disease driven by the presence of a genetic mutation and associated with both cardiomyopathy and polyneuropathy (ATTR-PN), and age-related wild type transthyretin amyloid CM (wtATTR-CM), which manifests predominantly as cardiomyopathy. Secreted from the liver (though also produced in the pancreas, choroid plexus, and retinal pigmented epithelium) into the blood, TTR is composed of four β-sheet monomers that circulate as a tetramer, serving as a carrier protein for thyroxine and holo-retinol binding protein (RBP). Both the hereditary and wild type forms of ATTR-CM result from kinetic instability of the secreted TTR tetramers. TTR misfolding, aggregation/deposition and direct myocardial toxicity contribute to cardiac tissue dysfunction and clinical phenotypes associated with heart failure. Over the last several years, an increasing number of studies identified wtATTR-CM as a common cause of heart failure with preserved ejection fraction (HFpEF). Conservative estimates indicate that wtATTR-CM plays a prominent pathogenic role in at least 8-13% of HFpEF patients; in the U.S. alone, this represents >200,000 patients, which is more prevalent than multiple myeloma. Associations with wtATTR-CM have also been observed for carpal tunnel syndrome (CTS), spontaneous biceps tendon rupture and lumbar spine stenosis, and aortic stenosis (AS); among the latter, 6%-15% of the entire AS population and ≤30% of the subset with low-flow, low-gradient pattern may have ATTR-CM.

Historically, ATTR-CM was underdiagnosed but advances in human genetics and imaging techniques are transforming both diagnosis rate and accuracy. For example, for hATTR, V122I is the most frequent TTR mutation in the U.S., observed almost exclusively in Black Americans, of which approximately 3.4% carry at least one copy of the variant allele. But the prevalence of ATTR-CM in Black Americans is unclear and traditional diagnostics, like endomyocardial biopsy, are impractical. Using Tc99-pyrophosphate (PYP) imaging, a highly accurate, non-biopsy strategy for ATTR-CM detection, a clinical trial is on-going to establish the true prevalence of ATTR-CM in elderly Black Americans and Hispanics; early studies using PYP suggest 10-15% of elderly patients with heart failure have ATTR-CM. The trial also includes testing the serum biomarker, RBP4, for its accuracy and relationship to TTR stability. Researchers are also uncovering prevalence rates of ATTR-CM with comorbidities, such as carpal tunnel syndrome. A study from the Cleveland Clinic reported that 10% of patients undergoing carpal tunnel release surgery exhibit amyloid deposits in tenosynovial tissue, thus concomitant cardiac evaluation may increase ATTR-CM diagnosis. Therefore, having once been considered rare due to underdiagnosis, ATTR-CM is on the cusp of being designated a viable therapeutic target for the greater, more heterogeneous patient populations.

Thus, there is a need for compositions and methods that treat cardiac disorders, such as transthyretin-associated cardiomyopathy (ATTR-CM) in affected individuals.

### SUMMARY OF THE INVENTION

The present disclosure provides an RNAi construct comprising a sense strand and an antisense strand, wherein the RNAi construct inhibits the expression of a Transthyretin (TTR) mRNA. In certain embodiments, the RNAi construct comprises a region having at least 15 contiguous nucleotides differing by no more than 3 nucleotides from an antisense sequence selected from the group consisting of: SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, and 1132, and wherein the RNAi construct inhibits the expression of a Transthyretin (TTR) mRNA.

In some embodiments, the sense strand of the RNAi constructs described herein comprises a sequence that is sufficiently complementary to the sequence of the antisense strand to form a duplex region of about 15 to about 30 base pairs in length. In these and other embodiments, the sense and antisense strands each are about 15 to about 30 nucleotides in length. In some embodiments, the RNAi constructs comprise at least one blunt end. In other embodiments, the RNAi constructs comprise at least one nucleotide overhang. Such nucleotide overhangs may comprise at least 1 to 6 unpaired nucleotides and can be located at the 3′ end of the sense strand, the 3′ end of the antisense strand, or the 3′ end of both the sense and antisense strand. In certain embodiments, the RNAi constructs comprise an overhang of two unpaired nucleotides at the 3′ end of the sense strand and the 3′ end of the antisense strand. In other embodiments, the RNAi constructs comprise an overhang of two unpaired nucleotides at the 3′ end of the antisense strand and a blunt end of the 3′ end of the sense strand/5′ end of the antisense strand.

The RNAi constructs of the invention may comprise one or more modified nucleotides, including nucleotides having modifications to the ribose ring, nucleobase, or phosphodiester backbone. In some embodiments, the RNAi constructs comprise one or more 2′-modified nucleotides. Such 2′-modified nucleotides can include 2′-fluoro modified nucleotides, 2′-O-methyl modified nucleotides, 2′-O-methoxyethyl modified nucleotides, 2′-O-allyl modified nucleotides, bicyclic nucleic acids (BNA), glycol nucleic acids (GNAs), inverted bases (e.g. inverted adenosine) or combinations thereof. In one particular embodiment, the RNAi constructs comprise one or more 2′-fluoro modified nucleotides, 2′-O-methyl modified nucleotides, or combinations thereof. In some embodiments, all of the nucleotides in the sense and antisense strand of the RNAi construct are modified nucleotides.

In some embodiments, the RNAi constructs comprise at least one backbone modification, such as a modified internucleotide or internucleoside linkage. In certain embodiments, the RNAi constructs described herein comprise at least one phosphorothioate internucleotide linkage. In particular embodiments, the phosphorothioate internucleotide linkages may be positioned at the 3′ or 5′ ends of the sense and/or antisense strands.

The disclosure also provides a composition comprising the aforementioned RNAi construct and a pharmaceutically acceptable carrier, excipient, or diluent, as well as methods of reducing the expression of TTR in a patient in need thereof comprising administering to the patient the aforementioned RNAi construct or composition.

### DETAILED DESCRIPTION

The present invention is based, in part, on the design and generation of RNAi constructs that target the Transthyretin (TTR) gene and reduce expression of TTR in liver cells. The inhibition of TTR expression is useful for treating or preventing conditions associated with TTR expression, including cardiac disorders, such as transthyretin-associated cardiomyopathy (ATTR-CM).

The disclosure provides compositions and methods for regulating the expression of the gene encoding TTR. In some embodiments, the gene may be within a cell or subject, such as a mammal (e.g., a human). In some embodiments, compositions of the invention comprise RNAi constructs that target a TTR mRNA and reduce TTR expression in a cell or mammal. Such RNAi constructs are useful for treating or preventing various forms of cardiac disorders, such as transthyretin-associated cardiomyopathy (ATTR-CM).

RNA interference (RNAi) is the process of introducing exogeneous RNA into a cell leading to specific degradation of the mRNA encoding the targeted protein with a resultant decrease in protein expression. Advances in both the RNAi technology and hepatic delivery, as well as growing positive outcomes with other RNAi-based therapies, suggest RNAi as a compelling means to therapeutically treat transthyretin-associated cardiomyopathy by directly targeting genes that regulate lipid synthesis in liver, such as TTR.

As used herein, the term “RNAi construct” refers to an agent comprising an RNA molecule that is capable of downregulating expression of a target gene (e.g., TTR) via an RNA interference mechanism when introduced into a cell. “RNA interference” is the process by which a nucleic acid molecule induces the cleavage and degradation of a target RNA molecule (e.g. messenger RNA or mRNA molecule) in a sequence-specific manner, e.g. through an RNA induced silencing complex (RISC) pathway. In some embodiments, the RNAi construct comprises a double-stranded RNA (dsRNA) molecule comprising two antiparallel strands of contiguous nucleotides that are sufficiently complementary to each other to hybridize to form a duplex region. A double-stranded RNAi construct also may be referred to as an RNAi “trigger.” The terms “hybridize” or “hybridization” refer to the pairing of complementary polynucleotides, typically via hydrogen bonding (e.g., Watson-Crick, Hoogsteen or reversed Hoogsteen hydrogen bonding) between complementary bases in the two polynucleotides. The strand comprising a region having a sequence that is substantially complementary to a target sequence (e.g., target mRNA) is referred to as the “antisense strand.” The “sense strand” refers to the strand that includes a region that is substantially complementary to a region of the antisense strand. In some embodiments, the sense strand may comprise a region that has a sequence that is substantially identical to the target sequence.

In certain embodiments, the sense strand and antisense strand of the double-stranded RNA may be two separate molecules that hybridize to form a duplex region but are otherwise unconnected. Such double-stranded RNA molecules formed from two separate strands are referred to as “small interfering RNAs” or “short interfering RNAs” (siRNAs). siRNAs are a class of non-coding, double-stranded RNA molecules that are typically about 20-27 base pairs and are central to RNAi. Thus, in some embodiments, the RNAi constructs of the invention comprise an siRNA. In other embodiments, the RNAi construct may be a microRNA (also known as “miRNA” or “mature miRNA”). miRNAs are small (approximately 18-24 nucleotides in length), non-coding RNA molecules present in plants, animals, and some viruses. miRNAs resemble siRNA, but miRNAs originate from hairpin mRNA structures. miRNAs regulate gene expression by base-pairing to complementary regions of target mRNAs.

In some embodiments, the disclosure provides an RNAi construct directed to TTR. In some embodiments, the RNAi construct is an siRNA that comprises a sense strand and an antisense strand, wherein the antisense strand comprises a region that is complementary to TTR mRNA sequence. The region of the RNAi antisense strand may be complementary to any suitable region of a TTR mRNA sequence. For example, the antisense strand may comprise a region that is complementary to the coding region or the 3′ untranslated region (UTR) of a TTR mRNA sequence

A double-stranded RNAi molecule may include chemical modifications to ribonucleotides, including modifications to the ribose sugar, base, or backbone components of the ribonucleotides, such as those described herein or known in the art. Any such modifications, as used in a double-stranded RNA molecule (e.g. siRNA, shRNA, or the like), are encompassed by the term “double-stranded RNA” for the purposes of this disclosure.

As used herein, a first sequence is “complementary” to a second sequence if a polynucleotide comprising the first sequence can hybridize to a polynucleotide comprising the second sequence to form a duplex region under certain conditions, such as physiological conditions. Other such conditions can include moderate or stringent hybridization conditions, which are known to those of skill in the art. A first sequence is considered to be fully complementary (100% complementary) to a second sequence if a polynucleotide comprising the first sequence base pairs with a polynucleotide comprising the second sequence over the entire length of one or both nucleotide sequences without any mismatches. A sequence is “substantially complementary” to a target sequence if the sequence is at least about 80%, 85%, 90%, 95%, 96%, 97%, 98%, 99% or 100% complementary to a target sequence. Percent complementarity can be calculated by dividing the number of bases in a first sequence that are complementary to bases at corresponding positions in a second or target sequence by the total length of the first sequence. A sequence may also be said to be substantially complementary to another sequence if there are no more than 5, 4, 3, 2, or 1 mismatch over a 30 base pair duplex region when the two sequences are hybridized. Generally, if any nucleotide overhangs, as defined herein, are present, the sequence of such overhangs is not considered in determining the degree of complementarity between two sequences. By way of example, a sense strand of 21 nucleotides in length and an antisense strand of 21 nucleotides in length that hybridize to form a 19 base pair duplex region with a 2-nucleotide overhang at the 3′ end of each strand would be considered to be fully complementary as the term is used herein.

In some embodiments, a region of the antisense strand comprises a sequence that is fully complementary to a region of the target RNA sequence (e.g., TTR mRNA). In such embodiments, the sense strand may comprise a sequence that is fully complementary to the sequence of the antisense strand. In other such embodiments, the sense strand may comprise a sequence that is substantially complementary to the sequence of the antisense strand, e.g., having 1, 2, 3, 4, or 5 mismatches in the duplex region formed by the sense and antisense strands. In certain embodiments, it is preferred that any mismatches occur within the terminal regions (e.g. within 6, 5, 4, 3, 2, or 1 nucleotides of the 5′ and/or 3′ ends of the strands). In one embodiment, any mismatches in the duplex region formed from the sense and antisense strands desirably occur within 6, 5, 4, 3, 2, or 1 nucleotides of the 5′ end of the antisense strand.

Where the two substantially complementary strands of a dsRNA are comprised of separate RNA molecules, those molecules need not, but can be, covalently connected. Where the two strands are connected covalently by means other than an uninterrupted chain of nucleotides between the 3 ‘-end of one strand and the 5’-end of the respective other strand forming the duplex structure, the connecting structure is referred to as a “linker.” The RNA strands may have the same or a different number of nucleotides. The maximum number of base pairs in the duplex is the number of nucleotides in the shortest strand of the dsRNA minus any overhangs that are present in the duplex. In addition to the duplex structure, an RNAi may comprise one or more nucleotide overhangs.

In other embodiments, the sense strand and the antisense strand that hybridize to form a duplex region may be part of a single RNA molecule, i.e., the sense and antisense strands are part of a self-complementary region of a single RNA molecule. In such cases, a single RNA molecule comprises a duplex region (also referred to as a stem region) and a loop region. The 3′ end of the sense strand is connected to the 5′ end of the antisense strand by a contiguous sequence of unpaired nucleotides, which will form the loop region. The loop region is typically of a sufficient length to allow the RNA molecule to fold back on itself such that the antisense strand can base pair with the sense strand to form the duplex or stem region. The loop region can comprise from about 3 to about 25, from about 5 to about 15, or from about 8 to about 12 unpaired nucleotides. As noted herein, such RNA molecules with at least partially self-complementary regions are referred to as “short hairpin RNAs” (shRNAs). In some embodiments, the loop region can comprise at least 1, 2, 3, 4, 5, 10, 20, or 25 unpaired nucleotides. In other embodiments, the loop region can have 10, 9, 8, 7, 6, 5, 4, 3, 2, or fewer unpaired nucleotides. In certain embodiments, the RNAi constructs of the invention comprise an shRNA. The length of a single, at least partially self-complementary RNA molecule can be from about 35 nucleotides to about 100 nucleotides, from about 45 nucleotides to about 85 nucleotides, or from about 50 to about 60 nucleotides and comprise a duplex region and loop region each having the lengths recited herein.

In some embodiments, the RNAi constructs of the invention comprise a sense strand and an antisense strand, wherein the antisense strand comprises a region having a sequence that is substantially or fully complementary to a TTR messenger RNA (mRNA) sequence. As used herein, a “TTR mRNA sequence” refers to any messenger RNA sequence, including splice variants, encoding a TTR protein, including TTR protein variants or isoforms from any species (e.g. mouse, rat, non-human primate, human). TTR also is known in the art as SREBF chaperone.

A TTR mRNA sequence also includes the transcript sequence expressed as its complementary DNA (cDNA) sequence. A cDNA sequence refers to the sequence of an mRNA transcript expressed as DNA bases (e.g. guanine, adenine, thymine, and cytosine) rather than RNA bases (e.g. guanine, adenine, uracil, and cytosine). Thus, the antisense strand of the RNAi constructs of the invention may comprise a region having a sequence that is substantially or fully complementary to a target TTR mRNA sequence or TTR cDNA sequence. A TTR mRNA or cDNA sequence can include, but is not limited to, any TTR mRNA or cDNA sequence such as can be derived from the NCBI Reference sequence NM_001320044.2 or NM_012235.4.

A region of the antisense strand can be substantially complementary or fully complementary to at least 15 consecutive nucleotides of the TTR mRNA sequence. In some embodiments, the target region of the TTR mRNA sequence to which the antisense strand comprises a region of complementarity can range from about 15 to about 30 consecutive nucleotides, from about 16 to about 28 consecutive nucleotides, from about 18 to about 26 consecutive nucleotides, from about 17 to about 24 consecutive nucleotides, from about 19 to about 25 consecutive nucleotides, from about 19 to about 23 (e.g., 19, 20, 21, 22, or 23) consecutive nucleotides, or from about 19 to about 21 consecutive nucleotides. In certain embodiments, the region of the antisense strand comprising a sequence that is substantially or fully complementary to a TTR mRNA sequence may, in some embodiments, comprise at least 19 contiguous nucleotides from an antisense sequence listed in Table 1. In some embodiments, the sense and/or antisense sequence comprises at least 15 nucleotides from a sequence listed in Table 1 with no more than 1, 2, or 3 nucleotide mismatches.

The sense strand of the RNAi construct typically comprises a sequence that is sufficiently complementary to the sequence of the antisense strand such that the two strands hybridize under physiological conditions to form a duplex region. A “duplex region” refers to the region in two complementary or substantially complementary polynucleotides that form base pairs with one another, either by Watson-Crick base pairing or other hydrogen bonding interaction, to create a duplex between the two polynucleotides. The duplex region of the RNAi construct should be of sufficient length to allow the RNAi construct to enter the RNA interference pathway, e.g. by engaging the Dicer enzyme and/or the RISC complex (described below). For instance, in some embodiments, the duplex region is about 15 to about 30 base pairs in length. Other lengths for the duplex region within this range are also suitable, such as about 15 to about 28 base pairs, about 15 to about 26 base pairs, about 15 to about 24 base pairs, about 15 to about 22 base pairs, about 17 to about 28 base pairs, about 17 to about 26 base pairs, about 17 to about 24 base pairs, about 17 to about 23 base pairs, about 17 to about 21 base pairs, about 19 to about 25 base pairs, about 19 to about 23 base pairs, or about 19 to about 21 base pairs. In one embodiment, the duplex region is about 17 to about 24 base pairs in length. In another embodiment, the duplex region is about 19 to about 21 base pairs in length. For example, the duplex region may be about 19 base pairs in length.

In some embodiments, an RNAi construct of the invention contains a duplex region of about 17 to about 24 nucleotides that interacts with a target RNA sequence, e.g., a TTR target mRNA sequence, to direct the cleavage of the target RNA. Without wishing to be bound by theory, long double-stranded RNA introduced into cells can be broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell, 107:309). Upon binding to the appropriate target mRNA, one or more endonucleases within the RISC cleave the target to induce silencing (Elbashir, et al., (2001) Genes Dev. 15:188).

For embodiments in which the sense strand and antisense strand are two separate molecules (e.g., an siRNA RNAi construct), the sense strand and antisense strand need not be the same length as the length of the duplex region. For instance, one or both strands maybe longer than the duplex region and have one or more unpaired nucleotides or mismatches flanking the duplex region. Thus, in some embodiments, the RNAi construct comprises at least one nucleotide overhang. As used herein, a “nucleotide overhang” refers to the unpaired nucleotide or nucleotides that extend beyond the duplex region at the terminal ends of the strands. Nucleotide overhangs are typically created when the 3′ end of one strand extends beyond the 5′ end of the other strand or when the 5′ end of one strand extends beyond the 3′ end of the other strand. The length of a nucleotide overhang generally is between 1 and 6 nucleotides, 1 and 5 nucleotides, 1 and 4 nucleotides, 1 and 3 nucleotides, 2 and 6 nucleotides, 2 and 5 nucleotides, or 2 and 4 nucleotides. In some embodiments, the nucleotide overhang comprises 1, 2, 3, 4, 5, or 6 nucleotides. In one particular embodiment, the nucleotide overhang comprises 1 to 4 nucleotides. In certain embodiments, the nucleotide overhang comprises 2 nucleotides. The nucleotides in the overhang can be ribonucleotides, deoxyribonucleotides, or modified nucleotides as described herein. In some embodiments, the overhang comprises a 5′-uridineuridine-3′ (5′-UU-3′) dinucleotide. In such embodiments, the UU dinucleotide may comprise ribonucleotides or modified nucleotides, e.g., 2′-modified nucleotides. In other embodiments, the overhang comprises a 5′-deoxythymidine-deoxythymidine-3′ (5′-dTdT-3′) dinucleotide.

The nucleotide overhang can be at the 5′ end or 3′ end of one or both strands. For example, in one embodiment, the RNAi construct comprises a nucleotide overhang at the 5′ end and the 3′ end of the antisense strand. In another embodiment, the RNAi construct comprises a nucleotide overhang at the 5′ end and the 3′ end of the sense strand. In some embodiments, the RNAi construct comprises a nucleotide overhang at the 5′ end of the sense strand and the 5′ end of the antisense strand. In other embodiments, the RNAi construct comprises a nucleotide overhang at the 3′ end of the sense strand and the 3′ end of the antisense strand.

The RNAi constructs may comprise a single nucleotide overhang at one end of the double-stranded RNA molecule and a blunt end at the other. A “blunt end” means that the sense strand and antisense strand are fully base-paired at the end of the molecule and there are no unpaired nucleotides that extend beyond the duplex region. In some embodiments, the RNAi construct comprises a nucleotide overhang at the 3′ end of the sense strand and a blunt end at the 5′ end of the sense strand and 3′ end of the antisense strand. In other embodiments, the RNAi construct comprises a nucleotide overhang at the 3′ end of the antisense strand and a blunt end at the 5′ end of the antisense strand and the 3′ end of the sense strand. In certain embodiments, the RNAi construct comprises a blunt end at both ends of the double-stranded RNA molecule. In such embodiments, the sense strand and antisense strand have the same length and the duplex region is the same length as the sense and antisense strands (i.e., the molecule is double-stranded over its entire length).

The sense strand and antisense strand can each independently be any suitable length, such as about 15 to about 30 nucleotides in length, about 18 to about 28 nucleotides in length, about 19 to about 27 nucleotides in length, about 19 to about 25 nucleotides in length, about 19 to about 23 nucleotides in length, about 21 to about 25 nucleotides in length, or about 21 to about 23 nucleotides in length. In certain embodiments, the sense strand and antisense strand are each about 18, about 19, about 20, about 21, about 22, about 23, about 24, or about 25 nucleotides in length. In some embodiments, the sense strand and antisense strand are of the same length but form a duplex region that is shorter than the strands such that the RNAi construct has two nucleotide overhangs. For instance, in one embodiment, the RNAi construct comprises (i) a sense strand and an antisense strand that are each 21 nucleotides in length, (ii) a duplex region that is 19 base pairs in length, and (iii) nucleotide overhangs of 2 unpaired nucleotides at both the 3′ end of the sense strand and the 3′ end of the antisense strand. In another embodiment, the RNAi construct comprises (i) a sense strand and an antisense strand that are each 23 nucleotides in length, (ii) a duplex region that is 21 base pairs in length, and (iii) nucleotide overhangs of 2 unpaired nucleotides at both the 3′ end of the sense strand and the 3′ end of the antisense strand. In other embodiments, the sense strand and antisense strand have the same length and form a duplex region over their entire length such that there are no nucleotide overhangs on either end of the double-stranded molecule. In one such embodiment, the RNAi construct is blunt ended and comprises (i) a sense strand and an antisense strand, each of which is 21 nucleotides in length, and (ii) a duplex region that is 21 base pairs in length. In another embodiment, the RNAi construct is blunt ended and comprises (i) a sense strand and an antisense strand, each of which is 23 nucleotides in length, and (ii) a duplex region that is 23 base pairs in length.

In other embodiments, the sense strand or the antisense strand is longer than the other strand and the two strands form a duplex region having a length equal to that of the shorter strand such that the RNAi construct comprises at least one nucleotide overhang. For example, in one embodiment, the RNAi construct comprises (i) a sense strand that is 19 nucleotides in length, (ii) an antisense strand that is 21 nucleotides in length, (iii) a duplex region of 19 base pairs in length, and (iv) a single nucleotide overhang of 2 unpaired nucleotides at the 3′ end of the antisense strand. In another embodiment, the RNAi construct comprises (i) a sense strand that is 21 nucleotides in length, (ii) an antisense strand that is 23 nucleotides in length, (iii) a duplex region of 21 base pairs in length, and (iv) a single nucleotide overhang of 2 unpaired nucleotides at the 3′ end of the antisense strand.

The antisense strand of the RNAi constructs of the invention can comprise the sequence of any one of the antisense sequences listed in Table 1 or the sequence of nucleotides 1-21 of any of these antisense sequences. Each of the antisense sequences listed in Table 1 comprises a sequence of 19 consecutive nucleotides (first 19 nucleotides counting from the 5′ end) that is complementary to a TTR mRNA sequence plus a two-nucleotide overhang sequence. Thus, in some embodiments, the antisense strand comprises a sequence of nucleotides 1-21 of any one of SEQ ID NOs: 148-294.

### Modified Nucleotides

The RNAi constructs of the invention may comprise one or more modified nucleotides. A “modified nucleotide” refers to a nucleotide that has one or more chemical modifications to the nucleoside, nucleobase, pentose ring, or phosphate group. As used herein, modified nucleotides do not encompass ribonucleotides containing adenosine monophosphate, guanosine monophosphate, uridine monophosphate, and cytidine monophosphate, and deoxyribonucleotides containing deoxyadenosine monophosphate, deoxyguanosine monophosphate, deoxythymidine monophosphate, and deoxycytidine monophosphate. However, the RNAi constructs may comprise combinations of modified nucleotides, ribonucleotides, and deoxyribonucleotides. Incorporation of modified nucleotides into one or both strands of double-stranded RNA molecules can improve the in vivo stability of the RNA molecules, e.g., by reducing the molecules' susceptibility to nucleases and other degradation processes. The potency of RNAi constructs for reducing expression of the target gene can also be enhanced by incorporation of modified nucleotides.

In certain embodiments, the modified nucleotides have a modification of the ribose sugar. These sugar modifications can include modifications at the 2′ and/or 5′ position of the pentose ring as well as bicyclic sugar modifications. A 2′-modified nucleotide refers to a nucleotide having a pentose ring with a substituent at the 2′ position other than H or OH. Such 2′ modifications include, but are not limited to, 2′-O-alkyl (e.g. O—C1-C10 or O—C1-C10 substituted alkyl), 2′-O-allyl (O—CH₂CH═CH²), 2′-C-allyl, 2′-fluoro, 2′-O-methyl (OCH₃), 2′-O-methoxyethyl (O—(CH₂)2OCH₃), 2′-OCF₃, 2′-O(CH₂)₂SCH₃, 2′-O-aminoalkyl, 2′-amino (e.g., NH₂), 2′-O-ethylamine, and 2′-azido. Modifications at the 5′ position of the pentose ring include, but are not limited to, 5′-methyl (R or S); 5′-vinyl, and 5′-methoxy.

A “bicyclic sugar modification” refers to a modification of the pentose ring where a bridge connects two atoms of the ring to form a second ring resulting in a bicyclic sugar structure. In some embodiments, the bicyclic sugar modification comprises a bridge between the 4′ and 2′ carbons of the pentose ring. Nucleotides comprising a sugar moiety with a bicyclic sugar modification are referred to herein as “bicyclic nucleic acids” or “BNAs.” Exemplary bicyclic sugar modifications include, but are not limited to, α-L-Methyleneoxy (4′-CH₂—O-2′) bicyclicnucleic acid (BNA); β-D-Methyleneoxy (4′-CH₂—O-2′) BNA (also referred to as a locked nucleic acid or LNA); Ethyleneoxy (4′-(CH₂)2-O-2′) BNA; Aminooxy (4′-CH₂—O—N(R)-2′) BNA; Oxyamino (4′-CH₂—N(R)—O-2′) BNA; Methyl(methyleneoxy) (4′-CH(CH₃)—O-2′) BNA (also referred to as constrained ethyl or cEt); methylene-thio (4′-CH₂—S-2′) BNA; methylene-amino (4′-CH₂—N(R)-2′) BNA; methyl carbocyclic (4′-CH₂—CH(CH3)-2′) BNA; propylene carbocyclic (4′-(CH₂)3-2′) BNA; and Methoxy (ethyleneoxy) (4′-CH(CH₂OMe)-O-2′)BNA (also referred to as constrained MOE or cMOE). These and other sugar-modified nucleotides that can be incorporated into the RNAi constructs of the invention are described in, e.g., U.S. Pat. No. 9,181,551, U.S. Patent Publication No. 2016/0122761, and Deleavey and Damha, Chemistry and Biology, 19:937-954 (2012).

In some embodiments, the RNAi constructs comprise one or more 2′-fluoro modified nucleotides, 2′-O-methyl modified nucleotides, 2′-O-methoxyethyl modified nucleotides, 2′-O-allyl modified nucleotides, bicyclic nucleic acids (BNAs), or combinations thereof. In certain embodiments, the RNAi constructs comprise one or more 2′-fluoro modified nucleotides, 2′-O-methyl modified nucleotides, 2′-O-methoxyethyl modified nucleotides, or combinations thereof. In one particular embodiment, the RNAi constructs comprise one or more 2′-fluoro modified nucleotides, 2′-O-methyl modified nucleotides, or combinations thereof.

Both the sense and antisense strands of the RNAi constructs can comprise one or multiple modified nucleotides. For instance, in some embodiments, the sense strand comprises 1, 2, 3, 4, 5, 6, 7, 8, 9, 10 or more modified nucleotides. In certain embodiments, all nucleotides in the sense strand are modified nucleotides. In some embodiments, the antisense strand comprises 1, 2, 3, 4, 5, 6, 7, 8, 9, 10 or more modified nucleotides. In other embodiments, all nucleotides in the antisense strand are modified nucleotides. In certain other embodiments, all nucleotides in the sense strand and all nucleotides in the antisense strand are modified nucleotides. In these and other embodiments, the modified nucleotides can be 2′-fluoro modified nucleotides, 2′-O-methyl modified nucleotides, or combinations thereof.

In some embodiments, all pyrimidine nucleotides preceding an adenosine nucleotide in the sense strand and/or in the antisense strand are modified nucleotides. For example, where the sequence 5′-CA-3′ or 5′-UA-3′ appears in either strand, the cytidine and uridine nucleotides are modified nucleotides, preferably 2′-O-methyl modified nucleotides. In certain embodiments, all pyrimidine nucleotides in the sense strand are modified nucleotides (e.g. 2′-O-methyl modified nucleotides), and the 5′ nucleotide in all occurrences of the sequence 5′-CA-3′ or 5′-UA-3′ in the antisense strand are modified nucleotides (e.g. 2′-O-methyl modified nucleotides). In other embodiments, all nucleotides in the duplex region are modified nucleotides. In such embodiments, the modified nucleotides are preferably 2′-O-methyl modified nucleotides, 2′-fluoro modified nucleotides, or combinations thereof.

In embodiments in which the RNAi construct comprises a nucleotide overhang, the nucleotides in the overhang can be ribonucleotides, deoxyribonucleotides, or modified nucleotides. In one embodiment, the nucleotides in the overhang are deoxyribonucleotides, e.g., deoxythymidine. In another embodiment, the nucleotides in the overhang are modified nucleotides. For instance, in some embodiments, the nucleotides in the overhang are 2′-O-methyl modified nucleotides, 2′-fluoro modified nucleotides, 2′-methoxyethyl modified nucleotides, or combinations thereof.

The RNAi constructs of the disclosure may also comprise one or more modified internucleotide linkages. As used herein, the term “modified internucleotide linkage” refers to an internucleotide linkage other than the natural 3′ to 5′ phosphodiester linkage. In some embodiments, the modified internucleotide linkage is a phosphorous-containing internucleotide linkage, such as a phosphotriester, an aminoalkyl phosphotriester, an alkylphosphonate (e.g., methylphosphonate, 3′-alkylene phosphonate), a phosphinate, a phosphoramidate (e.g., 3′-aminophosphoramidate and aminoalkylphosphoramidate), a phosphorothioate (P═S), a chiralphosphorothioate, a phosphorodithioate, a thionophosphoramidate, a thionoalkylphosphonate, athionoalkylphosphotriester, and a boranophosphate. In one embodiment, a modified internucleotide linkage is a 2′ to 5′ phosphodiester linkage. In other embodiments, the modified internucleotide linkage is a non-phosphorous-containing internucleotide linkage and thus can be referred to as a modified internucleoside linkage. Such non-phosphorous-containing linkages include, but are not limited to, morpholino linkages (formed in part from the sugar portion of a nucleoside); siloxane linkages (—O—Si(H)₂—O—); sulfide, sulfoxide and sulfone linkages; formacetyl and thioformacetyl linkages; alkene containing backbones; sulfamate backbones; methylenemethylimino (—CH₂—N(CH₃)—O—CH₂—) and methylenehydrazino linkages; sulfonate and sulfonamide linkages; amide linkages; and others having mixed N, O, S and CH₂ component parts. In one embodiment, the modified internucleoside linkage is a peptide-based linkage (e.g., aminoethylglycine) to create a peptide nucleic acid or PNA, such as those described in U.S. Pat. Nos. 5,539,082; 5,714,331; and 5,719,262. Other suitable modified internucleotide and internucleoside linkages that may be employed in the disclosed RNAi constructs are described in U.S. Pat. Nos. 6,693,187 and 9,181,551, U.S. Patent Publication No. 2016/0122761, and Deleavey and Damha, supra.

In certain embodiments, the RNAi constructs comprise one or more phosphorothioate internucleotide linkages. The phosphorothioate internucleotide linkages may be present in the sense strand, antisense strand, or both strands of the RNAi constructs. For instance, in some embodiments, the sense strand comprises 1, 2, 3, 4, 5, 6, 7, 8, or more phosphorothioate internucleotide linkages. In other embodiments, the antisense strand comprises 1, 2, 3, 4, 5, 6, 7, 8, or more phosphorothioate internucleotide linkages. In still other embodiments, both strands comprise 1, 2, 3, 4, 5, 6, 7, 8, or more phosphorothioate internucleotide linkages. The RNAi constructs can comprise one or more phosphorothioate internucleotide linkages at the 3′-end, the 5′-end, or both the 3′- and 5′-ends of the sense strand, the antisense strand, or both strands. For instance, in certain embodiments, the RNAi construct comprises about 1 to about 6 or more (e.g., about 1, 2, 3, 4, 5, 6 or more) consecutive phosphorothioate internucleotide linkages at the 3′-end of the sense strand, the antisense strand, or both strands. In other embodiments, the RNAi construct comprises about 1 to about 6 or more (e.g., about 1, 2, 3, 4, 5, 6 or more) consecutive phosphorothioate internucleotide linkages at the 5′-end of the sense strand, the antisense strand, or both strands. In one embodiment, the RNAi construct comprises a single phosphorothioate internucleotide linkage at the 3′ end of the sense strand and a single phosphorothioate internucleotide linkage at the 3′ end of the antisense strand. In another embodiment, the RNAi construct comprises two consecutive phosphorothioate internucleotide linkages at the 3′ end of the antisense strand (i.e., a phosphorothioate internucleotide linkage at the first and second internucleotide linkages at the 3′ end of the antisense strand). In another embodiment, the RNAi construct comprises two consecutive phosphorothioate internucleotide linkages at both the 3′ and 5′ ends of the antisense strand. In yet another embodiment, the RNAi construct comprises two consecutive phosphorothioate internucleotide linkages at both the 3′ and 5′ ends of the antisense strand and two consecutive phosphorothioate internucleotide linkages at the 5′ end of the sense strand. In still another embodiment, the RNAi construct comprises two consecutive phosphorothioate internucleotide linkages at both the 3′ and 5′ ends of the antisense strand and two consecutive phosphorothioate internucleotide linkages at both the 3′ and 5′ ends of the sense strand (i.e. a phosphorothioate internucleotide linkage at the first and second internucleotide linkages at both the 5′ and 3′ ends of the antisense strand and a phosphorothioate internucleotide linkage at the first and second internucleotide linkages at both the 5′ and 3′ ends of the sense strand). In any of the embodiments in which one or both strands comprise one or more phosphorothioate internucleotide linkages, the remaining internucleotide linkages within the strands can be the natural 3′ to 5′ phosphodiester linkages. For instance, in some embodiments, each internucleotide linkage of the sense and antisense strands is selected from phosphodiester and phosphorothioate, wherein at least one internucleotide linkage is a phosphorothioate.

In embodiments in which the RNAi construct comprises a nucleotide overhang, two or more of the unpaired nucleotides in the overhang can be connected by a phosphorothioate internucleotide linkage. In certain embodiments, all the unpaired nucleotides in a nucleotide overhang at the 3′ end of the antisense strand and/or the sense strand are connected by phosphorothioate internucleotide linkages. In other embodiments, all the unpaired nucleotides in a nucleotide overhang at the 5′ end of the antisense strand and/or the sense strand are connected by phosphorothioate internucleotide linkages. In still other embodiments, all the unpaired nucleotides in any nucleotide overhang are connected by phosphorothioate internucleotide linkages.

In certain embodiments, the modified nucleotides incorporated into one or both of the strands of the RNAi constructs of the invention have a modification of the nucleobase (also referred to herein as “base”). A “modified nucleobase” or “modified base” refers to a base other than the naturally occurring purine bases adenine (A) and guanine (G) and pyrimidine bases thymine (T), cytosine (C), and uracil (U). Modified nucleobases can be synthetic or naturally occurring modifications and include, but are not limited to, universal bases, 5-methylcytosine (5-me-C), 5-hydroxymethyl cytosine, xanthine (X), hypoxanthine (I), 2-aminoadenine, 6-methyladenine, 6-methylguanine, and other alkyl derivatives of adenine and guanine, 2-propyland other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine, and 3-deazaadenine.

In some embodiments, the modified base is a universal base. A “universal base” refers to a base analog that indiscriminately forms base pairs with all of the natural bases in RNA and DNA without altering the double helical structure of the resulting duplex region. Universal bases are known to those of skill in the art and include, but are not limited to, inosine, C-phenyl, C-naphthyl and other aromatic derivatives, azole carboxamides, and nitroazole derivatives, such as 3-nitropyrrole, 4-nitroindole, 5-nitroindole, and 6-nitroindole.

Other suitable modified bases that can be incorporated into the RNAi constructs of the invention include those described in, for example, Herdewijn, Antisense Nucleic Acid Drug Dev., 10: 297-310 (2000) and Peacock et al., J. Org. Chem., 76: 7295-7300 (2011). The skilled person is well aware that guanine, cytosine, adenine, thymine, and uracil may be replaced by other nucleobases, such as the modified nucleobases described above, without substantially altering the base pairing properties of a polynucleotide comprising a nucleotide bearing such replacement nucleobase.

In some embodiments, the 5′ end of the sense strand, antisense strand, or both the antisense and sense strands of the disclosed RNAi constructs comprises a phosphate moiety. As used herein, the term “phosphate moiety” refers to a terminal phosphate group that includes unmodified phosphates (—O—P—O)(OH)OH) as well as modified phosphates. Modified phosphates include phosphates in which one or more of the O and OH groups are replaced with H, O, S, N(R) or alkyl where R is H, an amino protecting group or unsubstituted or substituted alkyl. Exemplary phosphate moieties include, but are not limited to, 5′-monophosphate; 5′ diphosphate; 5′-triphosphate; 5′-guanosine cap (7-methylated or non-methylated); 5′-adenosinecap or any other modified or unmodified nucleotide cap structure; 5′-monothiophosphate (phosphorothioate); 5′-monodithiophosphate (phosphorodithioate); 5′-alpha-thiotriphosphate; 5′-gamma-thiotriphosphate, 5′-phosphoramidates; 5′-vinylphosphates; 5′-alkylphosphonates (wherein “alkyl” can be methyl, ethyl, isopropyl, propyl, etc.); and 5′-alkyletherphosphonates (wherein “alkylether” can be methoxymethyl, ethoxymethyl, etc.).

The modified nucleotides that can be incorporated into the RNAi constructs of the invention may have more than one chemical modification described herein. For instance, the modified nucleotide may have a modification to the ribose sugar as well as a modification to the nucleobase. By way of example, a modified nucleotide may comprise a 2′ sugar modification (e.g., 2′-fluoro or 2′-methyl) and comprise a modified base (e.g., 5-methyl cytosine or pseudouracil). In other embodiments, the modified nucleotide may comprise a sugar modification in combination with a modification to the 5′ phosphate that would create a modified internucleotide or internucleoside linkage when the modified nucleotide was incorporated into a polynucleotide. For instance, in some embodiments, the modified nucleotide may comprise a sugar modification, such as a 2′-fluoro modification, a 2′-O-methyl modification, or a bicyclic sugar modification, as well as a 5′ phosphorothioate group. Accordingly, in some embodiments, one or both strands of the RNAi constructs of the invention comprise a combination of 2′ modified nucleotides or BNAs and phosphorothioate internucleotide linkages. In certain embodiments, both the sense and antisense strands of the RNAi constructs of the invention comprise a combination of 2′-fluoro modified nucleotides, 2′-O-methyl modified nucleotides, and phosphorothioate internucleotide linkages. Exemplary RNAi constructs comprising modified nucleotides and internucleotide linkages are shown in Table 1 (the RNAi construct is any one of the duplexes comprising the sequences of: SEQ ID NO: 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32; 33 and 34; 35 and 36; 37 and 38; 39 and 40; 41 and 42; 43 and 44; 45 and 46; 47 and 48; 49 and 50; 51 and 52; 53 and 54; 55 and 56; 57 and 58; 59 and 60; 61 and 62; 63 and 64; 65 and 66; 67 and 68; 69 and 70; 71 and 72; 73 and 74; 75 and 76; 77 and 78; 79 and 80; 81 and 82; 83 and 84; 85 and 86; 87 and 88; 89 and 90; 91 and 92; 93 and 94; 95 and 96; 97 and 98; 99 and 100; 101 and 102; 103 and 104; 105 and 106; 107 and 108; 109 and 110; 111 and 112; 113 and 114; 115 and 116; 117 and 118; 119 and 120; 121 and 122; 123 and 124; 125 and 126; 127 and 128; 129 and 130; 131 and 132; 133 and 134; 135 and 136; 137 and 138; 139 and 140; 141 and 142; 143 and 144; 145 and 146; 147 and 148; 149 and 150; 151 and 152; 153 and 154; 155 and 156; 157 and 158; 159 and 160; 161 and 162; 163 and 164; 165 and 166; 167 and 168; 169 and 170; 171 and 172; 173 and 174; 175 and 176; 177 and 178; 179 and 180; 181 and 182; 183 and 184; 185 and 186; 187 and 188; 189 and 190; 191 and 192; 193 and 194; 195 and 196; 197 and 198; 199 and 200; 201 and 202; 203 and 204; 205 and 206; 207 and 208; 209 and 210; 211 and 212; 213 and 214; 215 and 216; 217 and 218; 219 and 220; 221 and 222; 223 and 224; 225 and 226; 227 and 228; 229 and 230; 231 and 232; 233 and 234; 235 and 236; 237 and 238; 239 and 240; 241 and 242; 243 and 244; 245 and 246; 247 and 248; 249 and 250; 251 and 252; 253 and 254; 255 and 256; 257 and 258; 259 and 260; 261 and 262; 263 and 264; 265 and 266; 267 and 268; 269 and 270; 271 and 272; 273 and 274; 275 and 276; 277 and 278; 279 and 280; 281 and 282; 283 and 284; 285 and 286; 287 and 288; 289 and 290; 291 and 292; 293 and 294; 295 and 296; 297 and 298; 299 and 300; 301 and 302; 303 and 304; 305 and 306; 307 and 308; 309 and 310; 311 and 312; 313 and 314; 315 and 316; 317 and 318; 319 and 320; 321 and 322; 323 and 324; 325 and 326; 327 and 328; 329 and 330; 331 and 332; 333 and 334; 335 and 336; 337 and 338; 339 and 340; 341 and 342; 343 and 344; 345 and 346; 347 and 348; 349 and 350; 351 and 352; 353 and 354; 355 and 356; 357 and 358; 359 and 360; 361 and 362; 363 and 364; 365 and 366; 367 and 368; 369 and 370; 371 and 372; 373 and 374; 375 and 376; 377 and 378; 379 and 380; 381 and 382; 383 and 384; 385 and 386; 387 and 388; 389 and 390; 391 and 392; 393 and 394; 395 and 396; 397 and 398; 399 and 400; 401 and 402; 403 and 404; 405 and 406; 407 and 408; 409 and 410; 411 and 412; 413 and 414; 415 and 416; 417 and 418; 419 and 420; 421 and 422; 423 and 424; 425 and 426; 427 and 428; 429 and 430; 431 and 432; 433 and 434; 435 and 436; 437 and 438; 439 and 440; 441 and 442; 443 and 444; 445 and 446; 447 and 448; 449 and 450; 451 and 452; 453 and 454; 455 and 456; 457 and 458; 459 and 460; 461 and 462; 463 and 464; 465 and 466; 467 and 468; 469 and 470; 471 and 472; 473 and 474; 475 and 476; 477 and 478; 479 and 480; 481 and 482; 483 and 484; 485 and 486; 487 and 488; 489 and 490; 491 and 492; 493 and 494; 495 and 496; 497 and 498; 499 and 500; 501 and 502; 503 and 504; 505 and 506; 507 and 508; 509 and 510; 511 and 512; 513 and 514; 515 and 516; 517 and 518; 519 and 520; 521 and 522; 523 and 524; 525 and 526; 527 and 528; 529 and 530; 531 and 532; 533 and 534; 535 and 536; 537 and 538; 539 and 540; 541 and 542; 543 and 544; 545 and 546; 547 and 548; 549 and 550; 551 and 552; 553 and 554; 555 and 556; 557 and 558; 559 and 560; 561 and 562; 563 and 564; or 565 and 566) and Table 2 (the RNAi construct is any one of the duplexes comprising the sequences of: SEQ ID NO: 567 and 568; 569 and 570; 571 and 572; 573 and 574; 575 and 576; 577 and 578; 579 and 580; 581 and 582; 583 and 584; 585 and 586; 587 and 588; 589 and 590; 591 and 592; 593 and 594; 595 and 596; 597 and 598; 599 and 600; 601 and 602; 603 and 604; 605 and 606; 607 and 608; 609 and 610; 611 and 612; 613 and 614; 615 and 616; 617 and 618; 619 and 620; 621 and 622; 623 and 624; 625 and 626; 627 and 628; 629 and 630; 631 and 632; 633 and 634; 635 and 636; 637 and 638; 639 and 640; 641 and 642; 643 and 644; 645 and 646; 647 and 648; 649 and 650; 651 and 652; 653 and 654; 655 and 656; 657 and 658; 659 and 660; 661 and 662; 663 and 664; 665 and 666; 667 and 668; 669 and 670; 671 and 672; 673 and 674; 675 and 676; 677 and 678; 679 and 680; 681 and 682; 683 and 684; 685 and 686; 687 and 688; 689 and 690; 691 and 692; 693 and 694; 695 and 696; 697 and 698; 699 and 700; 701 and 702; 703 and 704; 705 and 706; 707 and 708; 709 and 710; 711 and 712; 713 and 714; 715 and 716; 717 and 718; 719 and 720; 721 and 722; 723 and 724; 725 and 726; 727 and 728; 729 and 730; 731 and 732; 733 and 734; 735 and 736; 737 and 738; 739 and 740; 741 and 742; 743 and 744; 745 and 746; 747 and 748; 749 and 750; 751 and 752; 753 and 754; 755 and 756; 757 and 758; 759 and 760; 761 and 762; 763 and 764; 765 and 766; 767 and 768; 769 and 770; 771 and 772; 773 and 774; 775 and 776; 777 and 778; 779 and 780; 781 and 782; 783 and 784; 785 and 786; 787 and 788; 789 and 790; 791 and 792; 793 and 794; 795 and 796; 797 and 798; 799 and 800; 801 and 802; 803 and 804; 805 and 806; 807 and 808; 809 and 810; 811 and 812; 813 and 814; 815 and 816; 817 and 818; 819 and 820; 821 and 822; 823 and 824; 825 and 826; 827 and 828; 829 and 830; 831 and 832; 833 and 834; 835 and 836; 837 and 838; 839 and 840; 841 and 842; 843 and 844; 845 and 846; 847 and 848; 849 and 850; 851 and 852; 853 and 854; 855 and 856; 857 and 858; 859 and 860; 861 and 862; 863 and 864; 865 and 866; 867 and 868; 869 and 870; 871 and 872; 873 and 874; 875 and 876; 877 and 878; 879 and 880; 881 and 882; 883 and 884; 885 and 886; 887 and 888; 889 and 890; 891 and 892; 893 and 894; 895 and 896; 897 and 898; 899 and 900; 901 and 902; 903 and 904; 905 and 906; 907 and 908; 909 and 910; 911 and 912; 913 and 914; 915 and 916; 917 and 918; 919 and 920; 921 and 922; 923 and 924; 925 and 926; 927 and 928; 929 and 930; 931 and 932; 933 and 934; 935 and 936; 937 and 938; 939 and 940; 941 and 942; 943 and 944; 945 and 946; 947 and 948; 949 and 950; 951 and 952; 953 and 954; 955 and 956; 957 and 958; 959 and 960; 961 and 962; 963 and 964; 965 and 966; 967 and 968; 969 and 970; 971 and 972; 973 and 974; 975 and 976; 977 and 978; 979 and 980; 981 and 982; 983 and 984; 985 and 986; 987 and 988; 989 and 990; 991 and 992; 993 and 994; 995 and 996; 997 and 998; 999 and 1000; 1001 and 1002; 1003 and 1004; 1005 and 1006; 1007 and 1008; 1009 and 1010; 1011 and 1012; 1013 and 1014; 1015 and 1016; 1017 and 1018; 1019 and 1020; 1021 and 1022; 1023 and 1024; 1025 and 1026; 1027 and 1028; 1029 and 1030; 1031 and 1032; 1033 and 1034; 1035 and 1036; 1037 and 1038; 1039 and 1040; 1041 and 1042; 1043 and 1044; 1045 and 1046; 1047 and 1048; 1049 and 1050; 1051 and 1052; 1053 and 1054; 1055 and 1056; 1057 and 1058; 1059 and 1060; 1061 and 1062; 1063 and 1064; 1065 and 1066; 1067 and 1068; 1069 and 1070; 1071 and 1072; 1073 and 1074; 1075 and 1076; 1077 and 1078; 1079 and 1080; 1081 and 1082; 1083 and 1084; 1085 and 1086; 1087 and 1088; 1089 and 1090; 1091 and 1092; 1093 and 1094; 1095 and 1096; 1097 and 1098; 1099 and 1100; 1101 and 1102; 1103 and 1104; 1105 and 1106; 1107 and 1108; 1109 and 1110; 1111 and 1112; 1113 and 1114; 1115 and 1116; 1117 and 1118; 1119 and 1120; 1121 and 1122; 1123 and 1124; 1125 and 1126; 1127 and 1128; 1129 and 1130; 1131 and 1132; 1133 and 1134; 1135 and 1136; 1137 and 1138; 1139 and 1140; 1141 and 1142; 1143 and 1144; 1145 and 1146; 1147 and 1148; 1149 and 1150; 1151 and 1152; 1153 and 1154; 1155 and 1156; 1157 and 1158; 1159 and 1160; 1161 and 1162; 1163 and 1164; 1165 and 1166; 1167 and 1168; 1169 and 1170; 1171 and 1172; 1173 and 1174; 1175 and 1176; 1177 and 1178; 1179 and 1180; 1181 and 1182; 1183 and 1184; 1185 and 1186; 1187 and 1188; 1189 and 1190; 1191 and 1192; 1193 and 1194; 1195 and 1196; 1197 and 1198; 1199 and 1200; 1201 and 1202; 1203 and 1204; 1205 and 1206; 1207 and 1208; 1209 and 1210; 1211 and 1212; 1213 and 1214; 1215 and 1216; 1217 and 1218; 1219 and 1220; 1221 and 1222; 1223 and 1224; 1225 and 1226; 1227 and 1228; 1229 and 1230; 1231 and 1232; 1233 and 1234; 1235 and 1236; 1237 and 1238; 1239 and 1240; 1241 and 1242; 1243 and 1244; 1245 and 1246; 1247 and 1248; 1249 and 1250; 1251 and 1252; 1253 and 1254; 1255 and 1256; 1257 and 1258; 1259 and 1260; 1261 and 1262; 1263 and 1264; 1265 and 1266; 1267 and 1268; 1269 and 1270; 1271 and 1272; 1273 and 1274; 1275 and 1276; 1277 and 1278; 1279 and 1280; 1281 and 1282; 1283 and 1284; 1285 and 1286; 1287 and 1288; 1289 and 1290; 1291 and 1292; 1293 and 1294; 1295 and 1296; 1297 and 1298; 1299 and 1300; 1301 and 1302; 1303 and 1304; 1305 and 1306; 1307 and 1308; 1309 and 1310; 1311 and 1312; 1313 and 1314; 1315 and 1316; 1317 and 1318; 1319 and 1320; 1321 and 1322; 1323 and 1324; 1325 and 1326; 1327 and 1328; 1329 and 1330; 1331 and 1332; 1333 and 1334; 1335 and 1336; 1337 and 1338; 1339 and 1340; 1341 and 1342; 1343 and 1344; 1345 and 1346; 1347 and 1348; 1349 and 1350; 1351 and 1352; 1353 and 1354; 1355 and 1356; 1357 and 1358; 1359 and 1360; 1361 and 1362; 1363 and 1364; 1365 and 1366; 1367 and 1368; 1369 and 1370; 1371 and 1372; 1373 and 1374; 1375 and 1376; 1377 and 1378; 1379 and 1380; 1381 and 1382; 1383 and 1384; 1385 and 1386; 1387 and 1388; 1389 and 1390; 1391 and 1392; 1393 and 1394; 1395 and 1396; 1397 and 1398; 1399 and 1400; 1401 and 1402; 1403 and 1404; 1405 and 1406; 1407 and 1408; 1409 and 1410; 1411 and 1412; 1413 and 1414; 1415 and 1416; 1417 and 1418; 1419 and 1420; 1421 and 1422; 1423 and 1424; 1425 and 1426; 1427 and 1428; 1429 and 1430; 1431 and 1432; 1433 and 1434; 1435 and 1436; 1437 and 1438; 1439 and 1440; 1441 and 1442; 1443 and 1444; 1445 and 1446; 1447 and 1448; 1449 and 1450; 1451 and 1452; 1453 and 1454; 1455 and 1456; 1457 and 1458; 1459 and 1460; 1461 and 1462; 1463 and 1464; 1465 and 1466; 1467 and 1468; 1469 and 1470; 1471 and 1472; 1473 and 1474; 1475 and 1476; 1477 and 1478; 1479 and 1480; 1481 and 1482; 1483 and 1484; 1485 and 1486; 1487 and 1488; 1489 and 1490; 1491 and 1492; 1493 and 1494; 1495 and 1496; 1497 and 1498; 1499 and 1500; 1501 and 1502; 1503 and 1504; 1505 and 1506; 1507 and 1508; 1509 and 1510; 1511 and 1512; 1513 and 1514; 1515 and 1516; 1517 and 1518; 1519 and 1520; 1521 and 1522; 1523 and 1524; 1525 and 1526; 1527 and 1528; 1529 and 1530; 1531 and 1532; 1533 and 1534; 1535 and 1536; 1537 and 1538; 1539 and 1540; 1541 and 1542; 1543 and 1544; 1545 and 1546; 1547 and 1548; 1549 and 1550; 1551 and 1552; 1553 and 1554; 1555 and 1556; 1557 and 1558; 1559 and 1560; 1561 and 1562; 1563 and 1564; 1565 and 1566; 1567 and 1568; 1569 and 1570; 1571 and 1572; 1573 and 1574; 1575 and 1576; 1577 and 1578; 1579 and 1580; 1581 and 1582; 1583 and 1584; 1585 and 1586; 1587 and 1588; 1589 and 1590; 1591 and 1592; 1593 and 1594; 1595 and 1596; 1597 and 1598; 1599 and 1600; 1601 and 1602; 1603 and 1604; 1605 and 1606; 1607 and 1608; 1609 and 1610; 1611 and 1612; 1613 and 1614; 1615 and 1616; or 1617 and 1618).

Without being bound by theory, the transcript binding start location for each antisense strand is as follows (SEQ ID NO: followed by transcript start location): 2, 187; 4, 188; 6, 189; 8, 190; 10, 191; 12, 192; 14, 193; 16, 194; 18, 195; 20, 196; 22, 197; 24, 198; 26, 199; 28, 201; 30, 202; 32, 203; 34, 204; 36, 205; 38, 207; 40, 231; 42, 232; 44, 233; 46, 234; 48, 235; 50, 236; 52, 237; 54, 238; 56, 239; 58, 275; 60, 276; 62, 277; 64, 278; 66, 279; 68, 280; 70, 281; 72, 282; 74, 283; 76, 285; 78, 286; 80, 287; 82, 288; 84, 289; 86, 290; 88, 291; 90, 292; 92, 293; 94, 383; 96, 384; 98, 385; 100, 386; 102, 387; 104, 388; 106, 389; 108, 392; 110, 393; 112, 394; 114, 395; 116, 396; 118, 397; 120, 398; 122, 399; 124, 400; 126, 401; 128, 406; 130, 407; 132, 408; 134, 410; 136, 411; 138, 412; 140, 414; 142, 415; 144, 416; 146, 417; 148, 418; 150, 419; 152, 420; 154, 421; 156, 422; 158, 423; 160, 427; 162, 428; 164, 429; 166, 431; 168, 432; 170, 433; 172, 434; 174, 435; 176, 436; 178, 437; 180, 438; 182, 439; 184, 440; 186, 441; 188, 443; 190, 444; 192, 445; 194, 445; 196, 446; 198, 447; 200, 448; 202, 449; 204, 449; 206, 450; 208, 451; 210, 452; 212, 453; 214, 453; 216, 454; 218, 455; 220, 456; 222, 457; 224, 458; 226, 459; 228, 460; 230, 461; 232, 461; 234, 462; 236, 463; 238, 464; 240, 466; 242, 466; 244, 467; 246, 468; 248, 469; 250, 470; 252, 470; 254, 471; 256, 472; 258, 473; 260, 474; 262, 474; 264, 475; 266, 476; 268, 499; 270, 500; 272, 500; 274, 501; 276, 502; 278, 503; 280, 504; 282, 504; 284, 505; 286, 506; 288, 507; 290, 508; 292, 508; 294, 509; 296, 510; 298, 511; 300, 513; 302, 513; 304, 514; 306, 515; 308, 516; 310, 517; 312, 518; 314, 519; 316, 520; 318, 521; 320, 522; 322, 523; 324, 524; 326, 525; 328, 526; 330, 530; 332, 531; 334, 532; 336, 533; 338, 579; 340, 580; 342, 581; 344, 582; 346, 583; 348, 584; 350, 585; 352, 586; 354, 587; 356, 588; 358, 589; 360, 590; 362, 591; 364, 592; 366, 593; 368, 594; 370, 595; 372, 596; 374, 597; 376, 598; 378, 599; 380, 600; 382, 601; 384, 602; 386, 603; 388, 604; 390, 605; 392, 606; 394, 607; 396, 608; 398, 609; 400, 610; 402, 611; 404, 612; 406, 613; 408, 614; 410, 615; 412, 617; 414, 618; 416, 619; 418, 620; 420, 623; 422, 624; 424, 625; 426, 626; 428, 627; 430, 628; 432, 629; 434, 630; 436, 631; 438, 632; 440, 663; 442, 664; 444, 665; 446, 666; 448, 667; 450, 668; 452, 669; 454, 670; 456, 671; 458, 672; 460, 673; 462, 674; 464, 675; 466, 676; 468, 677; 470, 678; 472, 679; 474, 680; 476, 681; 478, 682; 480, 683; 482, 684; 484, 685; 486, 686; 488, 687; 490, 688; 492, 689; 494, 690; 496, 691; 498, 692; 500, 693; 502, 694; 504, 695; 506, 696; 508, 697; 510, 698; 512, 703; 514, 705; 516, 706; 518, 707; 520, 708; 522, 710; 524, 711; 526, 712; 528, 713; 530, 714; 532, 715; 534, 716; 536, 749; 538, 750; 540, 751; 542, 752; 544, 753; 546, 754; 548, 755; 550, 760; 552, 761; 554, 762; 556, 763; 558, 764; 560, 765; 562, 766; 564, 767; 566, 768; 568, 187; 570, 188; 572, 189; 574, 190; 576, 191; 578, 192; 580, 193; 582, 194; 584, 195; 586, 196; 588, 197; 590, 198; 592, 199; 594, 201; 596, 202; 598, 203; 600, 204; 602, 205; 604, 207; 606, 231; 608, 232; 610, 233; 612, 234; 614, 235; 616, 236; 618, 237; 620, 238; 622, 239; 624, 275; 626, 276; 628, 277; 630, 278; 632, 279; 634, 280; 636, 281; 638, 282; 640, 283; 642, 285; 644, 286; 646, 287; 648, 288; 650, 289; 652, 290; 654, 291; 656, 292; 658, 293; 660, 383; 662, 384; 664, 385; 666, 386; 668, 387; 670, 388; 672, 389; 674, 392; 676, 393; 678, 394; 680, 395; 682, 396; 684, 397; 686, 398; 688, 399; 690, 400; 692, 401; 694, 406; 696, 407; 698, 408; 700, 410; 702, 411; 704, 412; 706, 414; 708, 415; 710, 416; 712, 417; 714, 418; 716, 419; 718, 420; 720, 421; 722, 422; 724, 423; 726, 427; 728, 428; 730, 429; 732, 431; 734, 432; 736, 433; 738, 434; 740, 435; 742, 436; 744, 437; 746, 438; 748, 439; 750, 440; 752, 441; 754, 443; 756, 444; 758, 445; 760, 445; 762, 446; 764, 447; 766, 448; 768, 449; 770, 449; 772, 450; 774, 451; 776, 452; 778, 453; 780, 453; 782, 454; 784, 455; 786, 456; 788, 457; 790, 458; 792, 459; 794, 460; 796, 461; 798, 461; 800, 462; 802, 463; 804, 464; 806, 466; 808, 466; 810, 467; 812, 468; 814, 469; 816, 470; 818, 470; 820, 471; 822, 472; 824, 473; 826, 474; 828, 474; 830, 475; 832, 476; 834, 499; 836, 500; 838, 500; 840, 501; 842, 502; 844, 503; 846, 504; 848, 504; 850, 505; 852, 506; 854, 507; 856, 508; 858, 508; 860, 509; 862, 510; 864, 511; 866, 513; 868, 513; 870, 514; 872, 515; 874, 516; 876, 517; 878, 518; 880, 519; 882, 520; 884, 521; 886, 522; 888, 523; 890, 524; 892, 525; 894, 526; 896, 530; 898, 531; 900, 532; 902, 533; 904, 579; 906, 580; 908, 581; 910, 582; 912, 583; 914, 584; 916, 585; 918, 586; 920, 587; 922, 588; 924, 589; 926, 590; 928, 591; 930, 592; 932, 593; 934, 594; 936, 595; 938, 596; 940, 597; 942, 598; 944, 599; 946, 600; 948, 601; 950, 602; 952, 603; 954, 604; 956, 605; 958, 606; 960, 607; 962, 608; 964, 609; 966, 610; 968, 611; 970, 612; 972, 613; 974, 614; 976, 615; 978, 617; 980, 618; 982, 619; 984, 620; 986, 623; 988, 624; 990, 625; 992, 626; 994, 627; 996, 628; 998, 629; 1000, 630; 1002, 631; 1004, 632; 1006, 663; 1008, 664; 1010, 665; 1012, 666; 1014, 667; 1016, 668; 1018, 669; 1020, 670; 1022, 671; 1024, 672; 1026, 673; 1028, 674; 1030, 675; 1032, 676; 1034, 677; 1036, 678; 1038, 679; 1040, 680; 1042, 681; 1044, 682; 1046, 683; 1048, 684; 1050, 685; 1052, 686; 1054, 687; 1056, 688; 1058, 689; 1060, 690; 1062, 691; 1064, 692; 1066, 693; 1068, 694; 1070, 695; 1072, 696; 1074, 697; 1076, 698; 1078, 703; 1080, 705; 1082, 706; 1084, 707; 1086, 708; 1088, 710; 1090, 711; 1092, 712; 1094, 713; 1096, 714; 1098, 715; 1100, 716; 1102, 749; 1104, 750; 1106, 751; 1108, 752; 1110, 753; 1112, 754; 1114, 755; 1116, 760; 1118, 761; 1120, 762; 1122, 763; 1124, 764; 1126, 765; 1128, 766; 1130, 767; 1132, 768; 1133, 188; 1135, 188; 1137, 188; 1139, 188; 1141, 189; 1143, 191; 1145, 193; 1147, 194; 1149, 194; 1151, 195; 1153, 197; 1155, 198; 1157, 198; 1159, 198; 1161, 198; 1163, 198; 1165, 198; 1167, 198; 1169, 198; 1171, 198; 1173, 198; 1175, 198; 1177, 198; 1179, 198; 1181, 198; 1183, 198; 1185, 198; 1187, 198; 1189, 198; 1191, 198; 1193, 198; 1195, 198; 1197, 198; 1199, 198; 1201, 198; 1203, 198; 1205, 198; 1207, 198; 1209, 198; 1211, 201; 1213, 201; 1215, 202; 1217, 203; 1219, 204; 1221, 204; 1223, 205; 1225, 207; 1227, 207; 1229, 207; 1231, 207; 1233, 207; 1235, 207; 1237, 223; 1239, 223; 1241, 231; 1243, 231; 1245, 232; 1247, 233; 1249, 233; 1251, 233; 1253, 234; 1255, 236; 1257, 237; 1259, 239; 1261, 239; 1263, 239; 1265, 239; 1267, 239; 1269, 239; 1271, 275; 1273, 275; 1275, 277; 1277, 278; 1279, 278; 1281, 278; 1283, 279; 1285, 279; 1287, 279; 1289, 279; 1291, 279; 1293, 279; 1295, 279; 1297, 280; 1299, 280; 1301, 280; 1303, 280; 1305, 280; 1307, 280; 1309, 280; 1311, 280; 1313, 280; 1315, 280; 1317, 280; 1319, 280; 1321, 280; 1323, 280; 1325, 280; 1327, 280; 1329, 280; 1331, 280; 1333, 280; 1335, 280; 1337, 280; 1339, 280; 1341, 281; 1343, 281; 1345, 281; 1347, 281; 1349, 287; 1351, 287; 1353, 288; 1355, 288; 1357, 289; 1359, 289; 1361, 289; 1363, 289; 1365, 289; 1367, 290; 1369, 290; 1371, 290; 1373, 290; 1375, 290; 1377, 290; 1379, 290; 1381, 290; 1383, 290; 1385, 290; 1387, 290; 1389, 290; 1391, 290; 1393, 290; 1395, 291; 1397, 291; 1399, 291; 1401, 291; 1403, 291; 1405, 291; 1407, 291; 1409, 291; 1411, 291; 1413, 291; 1415, 291; 1417, 291; 1419, 291; 1421, 291; 1423, 293; 1425, 293; 1427, 293; 1429, 293; 1431, 293; 1433, 293; 1435, 293; 1437, 293; 1439, 293; 1441, 293; 1443, 293; 1445, 293; 1447, 293; 1449, 293; 1451, 293; 1453, 395; 1455, 445; 1457, 445; 1459, 448; 1461, 448; 1463, 448; 1465, 448; 1467, 448; 1469, 451; 1471, 451; 1473, 451; 1475, 451; 1477, 451; 1479, 451; 1481, 451; 1483, 451; 1485, 451; 1487, 451; 1489, 451; 1491, 451; 1493, 451; 1495, 451; 1497, 451; 1499, 451; 1501, 451; 1503, 451; 1505, 451; 1507, 451; 1509, 451; 1511, 451; 1513, 451; 1515, 451; 1517, 451; 1519, 451; 1521, 451; 1523, 451; 1525, 451; 1527, 451; 1529, 451; 1531, 462; 1533, 462; 1535, 464; 1537, 468; 1539, 468; 1541, 470; 1543, 470; 1545, 471; 1547, 471; 1549, 471; 1551, 472; 1553, 473; 1555, 473; 1557, 473; 1559, 474; 1561, 474; 1563, 475; 1565, 475; 1567, 475; 1569, 475; 1571, 475; 1573, 475; 1575, 476; 1577, 476; 1579, 476; 1581, 500; 1583, 506; 1585, 506; 1587, 506; 1589, 510; 1591, 510; 1593, 510; 1595, 510; 1597, 510; 1599, 510; 1601, 510; 1603, 510; 1605, 510; 1607, 510; 1609, 511; 1611, 511; 1613, 511; 1615, 511; 1617, 511.

As such, each duplex, based on the binding of the antisense strand, has a transcript binding start location as follows (Duplex No followed by transcript start location): D-1000, 187; D-1001, 188; D-1002, 189; D-1003, 190; D-1004, 191; D-1005, 192; D-1006, 193; D-1007, 194; D-1008, 195; D-1009, 196; D-1010, 197; D-1011, 198; D-1012, 199; D-1013, 201; D-1014, 202; D-1015, 203; D-1016, 204; D-1017, 205; D-1018, 207; D-1019, 231; D-1020, 232; D-1021, 233; D-1022, 234; D-1023, 235; D-1024, 236; D-1025, 237; D-1026, 238; D-1027, 239; D-1028, 275; D-1029, 276; D-1030, 277; D-1031, 278; D-1032, 279; D-1033, 280; D-1034, 281; D-1035, 282; D-1036, 283; D-1037, 285; D-1038, 286; D-1039, 287; D-1040, 288; D-1041, 289; D-1042, 290; D-1043, 291; D-1044, 292; D-1045, 293; D-1046, 383; D-1047, 384; D-1048, 385; D-1049, 386; D-1050, 387; D-1051, 388; D-1052, 389; D-1053, 392; D-1054, 393; D-1055, 394; D-1056, 395; D-1057, 396; D-1058, 397; D-1059, 398; D-1060, 399; D-1061, 400; D-1062, 401; D-1063, 406; D-1064, 407; D-1065, 408; D-1066, 410; D-1067, 411; D-1068, 412; D-1069, 414; D-1070, 415; D-1071, 416; D-1072, 417; D-1073, 418; D-1074, 419; D-1075, 420; D-1076, 421; D-1077, 422; D-1078, 423; D-1079, 427; D-1080, 428; D-1081, 429; D-1082, 431; D-1083, 432; D-1084, 433; D-1085, 434; D-1086, 435; D-1087, 436; D-1088, 437; D-1089, 438; D-1090, 439; D-1091, 440; D-1092, 441; D-1093, 443; D-1094, 444; D-1095, 445; D-1096, 445; D-1097, 446; D-1098, 447; D-1099, 448; D-1100, 449; D-1101, 449; D-1102, 450; D-1103, 451; D-1104, 452; D-1105, 453; D-1106, 453; D-1107, 454; D-1108, 455; D-1109, 456; D-1110, 457; D-1111, 458; D-1112, 459; D-1113, 460; D-1114, 461; D-1115, 461; D-1116, 462; D-1117, 463; D-1118, 464; D-1119, 466; D-1120, 466; D-1121, 467; D-1122, 468; D-1123, 469; D-1124, 470; D-1125, 470; D-1126, 471; D-1127, 472; D-1128, 473; D-1129, 474; D-1130, 474; D-1131, 475; D-1132, 476; D-1133, 499; D-1134, 500; D-1135, 500; D-1136, 501; D-1137, 502; D-1138, 503; D-1139, 504; D-1140, 504; D-1141, 505; D-1142, 506; D-1143, 507; D-1144, 508; D-1145, 508; D-1146, 509; D-1147, 510; D-1148, 511; D-1149, 513; D-1150, 513; D-1151, 514; D-1152, 515; D-1153, 516; D-1154, 517; D-1155, 518; D-1156, 519; D-1157, 520; D-1158, 521; D-1159, 522; D-1160, 523; D-1161, 524; D-1162, 525; D-1163, 526; D-1164, 530; D-1165, 531; D-1166, 532; D-1167, 533; D-1168, 579; D-1169, 580; D-1170, 581; D-1171, 582; D-1172, 583; D-1173, 584; D-1174, 585; D-1175, 586; D-1176, 587; D-1177, 588; D-1178, 589; D-1179, 590; D-1180, 591; D-1181, 592; D-1182, 593; D-1183, 594; D-1184, 595; D-1185, 596; D-1186, 597; D-1187, 598; D-1188, 599; D-1189, 600; D-1190, 601; D-1191, 602; D-1192, 603; D-1193, 604; D-1194, 605; D-1195, 606; D-1196, 607; D-1197, 608; D-1198, 609; D-1199, 610; D-1200, 611; D-1201, 612; D-1202, 613; D-1203, 614; D-1204, 615; D-1205, 617; D-1206, 618; D-1207, 619; D-1208, 620; D-1209, 623; D-1210, 624; D-1211, 625; D-1212, 626; D-1213, 627; D-1214, 628; D-1215, 629; D-1216, 630; D-1217, 631; D-1218, 632; D-1219, 663; D-1220, 664; D-1221, 665; D-1222, 666; D-1223, 667; D-1224, 668; D-1225, 669; D-1226, 670; D-1227, 671; D-1228, 672; D-1229, 673; D-1230, 674; D-1231, 675; D-1232, 676; D-1233, 677; D-1234, 678; D-1235, 679; D-1236, 680; D-1237, 681; D-1238, 682; D-1239, 683; D-1240, 684; D-1241, 685; D-1242, 686; D-1243, 687; D-1244, 688; D-1245, 689; D-1246, 690; D-1247, 691; D-1248, 692; D-1249, 693; D-1250, 694; D-1251, 695; D-1252, 696; D-1253, 697; D-1254, 698; D-1255, 703; D-1256, 705; D-1257, 706; D-1258, 707; D-1259, 708; D-1260, 710; D-1261, 711; D-1262, 712; D-1263, 713; D-1264, 714; D-1265, 715; D-1266, 716; D-1267, 749; D-1268, 750; D-1269, 751; D-1270, 752; D-1271, 753; D-1272, 754; D-1273, 755; D-1274, 760; D-1275, 761; D-1276, 762; D-1277, 763; D-1278, 764; D-1279, 765; D-1280, 766; D-1281, 767; D-1282, 768; D-2000, 187; D-2001, 188; D-2002, 189; D-2003, 190; D-2004, 191; D-2005, 192; D-2006, 193; D-2007, 194; D-2008, 195; D-2009, 196; D-2010, 197; D-2011, 198; D-2012, 199; D-2013, 201; D-2014, 202; D-2015, 203; D-2016, 204; D-2017, 205; D-2018, 207; D-2019, 231; D-2020, 232; D-2021, 233; D-2022, 234; D-2023, 235; D-2024, 236; D-2025, 237; D-2026, 238; D-2027, 239; D-2028, 275; D-2029, 276; D-2030, 277; D-2031, 278; D-2032, 279; D-2033, 280; D-2034, 281; D-2035, 282; D-2036, 283; D-2037, 285; D-2038, 286; D-2039, 287; D-2040, 288; D-2041, 289; D-2042, 290; D-2043, 291; D-2044, 292; D-2045, 293; D-2046, 383; D-2047, 384; D-2048, 385; D-2049, 386; D-2050, 387; D-2051, 388; D-2052, 389; D-2053, 392; D-2054, 393; D-2055, 394; D-2056, 395; D-2057, 396; D-2058, 397; D-2059, 398; D-2060, 399; D-2061, 400; D-2062, 401; D-2063, 406; D-2064, 407; D-2065, 408; D-2066, 410; D-2067, 411; D-2068, 412; D-2069, 414; D-2070, 415; D-2071, 416; D-2072, 417; D-2073, 418; D-2074, 419; D-2075, 420; D-2076, 421; D-2077, 422; D-2078, 423; D-2079, 427; D-2080, 428; D-2081, 429; D-2082, 431; D-2083, 432; D-2084, 433; D-2085, 434; D-2086, 435; D-2087, 436; D-2088, 437; D-2089, 438; D-2090, 439; D-2091, 440; D-2092, 441; D-2093, 443; D-2094, 444; D-2095, 445; D-2096, 445; D-2097, 446; D-2098, 447; D-2099, 448; D-2100, 449; D-2101, 449; D-2102, 450; D-2103, 451; D-2104, 452; D-2105, 453; D-2106, 453; D-2107, 454; D-2108, 455; D-2109, 456; D-2110, 457; D-2111, 458; D-2112, 459; D-2113, 460; D-2114, 461; D-2115, 461; D-2116, 462; D-2117, 463; D-2118, 464; D-2119, 466; D-2120, 466; D-2121, 467; D-2122, 468; D-2123, 469; D-2124, 470; D-2125, 470; D-2126, 471; D-2127, 472; D-2128, 473; D-2129, 474; D-2130, 474; D-2131, 475; D-2132, 476; D-2133, 499; D-2134, 500; D-2135, 500; D-2136, 501; D-2137, 502; D-2138, 503; D-2139, 504; D-2140, 504; D-2141, 505; D-2142, 506; D-2143, 507; D-2144, 508; D-2145, 508; D-2146, 509; D-2147, 510; D-2148, 511; D-2149, 513; D-2150, 513; D-2151, 514; D-2152, 515; D-2153, 516; D-2154, 517; D-2155, 518; D-2156, 519; D-2157, 520; D-2158, 521; D-2159, 522; D-2160, 523; D-2161, 524; D-2162, 525; D-2163, 526; D-2164, 530; D-2165, 531; D-2166, 532; D-2167, 533; D-2168, 579; D-2169, 580; D-2170, 581; D-2171, 582; D-2172, 583; D-2173, 584; D-2174, 585; D-2175, 586; D-2176, 587; D-2177, 588; D-2178, 589; D-2179, 590; D-2180, 591; D-2181, 592; D-2182, 593; D-2183, 594; D-2184, 595; D-2185, 596; D-2186, 597; D-2187, 598; D-2188, 599; D-2189, 600; D-2190, 601; D-2191, 602; D-2192, 603; D-2193, 604; D-2194, 605; D-2195, 606; D-2196, 607; D-2197, 608; D-2198, 609; D-2199, 610; D-2200, 611; D-2201, 612; D-2202, 613; D-2203, 614; D-2204, 615; D-2205, 617; D-2206, 618; D-2207, 619; D-2208, 620; D-2209, 623; D-2210, 624; D-2211, 625; D-2212, 626; D-2213, 627; D-2214, 628; D-2215, 629; D-2216, 630; D-2217, 631; D-2218, 632; D-2219, 663; D-2220, 664; D-2221, 665; D-2222, 666; D-2223, 667; D-2224, 668; D-2225, 669; D-2226, 670; D-2227, 671; D-2228, 672; D-2229, 673; D-2230, 674; D-2231, 675; D-2232, 676; D-2233, 677; D-2234, 678; D-2235, 679; D-2236, 680; D-2237, 681; D-2238, 682; D-2239, 683; D-2240, 684; D-2241, 685; D-2242, 686; D-2243, 687; D-2244, 688; D-2245, 689; D-2246, 690; D-2247, 691; D-2248, 692; D-2249, 693; D-2250, 694; D-2251, 695; D-2252, 696; D-2253, 697; D-2254, 698; D-2255, 703; D-2256, 705; D-2257, 706; D-2258, 707; D-2259, 708; D-2260, 710; D-2261, 711; D-2262, 712; D-2263, 713; D-2264, 714; D-2265, 715; D-2266, 716; D-2267, 749; D-2268, 750; D-2269, 751; D-2270, 752; D-2271, 753; D-2272, 754; D-2273, 755; D-2274, 760; D-2275, 761; D-2276, 762; D-2277, 763; D-2278, 764; D-2279, 765; D-2280, 766; D-2281, 767; D-2282, 768; D-2283, 188; D-2284, 188; D-2285, 188; D-2286, 188; D-2287, 189; D-2288, 191; D-2289, 193; D-2290, 194; D-2291, 194; D-2292, 195; D-2293, 197; D-2294, 198; D-2295, 198; D-2296, 198; D-2297, 198; D-2298, 198; D-2299, 198; D-2300, 198; D-2301, 198; D-2302, 198; D-2303, 198; D-2304, 198; D-2305, 198; D-2306, 198; D-2307, 198; D-2308, 198; D-2309, 198; D-2310, 198; D-2311, 198; D-2312, 198; D-2313, 198; D-2314, 198; D-2315, 198; D-2316, 198; D-2317, 198; D-2318, 198; D-2319, 198; D-2320, 198; D-2321, 198; D-2322, 201; D-2323, 201; D-2324, 202; D-2325, 203; D-2326, 204; D-2327, 204; D-2328, 205; D-2329, 207; D-2330, 207; D-2331, 207; D-2332, 207; D-2333, 207; D-2334, 207; D-2335, 223; D-2336, 223; D-2337, 231; D-2338, 231; D-2339, 232; D-2340, 233; D-2341, 233; D-2342, 233; D-2343, 234; D-2344, 236; D-2345, 237; D-2346, 239; D-2347, 239; D-2348, 239; D-2349, 239; D-2350, 239; D-2351, 239; D-2352, 275; D-2353, 275; D-2354, 277; D-2355, 278; D-2356, 278; D-2357, 278; D-2358, 279; D-2359, 279; D-2360, 279; D-2361, 279; D-2362, 279; D-2363, 279; D-2364, 279; D-2365, 280; D-2366, 280; D-2367, 280; D-2368, 280; D-2369, 280; D-2370, 280; D-2371, 280; D-2372, 280; D-2373, 280; D-2374, 280; D-2375, 280; D-2376, 280; D-2377, 280; D-2378, 280; D-2379, 280; D-2380, 280; D-2381, 280; D-2382, 280; D-2383, 280; D-2384, 280; D-2385, 280; D-2386, 280; D-2387, 281; D-2388, 281; D-2389, 281; D-2390, 281; D-2391, 287; D-2392, 287; D-2393, 288; D-2394, 288; D-2395, 289; D-2396, 289; D-2397, 289; D-2398, 289; D-2399, 289; D-2400, 290; D-2401, 290; D-2402, 290; D-2403, 290; D-2404, 290; D-2405, 290; D-2406, 290; D-2407, 290; D-2408, 290; D-2409, 290; D-2410, 290; D-2411, 290; D-2412, 290; D-2413, 290; D-2414, 291; D-2415, 291; D-2416, 291; D-2417, 291; D-2418, 291; D-2419, 291; D-2420, 291; D-2421, 291; D-2422, 291; D-2423, 291; D-2424, 291; D-2425, 291; D-2426, 291; D-2427, 291; D-2428, 293; D-2429, 293; D-2430, 293; D-2431, 293; D-2432, 293; D-2433, 293; D-2434, 293; D-2435, 293; D-2436, 293; D-2437, 293; D-2438, 293; D-2439, 293; D-2440, 293; D-2441, 293; D-2442, 293; D-2443, 395; D-2444, 445; D-2445, 445; D-2446, 448; D-2447, 448; D-2448, 448; D-2449, 448; D-2450, 448; D-2451, 451; D-2452, 451; D-2453, 451; D-2454, 451; D-2455, 451; D-2456, 451; D-2457, 451; D-2458, 451; D-2459, 451; D-2460, 451; D-2461, 451; D-2462, 451; D-2463, 451; D-2464, 451; D-2465, 451; D-2466, 451; D-2467, 451; D-2468, 451; D-2469, 451; D-2470, 451; D-2471, 451; D-2472, 451; D-2473, 451; D-2474, 451; D-2475, 451; D-2476, 451; D-2477, 451; D-2478, 451; D-2479, 451; D-2480, 451; D-2481, 451; D-2482, 462; D-2483, 462; D-2484, 464; D-2485, 468; D-2486, 468; D-2487, 470; D-2488, 470; D-2489, 471; D-2490, 471; D-2491, 471; D-2492, 472; D-2493, 473; D-2494, 473; D-2495, 473; D-2496, 474; D-2497, 474; D-2498, 475; D-2499, 475; D-2500, 475; D-2501, 475; D-2502, 475; D-2503, 475; D-2504, 476; D-2505, 476; D-2506, 476; D-2507, 500; D-2508, 506; D-2509, 506; D-2510, 506; D-2511, 510; D-2512, 510; D-2513, 510; D-2514, 510; D-2515, 510; D-2516, 510; D-2517, 510; D-2518, 510; D-2519, 510; D-2520, 510; D-2521, 511; D-2522, 511; D-2523, 511; D-2524, 511; D-2525, 511.

In some embodiments, the RNAi construct of the present invention comprises a sense strand and an antisense strand, wherein the antisense strand has a transcript binding start location at nucleotide 195 to 201, and comprises a region having at least 15 contiguous nucleotides differing by no more than 3 nucleotides from an antisense sequence of SEQ ID NO: 18, 20, 22, 24, 26, 28, 584, 586, 588, 590, 592, 594, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, or 1214. In some embodiments, the RNAi construct of the present invention comprise a sense strand and an antisense strand, wherein the antisense strand has a transcript binding start location at nucleotide 195 to 201, and comprises a region having at least 15 contiguous nucleotides differing by no more than 3 nucleotides from an antisense sequence and wherein the sense strand comprises a region having at least 15 contiguous nucleotides differing by no more than 3 nucleotides from a sense sequence, wherein the sense and antisense sequences comprise the sequence of SEQ ID NO: 17 and 18; 19 and 20; 21 and 22; 23 and 24; 25 and 26; 27 and 28; 583 and 584; 585 and 586; 587 and 588; 589 and 590; 591 and 592; 593 and 594; 1151 and 1152; 1153 and 1154; 1155 and 1156; 1157 and 1158; 1159 and 1160; 1161 and 1162; 1163 and 1164; 1165 and 1166; 1167 and 1168; 1169 and 1170; 1171 and 1172; 1173 and 1174; 1175 and 1176; 1177 and 1178; 1179 and 1180; 1181 and 1182; 1183 and 1184; 1185 and 1186; 1187 and 1188; 1189 and 1190; 1191 and 1192; 1193 and 1194; 1195 and 1196; 1197 and 1198; 1199 and 1200; 1201 and 1202; 1203 and 1204; 1205 and 1206; 1207 and 1208; 1209 and 1210; 1211 and 1212; or 1213 and 1214. In some embodiments, the RNAi construct of the present invention comprises a sense strand and an antisense strand, wherein the antisense strand has a transcript binding start location at nucleotide 195 to 201, and an antisense sequence of SEQ ID NO: 18, 20, 22, 24, 26, 28, 584, 586, 588, 590, 592, 594, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, or 1214. In some embodiments, the RNAi construct of the present invention comprise a sense strand and an antisense strand, wherein the antisense strand has a transcript binding start location at nucleotide 195 to 201, and an antisense sequence of SEQ ID NO: 18, 20, 22, 24, 26, 28, 584, 586, 588, 590, 592, 594, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, or 1214, and wherein the sense and antisense sequences comprise the sequence of SEQ ID NO: 17 and 18; 19 and 20; 21 and 22; 23 and 24; 25 and 26; 27 and 28; 583 and 584; 585 and 586; 587 and 588; 589 and 590; 591 and 592; 593 and 594; 1151 and 1152; 1153 and 1154; 1155 and 1156; 1157 and 1158; 1159 and 1160; 1161 and 1162; 1163 and 1164; 1165 and 1166; 1167 and 1168; 1169 and 1170; 1171 and 1172; 1173 and 1174; 1175 and 1176; 1177 and 1178; 1179 and 1180; 1181 and 1182; 1183 and 1184; 1185 and 1186; 1187 and 1188; 1189 and 1190; 1191 and 1192; 1193 and 1194; 1195 and 1196; 1197 and 1198; 1199 and 1200; 1201 and 1202; 1203 and 1204; 1205 and 1206; 1207 and 1208; 1209 and 1210; 1211 and 1212; or 1213 and 1214.

In some embodiments, the RNAi construct of the present invention comprises a sense strand and an antisense strand, wherein the antisense strand has a transcript binding start location at nucleotide 236 to 239, and comprises a region having at least 15 contiguous nucleotides differing by no more than 3 nucleotides from an antisense sequence of SEQ ID NO: 50, 52, 54, 56, 616, 618, 620, 622, 1256, 1258, 1260, 1262, 1264, 1266, 1268, or 1270. In some embodiments, the RNAi construct of the present invention comprise a sense strand and an antisense strand, wherein the antisense strand has a transcript binding start location at nucleotide 236 to 239, and comprises a region having at least 15 contiguous nucleotides differing by no more than 3 nucleotides from an antisense sequence and wherein the sense strand comprises a region having at least 15 contiguous nucleotides differing by no more than 3 nucleotides from a sense sequence, wherein the sense and antisense sequences comprise the sequence of SEQ ID NO: 49 and 50; 51 and 52; 53 and 54; 55 and 56; 615 and 616; 617 and 618; 619 and 620; 621 and 622; 1255 and 1256; 1257 and 1258; 1259 and 1260; 1261 and 1262; 1263 and 1264; 1265 and 1266; 1267 and 1268; or 1269 and 1270. In some embodiments, the RNAi construct of the present invention comprise a sense strand and an antisense strand, wherein the antisense strand has a transcript binding start location at nucleotide 236 to 239, and an antisense sequence of SEQ ID NO: 50, 52, 54, 56, 616, 618, 620, 622, 1256, 1258, 1260, 1262, 1264, 1266, 1268, or 1270. In some embodiments, the RNAi construct of the present invention comprise a sense strand and an antisense strand, wherein the antisense strand has a transcript binding start location at nucleotide 236 to 239, and an antisense sequence of SEQ ID NO: 50, 52, 54, 56, 616, 618, 620, 622, 1256, 1258, 1260, 1262, 1264, 1266, 1268, or 1270, and wherein the sense and antisense sequences comprise the sequence of SEQ ID NO: 49 and 50; 51 and 52; 53 and 54; 55 and 56; 615 and 616; 617 and 618; 619 and 620; 621 and 622; 1255 and 1256; 1257 and 1258; 1259 and 1260; 1261 and 1262; 1263 and 1264; 1265 and 1266; 1267 and 1268; or 1269 and 1270.

In some embodiments, the RNAi construct of the present invention comprises a sense strand and an antisense strand, wherein the antisense strand has a transcript binding start location at nucleotide 277 to 283, and comprises a region having at least 15 contiguous nucleotides differing by no more than 3 nucleotides from an antisense sequence of SEQ ID NO: 62, 64, 66, 68, 70, 72, 74, 628, 630, 632, 634, 636, 638, 640, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, or 1348. In some embodiments, the RNAi construct of the present invention comprise a sense strand and an antisense strand, wherein the antisense strand has a transcript binding start location at nucleotide 277 to 283, and comprises a region having at least 15 contiguous nucleotides differing by no more than 3 nucleotides from an antisense sequence and wherein the sense strand comprises a region having at least 15 contiguous nucleotides differing by no more than 3 nucleotides from a sense sequence, wherein the sense and antisense sequences comprise the sequence of SEQ ID NO: 61 and 62; 63 and 64; 65 and 66; 67 and 68; 69 and 70; 71 and 72; 73 and 74; 627 and 628; 629 and 630; 631 and 632; 633 and 634; 635 and 636; 637 and 638; 639 and 640; 1275 and 1276; 1277 and 1278; 1279 and 1280; 1281 and 1282; 1283 and 1284; 1285 and 1286; 1287 and 1288; 1289 and 1290; 1291 and 1292; 1293 and 1294; 1295 and 1296; 1297 and 1298; 1299 and 1300; 1301 and 1302; 1303 and 1304; 1305 and 1306; 1307 and 1308; 1309 and 1310; 1311 and 1312; 1313 and 1314; 1315 and 1316; 1317 and 1318; 1319 and 1320; 1321 and 1322; 1323 and 1324; 1325 and 1326; 1327 and 1328; 1329 and 1330; 1331 and 1332; 1333 and 1334; 1335 and 1336; 1337 and 1338; 1339 and 1340; 1341 and 1342; 1343 and 1344; 1345 and 1346; or 1347 and 1348. In some embodiments, the RNAi construct of the present invention comprise a sense strand and an antisense strand, wherein the antisense strand has a transcript binding start location at nucleotide 277 to 283, and an antisense sequence of SEQ ID NO: 62, 64, 66, 68, 70, 72, 74, 628, 630, 632, 634, 636, 638, 640, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, or 1348. In some embodiments, the RNAi construct of the present invention comprise a sense strand and an antisense strand, wherein the antisense strand has a transcript binding start location at nucleotide 277 to 283, and an antisense sequence of SEQ ID NO: 62, 64, 66, 68, 70, 72, 74, 628, 630, 632, 634, 636, 638, 640, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, or 1348, and wherein the sense and antisense sequences comprise the sequence of SEQ ID NO: 61 and 62; 63 and 64; 65 and 66; 67 and 68; 69 and 70; 71 and 72; 73 and 74; 627 and 628; 629 and 630; 631 and 632; 633 and 634; 635 and 636; 637 and 638; 639 and 640; 1275 and 1276; 1277 and 1278; 1279 and 1280; 1281 and 1282; 1283 and 1284; 1285 and 1286; 1287 and 1288; 1289 and 1290; 1291 and 1292; 1293 and 1294; 1295 and 1296; 1297 and 1298; 1299 and 1300; 1301 and 1302; 1303 and 1304; 1305 and 1306; 1307 and 1308; 1309 and 1310; 1311 and 1312; 1313 and 1314; 1315 and 1316; 1317 and 1318; 1319 and 1320; 1321 and 1322; 1323 and 1324; 1325 and 1326; 1327 and 1328; 1329 and 1330; 1331 and 1332; 1333 and 1334; 1335 and 1336; 1337 and 1338; 1339 and 1340; 1341 and 1342; 1343 and 1344; 1345 and 1346; or 1347 and 1348.

In one embodiment, the RNAi construct of the invention is a duplex comprising the sequences of SEQ ID NO: 1193 and 1194.

In another embodiment, the RNAi construct of the invention is a duplex comprising the sequences of SEQ ID NO: 1171 and 1172.

In another embodiment, the RNAi construct of the invention is a duplex comprising the sequences of SEQ ID NO: 1265 and 1266.

In another embodiment, the RNAi construct of the invention is a duplex comprising the sequences of SEQ ID NO: 1307 and 1308.

In another embodiment, the RNAi construct of the invention is a duplex comprising the sequences of SEQ ID NO: 1311 and 1312.

In another embodiment, the RNAi construct of the invention is a duplex comprising the sequences of SEQ ID NO: 1317 and 1318.

In another embodiment, the RNAi construct of the invention is a duplex comprising the sequences of SEQ ID NO: 1425 and 1426.

In another embodiment, the RNAi construct of the invention is a duplex comprising the sequences of SEQ ID NO: 1439 and 1440.

In another embodiment, the RNAi construct of the invention is a duplex comprising the sequences of SEQ ID NO: 1431 and 1432.

In another embodiment, the RNAi construct of the invention is a duplex comprising the sequences of SEQ ID NO: 1495 and 1496.

In another embodiment, the RNAi construct of the invention is a duplex comprising the sequences of SEQ ID NO: 1487 and 1488.

In another embodiment, the RNAi construct of the invention is a duplex comprising the sequences of SEQ ID NO: 1383 and 1384.

### Function of RNAi Constructs

The RNAi constructs of the invention desirably reduce or inhibit the expression of TTR in cells, particularly liver cells. Accordingly, in one embodiment, the present invention provides a method of reducing TTR expression in a cell by contacting the cell with any RNAi construct described herein. The cell may be in vitro or in vivo. TTR expression can be assessed by measuring the amount or level of TTR mRNA, TTR protein, or another biomarker linked to TTR expression. The reduction of TTR expression in cells or animals treated with an RNAi construct of the invention can be determined relative to the TTR expression in cells or animals not treated with the RNAi construct or treated with a control RNAi construct. For instance, in some embodiments, reduction of TTR expression is assessed by (a) measuring the amount or level of TTR mRNA in liver cells treated with a RNAi construct of the invention, (b) measuring the amount or level of TTR mRNA in liver cells treated with a control RNAi construct (e.g., RNAi construct directed to a RNA molecule not expressed in liver cells or a RNAi construct having a nonsense or scrambled sequence) or no construct, and (c) comparing the measured TTR mRNA levels from treated cells in (a) to the measured TTR mRNA levels from control cells in (b). The TTR mRNA levels in the treated cells and controls cells can be normalized to RNA levels for a control gene (e.g., 18S ribosomal RNA) prior to comparison. TTR mRNA levels can be measured by a variety of methods, including Northern blot analysis, nuclease protection assays, fluorescence in situ hybridization (FISH), reverse-transcriptase (RT)-PCR, real-time RT-PCR, quantitative PCR, and the like.

In other embodiments, reduction of TTR expression is assessed by (a) measuring the amount or level of TTR protein in liver cells treated with a RNAi construct of the invention, (b) measuring the amount or level of TTR protein in liver cells treated with a control RNAi construct (e.g., RNAi construct directed to a RNA molecule not expressed in liver cells or a RNAi construct having a nonsense or scrambled sequence) or no construct, and (c) comparing the measured TTR protein levels from treated cells in (a) to the measured TTR protein levels from control cells in (b). TTR protein levels can be measured using any suitable method known to those of skill in the art, including but not limited to, western blots, immunoassays (e.g., ELISA), and flow cytometry. Any suitable method of measuring TTR mRNA or protein can be used to assess the efficacy of the RNAi constructs of the invention.

In some embodiments, the methods to assess TTR expression levels are performed in vitro in cells that natively express TTR (e.g., liver cells) or cells that have been engineered to express TTR. In certain embodiments, the methods are performed in vitro in liver cells. Suitable liver cells include, but are not limited to, primary hepatocytes (e.g. human, non-human primate, or rodent hepatocytes), HepAD38 cells, HuH-6 cells, HuH-7 cells, HuH-5-2 cells, BNLCL2 cells, Hep3B cells, or HepG2 cells. In one embodiment, the liver cells are Hep3B cells. In another embodiment, the liver cells are HepG2 cells.

In other embodiments, the methods to assess TTR expression levels are performed in vivo. For example, the RNAi constructs and any control RNAi constructs can be administered to an animal (e.g., rodent or non-human primate), and TTR mRNA or protein levels may be assessed in liver tissue harvested from the animal following treatment. Alternatively or additionally, a biomarker or functional phenotype associated with TTR expression can be assessed in the treated animals.

In certain embodiments, expression of TTR is reduced in liver cells by at least at least 40%, at least 45%, or at least 50% by an RNAi construct of the invention. In some embodiments, expression of TTR is reduced in liver cells by at least 55%, at least 60%, at least 65%, at least 70%, at least 75%, at least 80%, or at least 85% by an RNAi construct of the invention. In other embodiments, the expression of TTR is reduced in liver cells by about 90% or more, e.g., 91%, 92%, 93%, 94%, 95%, 96%, 97%, 98%, 99%, or more by an RNAi construct of the invention. The percent reduction of TTR expression can be measured by any of the methods described herein or otherwise known in the art. For instance, in certain embodiments, the RNAi constructs of the invention inhibit at least 40% of TTR expression at 5 nM in Hep3B cells (contains wild type TTR) in vitro. In related embodiments, the RNAi constructs of the invention inhibit at least 45%, at least 50%, at least 55%, at least 60%, at least 65%, at least 70%, or at least 75% of TTR expression at 5 nM in Hep3B cells in vitro. In other embodiments, the RNAi constructs of the invention inhibit at least 80%, at least 85%, at least 90%, at least 92%, at least 94%, at least 96%, or at least 98% of TTR expression at 5 nM in Hep3B cells in vitro. In certain embodiments, the RNAi constructs of the invention inhibit at least 40% of TTR expression at 5 nM in HepG2 cells in vitro. In related embodiments, the RNAi constructs of the invention inhibit at least 45%, at least 50%, at least 55%, at least 60%, at least 65%, at least 70%, or at least 75% of TTR expression at 5 nM in HepG2 cells in vitro. In other embodiments, the RNAi constructs of the invention inhibit at least 80%, at least 85%, at least 90%, at least 92%, at least 94%, at least 96%, or at least 98% of TTR expression at 5 nM in HepG2 cells in vitro. In certain embodiments, the RNAi constructs of the invention inhibit at least 40% of TTR expression at 5 nM in CHO transfected cells expressing human TTR cells in vitro. In related embodiments, the RNAi constructs of the invention inhibit at least 45%, at least 50%, at least 55%, at least 60%, at least 65%, at least 70%, or at least 75% of TTR expression at 5 nM in CHO transfected cells expressing human in vitro. In other embodiments, the RNAi constructs of the invention inhibit at least 80%, at least 85%, at least 90%, at least 92%, at least 94%, at least 96%, or at least 98% of TTR expression at 5 nM in CHO transfected cells expressing human TTR in vitro. Reduction of TTR can be measured using a variety of techniques including, for example, RNA FISH or droplet digital PCR (see, e.g., Kamitaki et al., Digital PCR. Methods in Molecular Biology, 1768: 401-422 (2018). doi:10.1007/978-1-4939-7778-9_23).

In some embodiments, an IC50 value is calculated to assess the potency of an RNAi construct of the invention for inhibiting TTR expression in liver cells. An “IC50 value” is the dose/concentration required to achieve 50% inhibition of a biological or biochemical function. In other embodiments, the potency of an RNAi construct may be assessed by calculating an “AC50” value, which is the dose/concentration required to achieve 50% activation of a biological or biochemical function. The IC50 value or AC50 value of any substance or antagonist can be determined by constructing a dose-response curve and examining the effect of different concentrations of the substance or antagonist on expression levels or functional activity in any assay. IC50 values can be calculated for a given antagonist or substance by determining the concentration needed to inhibit half of the maximum biological response or native expression levels. Thus, the IC50 value for any RNAi construct can be calculated by determining the concentration of the RNAi construct needed to inhibit half of the native TTR expression level in liver cells (e.g., TTR expression level in control liver cells) in any assay, such as an immunoassay, RNA FISH assay, or a droplet digital PCR assay. Similarly, AC50 values can be calculated for a given substance by determining the concentration needed to activate half of the maximum biological response or native expression levels. The RNAi constructs of the invention may inhibit TTR expression in liver cells (e.g. Hep3B cells) with an IC50 of less than about 20 nM (e.g., less than about 15 nM, 10 nM, 5 nM, or 1 nM). For example, the disclosed RNAi constructs may inhibit TTR expression in liver cells with an IC50 of about 0.001 nM to about 20 nM, about 0.001 nM to about 10 nM, about 0.001 nM to about 5 nM, about 0.001 nM to about 1 nM, about 0.1 nM to about 10 nM, about 0.1 nM to about 5 nM, or about 0.1 nM to about 1 nM. In certain embodiments, the RNAi construct inhibits TTR expression in liver cells (e.g., Hep3B cells) with an IC50 of about 1 nM to about 10 nM (e.g., about 5 nM). The RNAi constructs of the invention may inhibit TTR expression in liver cells (e.g., HepG2 cells) with an IC50 of less than about 20 nM. For example, the RNAi constructs may inhibit TTR expression in liver cells with an IC50 of about 0.001 nM to about 20 nM, about 0.001 nM to about 10 nM, about 0.001 nM to about 5 nM, about 0.001 nM to about 1 nM, about 0.1 nM to about 10 nM, about 0.1 nM to about 5 nM, or about 0.1 nM to about 1 nM. In certain embodiments, the RNAi construct inhibits TTR expression in liver cells (e.g., HepG2 cells) with an IC50 of about 1 nM to about 10 nM (e.g., about 5 nM). The RNAi constructs of the invention may inhibit TTR expression in cells (e.g., CHO-transfected cells) expressing human TTR I148I or I148M with an IC50 of less than about 20 nM. For example, the RNAi constructs inhibit TTR expression in TTR I148I or I148M-expressing cells with an IC50 of about 0.001 nM to about 20 nM, about 0.001 nM to about 10 nM, about 0.001 nM to about 5 nM, about 0.001 nM to about 1 nM, about 0.1 nM to about 10 nM, about 0.1 nM to about 5 nM, or about 0.1 nM to about 1 nM. In certain embodiments, the RNAi construct inhibits TTR expression in TTR I148I or I148M-expressing cells with an IC50 of about 1 nM to about 10 nM (e.g., about 5 nM).

The RNAi constructs of the invention can readily be made using techniques known in the art, such as, for example, conventional nucleic acid solid phase synthesis. The polynucleotides of the RNAi constructs can be assembled on a suitable nucleic acid synthesizer utilizing standard nucleotide or nucleoside precursors (e.g., phosphoramidites). Automated nucleic acid synthesizers are sold commercially by several vendors, including DNA/RNA synthesizers from Applied Biosystems (Foster City, CA), MerMade synthesizers from BioAutomation (Irving, TX), and OligoPilot synthesizers from GE Healthcare Life Sciences (Pittsburgh, PA).

The 2′ silyl protecting group can be used in conjunction with acid labile dimethoxytrityl (DMT) at the 5′ position of ribonucleosides to synthesize oligonucleotides via phosphoramidite chemistry. Final deprotection conditions are known not to significantly degrade RNA products. All syntheses can be conducted in any automated or manual synthesizer on large, medium, or small scale. The syntheses may also be carried out in multiple well plates, columns, or glass slides.

The 2′-O-silyl group can be removed via exposure to fluoride ions, which can include any source of fluoride ion, e.g., those salts containing fluoride ion paired with inorganic counterions, e.g., cesium fluoride and potassium fluoride or those salts containing fluoride ion paired with an organic counterion, e.g., a tetraalkylammonium fluoride. A crown ether catalyst can be utilized in combination with the inorganic fluoride in the deprotection reaction. Exemplary fluoride ion sources include, but are not limited to, tetrabutylammonium fluoride or aminohydrofluorides (e.g., combining aqueous HF with triethylamine in a dipolar aprotic solvent, e.g., dimethylformamide).

The choice of protecting groups for use on the phosphite triesters and phosphotriesters can alter the stability of the triesters towards fluoride. Methyl protection of the phosphotriester or phosphite triester can stabilize the linkage against fluoride ions and improve process yields.

Since ribonucleosides have a reactive 2′ hydroxyl substituent, it may be desirable to protect the reactive 2′ position in RNA with a protecting group that is orthogonal to a 5′-O-dimethoxytrityl protecting group, e.g., one stable to treatment with acid. Silyl protecting groups meet this criterion and can be readily removed in a final fluoride deprotection step that can result in minimal RNA degradation.

Tetrazole catalysts can be used in the standard phosphoramidite coupling reaction. Exemplary catalysts include, e.g., tetrazole, S-ethyl-tetrazole, benzylthiotetrazole, and pnitrophenyltetrazole.

Additional methods of synthesizing the RNAi constructs described herein will be evident to those of ordinary skill in the art. Additionally, the various synthetic steps may be performed in an alternate sequence or order to give the desired compounds. Other synthetic chemistry transformations, protecting groups (e.g., for hydroxyl, amino, etc., present on the bases) and protecting group methodologies (protection and deprotection) useful in synthesizing the RNAi constructs described herein are known in the art and include, for example, those described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. Custom synthesis of RNAi constructs is also available from several commercial vendors, including Dharmacon, Inc. (Lafayette, CO), AxoLabs GmbH (Kulmbach, Germany), and Ambion, Inc. (Foster City, CA).

The RNAi constructs of the invention may comprise a ligand. As used herein, a “ligand” refers to any compound or molecule that can interact with another compound or molecule, either directly or indirectly. The interaction of a ligand with another compound or molecule may elicit a biological response (e.g., initiate a signal transduction cascade, induce receptor mediated endocytosis) or may just be a physical association. The ligand can modify one or more properties of the double-stranded RNA molecule to which is attached, such as the pharmacodynamic, pharmacokinetic, binding, absorption, cellular distribution, cellular uptake, charge and/or clearance properties of the RNA molecule.

The ligand may comprise a serum protein (e.g., human serum albumin, low-density lipoprotein, globulin), a cholesterol moiety, a vitamin (e.g., biotin, vitamin E, vitamin B12), a folate moiety, a steroid, a bile acid (e.g., cholic acid), a fatty acid (e.g., palmitic acid, myristic acid), a carbohydrate (e.g., a dextran, pullulan, chitin, chitosan, inulin, cyclodextrin or hyaluronic acid), a glycoside, a phospholipid, or an antibody or binding fragment thereof (e.g., a whole antibody or binding fragment that targets the RNAi construct to a specific cell type, such as liver cells). Other examples of ligands include dyes, intercalating agents (e.g., acridines), cross-linkers (e.g., psoralene, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, adamantane acetic acid, 1-pyrene butyric acid, dihydrotestosterone, 1,3-BisO(hexadecyl)glycerol, geranyloxyhexyl group, hexadecylglycerol, borneol, menthol, 1,3-propanediol, heptadecyl group, 03-(oleoyl) lithocholic acid, 03-(oleoyl) cholenic acid, dimethoxytrityl, or phenoxazine), peptides (e.g., antennapedia peptide, Tat peptide, RGD peptides), alkylating agents, polymers (e.g., polyethylene glycol (PEG), PEG-40K), poly amino acids, and polyamines (e.g., spermine, spermidine).

In certain embodiments, the ligands have endosomolytic properties. The endosomolytic ligands promote the lysis of the endosome and/or transport of the RNAi construct of the invention, or its components, from the endosome to the cytoplasm of the cell. The endosomolytic ligand may be a polycationic peptide or peptidomimetic which shows pH-dependent membrane activity and fusogenicity. In one embodiment, the endosomolytic ligand assumes its active conformation at endosomal pH. The “active” conformation is that conformation in which the endosomolytic ligand promotes lysis of the endosome and/or transport of the RNAi construct of the invention, or its components, from the endosome to the cytoplasm of the cell. Exemplary endosomolytic ligands include the GALA peptide (Subbarao et al., Biochemistry, Vol. 26:2964-2972, 1987), the EALA peptide (Vogel et al., J. Am. Chem. Soc., Vol. 118:1581-1586, 1996), and their derivatives (Turk et al., Biochem. Biophys. Acta, Vol. 1559:56-68, 2002). In one embodiment, the endosomolytic component may contain a chemical group (e.g., an amino acid) which will undergo a change in charge or protonation in response to a change in pH. The endosomolytic component may be linear or branched.

In some embodiments, the ligand comprises a lipid or other hydrophobic molecule. In one embodiment, the ligand comprises a cholesterol moiety or other steroid. Cholesterol conjugated oligonucleotides have been reported to be more active than their unconjugated counterparts (Manoharan, Antisense Nucleic Acid Drug Development, Vol. 12:103-228, 2002). Ligands comprising cholesterol moieties and other lipids for conjugation to nucleic acid molecules have also been described in U.S. Pat. Nos. 7,851,615; 7,745,608; and 7,833,992. In another embodiment, the ligand may comprise a folate moiety. Polynucleotides conjugated to folate moieties can be taken up by cells via a receptor-mediated endocytosis pathway. Such folate-polynucleotide conjugates are described in, e.g., U.S. Pat. No. 8,188,247.

Given that TTR is expressed in liver cells (e.g., hepatocytes), in certain embodiments, it is desirable to specifically deliver the RNAi construct to liver cells. In some embodiments, RNAi constructs can be specifically targeted to the liver by employing ligands that bind to or interact with proteins expressed on the surface of liver cells. For example, in certain embodiments, a ligand may comprise one or more antigen binding proteins (e.g. antibodies or binding fragments thereof (e.g. Fab, scFv)) that specifically bind to a receptor expressed on hepatocytes.

In certain embodiments, the ligand comprises a carbohydrate. A “carbohydrate” refers to a compound made up of one or more monosaccharide units having at least 6 carbon atoms (which can be linear, branched, or cyclic) with an oxygen, nitrogen or sulfur atom bonded to each carbon atom. Carbohydrates include, but are not limited to, sugars (e.g., monosaccharides, disaccharides, trisaccharides, tetrasaccharides, and oligosaccharides containing from about 4, 5, 6, 7, 8, or 9 monosaccharide units), and polysaccharides, such as starches, glycogen, cellulose, and polysaccharide gums. In some embodiments, the carbohydrate incorporated into the ligand is a monosaccharide selected from a pentose, hexose, or heptose and di- and tri-saccharides including such monosaccharide units. In other embodiments, the carbohydrate incorporated into the ligand is an amino sugar, such as galactosamine, glucosamine, N-acetylgalactosamine, and N-acetylglucosamine.

In some embodiments, the ligand comprises a hexose or hexosamine. The hexose may be selected from glucose, galactose, mannose, fucose, or fructose. The hexosamine may be selected from fructosamine, galactosamine, glucosamine, or mannosamine. In certain embodiments, the ligand comprises glucose, galactose, galactosamine, or glucosamine. In one embodiment, the ligand comprises glucose, glucosamine, or N-acetylglucosamine. In another embodiment, the ligand comprises galactose, galactosamine, or N-acetyl-galactosamine. In particular embodiments, the ligand comprises N-acetyl-galactosamine. Ligands comprising glucose, galactose, and N-acetyl-galactosamine (GalNAc) are particularly effective in targeting compounds to liver cells (see, e.g., D'Souza and Devarajan, J. Control Release, Vol. 203: 126-139, 2015). Examples of GalNAc- or galactose-containing ligands that can be incorporated into the RNAi constructs of the invention are described in U.S. Pat. Nos. 7,491,805; 8,106,022; and 8,877,917; U.S. Patent Publication No. 2003/0130186; and WIPO Publication No. WO 2013/166155.

In certain embodiments, the ligand comprises a multivalent carbohydrate moiety. As used herein, a “multivalent carbohydrate moiety” refers to a moiety comprising two or more carbohydrate units capable of independently binding or interacting with other molecules. For example, a multivalent carbohydrate moiety comprises two or more binding domains comprised of carbohydrates that can bind to two or more different molecules or two or more different sites on the same molecule. The valency of the carbohydrate moiety denotes the number of individual binding domains within the carbohydrate moiety. For instance, the terms “monovalent,” “bivalent,” “trivalent,” and “tetravalent” with reference to the carbohydrate moiety refer to carbohydrate moieties with one, two, three, and four binding domains, respectively. The multivalent carbohydrate moiety may comprise a multivalent lactose moiety, a multivalent galactose moiety, a multivalent glucose moiety, a multivalent N-acetyl-galactosamine moiety, a multivalent N-acetyl-glucosamine moiety, a multivalent mannose moiety, or a multivalent fucose moiety. In some embodiments, the ligand comprises a multivalent galactose moiety. In other embodiments, the ligand comprises a multivalent N-acetyl-galactosamine moiety. In these and other embodiments, the multivalent carbohydrate moiety is bivalent, trivalent, or tetravalent. In such embodiments, the multivalent carbohydrate moiety can be bi-antennary or tri-antennary. In one particular embodiment, the multivalent N-acetyl-galactosamine moiety is trivalent or tetravalent. In another particular embodiment, the multivalent galactose moiety is trivalent or tetravalent. Exemplary trivalent and tetravalent GalNAc-containing ligands for incorporation into the RNAi constructs of the invention are described in detail below.

The ligand can be attached or conjugated to the RNA molecule of the RNAi construct directly or indirectly. For instance, in some embodiments, the ligand is covalently attached directly to the sense or antisense strand of the RNAi construct. In other embodiments, the ligand is covalently attached via a linker to the sense or antisense strand of the RNAi construct. The ligand can be attached to nucleobases, sugar moieties, or internucleotide linkages of polynucleotides (e.g., sense strand or antisense strand) of the RNAi constructs of the invention. Conjugation or attachment to purine nucleobases or derivatives thereof can occur at any position including, endocyclic and exocyclic atoms. In certain embodiments, the 2-, 6-, 7-, or 8-positions of a purine nucleobase are attached to a ligand. Conjugation or attachment to pyrimidine nucleobases or derivatives thereof can also occur at any position. In some embodiments, the 2, 5-, and 6-positions of a pyrimidine nucleobase can be attached to a ligand. Conjugation or attachment to sugar moieties of nucleotides can occur at any carbon atom. Exemplary carbon atoms of a sugar moiety that can be attached to a ligand include the 2′, 3′, and 5′ carbon atoms. The 1′ position also can be attached to a ligand, such as in a basic residue. Internucleotide linkages can also support ligand attachments. For phosphorus-containing linkages (e.g., phosphodiester, phosphorothioate, phosphorodithiotate, phosphoroamidate, and the like), the ligand can be attached directly to the phosphorus atom or to an O, N, or S atom bound to the phosphorus atom. For amine- or amide-containing internucleoside linkages (e.g., PNA), the ligand can be attached to the nitrogen atom of the amine or amide or to an adjacent carbon atom.

In certain embodiments, the ligand may be attached to the 3′ or 5′ end of either the sense or antisense strand. In certain embodiments, the ligand is covalently attached to the 5′ end of the sense strand. In other embodiments, the ligand is covalently attached to the 3′ end of the sense strand. For example, in some embodiments, the ligand is attached to the 3′-terminal nucleotide of the sense strand. In certain such embodiments, the ligand is attached at the 3′-position of the 3′-terminal nucleotide of the sense strand. In alternative embodiments, the ligand is attached near the 3′ end of the sense strand, but before one or more terminal nucleotides (i.e. before 1, 2, 3, or 4 terminal nucleotides). In some embodiments, the ligand is attached at the 2′-position of the sugar of the 3′-terminal nucleotide of the sense strand.

In certain embodiments, the ligand is attached to the sense or antisense strand via a linker. A “linker” is an atom or group of atoms that covalently joins a ligand to a polynucleotide component of the RNAi construct. The linker may be from about 1 to about 30 atoms in length, from about 2 to about 28 atoms in length, from about 3 to about 26 atoms in length, from about 4 to about 24 atoms in length, from about 6 to about 20 atoms in length, from about 7 to about 20 atoms in length, from about 8 to about 20 atoms in length, from about 8 to about 18 atoms in length, from about 10 to about 18 atoms in length, and from about 12 to about 18 atoms in length. In some embodiments, the linker may comprise a bifunctional linking moiety, which generally comprises an alkyl moiety with two functional groups. One of the functional groups is selected to bind to the compound of interest (e.g., sense or antisense strand of the RNAi construct) and the other is selected to bind essentially any selected group, such as a ligand as described herein. In certain embodiments, the linker comprises a chain structure or an oligomer of repeating units, such as ethylene glycol or amino acid units. Examples of functional groups that are typically employed in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Linkers that may be used to attach a ligand to the sense or antisense strand in the RNAi constructs of the invention include, but are not limited to, pyrrolidine, 8-amino-3,6-di oxaoctanoic acid, succinimidyl 4-(N-maleimidomethyl)cyclohexane-1-carboxylate, 6-aminohexanoic acid, substituted C₁-C₁₀ alkyl, substituted or unsubstituted C₂-C₁₀ alkenyl or substituted or unsubstituted C₂-C₁₀ alkynyl. Preferred substituent groups for such linkers include, but are not limited to, hydroxyl, amino, alkoxy, carboxy, benzyl, phenyl, nitro, thiol, thioalkoxy, halogen, alkyl, aryl, alkenyl, and alkynyl.

In certain embodiments, the linkers are cleavable. A cleavable linker is one which is sufficiently stable outside the cell, but which upon entry into a target cell is cleaved to release the two parts the linker is holding together. In some embodiments, the cleavable linker is cleaved at least 10 times, 20 times, 30 times, 40 times, 50 times, 60 times, 70 times, 80 times, 90 times, or more, or at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linkers are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linker by reduction; esterases; endosomes or agents that can create an acidic environment, e.g., those that result in a pH of five or lower; enzymes that can hydrolyze or degrade an acid cleavable linker by acting as a general acid, peptidases (which can be substrate specific), and phosphatases.

A cleavable linker may comprise a moiety that is susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable group that is cleaved at a preferred pH, thereby releasing the RNA molecule from the ligand inside the cell, or into the desired compartment of the cell.

A linker can include a cleavable group that is cleavable by a particular enzyme. The type of cleavable group incorporated into a linker can depend on the cell to be targeted. For example, liver-targeting ligands can be linked to RNA molecules through a linker that includes an ester group. Liver cells are rich in esterases, and therefore the linker will be cleaved more efficiently in liver cells than in cell types that are not esterase-rich. Other types of cells rich in esterases include cells of the lung, renal cortex, and testis. Linkers that contain peptide bonds can be used when targeting cells rich in peptidases, such as liver cells and synoviocytes.

In general, the suitability of a candidate cleavable linker can be evaluated by testing the ability of a degradative agent (or condition) to cleave the candidate linker. It will also be desirable to also test the candidate cleavable linker for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In some embodiments, useful candidate linkers are cleaved at least 2, 4, 10, 20, 50, 70, or 100 times faster in the cell (or under in vitro conditions selected to mimic intracellular conditions) as compared to blood or serum (or under in vitro conditions selected to mimic extracellular conditions).

In other embodiments, redox cleavable linkers are utilized. Redox cleavable linkers are cleaved upon reduction or oxidation. An example of reductively cleavable group is a disulfide linking group (—S—S—). To determine if a candidate cleavable linker is a suitable “reductively cleavable linker,” or, for example, is suitable for use with a particular RNAi construct and particular ligand, one or more methods described herein can be used. For example, a candidate linker can be evaluated by incubation with dithiothreitol (DTT), or other reducing agent known in the art, which mimics the rate of cleavage that would be observed in a cell, e.g., a target cell. The candidate linkers can also be evaluated under conditions which are selected to mimic blood or serum conditions. In a specific embodiment, candidate linkers are cleaved by at most 10% in the blood. In other embodiments, useful candidate linkers are degraded at least 2, 4, 10, 20, 50, 70, or 100 times faster in the cell (or under in vitro conditions selected to mimic intracellular conditions) as compared to blood (or under in vitro conditions selected to mimic extracellular conditions).

In yet other embodiments, phosphate-based cleavable linkers are cleaved by agents that degrade or hydrolyze the phosphate group. An example of an agent that hydrolyzes phosphate groups in cells are enzymes, such as phosphatases in cells. Examples of phosphate-based cleavable groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Specific embodiments include —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —SP(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. Another specific embodiment is —O—P(O)(OH)—O—. These candidate linkers can be evaluated using methods analogous to those described above.

In other embodiments, the linkers may comprise acid cleavable groups, which are groups that are cleaved under acidic conditions. In some embodiments, acid cleavable groups are cleaved in an acidic environment with a pH of about 6.5 or lower (e.g., about 6.0, 5.5, 5.0, or lower), or by agents, such as enzymes that can act as a general acid. In a cell, specific low pH organelles, such as endosomes and lysosomes, can provide a cleaving environment for acid cleavable groups. Examples of acid cleavable linking groups include, but are not limited to, hydrazones, esters, and esters of amino acids. Acid cleavable groups can have the general formula —C═NN—, C(O)O, or —OC(O). A specific embodiment is when the carbon attached to the oxygen of the ester (the alkoxy group) is an aryl group, substituted alkyl group, or tertiaryalkyl group such as dimethyl, pentyl or t-butyl. These candidates can be evaluated using methods analogous to those described above.

In other embodiments, the linkers may comprise ester-based cleavable groups, which are cleaved by enzymes, such as esterases and amidases in cells. Examples of ester-based cleavable groups include, but are not limited to, esters of alkylene, alkenylene and alkynylene groups. Ester cleavable groups have the general formula —C(O)O—, or —OC(O)—. These candidate linkers can be evaluated using methods analogous to those described above.

In further embodiments, the linkers may comprise peptide-based cleavable groups, which are cleaved by enzymes, such as peptidases and proteases in cells. Peptide-based cleavable groups are peptide bonds formed between amino acids to yield oligopeptides (e.g., dipeptides, tripeptides etc.) and polypeptides. Peptide-based cleavable groups do not include the amide group (—C(O) NH—). The amide group can be formed between any alkylene, alkenylene or alkynelene. A peptide bond is a special type of amide bond formed between amino acids to yield peptides and proteins. The peptide-based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

Other types of linkers suitable for attaching ligands to the sense or antisense strands in the RNAi constructs described herein are known in the art and can include the linkers described in, e.g., U.S. Pat. Nos. 7,723,509; 8,017,762; 8,828,956; 8,877,917; and 9,181,551.

In certain embodiments, the ligand covalently attached to the sense or antisense strand of the RNAi constructs of the invention comprises a GalNAc moiety, e.g, a multivalent GalNAc moiety. In some embodiments, the multivalent GalNAc moiety is a trivalent GalNAc moiety and is attached to the 3′ end of the sense strand. In other embodiments, the multivalent GalNAc moiety is a trivalent GalNAc moiety and is attached to the 5′ end of the sense strand. In yet other embodiments, the multivalent GalNAc moiety is a tetravalent GalNAc moiety and is attached to the 3′ end of the sense strand. In still other embodiments, the multivalent GalNAc moiety is a tetravalent GalNAc moiety and is attached to the 5′ end of the sense strand.

In some embodiments, the RNAi constructs of the invention may be delivered to a cell or tissue of interest by administering a vector that encodes and controls the intracellular expression of the RNAi construct. A “vector” (also referred to herein as an “expression vector”) is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated viral vectors, retroviral vectors, and the like. A vector can be replicated in a living cell, or it can be made synthetically.

Generally, a vector for expressing an RNAi construct of the invention will comprise one or more promoters operably linked to sequences encoding the RNAi construct. The phrases “operably linked,” “operatively linked,” or “under transcriptional control” may be used interchangeably herein to indicate when a promoter is in the correct location and orientation in relation to a polynucleotide sequence to control the initiation of transcription by RNA polymerase and expression of the polynucleotide sequence. A “promoter” refers to a sequence recognized by the synthetic machinery of the cell, or introduced synthetic machinery, required to initiate the specific transcription of a gene sequence. Suitable promoters include, but are not limited to, RNA pol I, pol II, HI or U6 RNA pol III, and viral promoters (e.g., human cytomegalovirus (CMV) immediate early gene promoter, the SV40 early promoter, and the Rous sarcoma virus long terminal repeat). In some embodiments, an HI or U6RNA pol III promoter is employed. The promoter can be a tissue-specific or inducible promoter. Of particular interest are liver-specific promoters, such as promoter sequences from the human alpha-1 antitrypsin gene, albumin gene, hemopexin gene, and hepatic lipase gene. Inducible promoters include, for example, promoters regulated by ecdysone, estrogen, progesterone, tetracycline, and isopropyl-PD1-thiogalactopyranoside (IPTG).

When the RNAi construct comprises an siRNA, the two separate strands (sense and antisense strand) can be expressed from a single vector or two separate vectors. For example, in some embodiments, the sequence encoding the sense strand is operably linked to a promoter on a first vector and the sequence encoding the antisense strand is operably linked to a promoter on a second vector. In such an embodiment, the first and second vectors are co-introduced, e.g., by infection or transfection, into a target cell, such that the sense and antisense strands, once transcribed, will hybridize intracellularly to form the siRNA molecule. In another embodiment, the sense and antisense strands are transcribed from two separate promoters located in a single vector. In such embodiments, the sequence encoding the sense strand may be operably linked to a first promoter and the sequence encoding the antisense strand may be operably linked to a second promoter, wherein the first and second promoters are located in a single vector. In one embodiment, the vector comprises a first promoter operably linked to a sequence encoding the siRNA molecule, and a second promoter operably linked to the same sequence in the opposite direction, such that transcription of the sequence from the first promoter results in the synthesis of the sense strand of the siRNA molecule and transcription of the sequence from the second promoter results in synthesis of the antisense strand of the siRNA molecule.

When the RNAi construct comprises a shRNA, a sequence encoding the single, at least partially self-complementary RNA molecule is operably linked to a promoter to produce a single transcript. In some embodiments, the sequence encoding the shRNA comprises an inverted repeat joined by a linker polynucleotide sequence to produce the stem and loop structure of the shRNA following transcription.

In some embodiments, the vector encoding an RNAi construct of the invention is a viral vector. Various viral vector systems that are suitable to express the RNAi constructs described herein include, but are not limited to, adenoviral vectors, retroviral vectors (e.g., lentiviral vectors, maloney murine leukemia virus), adeno-associated viral vectors; herpes simplex viral vectors; SV40 vectors; polyoma viral vectors; papilloma viral vectors; picornaviral vectors; and pox viral vectors (e.g., vaccinia virus). In certain embodiments, the viral vector is a retroviral vector (e.g., lentiviral vector).

Various vectors suitable for use in the invention, methods for inserting nucleic acid sequences encoding siRNA or shRNA molecules into vectors, and methods of delivering the vectors to the cells of interest are known in the art (see, e.g., Dornburg, Gene Therap., Vol. 2: 301-310, 1995; Eglitis, Biotechniques, Vol. 6: 608-614, 1988; Miller, HumGene Therap., Vol. 1: 5-14, 1990; Anderson, Nature, Vol. 392: 25-30, 1998; Rubinson D A et al., Nat. Genet., Vol. 33: 401-406, 2003; Brummelkamp et al., Science, Vol. 296: 550-553, 2002; Brummelkamp et al., Cancer Cell, Vol. 2: 243-247, 2002; Lee et al., Nat Biotechnol, Vol. 20: 500-505, 2002; Miyagishi et al., Nat Biotechnol, Vol. 20: 497-500, 2002; Paddison et al., GenesDev, Vol. 16: 948-958, 2002; Paul et al., Nat Biotechnol, Vol. 20: 505-508, 2002; Sui et al., Proc Natl Acad Sci USA, Vol. 99: 5515-5520, 2002; and Yu et al., Proc Natl Acad Sci USA, Vol. 99: 6047-6052, 2002).

### Compositions

The disclosure also provides compositions and formulations comprising the RNAi constructs described herein and pharmaceutically acceptable carriers, excipients, or diluents. Such compositions and formulations are useful for reducing expression of TTR in a subject in need thereof. Where clinical applications are contemplated, pharmaceutical compositions and formulations will be prepared in a form appropriate for the intended application. Generally, this will entail preparing compositions that are essentially free of pyrogens, as well as other impurities that could be harmful to humans or animals.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable carrier, excipient, or diluent” includes solvents, buffers, solutions, dispersion media, coatings, antibacterial and antifungal agents, isotonic and absorption delaying agents, etc., acceptable for use in formulating pharmaceuticals, such as pharmaceuticals suitable for administration to humans. The use of such media and agents for pharmaceutically active substances is well known in the art. Except insofar as any conventional media or agent is incompatible with the RNAi constructs of the present invention, its use in therapeutic compositions is contemplated. Supplementary active ingredients also can be incorporated into the compositions, provided they do not inactivate the vectors or RNAi constructs of the compositions.

Compositions and methods for the formulation of pharmaceutical compositions depend on several criteria, including, but not limited to, route of administration, type and extent of disease or disorder to be treated, and dose to be administered. In some embodiments, the pharmaceutical compositions are formulated based on the intended route of delivery. For instance, in certain embodiments, the pharmaceutical compositions are formulated for parenteral delivery. Parenteral forms of delivery include intravenous, intraarterial, subcutaneous, intrathecal, intraperitoneal, and intramuscular injection or infusion. In one embodiment, the pharmaceutical composition is formulated for intravenous delivery. In such an embodiment, the pharmaceutical composition may include a lipid-based delivery vehicle. In another embodiment, the pharmaceutical composition is formulated for subcutaneous delivery. In such an embodiment, the pharmaceutical composition may include a targeting ligand (e.g., GalNAc-containing ligands described herein).

In some embodiments, the pharmaceutical compositions comprise an effective amount of an RNAi construct described herein. An “effective amount” is an amount sufficient to produce a beneficial or desired clinical result. In some embodiments, an effective amount is an amount sufficient to reduce TTR expression in hepatocytes of a subject. In some embodiments, an effective amount may be an amount sufficient to only partially reduce TTR expression, for example, to a level comparable to expression of the wild-type TTR allele in human heterozygotes.

An effective amount of an RNAi construct of the invention may be from about 0.01 mg/kg body weight to about 100 mg/kg body weight, about 0.05 mg/kg body weight to about 75 mg/kg body weight, about 0.1 mg/kg body weight to about 50 mg/kg body weight, about 1 mg/kg to about 30 mg/kg body weight, about 2.5 mg/kg of body weight to about 20 mg/kg bodyweight, or about 5 mg/kg body weight to about 15 mg/kg body weight. In certain embodiments, a single effective dose of an RNAi construct of the invention may be about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. The pharmaceutical composition comprising an effective amount of RNAi construct can be administered weekly, biweekly, monthly, quarterly, or biannually. The precise determination of what would be considered an effective amount and frequency of administration may be based on several factors, including a patient's size, age, gender, type of disorder to be treated (e.g., myocardial infarction, heart failure, coronary artery disease, hypercholesterolemia), particular RNAi construct employed, and route of administration. Estimates of effective dosages and in vivo half-lives for any particular RNAi construct of the invention can be ascertained using conventional methods and/or testing in appropriate animal models.

Colloidal dispersion systems, such as macromolecule complexes, nanocapsules, microspheres, beads, and lipid-based systems, including oil-in-water emulsions, micelles, mixed micelles, and liposomes, may be used as delivery vehicles for the RNAi constructs of the invention or vectors encoding such constructs. Commercially available fat emulsions that are suitable for delivering the nucleic acids of the invention include INTRALIPID®, LIPOSYN®, LIPOSYN® II, LIPOSYN® III, NUTRILIPID, and other similar lipid emulsions. A preferred colloidal system for use as a delivery vehicle in vivo is a liposome (i.e., an artificial membrane vesicle). The RNAi constructs of the invention may be encapsulated within liposomes, such as cationic liposomes. Alternatively, RNAi constructs of the invention may be complexed to lipids, such as cationic lipids. Suitable lipids and liposomes include neutral (e.g., dioleoylphosphatidyl ethanolamine (DOPE), dimyristoylphosphatidyl choline (DMPC), and dipalmitoyl phosphatidylcholine (DPPC)), distearolyphosphatidyl choline), negative (e.g., dimyristoylphosphatidyl glycerol (DMPG)), and cationic (e.g., dioleoyltetramethylaminopropyl (DOTAP) and dioleoylphosphatidyl ethanolamine (DOTMA)). The preparation and use of such colloidal dispersion systems is well known in the art. Exemplary formulations also are disclosed in, e.g., U.S. Pat. Nos. 5,783,565; 5,837,533; 5,981,505; 6,127,170; 6,217,900; 6,379,965; 6,383,512; 6,747,014; 7,202,227; and WO 03/093449.

In some embodiments, the RNAi constructs of the invention are fully encapsulated in a lipid formulation, e.g., to form a SPLP, pSPLP, SNALP, or other nucleic acid-lipid particle. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a noncationic lipid, and a lipid that prevents aggregation of the particle (e.g., a PEG-lipid conjugate). SNALPs and SPLPs are exceptionally useful for systemic applications, as they exhibit extended circulation lifetimes following intravenous injection and accumulate at distal sites (e.g., sites physically separated from the administration site). SPLPs include “pSPLP,” which include an encapsulated condensing agent-nucleic acid complex as set forth in PCT Publication No. WO 00/03683. The nucleic acid-lipid particles typically have a mean diameter of about 50 nm to about 150 nm, about 60 nm to about 130 nm, about 70 nm to about 110 nm, or about 70 nm to about 90 nm, and are substantially nontoxic. In addition, the nucleic acids present in the nucleic acid-lipid particles desirably are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; and 6,815,432; and PCT Publication No. WO 96/40964.

Pharmaceutical compositions suitable for injections include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, liquid polyethylene glycol, and the like), suitable mixtures thereof, and vegetable oils. The proper fluidity can be maintained, for example, by using a coating (such as lecithin), by maintaining the required particle size (in the case of dispersion), and/or by using surfactants. The prevention of the action of microorganisms can be brought about by various antibacterial and antifungal agents, such as, for example, parabens, chlorobutanol, phenol, sorbic acid, thimerosal, and the like. In many cases, isotonic agents (e.g., sugars or sodium chloride) may be included in the composition. Prolonged absorption of the injectable compositions can be brought about by including absorption-delaying agents, such as, for example, aluminum monostearate and gelatin.

Sterile injectable solutions may be prepared by incorporating an appropriate amount of the RNAi construct (alone or complexed with a ligand) into a solvent along with any other ingredients (such as described above) as desired, followed by filtered sterilization. Generally, dispersions are prepared by incorporating the various sterilized active ingredients into a sterile vehicle which contains the basic dispersion medium and the desired other ingredients. In the case of sterile powders for the preparation of sterile injectable solutions, suitable methods of preparation include vacuum-drying and freeze-drying techniques which yield a powder of the active ingredient(s) plus any additional desired ingredient from a previously sterile-filtered solution thereof.

The compositions provided herein may be formulated in a neutral or salt form. Pharmaceutically-acceptable salts include, for example, acid addition salts (formed with free amino groups) derived from inorganic acids (e.g., hydrochloric or phosphoric acids), or from organic acids (e.g., acetic, oxalic, tartaric, mandelic, and the like). Salts formed with free carboxyl groups can also be derived from inorganic bases (e.g., sodium, potassium, ammonium, calcium, or ferric hydroxides) or from organic bases (e.g., isopropylamine, trimethylamine, histidine, procaine, and the like).

For parenteral administration in an aqueous solution, for example, a solution generally is suitably buffered, and a liquid diluent is first rendered isotonic with, e.g., sufficient saline or glucose. Such aqueous solutions may be used, for example, for intravenous, intramuscular, subcutaneous, and intraperitoneal administration. Sterile aqueous media desirably are employed as is known to those of skill in the art. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). For human administration, preparations should meet sterility, pyrogenicity, general safety and purity standards as required by FDA standards. In certain embodiments, a pharmaceutical composition of the invention comprises or consists of a sterile saline solution and an RNAi construct described herein. In other embodiments, a pharmaceutical composition of the invention comprises or consists of an RNAi construct described herein and sterile water (e.g., water for injection, WFI). In still other embodiments, a pharmaceutical composition of the invention comprises or consists of an RNAi construct described herein and phosphate-buffered saline (PBS).

In some embodiments, the pharmaceutical compositions of the invention are packaged with or stored within a device for administration. Devices for injectable formulations include, but are not limited to, injection ports, pre-filled syringes, auto injectors, injection pumps, on-body injectors, and injection pens. Devices for aerosolized or powder formulations include, but are not limited to, inhalers, insufflators, aspirators, and the like. Thus, the present invention includes administration devices comprising a pharmaceutical composition of the invention for treating or preventing one or more of the disorders described herein.

### Methods for Inhibiting TTR Expression

The present disclosure also provides methods of inhibiting expression of a TTR gene in a cell. The methods include contacting a cell with an RNAi construct, e.g., double-stranded RNAi construct, in an amount effective to inhibit expression of TTR in the cell. Contacting a cell with an RNAi construct, e.g., a double-stranded RNAi construct, may be done in vitro or in vivo. Contacting a cell in vivo with the RNAi construct includes contacting a cell or group of cells within a subject, e.g., a human subject, with the RNAi construct. Combinations of in vitro and in vivo methods of contacting a cell also are within the scope of the present disclosure.

The present invention provides methods for reducing or inhibiting expression of TTR in a subject in need thereof as well as methods of treating or preventing conditions, diseases, or disorders associated with TTR expression or activity. A “condition, disease, or disorder associated with TTR expression” refers to conditions, diseases, or disorders in which TTR expression levels are altered or where elevated expression levels of TTR are associated with an increased risk of developing the condition, disease, or disorder.

Contacting a cell may be direct or indirect, as discussed above. Furthermore, contacting a cell may be accomplished via a targeting ligand, including any ligand described herein or known in the art. In preferred embodiments, the targeting ligand is a carbohydrate moiety, e.g., a GalNAc3 ligand, or any other ligand that directs the RNAi construct to a site of interest.

In one embodiment, contacting a cell with an RNAi includes “introducing” or “delivering the RNAi into the cell” by facilitating or effecting uptake or absorption into the cell. Absorption or uptake of an RNAi can occur through unaided diffusive or active cellular processes, or by auxiliary agents or devices. For in vivo introduction, for example, RNAi can be injected into a tissue site or administered systemically. In vitro introduction into a cell may be accomplished using methods known in the art, such as electroporation and lipofection. Additional approaches are described herein below and/or are known in the art.

The term “inhibiting,” as used herein, is used interchangeably with “reducing,” “silencing,” “downregulating”, “suppressing”, and other similar terms, and includes any level of inhibition.

The phrase “inhibiting expression of a TTR” is intended to refer to inhibition of expression of any TTR gene (such as, e.g., a mouse TTR gene, a rat TTR gene, a monkey TTR gene, or a human TTR gene) as well as variants or mutants of a TTR gene. Thus, the TTR gene may be a wild-type TTR gene, a mutant TTR gene (such as a mutant TTR gene giving rise to amyloid deposition), or a transgenic TTR gene in the context of a genetically manipulated cell, group of cells, or organism.

“Inhibiting expression of a TTR gene” includes any level of inhibition of a TTR gene, e.g., at least partial suppression of the expression of a TTR gene. The expression of the TTR gene may be assessed based on the level, or the change in the level, of any variable associated with TTR gene expression, e.g., TTR mRNA level, TTR protein level, or the number or extent of amyloid deposits. This level may be assessed in an individual cell or in a group of cells, including, for example, a sample derived from a subject.

Inhibition may be assessed by a decrease in an absolute or relative level of one or more variables that are associated with TTR expression compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control). In some embodiments, expression of a TTR gene is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

Inhibition of the expression of a TTR gene may be manifested by a reduction of the amount of mRNA expressed by a first cell or group of cells (such cells may be present, for example, in a sample derived from a subject) in which a TTR gene is transcribed and which has or have been treated (e.g., by contacting the cell or cells with an RNAi construct of the invention, or by administering an RNAi construct of the invention to a subject in which the cells are or were present), such that the expression of a TTR gene is inhibited, as compared to a second cell or group of cells substantially identical to the first cell or group of cells but which has not or have not been so treated (control cell(s)). Inhibition may be assessed by expressing the level of mRNA in treated cells as a percentage of the level of mRNA in control cells, using the following formula:

$$\frac{( mRNA in control cells ) - ( mRNA in treated cells )}{mRNA in control cells} \times 100 \%$$

Alternatively, inhibition of the expression of a TTR gene may be assessed in terms of a reduction of a parameter that is functionally linked to TTR gene expression, e.g., TTR protein expression or SREBP protein family activities. TTR gene silencing may be determined in any cell expressing TTR, either endogenously or recombinantly, by any assay known in the art.

Inhibition of the expression of a TTR protein may be manifested by a reduction in the level of the TTR protein that is expressed by a cell or group of cells (e.g., the level of protein expressed in a sample obtained from a subject). As explained above, for the assessment of mRNA suppression, the inhibition of protein expression levels in a treated cell or group of cells may similarly be expressed as a percentage of the level of protein in a control cell or group of cells.

A control cell or group of cells that may be used to assess the inhibition of the expression of a TTR gene includes a cell or group of cells that has not yet been contacted with an RNAi construct of the invention. For example, the control cell or group of cells may be derived from an individual subject (e.g., a human or animal subject) prior to treatment of the subject with an RNAi construct.

The level of TTR mRNA that is expressed by a cell or group of cells, or the level of circulating TTR mRNA, may be determined using any method known in the art for assessing mRNA expression, such as those mentioned above. In some embodiments, the level of expression of TTR in a sample is determined by detecting a transcribed polynucleotide, or portion thereof, e.g., mRNA of the TTR gene. In this regard, for example, RNA may be extracted from cells using RNA extraction techniques including, for example, acid phenol/guanidine isothiocyanate extraction (RNAzol B; Biogenesis), RNeasy RNA preparation kits (Qiagen), or PAXgene (PreAnalytix, Switzerland). Typical assay formats utilizing ribonucleic acid hybridization include nuclear run-on assays, RT-PCR, RNase protection assays (Melton et al., Nuc. Acids Res., 12:7035), northern blotting, in situ hybridization, and microarray analysis. Circulating TTR mRNA may be detected using methods described in WO 2012/177906.

In one embodiment, the level of expression of TTR is determined using a nucleic acid probe. The term “probe,” as used herein, refers to any molecule that is capable of selectively binding to a specific TTR sequence. Probes can be synthesized by one of skill in the art or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses, and probe arrays. One method for the determination of mRNA levels involves contacting isolated mRNA with a nucleic acid molecule (probe) that can hybridize to TTR mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in determining the level of TTR mRNA.

An alternative method for determining the level of expression of TTR in a sample involves the process of nucleic acid amplification and/or reverse transcriptase (to prepare cDNA) of for example mRNA in the sample, e.g., by RT-PCR (see, e.g., U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., supra; and U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In some aspects of the invention, the level of expression of TTR may be determined by quantitative fluorogenic RT-PCR {i.e., the TAQMAN™ System). The expression levels of TTR mRNA may be monitored using a membrane blot (such as used in hybridization analysis such as northern, Southern, dot, and the like), or microwells, sample tubes, gels, beads or fibers (or any solid support comprising bound nucleic acids) (see, e.g., U.S. Pat. Nos. 5,445,934; 5,677,195; 5,770,722; 5,744,305; and 5,874,219). The determination of TTR expression level may also comprise using nucleic acid probes in solution. In certain embodiments, the level of mRNA expression is assessed using branched DNA (bDNA) assays or real time PCR (qPCR).

The level of TTR protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, colorimetric assays, spectrophotometric assays, flow cytometry, immunodiffusion (single or double), immunoelectrophoresis, Western blotting, radioimmunoassay (RIA), enzyme-linked immunosorbent assays (ELISAs), immunofluorescent assays, electrochemiluminescence assays, etc.

In some embodiments, the efficacy of the methods of the invention can be monitored by detecting or monitoring a reduction in a symptom of a TTR-associated disease, such as gastrointestinal pain, difficulty breathing, high blood pressure, or swelling of the extremities, face, larynx, upper respiratory tract, abdomen, trunk, and genitals. These symptoms may be assessed in vitro or in vivo using any method known in the art.

In some embodiments, the RNAi construct or a composition comprising the RNAi construct is administered to a subject such that the RNAi construct is delivered to a specific site within the subject. The inhibition of expression of TTR may be assessed using measurements of the level or change in the level of TTR mRNA or TTR protein in a sample derived from fluid or tissue from the specific site within the subject. In some embodiments, the RNAi construct may be delivered to a site such as the liver, choroid plexus, retina, and pancreas. The site may also be a subsection or subgroup of cells from any one of the aforementioned sites. The site may also include cells that express a particular type of receptor.

### Methods of Treating or Preventing TTR-Associated Diseases

The present invention provides therapeutic and prophylactic methods which include administering to a subject with a TTR-associated disease, disorder, and/or condition, or prone to developing a TTR-associated disease, disorder, and/or condition, an RNAi construct, compositions (e.g., pharmaceutical compositions) comprising an RNAi construct, or vectors comprising an RNAi construct as described herein. Non-limiting examples of TTR-associated diseases include, for example, transthyretin-associated cardiomyopathy, amyloidosis, and hereditary amyloidosis (hATTR).

In certain embodiments, the present invention provides a method for reducing the expression of TTR in a patient in need thereof comprising administering to the patient any of the RNAi constructs described herein. The term “patient,” as used herein, refers to a mammal, including humans, and can be used interchangeably with the term “subject.” The expression level of TTR in hepatocytes in the patient desirably is reduced following administration of the RNAi construct as compared to the TTR expression level in a patient not receiving the RNAi construct.

The methods of the invention are useful for treating a subject having a TTR-associated disease, e.g., a subject that would benefit from reduction in TTR gene expression and/or TTR protein production. In one aspect, the present invention provides methods of reducing the level of TTR gene expression in a subject having nonalcoholic fatty liver disease (transthyretin-associated cardiomyopathy). In another aspect, the present invention provides methods of reducing the level of TTR protein in a subject with transthyretin-associated cardiomyopathy.

The treatment methods (and uses) of the invention include administering to the subject, e.g., a human, a therapeutically effective amount of the disclosed RNAi construct targeting a TTR gene, a pharmaceutical composition comprising the RNAi construct, or a vector comprising the RNAi construct.

In one aspect, the invention provides methods of preventing at least one symptom in a subject having transthyretin-associated cardiomyopathy, e.g., the presence of elevated hedgehog signaling pathways, fatigue, weakness, weight loss, loss of appetite, nausea, abdominal pain, spider-like blood vessels, yellowing of the skin and eyes (jaundice), itching, fluid buildup and swelling of the legs (edema), abdomen swelling (ascites), and mental confusion. The methods include administering to the subject a prophylactically effective amount of the RNAi construct, e.g., dsRNA, pharmaceutical compositions comprising the RNAi construct, or vectors encoding the RNAi construct, thereby preventing at least one symptom in the subject having a disorder that would benefit from reduction in TTR gene expression. A “prophylactically effective amount” refers to an amount effective, at dosages and for periods of time necessary, to achieve a desired prophylactic result (e.g., prevention of disease onset).

In another aspect, the present invention provides uses of a therapeutically effective amount of an RNAi construct of the invention for treating a subject, e.g., a subject that would benefit from a reduction and/or inhibition of TTR gene expression. In a further aspect, the present invention provides uses of an RNAi construct, e.g., a dsRNA, of the invention targeting an TTR gene or pharmaceutical composition comprising an RNAi construct targeting an TTR gene in the manufacture of a medicament for treating a subject, e.g., a subject that would benefit from a reduction and/or inhibition of TTR gene expression and/or TTR protein production, such as a subject having a disorder that would benefit from reduction in TTR gene expression, e.g., a TTR-associated disease.

The disclosure provides uses of an RNAi construct, e.g., a dsRNA, of the invention for preventing at least one symptom in a subject suffering from a disorder that would benefit from a reduction and/or inhibition of TTR gene expression and/or TTR protein production. For example, the disclosure provides uses of the RNAi construct described herein, compositions comprising same, and vectors comprising same, in the treatment of transthyretin-associated cardiomyopathy.

In a further aspect, the present invention provides uses of the disclosed RNAi construct, compositions comprising same, or a vector comprising same, in the manufacture of a medicament for preventing at least one symptom in a subject suffering from a disorder that would benefit from a reduction and/or inhibition of TTR gene expression and/or TTR protein production, such as a TTR-associated disease.

In one embodiment, an RNAi construct targeting TTR is administered to a subject having a TTR-associated disease, e.g., nonalcoholic fatty liver disease (transthyretin-associated cardiomyopathy), such that the expression of a TTR gene, e.g., in a cell, tissue, blood or other tissue or fluid of the subject is reduced by at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 62%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more when the RNAi construct is administered to the subject.

The methods and uses of the invention include administering a composition described herein such that expression of the target TTR gene is decreased for any suitable amount of time, such as for about 1, 2, 3, 4 5, 6, 7, 8, 12, 16, 18, 24, 28, 32, 36, 40, 44, 48, 52, 56, 60, 64, 68, 72, 76, or about 80 hours. In one embodiment, expression of the target TTR gene is decreased for an extended duration, e.g., at least about two, three, four, five, six, seven days or more, e.g., about one week, two weeks, three weeks, or about four weeks or longer.

Administration of the RNAi construct according to the methods and uses of the invention may result in a reduction of the severity, signs, symptoms, and/or markers of such diseases or disorders in a patient with a TTR-associated disease, e.g., transthyretin-associated cardiomyopathy. By “reduction” in this context is meant a statistically significant decrease in such level. The reduction can be, for example, at least about 5%, 10%, 15%, 20%, 25%, 30%, 35%, 40%, 45%, 50%, 55%, 60%, 65%, 70%, 75%, 80%, 85%, 90%, 95%, or about 100%. Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of transthyretin-associated cardiomyopathy may be assessed, for example, by periodic monitoring of transthyretin-associated cardiomyopathy symptoms, liver fat levels, or expression of downstream genes. Comparison of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an RNAi targeting TTR or pharmaceutical composition thereof, “effective against” an TTR-associated disease indicates that administration in a clinically appropriate manner results in a beneficial effect for at least a statistically significant fraction of patients, such as improvement of symptoms, a cure, a reduction in disease, extension of life, improvement in quality of life, or other effect generally recognized as positive by medical doctors familiar with treating transthyretin-associated cardiomyopathy and/or an TTR-associated disease and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given RNAi drug or formulation of that drug can also be judged using an experimental animal model for the given disease as known in the art. When using an experimental animal model, efficacy of treatment is evidenced when a statistically significant reduction in a marker or symptom is observed.

Subjects can be administered any therapeutically effective amount of the RNAi construct. Exemplary therapeutically effective amounts of the RNAi construct include, but are not limited to, 0.01 mg/kg, 0.02 mg/kg, 0.03 mg/kg, 0.04 mg/kg, 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg dsRNA, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 9.0 mg/kg, 10 mg/kg, 15 mg/kg, 20 mg/kg, 25 mg/kg, 30 mg/kg, 35 mg/kg, 40 mg/kg, 45 mg/kg, or about 50 mg/kg. In one embodiment, subjects can be administered 0.5 mg/kg of the RNAi construct. Values and ranges intermediate to the recited values also are encompassed by the present disclosure.

Administration of the RNAi construct, or a composition comprising same, can reduce the presence of TTR protein levels, e.g., in a cell, tissue, blood, urine or other compartment of the patient by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more.

Before administration of a full dose of the RNAi, patients can be administered a smaller dose, such as a 5% infusion, and monitored for adverse effects, such as an allergic reaction. In another example, the patient can be monitored for unwanted immunostimulatory effects, such as increased cytokine (e.g., TNF-alpha or INF-alpha) levels.

Owing to the inhibitory effects on TTR expression, a composition according to the invention or a pharmaceutical composition prepared therefrom can enhance the quality of life of a patient suffering from a TTR-associated disease (e.g., transthyretin-associated cardiomyopathy).

An RNAi of the invention may be administered in “naked” form, where the modified or unmodified RNAi construct is directly suspended in aqueous or suitable buffer solvent, as a “free RNAi.” A free RNAi is administered in the absence of a pharmaceutical composition. The free RNAi may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolality of the buffer solution containing the RNAi can be adjusted such that it is suitable for administering to a subject.

Alternatively, an RNAi construct of the invention may be administered as a pharmaceutical composition, such as a dsRNA liposomal formulation.

Subjects that would benefit from a reduction and/or inhibition of TTR gene expression are those having nonalcoholic fatty liver disease (transthyretin-associated cardiomyopathy) and/or another TTR-associated disease or disorder as described herein or otherwise known in the art.

The invention further provides methods and uses of an RNAi construct or a pharmaceutical composition thereof for treating a subject that would benefit from reduction and/or inhibition of TTR gene expression, e.g., a subject having a TTR-associated disease, in combination with other pharmaceuticals and/or other therapeutic methods, e.g., with known pharmaceuticals and/or known therapeutic methods, such as, for example, those which are currently employed for treating these disorders.

For example, in certain embodiments, an RNAi construct targeting a TTR gene is administered in combination with, e.g., an agent useful in treating a TTR-associated disease. For example, additional therapeutics and therapeutic methods suitable for treating a subject that would benefit from reduction in TTR expression, e.g., a subject having a TTR-associated disease, include an RNAi construct targeting a different portion of the TTR gene, a therapeutic agent, and/or procedures for treating a TTR-associated disease or a combination of any of the foregoing.

In certain embodiments, a first RNAi construct targeting a portion of a TTR gene is administered in combination with a second RNAi construct targeting a different portion of the TTR gene. For example, the first RNAi construct may comprise a first sense strand and a first antisense strand forming a double stranded region, wherein substantially all of the nucleotides of said first sense strand and substantially all of the nucleotides of the first antisense strand are modified nucleotides, wherein said first sense strand is conjugated to a ligand attached at the 3′-terminus, and wherein the ligand is one or more GalNAc derivatives attached through a bivalent or trivalent branched linker; and the second RNAi construct may comprise a second sense strand and a second antisense strand forming a double stranded region, wherein substantially all of the nucleotides of the second sense strand and substantially all of the nucleotides of the second antisense strand are modified nucleotides, wherein the second sense strand is conjugated to a ligand attached at the 3′-terminus, and wherein the ligand is one or more GalNAc derivatives attached through a bivalent or trivalent branched linker. In one embodiment, all of the nucleotides of the first and second sense strand and/or all of the nucleotides of the first and second antisense strand comprise a modification. The modified nucleotides may be any one or combination of the modified nucleotides described herein.

The RNAi construct and an additional therapeutic agent and/or treatment may be administered at the same time and/or in the same combination, e.g., parenterally, or the additional therapeutic agent can be administered as part of a separate composition or at separate times and/or by another method known in the art or described herein.

The present invention also provides methods of using an RNAi construct of the invention and/or a composition containing an RNAi construct of the invention to reduce and/or inhibit TTR expression (gene or protein expression) in a cell. In yet other aspects, use of an RNAi construct of the invention and/or a composition comprising an RNAi construct of the invention for the manufacture of a medicament for reducing and/or inhibiting TTR gene expression in a cell are provided. In still other aspects, the present invention provides an RNAi of the invention and/or a composition comprising an RNAi construct of the invention for use in reducing and/or inhibiting TTR protein production in a cell. In yet other aspects, use of an RNAi construct of the invention and/or a composition comprising an RNAi construct of the invention for the manufacture of a medicament for reducing and/or inhibiting TTR protein production in a cell are provided. The methods and uses include contacting the cell with an RNAi construct, e.g., a dsRNA, of the invention and maintaining the cell for a time sufficient to obtain degradation of the mRNA transcript of a TTR gene, thereby inhibiting expression of the TTR gene or inhibiting TTR protein production in the cell. Reduction in gene expression can be assessed by any methods known in the art or described herein for determining mRNA or protein levels.

In the methods and uses of the invention the cell may be contacted in vitro or in vivo, i.e., the cell may be outside (e.g., in cell culture) or within a subject. A cell suitable for treatment using the methods of the invention may be any cell that expresses an TTR gene, e.g., a cell from a subject having transthyretin-associated cardiomyopathy or a cell comprising an expression vector comprising a TTR gene or portion of a TTR gene. A suitable cell for use in the disclosed methods includes, for example, a mammalian cell, e.g., a primate cell (such as a human cell or a non-human primate cell, e.g., a monkey cell or a chimpanzee cell), a non-primate cell (such as a cow cell, a pig cell, a camel cell, a llama cell, a horse cell, a goat cell, a rabbit cell, a sheep cell, a hamster, a guinea pig cell, a cat cell, a dog cell, a rat cell, a mouse cell, a lion cell, a tiger cell, a bear cell, or a buffalo cell), a bird cell (e.g., a duck cell or a goose cell), or a whale cell. In one embodiment, the cell is a human cell.

TTR gene expression may be inhibited in the cell by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%.

TTR protein production may be inhibited in the cell by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%.

The in vivo methods and uses of the invention may include administering to a subject a composition containing an RNAi construct, where the RNAi construct includes a nucleotide sequence that is complementary to at least a part of an RNA transcript of the TTR gene of the subject. When the organism to be treated is a human, the composition can be administered by any means known in the art including, but not limited to subcutaneous, intravenous, oral, intraperitoneal, or parenteral routes, including intracranial (e.g., intraventricular, intraparenchymal, and intrathecal), intramuscular, transdermal, airway (aerosol), nasal, rectal, and topical (including buccal and sublingual) administration. In certain embodiments, the compositions are administered by subcutaneous or intravenous infusion or injection. In one embodiment, the compositions are administered by subcutaneous injection.

In some embodiments, the invention is a method for reducing the expression of TTR in vivo comprising administering to the animal an RNAi construct or composition, as described throughout the specification. The method can be where expression of TTR (e.g. human TTR) is knocked down by at least 45%, 50%, 60%, 70%, or 80% in an animal expressing hTTR by associated adenovirus (AAV) injection, and wherein the animal is administered the RNAi construct or the composition at 0.5 milligrams per kilogram of animal. The method can be where expression of TTR (e.g. human TTR) is knocked down by at least 60%, 70%, 80%, or 90% in an animal expressing hTTR by associated adenovirus (AAV) injection, and wherein the animal is administered the RNAi construct or the composition at 1.0 milligrams per kilogram of animal. The method can be where expression of TTR (e.g. human TTR) is knocked down by at least 80%, 85%, 90%, or 95% in an animal expressing hTTR by associated adenovirus (AAV) injection, and wherein the animal is administered the RNAi construct or the composition at 3.0 milligrams per kilogram of animal, as described in the Examples.

In some embodiments, the administration is via a depot injection. A depot injection may release the RNAi construct in a consistent way over a prolonged time period. Thus, a depot injection may reduce the frequency of dosing needed to obtain a desired effect, e.g., a desired inhibition of TTR, or a therapeutic or prophylactic effect. A depot injection may also provide more consistent serum concentrations. Depot injections may include subcutaneous injections or intramuscular injections. In some embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intravenous, subcutaneous, arterial, or epidural infusions. In preferred embodiments, the infusion pump is a subcutaneous infusion pump. In other embodiments, the pump is a surgically implanted pump that delivers the RNAi construct to the subject.

The mode of administration may be chosen based upon whether local or systemic treatment is desired and based upon the area to be treated. The route and site of administration may be chosen to enhance targeting.

The methods and uses include administering to the mammal, e.g., a human, a composition comprising an RNAi construct, e.g., an siRNA, that targets a TTR gene in a cell of the mammal and maintaining the mammal for a time sufficient to obtain degradation of the mRNA transcript of the TTR gene, thereby inhibiting expression of the TTR gene in the mammal. Reduction in gene expression and/or protein expression can be assessed in a sample obtained from the RNAi construct-administered subject by any method known in the art or described herein. In one embodiment, a tissue sample serves as the tissue material for monitoring the reduction in TTR gene and/or protein expression. In another embodiment, a blood sample serves as the tissue material for monitoring the reduction in TTR gene and/or protein expression.

In some embodiments, verification of RISC-mediated cleavage of a target mRNA (e.g., TTR mRNA) in vivo following administration of an RNAi construct may be assessed by performing 5′-RACE or modifications of the protocol as known in the art (Lasham A et al., (2010) Nucleic Acid Res., 38 (3) p-el9; and Zimmermann et al. (2006) Nature 441: 111-4).

It is understood that all ribonucleic acid sequences disclosed herein can be converted to deoxyribonucleic acid sequences by substituting a thymine base for a uracil base in the sequence. Likewise, all deoxyribonucleic acid sequences disclosed herein can be converted to ribonucleic acid sequences by substituting a uracil base for a thymine base in the sequence. Deoxyribonucleic acid sequences, ribonucleic acid sequences, and sequences containing mixtures of deoxyribonucleotides and ribonucleotides of all sequences disclosed herein are encompassed by the present invention.

Additionally, any nucleic acid sequences disclosed herein may be modified with any combination of chemical modifications. One of skill in the art will readily appreciate that such designation as “RNA” or “DNA” to describe modified polynucleotides is, in certain instances, arbitrary. For example, a polynucleotide comprising a nucleotide having a 2′-OH substituent on the ribose sugar and a thymine base could be described as a DNA molecule having a modified sugar (2′-OH for the natural 2′-H of DNA) or as an RNA molecule having a modified base (thymine (methylated uracil) for natural uracil of RNA).

Accordingly, nucleic acid sequences provided herein, including but not limited to those set forth in the sequence listing, are intended to encompass nucleic acids containing any combination of natural or modified RNA and/or DNA, including, but not limited to, such nucleic acids having modified nucleobases. By way of a further example and without limitation, a polynucleotide having the sequence “ATCGATCG” encompasses any polynucleotides having such a sequence, whether modified or unmodified, including, but not limited to, such compounds comprising RNA bases, such as those having sequence “AUCGAUCG” and those having some DNA bases and some RNA bases such as “AUCGATCG,” and polynucleotides having other modified bases, such as “ATmeCGAUCG,” wherein meC indicates a cytosine base comprising a methyl group at the 5-position.

In some embodiments, the invention is the following:

1. An RNAi construct comprising a sense strand and an antisense strand, wherein the antisense strand comprises a region having at least 15 contiguous nucleotides differing by no more than 3 nucleotides from an antisense sequence of: SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, or 1132, and wherein the RNAi construct inhibits the expression of a Transthyretin (TTR) mRNA.

2. An RNAi construct comprising a sense strand and an antisense strand, wherein the antisense strand has a transcript binding start location at nucleotide 195 to 201, and comprises a region having at least 15 contiguous nucleotides differing by no more than 3 nucleotides from an antisense sequence of SEQ ID NO: 18, 20, 22, 24, 26, 28, 584, 586, 588, 590, 592, 594, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, or 1214.

3. An RNAi construct comprising a sense strand and an antisense strand, wherein the antisense strand has a transcript binding start location at nucleotide 195 to 201, and comprises a region having at least 15 contiguous nucleotides differing by no more than 3 nucleotides from an antisense sequence and wherein the sense strand comprises a region having at least 15 contiguous nucleotides differing by no more than 3 nucleotides from a sense sequence, wherein the sense and antisense sequences comprise the sequence of SEQ ID NO: 17 and 18; 19 and 20; 21 and 22; 23 and 24; 25 and 26; 27 and 28; 583 and 584; 585 and 586; 587 and 588; 589 and 590; 591 and 592; 593 and 594; 1151 and 1152; 1153 and 1154; 1155 and 1156; 1157 and 1158; 1159 and 1160; 1161 and 1162; 1163 and 1164; 1165 and 1166; 1167 and 1168; 1169 and 1170; 1171 and 1172; 1173 and 1174; 1175 and 1176; 1177 and 1178; 1179 and 1180; 1181 and 1182; 1183 and 1184; 1185 and 1186; 1187 and 1188; 1189 and 1190; 1191 and 1192; 1193 and 1194; 1195 and 1196; 1197 and 1198; 1199 and 1200; 1201 and 1202; 1203 and 1204; 1205 and 1206; 1207 and 1208; 1209 and 1210; 1211 and 1212; or 1213 and 1214.

4. An RNAi construct comprising a sense strand and an antisense strand, wherein the antisense strand has a transcript binding start location at nucleotide 236 to 239, and comprises a region having at least 15 contiguous nucleotides differing by no more than 3 nucleotides from an antisense sequence of SEQ ID NO: 50, 52, 54, 56, 616, 618, 620, 622, 1256, 1258, 1260, 1262, 1264, 1266, 1268, or 1270.

5. An RNAi construct comprising a sense strand and an antisense strand, wherein the antisense strand has a transcript binding start location at nucleotide 236 to 239, and comprises a region having at least 15 contiguous nucleotides differing by no more than 3 nucleotides from an antisense sequence and wherein the sense strand comprises a region having at least 15 contiguous nucleotides differing by no more than 3 nucleotides from a sense sequence, wherein the sense and antisense sequences comprise the sequence of SEQ ID NO: 49 and 50; 51 and 52; 53 and 54; 55 and 56; 615 and 616; 617 and 618; 619 and 620; 621 and 622; 1255 and 1256; 1257 and 1258; 1259 and 1260; 1261 and 1262; 1263 and 1264; 1265 and 1266; 1267 and 1268; or 1269 and 1270.

6. An RNAi construct comprising a sense strand and an antisense strand, wherein the antisense strand has a transcript binding start location at nucleotide 277 to 283, and comprises a region having at least 15 contiguous nucleotides differing by no more than 3 nucleotides from an antisense sequence of SEQ ID NO: 62, 64, 66, 68, 70, 72, 74, 628, 630, 632, 634, 636, 638, 640, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, or 1348.

7. An RNAi construct comprising a sense strand and an antisense strand, wherein the antisense strand has a transcript binding start location at nucleotide 277 to 283, and comprises a region having at least 15 contiguous nucleotides differing by no more than 3 nucleotides from an antisense sequence and wherein the sense strand comprises a region having at least 15 contiguous nucleotides differing by no more than 3 nucleotides from a sense sequence, wherein the sense and antisense sequences comprise the sequence of SEQ ID NO: 61 and 62; 63 and 64; 65 and 66; 67 and 68; 69 and 70; 71 and 72; 73 and 74; 627 and 628; 629 and 630; 631 and 632; 633 and 634; 635 and 636; 637 and 638; 639 and 640; 1275 and 1276; 1277 and 1278; 1279 and 1280; 1281 and 1282; 1283 and 1284; 1285 and 1286; 1287 and 1288; 1289 and 1290; 1291 and 1292; 1293 and 1294; 1295 and 1296; 1297 and 1298; 1299 and 1300; 1301 and 1302; 1303 and 1304; 1305 and 1306; 1307 and 1308; 1309 and 1310; 1311 and 1312; 1313 and 1314; 1315 and 1316; 1317 and 1318; 1319 and 1320; 1321 and 1322; 1323 and 1324; 1325 and 1326; 1327 and 1328; 1329 and 1330; 1331 and 1332; 1333 and 1334; 1335 and 1336; 1337 and 1338; 1339 and 1340; 1341 and 1342; 1343 and 1344; 1345 and 1346; or 1347 and 1348.

8. The RNAi construct of any one of embodiments 1-7, wherein the sense strand comprises a sequence that is sufficiently complementary to the sequence of the antisense strand to form a duplex region of about 15 to about 30 base pairs in length.

9. The RNAi construct of any one of embodiments 1-8, wherein the duplex region is about 17 to about 24 base pairs in length.

10. The RNAi construct of any one of embodiments 1-9, wherein the duplex region is about 19 to about 21 base pairs in length.

11. The RNAi construct of embodiment 10, wherein the duplex region is 19 base pairs in length.

12. The RNAi construct of any one of embodiments 1-11, wherein the sense strand and the antisense strand are each about 15 to about 30 nucleotides in length.

13. The RNAi construct of embodiment 12, wherein the sense strand and the antisense strand are each about 19 to about 27 nucleotides in length.

14. The RNAi construct of embodiment 13, wherein the sense strand and the antisense strand are each about 21 to about 25 nucleotides in length.

15. The RNAi construct of embodiment 14, wherein the sense strand and the antisense strand are each about 21 to about 23 nucleotides in length.

16. The RNAi construct of any one of embodiments 1-15, which comprises at least one blunt end.

17. The RNAi construct of any one of embodiments 1-16, which comprises at least one nucleotide overhang of 1 to 4 unpaired nucleotides.

18. The RNAi construct of embodiment 17, wherein the nucleotide overhang has two unpaired nucleotides.

19. The RNAi construct of embodiment 17 or 18, wherein the RNAi construct comprises a nucleotide overhang at the 3′ end of the sense strand, the 3′ end of the antisense strand, or the 3′ end of both the sense strand and the antisense strand.

20. The RNAi construct of any one of embodiments 17-19, wherein the nucleotide overhang comprises a 5′-UU-3′ dinucleotide or a 5′-dTdT-3′ dinucleotide.

21. The RNAi construct of any one of embodiments 1-20, wherein the RNAi construct comprises at least one modified nucleotide.

22. The RNAi construct of embodiment 21, wherein the modified nucleotide is a 2′-modified nucleotide.

23. The RNAi construct of embodiment 21, wherein the modified nucleotide is a 2′-fluoro modified nucleotide, a 2′-O-methyl modified nucleotide, a 2′-O-methoxyethyl modified nucleotide, a 2′-O-allyl modified nucleotide, a bicyclic nucleic acid (BNA), a glycol nucleic acid, an inverted base, or combinations thereof.

24. The RNAi construct of embodiment 23, wherein the modified nucleotide is a 2′-O-methyl modified nucleotide, a 2′-O-methoxyethyl modified nucleotide, a 2′-fluoro modified nucleotide, or combinations thereof.

25. The RNAi construct of any one of embodiments 21-24, wherein all of the nucleotides in the sense and antisense strands are modified nucleotides.

26. The RNAi construct of embodiment 25, wherein the modified nucleotides are 2′-O-methylmodified nucleotides, 2′-fluoro modified nucleotides, or combinations thereof.

27. The RNAi construct of any one of embodiments 1-26, which comprises at least one phosphorothioate internucleotide linkage.

28. The RNAi construct of embodiment 27, wherein the RNAi construct comprises two consecutive phosphorothioate internucleotide linkages at the 3′ end of the antisense strand.

29. The RNAi construct of embodiment 27, wherein the RNAi construct comprises two consecutive phosphorothioate internucleotide linkages at both the 3′ and 5′ ends of the antisense strand and two consecutive phosphorothioate internucleotide linkages at the 5′ end of the sense strand.

30. The RNAi construct of any one of embodiments 1-29, wherein the antisense strand comprises a sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, or 566.

31. The RNAi construct of any one of embodiments 1-29, wherein the antisense strand comprises a sequence of SEQ ID NO: 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, or 1132.

32. The RNAi construct of any one of embodiments 1-31, wherein the sense strand comprises a sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, or 565.

33. The RNAi construct of any one of embodiments 1-31, wherein the sense strand comprises a sequence of SEQ ID NO: 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1471, 1473, 1475, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, or 1617.

34. The RNAi construct of any one of embodiments 1-33, wherein the RNAi construct is any one of the duplex compounds of: SEQ ID NO: 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32; 33 and 34; 35 and 36; 37 and 38; 39 and 40; 41 and 42; 43 and 44; 45 and 46; 47 and 48; 49 and 50; 51 and 52; 53 and 54; 55 and 56; 57 and 58; 59 and 60; 61 and 62; 63 and 64; 65 and 66; 67 and 68; 69 and 70; 71 and 72; 73 and 74; 75 and 76; 77 and 78; 79 and 80; 81 and 82; 83 and 84; 85 and 86; 87 and 88; 89 and 90; 91 and 92; 93 and 94; 95 and 96; 97 and 98; 99 and 100; 101 and 102; 103 and 104; 105 and 106; 107 and 108; 109 and 110; 111 and 112; 113 and 114; 115 and 116; 117 and 118; 119 and 120; 121 and 122; 123 and 124; 125 and 126; 127 and 128; 129 and 130; 131 and 132; 133 and 134; 135 and 136; 137 and 138; 139 and 140; 141 and 142; 143 and 144; 145 and 146; 147 and 148; 149 and 150; 151 and 152; 153 and 154; 155 and 156; 157 and 158; 159 and 160; 161 and 162; 163 and 164; 165 and 166; 167 and 168; 169 and 170; 171 and 172; 173 and 174; 175 and 176; 177 and 178; 179 and 180; 181 and 182; 183 and 184; 185 and 186; 187 and 188; 189 and 190; 191 and 192; 193 and 194; 195 and 196; 197 and 198; 199 and 200; 201 and 202; 203 and 204; 205 and 206; 207 and 208; 209 and 210; 211 and 212; 213 and 214; 215 and 216; 217 and 218; 219 and 220; 221 and 222; 223 and 224; 225 and 226; 227 and 228; 229 and 230; 231 and 232; 233 and 234; 235 and 236; 237 and 238; 239 and 240; 241 and 242; 243 and 244; 245 and 246; 247 and 248; 249 and 250; 251 and 252; 253 and 254; 255 and 256; 257 and 258; 259 and 260; 261 and 262; 263 and 264; 265 and 266; 267 and 268; 269 and 270; 271 and 272; 273 and 274; 275 and 276; 277 and 278; 279 and 280; 281 and 282; 283 and 284; 285 and 286; 287 and 288; 289 and 290; 291 and 292; 293 and 294; 295 and 296; 297 and 298; 299 and 300; 301 and 302; 303 and 304; 305 and 306; 307 and 308; 309 and 310; 311 and 312; 313 and 314; 315 and 316; 317 and 318; 319 and 320; 321 and 322; 323 and 324; 325 and 326; 327 and 328; 329 and 330; 331 and 332; 333 and 334; 335 and 336; 337 and 338; 339 and 340; 341 and 342; 343 and 344; 345 and 346; 347 and 348; 349 and 350; 351 and 352; 353 and 354; 355 and 356; 357 and 358; 359 and 360; 361 and 362; 363 and 364; 365 and 366; 367 and 368; 369 and 370; 371 and 372; 373 and 374; 375 and 376; 377 and 378; 379 and 380; 381 and 382; 383 and 384; 385 and 386; 387 and 388; 389 and 390; 391 and 392; 393 and 394; 395 and 396; 397 and 398; 399 and 400; 401 and 402; 403 and 404; 405 and 406; 407 and 408; 409 and 410; 411 and 412; 413 and 414; 415 and 416; 417 and 418; 419 and 420; 421 and 422; 423 and 424; 425 and 426; 427 and 428; 429 and 430; 431 and 432; 433 and 434; 435 and 436; 437 and 438; 439 and 440; 441 and 442; 443 and 444; 445 and 446; 447 and 448; 449 and 450; 451 and 452; 453 and 454; 455 and 456; 457 and 458; 459 and 460; 461 and 462; 463 and 464; 465 and 466; 467 and 468; 469 and 470; 471 and 472; 473 and 474; 475 and 476; 477 and 478; 479 and 480; 481 and 482; 483 and 484; 485 and 486; 487 and 488; 489 and 490; 491 and 492; 493 and 494; 495 and 496; 497 and 498; 499 and 500; 501 and 502; 503 and 504; 505 and 506; 507 and 508; 509 and 510; 511 and 512; 513 and 514; 515 and 516; 517 and 518; 519 and 520; 521 and 522; 523 and 524; 525 and 526; 527 and 528; 529 and 530; 531 and 532; 533 and 534; 535 and 536; 537 and 538; 539 and 540; 541 and 542; 543 and 544; 545 and 546; 547 and 548; 549 and 550; 551 and 552; 553 and 554; 555 and 556; 557 and 558; 559 and 560; 561 and 562; 563 and 564; or 565 and 566.

35. The RNAi construct of any one of embodiments 1-33, wherein the RNAi construct is any one of the duplex compounds of: SEQ ID NO: 567 and 568; 569 and 570; 571 and 572; 573 and 574; 575 and 576; 577 and 578; 579 and 580; 581 and 582; 583 and 584; 585 and 586; 587 and 588; 589 and 590; 591 and 592; 593 and 594; 595 and 596; 597 and 598; 599 and 600; 601 and 602; 603 and 604; 605 and 606; 607 and 608; 609 and 610; 611 and 612; 613 and 614; 615 and 616; 617 and 618; 619 and 620; 621 and 622; 623 and 624; 625 and 626; 627 and 628; 629 and 630; 631 and 632; 633 and 634; 635 and 636; 637 and 638; 639 and 640; 641 and 642; 643 and 644; 645 and 646; 647 and 648; 649 and 650; 651 and 652; 653 and 654; 655 and 656; 657 and 658; 659 and 660; 661 and 662; 663 and 664; 665 and 666; 667 and 668; 669 and 670; 671 and 672; 673 and 674; 675 and 676; 677 and 678; 679 and 680; 681 and 682; 683 and 684; 685 and 686; 687 and 688; 689 and 690; 691 and 692; 693 and 694; 695 and 696; 697 and 698; 699 and 700; 701 and 702; 703 and 704; 705 and 706; 707 and 708; 709 and 710; 711 and 712; 713 and 714; 715 and 716; 717 and 718; 719 and 720; 721 and 722; 723 and 724; 725 and 726; 727 and 728; 729 and 730; 731 and 732; 733 and 734; 735 and 736; 737 and 738; 739 and 740; 741 and 742; 743 and 744; 745 and 746; 747 and 748; 749 and 750; 751 and 752; 753 and 754; 755 and 756; 757 and 758; 759 and 760; 761 and 762; 763 and 764; 765 and 766; 767 and 768; 769 and 770; 771 and 772; 773 and 774; 775 and 776; 777 and 778; 779 and 780; 781 and 782; 783 and 784; 785 and 786; 787 and 788; 789 and 790; 791 and 792; 793 and 794; 795 and 796; 797 and 798; 799 and 800; 801 and 802; 803 and 804; 805 and 806; 807 and 808; 809 and 810; 811 and 812; 813 and 814; 815 and 816; 817 and 818; 819 and 820; 821 and 822; 823 and 824; 825 and 826; 827 and 828; 829 and 830; 831 and 832; 833 and 834; 835 and 836; 837 and 838; 839 and 840; 841 and 842; 843 and 844; 845 and 846; 847 and 848; 849 and 850; 851 and 852; 853 and 854; 855 and 856; 857 and 858; 859 and 860; 861 and 862; 863 and 864; 865 and 866; 867 and 868; 869 and 870; 871 and 872; 873 and 874; 875 and 876; 877 and 878; 879 and 880; 881 and 882; 883 and 884; 885 and 886; 887 and 888; 889 and 890; 891 and 892; 893 and 894; 895 and 896; 897 and 898; 899 and 900; 901 and 902; 903 and 904; 905 and 906; 907 and 908; 909 and 910; 911 and 912; 913 and 914; 915 and 916; 917 and 918; 919 and 920; 921 and 922; 923 and 924; 925 and 926; 927 and 928; 929 and 930; 931 and 932; 933 and 934; 935 and 936; 937 and 938; 939 and 940; 941 and 942; 943 and 944; 945 and 946; 947 and 948; 949 and 950; 951 and 952; 953 and 954; 955 and 956; 957 and 958; 959 and 960; 961 and 962; 963 and 964; 965 and 966; 967 and 968; 969 and 970; 971 and 972; 973 and 974; 975 and 976; 977 and 978; 979 and 980; 981 and 982; 983 and 984; 985 and 986; 987 and 988; 989 and 990; 991 and 992; 993 and 994; 995 and 996; 997 and 998; 999 and 1000; 1001 and 1002; 1003 and 1004; 1005 and 1006; 1007 and 1008; 1009 and 1010; 1011 and 1012; 1013 and 1014; 1015 and 1016; 1017 and 1018; 1019 and 1020; 1021 and 1022; 1023 and 1024; 1025 and 1026; 1027 and 1028; 1029 and 1030; 1031 and 1032; 1033 and 1034; 1035 and 1036; 1037 and 1038; 1039 and 1040; 1041 and 1042; 1043 and 1044; 1045 and 1046; 1047 and 1048; 1049 and 1050; 1051 and 1052; 1053 and 1054; 1055 and 1056; 1057 and 1058; 1059 and 1060; 1061 and 1062; 1063 and 1064; 1065 and 1066; 1067 and 1068; 1069 and 1070; 1071 and 1072; 1073 and 1074; 1075 and 1076; 1077 and 1078; 1079 and 1080; 1081 and 1082; 1083 and 1084; 1085 and 1086; 1087 and 1088; 1089 and 1090; 1091 and 1092; 1093 and 1094; 1095 and 1096; 1097 and 1098; 1099 and 1100; 1101 and 1102; 1103 and 1104; 1105 and 1106; 1107 and 1108; 1109 and 1110; 1111 and 1112; 1113 and 1114; 1115 and 1116; 1117 and 1118; 1119 and 1120; 1121 and 1122; 1123 and 1124; 1125 and 1126; 1127 and 1128; 1129 and 1130; 1131 and 1132; 1133 and 1134; 1135 and 1136; 1137 and 1138; 1139 and 1140; 1141 and 1142; 1143 and 1144; 1145 and 1146; 1147 and 1148; 1149 and 1150; 1151 and 1152; 1153 and 1154; 1155 and 1156; 1157 and 1158; 1159 and 1160; 1161 and 1162; 1163 and 1164; 1165 and 1166; 1167 and 1168; 1169 and 1170; 1171 and 1172; 1173 and 1174; 1175 and 1176; 1177 and 1178; 1179 and 1180; 1181 and 1182; 1183 and 1184; 1185 and 1186; 1187 and 1188; 1189 and 1190; 1191 and 1192; 1193 and 1194; 1195 and 1196; 1197 and 1198; 1199 and 1200; 1201 and 1202; 1203 and 1204; 1205 and 1206; 1207 and 1208; 1209 and 1210; 1211 and 1212; 1213 and 1214; 1215 and 1216; 1217 and 1218; 1219 and 1220; 1221 and 1222; 1223 and 1224; 1225 and 1226; 1227 and 1228; 1229 and 1230; 1231 and 1232; 1233 and 1234; 1235 and 1236; 1237 and 1238; 1239 and 1240; 1241 and 1242; 1243 and 1244; 1245 and 1246; 1247 and 1248; 1249 and 1250; 1251 and 1252; 1253 and 1254; 1255 and 1256; 1257 and 1258; 1259 and 1260; 1261 and 1262; 1263 and 1264; 1265 and 1266; 1267 and 1268; 1269 and 1270; 1271 and 1272; 1273 and 1274; 1275 and 1276; 1277 and 1278; 1279 and 1280; 1281 and 1282; 1283 and 1284; 1285 and 1286; 1287 and 1288; 1289 and 1290; 1291 and 1292; 1293 and 1294; 1295 and 1296; 1297 and 1298; 1299 and 1300; 1301 and 1302; 1303 and 1304; 1305 and 1306; 1307 and 1308; 1309 and 1310; 1311 and 1312; 1313 and 1314; 1315 and 1316; 1317 and 1318; 1319 and 1320; 1321 and 1322; 1323 and 1324; 1325 and 1326; 1327 and 1328; 1329 and 1330; 1331 and 1332; 1333 and 1334; 1335 and 1336; 1337 and 1338; 1339 and 1340; 1341 and 1342; 1343 and 1344; 1345 and 1346; 1347 and 1348; 1349 and 1350; 1351 and 1352; 1353 and 1354; 1355 and 1356; 1357 and 1358; 1359 and 1360; 1361 and 1362; 1363 and 1364; 1365 and 1366; 1367 and 1368; 1369 and 1370; 1371 and 1372; 1373 and 1374; 1375 and 1376; 1377 and 1378; 1379 and 1380; 1381 and 1382; 1383 and 1384; 1385 and 1386; 1387 and 1388; 1389 and 1390; 1391 and 1392; 1393 and 1394; 1395 and 1396; 1397 and 1398; 1399 and 1400; 1401 and 1402; 1403 and 1404; 1405 and 1406; 1407 and 1408; 1409 and 1410; 1411 and 1412; 1413 and 1414; 1415 and 1416; 1417 and 1418; 1419 and 1420; 1421 and 1422; 1423 and 1424; 1425 and 1426; 1427 and 1428; 1429 and 1430; 1431 and 1432; 1433 and 1434; 1435 and 1436; 1437 and 1438; 1439 and 1440; 1441 and 1442; 1443 and 1444; 1445 and 1446; 1447 and 1448; 1449 and 1450; 1451 and 1452; 1453 and 1454; 1455 and 1456; 1457 and 1458; 1459 and 1460; 1461 and 1462; 1463 and 1464; 1465 and 1466; 1467 and 1468; 1469 and 1470; 1471 and 1472; 1473 and 1474; 1475 and 1476; 1477 and 1478; 1479 and 1480; 1481 and 1482; 1483 and 1484; 1485 and 1486; 1487 and 1488; 1489 and 1490; 1491 and 1492; 1493 and 1494; 1495 and 1496; 1497 and 1498; 1499 and 1500; 1501 and 1502; 1503 and 1504; 1505 and 1506; 1507 and 1508; 1509 and 1510; 1511 and 1512; 1513 and 1514; 1515 and 1516; 1517 and 1518; 1519 and 1520; 1521 and 1522; 1523 and 1524; 1525 and 1526; 1527 and 1528; 1529 and 1530; 1531 and 1532; 1533 and 1534; 1535 and 1536; 1537 and 1538; 1539 and 1540; 1541 and 1542; 1543 and 1544; 1545 and 1546; 1547 and 1548; 1549 and 1550; 1551 and 1552; 1553 and 1554; 1555 and 1556; 1557 and 1558; 1559 and 1560; 1561 and 1562; 1563 and 1564; 1565 and 1566; 1567 and 1568; 1569 and 1570; 1571 and 1572; 1573 and 1574; 1575 and 1576; 1577 and 1578; 1579 and 1580; 1581 and 1582; 1583 and 1584; 1585 and 1586; 1587 and 1588; 1589 and 1590; 1591 and 1592; 1593 and 1594; 1595 and 1596; 1597 and 1598; 1599 and 1600; 1601 and 1602; 1603 and 1604; 1605 and 1606; 1607 and 1608; 1609 and 1610; 1611 and 1612; 1613 and 1614; 1615 and 1616; or 1617 and 1618.

36. The RNAi construct of any one of embodiments 1-35, which is a small interfering RNA (siRNA).

37. The RNAi construct of any one of embodiments 1-36, wherein the RNAi construct reduces the expression level of TTR in liver cells following incubation with the RNAi construct as compared to the TTR expression level in liver cells that have been incubated with a control RNAi construct.

38. The RNAi construct of embodiment 37, wherein the liver cells are Hep3B or HepG2 cells.

39. The RNAi construct of any one of embodiments 1-38, wherein the RNAi construct inhibits at least 40% of TTR expression at 5 nM in Hep3B cells in vitro.

40. The RNAi construct of any one of embodiments 1-38, wherein the RNAi construct inhibits at least 40% of TTR expression at 5 nM in HepG2 cells in vitro.

41. The RNAi construct of any one of embodiments 1-40, wherein the RNAi construct inhibits TTR expression in Hep3B cells with an IC50 of less than about 5 nM.

42. The RNAi construct of any one of embodiments 1-40, wherein the RNAi construct inhibits TTR expression in HepG2 cells with an IC50 of less than about 5 nM.

43. The RNAi construct of any one of embodiments 1-42, further comprising a ligand that binds to one or more proteins expressed on the surface of liver cells.

44. A composition comprising the RNAi construct of any one of embodiments 1-43 and a pharmaceutically acceptable carrier, excipient, or diluent.

45. A method for reducing the expression of TTR in a patient in need thereof comprising administering to the patient the RNAi construct of any one of embodiments 1-43 or the composition of embodiment 44.

46. The method of embodiment 45, wherein the expression level of TTR in hepatocytes is reduced in the patient following administration of an effective amount of the RNAi construct as compared to the TTR expression level in a patient not receiving the RNAi construct.

47. The method of any one of embodiments 45-46, wherein the patient suffers from transthyretin-associated cardiomyopathy (ATTR-CM).

48. A method for reducing the expression of TTR in vivo comprising administering to the animal the RNAi construct of any one of embodiments 1-43 or the composition of embodiment 44.

49. The method of embodiment 48, wherein the TTR is human TTR (hTTR).

50. The method of any one of embodiments 48 or 49, wherein expression of TTR is knocked down by at least 45% in an animal expressing hTTR by associated adenovirus (AAV) injection, and wherein the animal is administered the RNAi construct or the composition at 0.5 milligrams per kilogram of animal.

51. The method of embodiment 50, wherein the expression of TTR is knocked down by at least 50%, by at least 60%, or by at least 70%.

52. The method of any one of embodiments 48 or 49, wherein expression of TTR is knocked down by at least 60% in an animal expressing hTTR by associated adenovirus (AAV) injection, and wherein the animal is administered the RNAi construct or the composition at 1.0 milligrams per kilogram of animal.

53. The method of embodiment 52, wherein the expression of TTR is knocked down by at least 80%.

54. The method of any one of embodiments 48 or 49, wherein expression of TTR is knocked down by at least 90% in an animal expressing hTTR by associated adenovirus (AAV) injection, and wherein the animal is administered the RNAi construct or the composition at 0.5 milligrams per kilogram of animal.

55. The method of embodiment 52, wherein the expression of TTR is knocked down by at least 95%.

56. An RNAi construct of any one of embodiments 1-43 or a composition of embodiment 44 for use in the treatment of transthyretin-associated cardiomyopathy (ATTR-CM).

57. An RNAi construct of any one of embodiments 1-43 or a composition of embodiment 44 for use in decreasing expression of TTR by at least 45% in an animal expressing hTTR by associated adenovirus (AAV) injection, and wherein the animal is administered the RNAi construct or the composition at 0.5 milligrams per kilogram of animal.

58. The RNAi construct or composition of embodiment 57, wherein the expression of TTR is knocked down by at least 50%, by at least 60%, or by at least 70%.

59. An RNAi construct of any one of embodiments 1-43 or a composition of embodiment 44 for use in decreasing expression of TTR by at least 60% in an animal expressing hTTR by associated adenovirus (AAV) injection, and wherein the animal is administered the RNAi construct or the composition at 1.0 milligrams per kilogram of animal.

60. The RNAi construct or composition of embodiment 59, wherein the expression of TTR is knocked down by at least 80%.

61. An RNAi construct of any one of embodiments 1-43 or a composition of embodiment 44 for use in decreasing expression of TTR by at least 90% in an animal expressing hTTR by associated adenovirus (AAV) injection, and wherein the animal is administered the RNAi construct or the composition at 0.5 milligrams per kilogram of animal.

62. The RNAi construct or composition of embodiment 61, wherein the expression of TTR is knocked down by at least 95%.

63. Use of an RNAi construct of any one of embodiments 1-43 or a composition of embodiment 44 in the manufacture of a medicament for the treatment of transthyretin-associated cardiomyopathy (ATTR-CM).

64. Use of an RNAi construct of any one of embodiments 1-43 or a composition of embodiment 44 in the manufacture of a medicament for decreasing expression of TTR by at least 45% in an animal expressing hTTR by associated adenovirus (AAV) injection, and wherein the animal is administered the RNAi construct or the composition at 0.5 milligrams per kilogram of animal.

65. The use of embodiment 64, wherein the embodiment of TTR is knocked down by at least 50%, by at least 60%, or by at least 70%.

66. Use of an RNAi construct of any one of embodiments 1-43 or a composition of embodiment 44 in the manufacture of a medicament for decreasing expression of TTR by at least 60% in an animal expressing hTTR by associated adenovirus (AAV) injection, and wherein the animal is administered the RNAi construct or the composition at 1.0 milligrams per kilogram of animal.

67. The use of embodiment 66, wherein the expression of TTR is knocked down by at least 80%.

68. Use of an RNAi construct of any one of embodiments 1-43 or a composition of embodiment 44 in the manufacture of a medicament for decreasing expression of TTR by at least 90% in an animal expressing hTTR by associated adenovirus (AAV) injection, and wherein the animal is administered the RNAi construct or the composition at 0.5 milligrams per kilogram of animal.

69. The use of embodiment 68, wherein the expression of TTR is knocked down by at least 95%.

The following examples, including the experiments conducted and the results achieved, are provided for illustrative purposes only and are not to be construed as limiting the scope of the appended claims.

### EXAMPLES

### Example 1: Selection, Design and Synthesis of Modified TTR siRNA Molecules

The identification and selection of optimal sequences for therapeutic siRNA molecules targeting transthyretin (TTR) were identified using bioinformatics analysis of a human TTR transcript (Ensembl transcript ENST00000237014.7). Table 1 shows sequences identified as having therapeutic properties. Throughout the various sequences, “invAb” is an inverted abasic nucleotide.

TABLE 1
siRNA sequences directed to TTR:

| | | | | SEQ ID |
|---|---|---|---|---|
| | | SEQ ID | | NO: |
| Duplex | | NO: | | (anti- |
| No. | Sense sequence (5′-3′) | (sense) | Antisense sequence (5′-3′) | sense) |
| D-1000 | UUCUUGGCAGGAUGGCUUCU{invAb} | 1 | AAGAAGCCAUCCUGCCAAGAAUU | 2 |
| D-1001 | UCUUGGCAGGAUGGCUUCUC{invAb} | 3 | UGAGAAGCCAUCCUGCCAAGAUU | 4 |
| D-1002 | CUUGGCAGGAUGGCUUCUCA{invAb} | 5 | AUGAGAAGCCAUCCUGCCAAGUU | 6 |
| D-1003 | UUGGCAGGAUGGCUUCUCAU{invAb} | 7 | AAUGAGAAGCCAUCCUGCCAAUU | 8 |
| D-1004 | UGGCAGGAUGGCUUCUCAUC{invAb} | 9 | AGAUGAGAAGCCAUCCUGCCAUU | 10 |
| D-1005 | GGCAGGAUGGCUUCUCAUCG{invAb} | 11 | ACGAUGAGAAGCCAUCCUGCCUU | 12 |
| D-1006 | GCAGGAUGGCUUCUCAUCGU{invAb} | 13 | AACGAUGAGAAGCCAUCCUGCUU | 14 |
| D-1007 | CAGGAUGGCUUCUCAUCGUC{invAb} | 15 | AGACGAUGAGAAGCCAUCCUGUU | 16 |
| D-1008 | AGGAUGGCUUCUCAUCGUCU{invAb} | 17 | AAGACGAUGAGAAGCCAUCCUUU | 18 |
| D-1009 | GGAUGGCUUCUCAUCGUCUG{invAb} | 19 | ACAGACGAUGAGAAGCCAUCCUU | 20 |
| D-1010 | GAUGGCUUCUCAUCGUCUGC{invAb} | 21 | AGCAGACGAUGAGAAGCCAUCUU | 22 |
| D-1011 | AUGGCUUCUCAUCGUCUGCU{invAb} | 23 | AAGCAGACGAUGAGAAGCCAUUU | 24 |
| D-1012 | UGGCUUCUCAUCGUCUGCUC{invAb} | 25 | AGAGCAGACGAUGAGAAGCCAUU | 26 |
| D-1013 | GCUUCUCAUCGUCUGCUCCU{invAb} | 27 | AAGGAGCAGACGAUGAGAAGCUU | 28 |
| D-1014 | CUUCUCAUCGUCUGCUCCUC{invAb} | 29 | AGAGGAGCAGACGAUGAGAAGUU | 30 |
| D-1015 | UUCUCAUCGUCUGCUCCUCC{invAb} | 31 | AGGAGGAGCAGACGAUGAGAAUU | 32 |
| D-1016 | UCUCAUCGUCUGCUCCUCCU{invAb} | 33 | AAGGAGGAGCAGACGAUGAGAUU | 34 |
| D-1017 | CUCAUCGUCUGCUCCUCCUC{invAb} | 35 | AGAGGAGGAGCAGACGAUGAGUU | 36 |
| D-1018 | CAUCGUCUGCUCCUCCUCUG{invAb} | 37 | ACAGAGGAGGAGCAGACGAUGUU | 38 |
| D-1019 | GCUGGACUGGUAUUUGUGUC{invAb} | 39 | AGACACAAAUACCAGUCCAGCUU | 40 |
| D-1020 | CUGGACUGGUAUUUGUGUCU{invAb} | 41 | AAGACACAAAUACCAGUCCAGUU | 42 |
| D-1021 | UGGACUGGUAUUUGUGUCUG{invAb} | 43 | UCAGACACAAAUACCAGUCCAUU | 44 |
| D-1022 | GGACUGGUAUUUGUGUCUGA{invAb} | 45 | AUCAGACACAAAUACCAGUCCUU | 46 |
| D-1023 | GACUGGUAUUUGUGUCUGAG{invAb} | 47 | ACUCAGACACAAAUACCAGUCUU | 48 |
| D-1024 | ACUGGUAUUUGUGUCUGAGG{invAb} | 49 | ACCUCAGACACAAAUACCAGUUU | 50 |
| D-1025 | CUGGUAUUUGUGUCUGAGGC{invAb} | 51 | AGCCUCAGACACAAAUACCAGUU | 52 |
| D-1026 | UGGUAUUUGUGUCUGAGGCU{invAb} | 53 | AAGCCUCAGACACAAAUACCAUU | 54 |
| D-1027 | GGUAUUUGUGUCUGAGGCUG{invAb} | 55 | ACAGCCUCAGACACAAAUACCUU | 56 |
| D-1028 | UGAAUCCAAGUGUCCUCUGA{invAb} | 57 | AUCAGAGGACACUUGGAUUCAUU | 58 |
| D-1029 | GAAUCCAAGUGUCCUCUGAU{invAb} | 59 | AAUCAGAGGACACUUGGAUUCUU | 60 |
| D-1030 | AAUCCAAGUGUCCUCUGAUG{invAb} | 61 | ACAUCAGAGGACACUUGGAUUUU | 62 |
| D-1031 | AUCCAAGUGUCCUCUGAUGG{invAb} | 63 | ACCAUCAGAGGACACUUGGAUUU | 64 |
| D-1032 | UCCAAGUGUCCUCUGAUGGU{invAb} | 65 | AACCAUCAGAGGACACUUGGAUU | 66 |
| D-1033 | CCAAGUGUCCUCUGAUGGUC{invAb} | 67 | UGACCAUCAGAGGACACUUGGUU | 68 |
| D-1034 | CAAGUGUCCUCUGAUGGUCA{invAb} | 69 | UUGACCAUCAGAGGACACUUGUU | 70 |
| D-1035 | AAGUGUCCUCUGAUGGUCAA{invAb} | 71 | UUUGACCAUCAGAGGACACUUUU | 72 |
| D-1036 | AGUGUCCUCUGAUGGUCAAA{invAb} | 73 | AUUUGACCAUCAGAGGACACUUU | 74 |
| D-1037 | UGUCCUCUGAUGGUCAAAGU{invAb} | 75 | AACUUUGACCAUCAGAGGACAUU | 76 |
| D-1038 | GUCCUCUGAUGGUCAAAGUU{invAb} | 77 | AAACUUUGACCAUCAGAGGACUU | 78 |
| D-1039 | UCCUCUGAUGGUCAAAGUUC{invAb} | 79 | AGAACUUUGACCAUCAGAGGAUU | 80 |
| D-1040 | CCUCUGAUGGUCAAAGUUCU{invAb} | 81 | UAGAACUUUGACCAUCAGAGGUU | 82 |
| D-1041 | CUCUGAUGGUCAAAGUUCUA{invAb} | 83 | AUAGAACUUUGACCAUCAGAGUU | 84 |
| D-1042 | UCUGAUGGUCAAAGUUCUAG{invAb} | 85 | UCUAGAACUUUGACCAUCAGAUU | 86 |
| D-1043 | CUGAUGGUCAAAGUUCUAGA{invAb} | 87 | AUCUAGAACUUUGACCAUCAGUU | 88 |
| D-1044 | UGAUGGUCAAAGUUCUAGAU{invAb} | 89 | AAUCUAGAACUUUGACCAUCAUU | 90 |
| D-1045 | GAUGGUCAAAGUUCUAGAUG{invAb} | 91 | ACAUCUAGAACUUUGACCAUCUU | 92 |
| D-1046 | GCCAUUUGCCUCUGGGAAAA{invAb} | 93 | AUUUUCCCAGAGGCAAAUGGCUU | 94 |
| D-1047 | CCAUUUGCCUCUGGGAAAAC{invAb} | 95 | AGUUUUCCCAGAGGCAAAUGGUU | 96 |
| D-1048 | CAUUUGCCUCUGGGAAAACC{invAb} | 97 | UGGUUUUCCCAGAGGCAAAUGUU | 98 |
| D-1049 | AUUUGCCUCUGGGAAAACCA{invAb} | 99 | AUGGUUUUCCCAGAGGCAAAUUU | 100 |
| D-1050 | UUUGCCUCUGGGAAAACCAG{invAb} | 101 | ACUGGUUUUCCCAGAGGCAAAUU | 102 |
| D-1051 | UUGCCUCUGGGAAAACCAGU{invAb} | 103 | AACUGGUUUUCCCAGAGGCAAUU | 104 |
| D-1052 | UGCCUCUGGGAAAACCAGUG{invAb} | 105 | UCACUGGUUUUCCCAGAGGCAUU | 106 |
| D-1053 | CUCUGGGAAAACCAGUGAGU{invAb} | 107 | AACUCACUGGUUUUCCCAGAGUU | 108 |
| D-1054 | UCUGGGAAAACCAGUGAGUC{invAb} | 109 | AGACUCACUGGUUUUCCCAGAUU | 110 |
| D-1055 | CUGGGAAAACCAGUGAGUCU{invAb} | 111 | AAGACUCACUGGUUUUCCCAGUU | 112 |
| D-1056 | UGGGAAAACCAGUGAGUCUG{invAb} | 113 | ACAGACUCACUGGUUUUCCCAUU | 114 |
| D-1057 | GGGAAAACCAGUGAGUCUGG{invAb} | 115 | UCCAGACUCACUGGUUUUCCCUU | 116 |
| D-1058 | GGAAAACCAGUGAGUCUGGA{invAb} | 117 | AUCCAGACUCACUGGUUUUCCUU | 118 |
| D-1059 | GAAAACCAGUGAGUCUGGAG{invAb} | 119 | UCUCCAGACUCACUGGUUUUCUU | 120 |
| D-1060 | AAAACCAGUGAGUCUGGAGA{invAb} | 121 | AUCUCCAGACUCACUGGUUUUUU | 122 |
| D-1061 | AAACCAGUGAGUCUGGAGAG{invAb} | 123 | ACUCUCCAGACUCACUGGUUUUU | 124 |
| D-1062 | AACCAGUGAGUCUGGAGAGC{invAb} | 125 | AGCUCUCCAGACUCACUGGUUUU | 126 |
| D-1063 | GUGAGUCUGGAGAGCUGCAU{invAb} | 127 | AAUGCAGCUCUCCAGACUCACUU | 128 |
| D-1064 | UGAGUCUGGAGAGCUGCAUG{invAb} | 129 | ACAUGCAGCUCUCCAGACUCAUU | 130 |
| D-1065 | GAGUCUGGAGAGCUGCAUGG{invAb} | 131 | ACCAUGCAGCUCUCCAGACUCUU | 132 |
| D-1066 | GUCUGGAGAGCUGCAUGGGC{invAb} | 133 | AGCCCAUGCAGCUCUCCAGACUU | 134 |
| D-1067 | UCUGGAGAGCUGCAUGGGCU{invAb} | 135 | AAGCCCAUGCAGCUCUCCAGAUU | 136 |
| D-1068 | CUGGAGAGCUGCAUGGGCUC{invAb} | 137 | UGAGCCCAUGCAGCUCUCCAGUU | 138 |
| D-1069 | GGAGAGCUGCAUGGGCUCAC{invAb} | 139 | UGUGAGCCCAUGCAGCUCUCCUU | 140 |
| D-1070 | GAGAGCUGCAUGGGCUCACA{invAb} | 141 | UUGUGAGCCCAUGCAGCUCUCUU | 142 |
| D-1071 | AGAGCUGCAUGGGCUCACAA{invAb} | 143 | AUUGUGAGCCCAUGCAGCUCUUU | 144 |
| D-1072 | GAGCUGCAUGGGCUCACAAC{invAb} | 145 | AGUUGUGAGCCCAUGCAGCUCUU | 146 |
| D-1073 | AGCUGCAUGGGCUCACAACU{invAb} | 147 | AAGUUGUGAGCCCAUGCAGCUUU | 148 |
| D-1074 | GCUGCAUGGGCUCACAACUG{invAb} | 149 | UCAGUUGUGAGCCCAUGCAGCUU | 150 |
| D-1075 | CUGCAUGGGCUCACAACUGA{invAb} | 151 | AUCAGUUGUGAGCCCAUGCAGUU | 152 |
| D-1076 | UGCAUGGGCUCACAACUGAG{invAb} | 153 | ACUCAGUUGUGAGCCCAUGCAUU | 154 |
| D-1077 | GCAUGGGCUCACAACUGAGG{invAb} | 155 | UCCUCAGUUGUGAGCCCAUGCUU | 156 |
| D-1078 | CAUGGGCUCACAACUGAGGA{invAb} | 157 | AUCCUCAGUUGUGAGCCCAUGUU | 158 |
| D-1079 | GGCUCACAACUGAGGAGGAA{invAb} | 159 | AUUCCUCCUCAGUUGUGAGCCUU | 160 |
| D-1080 | GCUCACAACUGAGGAGGAAU{invAb} | 161 | AAUUCCUCCUCAGUUGUGAGCUU | 162 |
| D-1081 | CUCACAACUGAGGAGGAAUU{invAb} | 163 | AAAUUCCUCCUCAGUUGUGAGUU | 164 |
| D-1082 | CACAACUGAGGAGGAAUUUG{invAb} | 165 | ACAAAUUCCUCCUCAGUUGUGUU | 166 |
| D-1083 | ACAACUGAGGAGGAAUUUGU{invAb} | 167 | UACAAAUUCCUCCUCAGUUGUUU | 168 |
| D-1084 | CAACUGAGGAGGAAUUUGUA{invAb} | 169 | AUACAAAUUCCUCCUCAGUUGUU | 170 |
| D-1085 | AACUGAGGAGGAAUUUGUAG{invAb} | 171 | UCUACAAAUUCCUCCUCAGUUUU | 172 |
| D-1086 | ACUGAGGAGGAAUUUGUAGA{invAb} | 173 | UUCUACAAAUUCCUCCUCAGUUU | 174 |
| D-1087 | CUGAGGAGGAAUUUGUAGAA{invAb} | 175 | AUUCUACAAAUUCCUCCUCAGUU | 176 |
| D-1088 | UGAGGAGGAAUUUGUAGAAG{invAb} | 177 | ACUUCUACAAAUUCCUCCUCAUU | 178 |
| D-1089 | GAGGAGGAAUUUGUAGAAGG{invAb} | 179 | ACCUUCUACAAAUUCCUCCUCUU | 180 |
| D-1090 | AGGAGGAAUUUGUAGAAGGG{invAb} | 181 | UCCCUUCUACAAAUUCCUCCUUU | 182 |
| D-1091 | GGAGGAAUUUGUAGAAGGGA{invAb} | 183 | AUCCCUUCUACAAAUUCCUCCUU | 184 |
| D-1092 | GAGGAAUUUGUAGAAGGGAU{invAb} | 185 | UAUCCCUUCUACAAAUUCCUCUU | 186 |
| D-1093 | GGAAUUUGUAGAAGGGAUAU{invAb} | 187 | UAUAUCCCUUCUACAAAUUCCUU | 188 |
| D-1094 | GAAUUUGUAGAAGGGAUAUA{invAb} | 189 | AUAUAUCCCUUCUACAAAUUCUU | 190 |
| D-1095 | AAUUUGUAGAAGGGAUAUAC{invAb} | 191 | UGUAUAUCCCUUCUACAAAUUUU | 192 |
| D-1096 | AAUUUGUAGAAGGGAUAUAC{invAb} | 193 | UGUAUAUCCCUUCUACAAAUUUU | 194 |
| D-1097 | AUUUGUAGAAGGGAUAUACA{invAb} | 195 | UUGUAUAUCCCUUCUACAAAUUU | 196 |
| D-1098 | UUUGUAGAAGGGAUAUACAA{invAb} | 197 | UUUGUAUAUCCCUUCUACAAAUU | 198 |
| D-1099 | UUGUAGAAGGGAUAUACAAA{invAb} | 199 | AUUUGUAUAUCCCUUCUACAAUU | 200 |
| D-1100 | UGUAGAAGGGAUAUACAAAG{invAb} | 201 | ACUUUGUAUAUCCCUUCUACAUU | 202 |
| D-1101 | UGUAGAAGGGAUAUACAAAG{invAb} | 203 | ACUUUGUAUAUCCCUUCUACAUU | 204 |
| D-1102 | GUAGAAGGGAUAUACAAAGU{invAb} | 205 | AACUUUGUAUAUCCCUUCUACUU | 206 |
| D-1103 | UAGAAGGGAUAUACAAAGUG{invAb} | 207 | ACACUUUGUAUAUCCCUUCUAUU | 208 |
| D-1104 | AGAAGGGAUAUACAAAGUGG{invAb} | 209 | UCCACUUUGUAUAUCCCUUCUUU | 210 |
| D-1105 | GAAGGGAUAUACAAAGUGGA{invAb} | 211 | UUCCACUUUGUAUAUCCCUUCUU | 212 |
| D-1106 | GAAGGGAUAUACAAAGUGGA{invAb} | 213 | UUCCACUUUGUAUAUCCCUUCUU | 214 |
| D-1107 | AAGGGAUAUACAAAGUGGAA{invAb} | 215 | UUUCCACUUUGUAUAUCCCUUUU | 216 |
| D-1108 | AGGGAUAUACAAAGUGGAAA{invAb} | 217 | AUUUCCACUUUGUAUAUCCCUUU | 218 |
| D-1109 | GGGAUAUACAAAGUGGAAAU{invAb} | 219 | UAUUUCCACUUUGUAUAUCCCUU | 220 |
| D-1110 | GGAUAUACAAAGUGGAAAUA{invAb} | 221 | AUAUUUCCACUUUGUAUAUCCUU | 222 |
| D-1111 | GAUAUACAAAGUGGAAAUAG{invAb} | 223 | UCUAUUUCCACUUUGUAUAUCUU | 224 |
| D-1112 | AUAUACAAAGUGGAAAUAGA{invAb} | 225 | AUCUAUUUCCACUUUGUAUAUUU | 226 |
| D-1113 | UAUACAAAGUGGAAAUAGAC{invAb} | 227 | UGUCUAUUUCCACUUUGUAUAUU | 228 |
| D-1114 | AUACAAAGUGGAAAUAGACA{invAb} | 229 | AUGUCUAUUUCCACUUUGUAUUU | 230 |
| D-1115 | AUACAAAGUGGAAAUAGACA{invAb} | 231 | AUGUCUAUUUCCACUUUGUAUUU | 232 |
| D-1116 | UACAAAGUGGAAAUAGACAC{invAb} | 233 | AGUGUCUAUUUCCACUUUGUAUU | 234 |
| D-1117 | ACAAAGUGGAAAUAGACACC{invAb} | 235 | UGGUGUCUAUUUCCACUUUGUUU | 236 |
| D-1118 | CAAAGUGGAAAUAGACACCA{invAb} | 237 | UUGGUGUCUAUUUCCACUUUGUU | 238 |
| D-1119 | AAGUGGAAAUAGACACCAAA{invAb} | 239 | AUUUGGUGUCUAUUUCCACUUUU | 240 |
| D-1120 | AAGUGGAAAUAGACACCAAA{invAb} | 241 | AUUUGGUGUCUAUUUCCACUUUU | 242 |
| D-1121 | AGUGGAAAUAGACACCAAAU{invAb} | 243 | AAUUUGGUGUCUAUUUCCACUUU | 244 |
| D-1122 | GUGGAAAUAGACACCAAAUC{invAb} | 245 | AGAUUUGGUGUCUAUUUCCACUU | 246 |
| D-1123 | UGGAAAUAGACACCAAAUCU{invAb} | 247 | AAGAUUUGGUGUCUAUUUCCAUU | 248 |
| D-1124 | GGAAAUAGACACCAAAUCUU{invAb} | 249 | UAAGAUUUGGUGUCUAUUUCCUU | 250 |
| D-1125 | GGAAAUAGACACCAAAUCUU{invAb} | 251 | UAAGAUUUGGUGUCUAUUUCCUU | 252 |
| D-1126 | GAAAUAGACACCAAAUCUUA{invAb} | 253 | AUAAGAUUUGGUGUCUAUUUCUU | 254 |
| D-1127 | AAAUAGACACCAAAUCUUAC{invAb} | 255 | AGUAAGAUUUGGUGUCUAUUUUU | 256 |
| D-1128 | AAUAGACACCAAAUCUUACU{invAb} | 257 | AAGUAAGAUUUGGUGUCUAUUUU | 258 |
| D-1129 | AUAGACACCAAAUCUUACUG{invAb} | 259 | ACAGUAAGAUUUGGUGUCUAUUU | 260 |
| D-1130 | AUAGACACCAAAUCUUACUG{invAb} | 261 | ACAGUAAGAUUUGGUGUCUAUUU | 262 |
| D-1131 | UAGACACCAAAUCUUACUGG{invAb} | 263 | UCCAGUAAGAUUUGGUGUCUAUU | 264 |
| D-1132 | AGACACCAAAUCUUACUGGA{invAb} | 265 | UUCCAGUAAGAUUUGGUGUCUUU | 266 |
| D-1133 | CACUUGGCAUCUCCCCAUUC{invAb} | 267 | AGAAUGGGGAGAUGCCAAGUGUU | 268 |
| D-1134 | ACUUGGCAUCUCCCCAUUCC{invAb} | 269 | UGGAAUGGGGAGAUGCCAAGUUU | 270 |
| D-1135 | ACUUGGCAUCUCCCCAUUCC{invAb} | 271 | UGGAAUGGGGAGAUGCCAAGUUU | 272 |
| D-1136 | CUUGGCAUCUCCCCAUUCCA{invAb} | 273 | AUGGAAUGGGGAGAUGCCAAGUU | 274 |
| D-1137 | UUGGCAUCUCCCCAUUCCAU{invAb} | 275 | AAUGGAAUGGGGAGAUGCCAAUU | 276 |
| D-1138 | UGGCAUCUCCCCAUUCCAUG{invAb} | 277 | UCAUGGAAUGGGGAGAUGCCAUU | 278 |
| D-1139 | GGCAUCUCCCCAUUCCAUGA{invAb} | 279 | AUCAUGGAAUGGGGAGAUGCCUU | 280 |
| D-1140 | GGCAUCUCCCCAUUCCAUGA{invAb} | 281 | AUCAUGGAAUGGGGAGAUGCCUU | 282 |
| D-1141 | GCAUCUCCCCAUUCCAUGAG{invAb} | 283 | ACUCAUGGAAUGGGGAGAUGCUU | 284 |
| D-1142 | CAUCUCCCCAUUCCAUGAGC{invAb} | 285 | UGCUCAUGGAAUGGGGAGAUGUU | 286 |
| D-1143 | AUCUCCCCAUUCCAUGAGCA{invAb} | 287 | AUGCUCAUGGAAUGGGGAGAUUU | 288 |
| D-1144 | UCUCCCCAUUCCAUGAGCAU{invAb} | 289 | AAUGCUCAUGGAAUGGGGAGAUU | 290 |
| D-1145 | UCUCCCCAUUCCAUGAGCAU{invAb} | 291 | AAUGCUCAUGGAAUGGGGAGAUU | 292 |
| D-1146 | CUCCCCAUUCCAUGAGCAUG{invAb} | 293 | ACAUGCUCAUGGAAUGGGGAGUU | 294 |
| D-1147 | UCCCCAUUCCAUGAGCAUGC{invAb} | 295 | UGCAUGCUCAUGGAAUGGGGAUU | 296 |
| D-1148 | CCCCAUUCCAUGAGCAUGCA{invAb} | 297 | AUGCAUGCUCAUGGAAUGGGGUU | 298 |
| D-1149 | CCAUUCCAUGAGCAUGCAGA{invAb} | 299 | AUCUGCAUGCUCAUGGAAUGGUU | 300 |
| D-1150 | CCAUUCCAUGAGCAUGCAGA{invAb} | 301 | AUCUGCAUGCUCAUGGAAUGGUU | 302 |
| D-1151 | CAUUCCAUGAGCAUGCAGAG{invAb} | 303 | ACUCUGCAUGCUCAUGGAAUGUU | 304 |
| D-1152 | AUUCCAUGAGCAUGCAGAGG{invAb} | 305 | ACCUCUGCAUGCUCAUGGAAUUU | 306 |
| D-1153 | UUCCAUGAGCAUGCAGAGGU{invAb} | 307 | AACCUCUGCAUGCUCAUGGAAUU | 308 |
| D-1154 | UCCAUGAGCAUGCAGAGGUG{invAb} | 309 | ACACCUCUGCAUGCUCAUGGAUU | 310 |
| D-1155 | CCAUGAGCAUGCAGAGGUGG{invAb} | 311 | ACCACCUCUGCAUGCUCAUGGUU | 312 |
| D-1156 | CAUGAGCAUGCAGAGGUGGU{invAb} | 313 | UACCACCUCUGCAUGCUCAUGUU | 314 |
| D-1157 | AUGAGCAUGCAGAGGUGGUA{invAb} | 315 | AUACCACCUCUGCAUGCUCAUUU | 316 |
| D-1158 | UGAGCAUGCAGAGGUGGUAU{invAb} | 317 | AAUACCACCUCUGCAUGCUCAUU | 318 |
| D-1159 | GAGCAUGCAGAGGUGGUAUU{invAb} | 319 | AAAUACCACCUCUGCAUGCUCUU | 320 |
| D-1160 | AGCAUGCAGAGGUGGUAUUC{invAb} | 321 | UGAAUACCACCUCUGCAUGCUUU | 322 |
| D-1161 | GCAUGCAGAGGUGGUAUUCA{invAb} | 323 | AUGAAUACCACCUCUGCAUGCUU | 324 |
| D-1162 | CAUGCAGAGGUGGUAUUCAC{invAb} | 325 | UGUGAAUACCACCUCUGCAUGUU | 326 |
| D-1163 | AUGCAGAGGUGGUAUUCACA{invAb} | 327 | AUGUGAAUACCACCUCUGCAUUU | 328 |
| D-1164 | AGAGGUGGUAUUCACAGCCA{invAb} | 329 | UUGGCUGUGAAUACCACCUCUUU | 330 |
| D-1165 | GAGGUGGUAUUCACAGCCAA{invAb} | 331 | AUUGGCUGUGAAUACCACCUCUU | 332 |
| D-1166 | AGGUGGUAUUCACAGCCAAC{invAb} | 333 | AGUUGGCUGUGAAUACCACCUUU | 334 |
| D-1167 | GGUGGUAUUCACAGCCAACG{invAb} | 335 | UCGUUGGCUGUGAAUACCACCUU | 336 |
| D-1168 | GCCGCCCUGCUGAGCCCCUA{invAb} | 337 | AUAGGGGCUCAGCAGGGCGGCUU | 338 |
| D-1169 | CCGCCCUGCUGAGCCCCUAC{invAb} | 339 | AGUAGGGGCUCAGCAGGGCGGUU | 340 |
| D-1170 | CGCCCUGCUGAGCCCCUACU{invAb} | 341 | AAGUAGGGGCUCAGCAGGGCGUU | 342 |
| D-1171 | GCCCUGCUGAGCCCCUACUC{invAb} | 343 | AGAGUAGGGGCUCAGCAGGGCUU | 344 |
| D-1172 | CCCUGCUGAGCCCCUACUCC{invAb} | 345 | AGGAGUAGGGGCUCAGCAGGGUU | 346 |
| D-1173 | CCUGCUGAGCCCCUACUCCU{invAb} | 347 | UAGGAGUAGGGGCUCAGCAGGUU | 348 |
| D-1174 | CUGCUGAGCCCCUACUCCUA{invAb} | 349 | AUAGGAGUAGGGGCUCAGCAGUU | 350 |
| D-1175 | UGCUGAGCCCCUACUCCUAU{invAb} | 351 | AAUAGGAGUAGGGGCUCAGCAUU | 352 |
| D-1176 | GCUGAGCCCCUACUCCUAUU{invAb} | 353 | AAAUAGGAGUAGGGGCUCAGCUU | 354 |
| D-1177 | CUGAGCCCCUACUCCUAUUC{invAb} | 355 | AGAAUAGGAGUAGGGGCUCAGUU | 356 |
| D-1178 | UGAGCCCCUACUCCUAUUCC{invAb} | 357 | UGGAAUAGGAGUAGGGGCUCAUU | 358 |
| D-1179 | GAGCCCCUACUCCUAUUCCA{invAb} | 359 | AUGGAAUAGGAGUAGGGGCUCUU | 360 |
| D-1180 | AGCCCCUACUCCUAUUCCAC{invAb} | 361 | AGUGGAAUAGGAGUAGGGGCUUU | 362 |
| D-1181 | GCCCCUACUCCUAUUCCACC{invAb} | 363 | UGGUGGAAUAGGAGUAGGGGCUU | 364 |
| D-1182 | CCCCUACUCCUAUUCCACCA{invAb} | 365 | AUGGUGGAAUAGGAGUAGGGGUU | 366 |
| D-1183 | CCCUACUCCUAUUCCACCAC{invAb} | 367 | AGUGGUGGAAUAGGAGUAGGGUU | 368 |
| D-1184 | CCUACUCCUAUUCCACCACG{invAb} | 369 | ACGUGGUGGAAUAGGAGUAGGUU | 370 |
| D-1185 | CUACUCCUAUUCCACCACGG{invAb} | 371 | ACCGUGGUGGAAUAGGAGUAGUU | 372 |
| D-1186 | UACUCCUAUUCCACCACGGC{invAb} | 373 | AGCCGUGGUGGAAUAGGAGUAUU | 374 |
| D-1187 | ACUCCUAUUCCACCACGGCU{invAb} | 375 | AAGCCGUGGUGGAAUAGGAGUUU | 376 |
| D-1188 | CUCCUAUUCCACCACGGCUG{invAb} | 377 | ACAGCCGUGGUGGAAUAGGAGUU | 378 |
| D-1189 | UCCUAUUCCACCACGGCUGU{invAb} | 379 | AACAGCCGUGGUGGAAUAGGAUU | 380 |
| D-1190 | CCUAUUCCACCACGGCUGUC{invAb} | 381 | AGACAGCCGUGGUGGAAUAGGUU | 382 |
| D-1191 | CUAUUCCACCACGGCUGUCG{invAb} | 383 | ACGACAGCCGUGGUGGAAUAGUU | 384 |
| D-1192 | UAUUCCACCACGGCUGUCGU{invAb} | 385 | AACGACAGCCGUGGUGGAAUAUU | 386 |
| D-1193 | AUUCCACCACGGCUGUCGUC{invAb} | 387 | UGACGACAGCCGUGGUGGAAUUU | 388 |
| D-1194 | UUCCACCACGGCUGUCGUCA{invAb} | 389 | AUGACGACAGCCGUGGUGGAAUU | 390 |
| D-1195 | UCCACCACGGCUGUCGUCAC{invAb} | 391 | AGUGACGACAGCCGUGGUGGAUU | 392 |
| D-1196 | CCACCACGGCUGUCGUCACC{invAb} | 393 | UGGUGACGACAGCCGUGGUGGUU | 394 |
| D-1197 | CACCACGGCUGUCGUCACCA{invAb} | 395 | UUGGUGACGACAGCCGUGGUGUU | 396 |
| D-1198 | ACCACGGCUGUCGUCACCAA{invAb} | 397 | AUUGGUGACGACAGCCGUGGUUU | 398 |
| D-1199 | CCACGGCUGUCGUCACCAAU{invAb} | 399 | AAUUGGUGACGACAGCCGUGGUU | 400 |
| D-1200 | CACGGCUGUCGUCACCAAUC{invAb} | 401 | AGAUUGGUGACGACAGCCGUGUU | 402 |
| D-1201 | ACGGCUGUCGUCACCAAUCC{invAb} | 403 | AGGAUUGGUGACGACAGCCGUUU | 404 |
| D-1202 | CGGCUGUCGUCACCAAUCCC{invAb} | 405 | UGGGAUUGGUGACGACAGCCGUU | 406 |
| D-1203 | GGCUGUCGUCACCAAUCCCA{invAb} | 407 | UUGGGAUUGGUGACGACAGCCUU | 408 |
| D-1204 | GCUGUCGUCACCAAUCCCAA{invAb} | 409 | AUUGGGAUUGGUGACGACAGCUU | 410 |
| D-1205 | UGUCGUCACCAAUCCCAAGG{invAb} | 411 | UCCUUGGGAUUGGUGACGACAUU | 412 |
| D-1206 | GUCGUCACCAAUCCCAAGGA{invAb} | 413 | UUCCUUGGGAUUGGUGACGACUU | 414 |
| D-1207 | UCGUCACCAAUCCCAAGGAA{invAb} | 415 | AUUCCUUGGGAUUGGUGACGAUU | 416 |
| D-1208 | CGUCACCAAUCCCAAGGAAU{invAb} | 417 | AAUUCCUUGGGAUUGGUGACGUU | 418 |
| D-1209 | CACCAAUCCCAAGGAAUGAG{invAb} | 419 | ACUCAUUCCUUGGGAUUGGUGUU | 420 |
| D-1210 | ACCAAUCCCAAGGAAUGAGG{invAb} | 421 | ACCUCAUUCCUUGGGAUUGGUUU | 422 |
| D-1211 | CCAAUCCCAAGGAAUGAGGG{invAb} | 423 | UCCCUCAUUCCUUGGGAUUGGUU | 424 |
| D-1212 | CAAUCCCAAGGAAUGAGGGA{invAb} | 425 | AUCCCUCAUUCCUUGGGAUUGUU | 426 |
| D-1213 | AAUCCCAAGGAAUGAGGGAC{invAb} | 427 | AGUCCCUCAUUCCUUGGGAUUUU | 428 |
| D-1214 | AUCCCAAGGAAUGAGGGACU{invAb} | 429 | AAGUCCCUCAUUCCUUGGGAUUU | 430 |
| D-1215 | UCCCAAGGAAUGAGGGACUU{invAb} | 431 | AAAGUCCCUCAUUCCUUGGGAUU | 432 |
| D-1216 | CCCAAGGAAUGAGGGACUUC{invAb} | 433 | AGAAGUCCCUCAUUCCUUGGGUU | 434 |
| D-1217 | CCAAGGAAUGAGGGACUUCU{invAb} | 435 | AAGAAGUCCCUCAUUCCUUGGUU | 436 |
| D-1218 | CAAGGAAUGAGGGACUUCUC{invAb} | 437 | AGAGAAGUCCCUCAUUCCUUGUU | 438 |
| D-1219 | CUGAAGGACGAGGGAUGGGA{invAb} | 439 | AUCCCAUCCCUCGUCCUUCAGUU | 440 |
| D-1220 | UGAAGGACGAGGGAUGGGAU{invAb} | 441 | AAUCCCAUCCCUCGUCCUUCAUU | 442 |
| D-1221 | GAAGGACGAGGGAUGGGAUU{invAb} | 443 | AAAUCCCAUCCCUCGUCCUUCUU | 444 |
| D-1222 | AAGGACGAGGGAUGGGAUUU{invAb} | 445 | AAAAUCCCAUCCCUCGUCCUUUU | 446 |
| D-1223 | AGGACGAGGGAUGGGAUUUC{invAb} | 447 | UGAAAUCCCAUCCCUCGUCCUUU | 448 |
| D-1224 | GGACGAGGGAUGGGAUUUCA{invAb} | 449 | AUGAAAUCCCAUCCCUCGUCCUU | 450 |
| D-1225 | GACGAGGGAUGGGAUUUCAU{invAb} | 451 | AAUGAAAUCCCAUCCCUCGUCUU | 452 |
| D-1226 | ACGAGGGAUGGGAUUUCAUG{invAb} | 453 | ACAUGAAAUCCCAUCCCUCGUUU | 454 |
| D-1227 | CGAGGGAUGGGAUUUCAUGU{invAb} | 455 | UACAUGAAAUCCCAUCCCUCGUU | 456 |
| D-1228 | GAGGGAUGGGAUUUCAUGUA{invAb} | 457 | UUACAUGAAAUCCCAUCCCUCUU | 458 |
| D-1229 | AGGGAUGGGAUUUCAUGUAA{invAb} | 459 | AUUACAUGAAAUCCCAUCCCUUU | 460 |
| D-1230 | GGGAUGGGAUUUCAUGUAAC{invAb} | 461 | AGUUACAUGAAAUCCCAUCCCUU | 462 |
| D-1231 | GGAUGGGAUUUCAUGUAACC{invAb} | 463 | UGGUUACAUGAAAUCCCAUCCUU | 464 |
| D-1232 | GAUGGGAUUUCAUGUAACCA{invAb} | 465 | UUGGUUACAUGAAAUCCCAUCUU | 466 |
| D-1233 | AUGGGAUUUCAUGUAACCAA{invAb} | 467 | AUUGGUUACAUGAAAUCCCAUUU | 468 |
| D-1234 | UGGGAUUUCAUGUAACCAAG{invAb} | 469 | UCUUGGUUACAUGAAAUCCCAUU | 470 |
| D-1235 | GGGAUUUCAUGUAACCAAGA{invAb} | 471 | AUCUUGGUUACAUGAAAUCCCUU | 472 |
| D-1236 | GGAUUUCAUGUAACCAAGAG{invAb} | 473 | ACUCUUGGUUACAUGAAAUCCUU | 474 |
| D-1237 | GAUUUCAUGUAACCAAGAGU{invAb} | 475 | UACUCUUGGUUACAUGAAAUCUU | 476 |
| D-1238 | AUUUCAUGUAACCAAGAGUA{invAb} | 477 | AUACUCUUGGUUACAUGAAAUUU | 478 |
| D-1239 | UUUCAUGUAACCAAGAGUAU{invAb} | 479 | AAUACUCUUGGUUACAUGAAAUU | 480 |
| D-1240 | UUCAUGUAACCAAGAGUAUU{invAb} | 481 | AAAUACUCUUGGUUACAUGAAUU | 482 |
| D-1241 | UCAUGUAACCAAGAGUAUUC{invAb} | 483 | AGAAUACUCUUGGUUACAUGAUU | 484 |
| D-1242 | CAUGUAACCAAGAGUAUUCC{invAb} | 485 | UGGAAUACUCUUGGUUACAUGUU | 486 |
| D-1243 | AUGUAACCAAGAGUAUUCCA{invAb} | 487 | AUGGAAUACUCUUGGUUACAUUU | 488 |
| D-1244 | UGUAACCAAGAGUAUUCCAU{invAb} | 489 | AAUGGAAUACUCUUGGUUACAUU | 490 |
| D-1245 | GUAACCAAGAGUAUUCCAUU{invAb} | 491 | AAAUGGAAUACUCUUGGUUACUU | 492 |
| D-1246 | UAACCAAGAGUAUUCCAUUU{invAb} | 493 | AAAAUGGAAUACUCUUGGUUAUU | 494 |
| D-1247 | AACCAAGAGUAUUCCAUUUU{invAb} | 495 | AAAAAUGGAAUACUCUUGGUUUU | 496 |
| D-1248 | ACCAAGAGUAUUCCAUUUUU{invAb} | 497 | UAAAAAUGGAAUACUCUUGGUUU | 498 |
| D-1249 | CCAAGAGUAUUCCAUUUUUA{invAb} | 499 | AUAAAAAUGGAAUACUCUUGGUU | 500 |
| D-1250 | CAAGAGUAUUCCAUUUUUAC{invAb} | 501 | AGUAAAAAUGGAAUACUCUUGUU | 502 |
| D-1251 | AAGAGUAUUCCAUUUUUACU{invAb} | 503 | UAGUAAAAAUGGAAUACUCUUUU | 504 |
| D-1252 | AGAGUAUUCCAUUUUUACUA{invAb} | 505 | UUAGUAAAAAUGGAAUACUCUUU | 506 |
| D-1253 | GAGUAUUCCAUUUUUACUAA{invAb} | 507 | UUUAGUAAAAAUGGAAUACUCUU | 508 |
| D-1254 | AGUAUUCCAUUUUUACUAAA{invAb} | 509 | AUUUAGUAAAAAUGGAAUACUUU | 510 |
| D-1255 | UCCAUUUUUACUAAAGCAGU{invAb} | 511 | AACUGCUUUAGUAAAAAUGGAUU | 512 |
| D-1256 | CAUUUUUACUAAAGCAGUGU{invAb} | 513 | AACACUGCUUUAGUAAAAAUGUU | 514 |
| D-1257 | AUUUUUACUAAAGCAGUGUU{invAb} | 515 | AAACACUGCUUUAGUAAAAAUUU | 516 |
| D-1258 | UUUUUACUAAAGCAGUGUUU{invAb} | 517 | AAAACACUGCUUUAGUAAAAAUU | 518 |
| D-1259 | UUUUACUAAAGCAGUGUUUU{invAb} | 519 | AAAAACACUGCUUUAGUAAAAUU | 520 |
| D-1260 | UUACUAAAGCAGUGUUUUCA{invAb} | 521 | AUGAAAACACUGCUUUAGUAAUU | 522 |
| D-1261 | UACUAAAGCAGUGUUUUCAC{invAb} | 523 | AGUGAAAACACUGCUUUAGUAUU | 524 |
| D-1262 | ACUAAAGCAGUGUUUUCACC{invAb} | 525 | AGGUGAAAACACUGCUUUAGUUU | 526 |
| D-1263 | CUAAAGCAGUGUUUUCACCU{invAb} | 527 | AAGGUGAAAACACUGCUUUAGUU | 528 |
| D-1264 | UAAAGCAGUGUUUUCACCUC{invAb} | 529 | UGAGGUGAAAACACUGCUUUAUU | 530 |
| D-1265 | AAAGCAGUGUUUUCACCUCA{invAb} | 531 | AUGAGGUGAAAACACUGCUUUUU | 532 |
| D-1266 | AAGCAGUGUUUUCACCUCAU{invAb} | 533 | UAUGAGGUGAAAACACUGCUUUU | 534 |
| D-1267 | AGUCCAGGCAGAGACAAUAA{invAb} | 535 | UUUAUUGUCUCUGCCUGGACUUU | 536 |
| D-1268 | GUCCAGGCAGAGACAAUAAA{invAb} | 537 | UUUUAUUGUCUCUGCCUGGACUU | 538 |
| D-1269 | UCCAGGCAGAGACAAUAAAA{invAb} | 539 | AUUUUAUUGUCUCUGCCUGGAUU | 540 |
| D-1270 | CCAGGCAGAGACAAUAAAAC{invAb} | 541 | UGUUUUAUUGUCUCUGCCUGGUU | 542 |
| D-1271 | CAGGCAGAGACAAUAAAACA{invAb} | 543 | AUGUUUUAUUGUCUCUGCCUGUU | 544 |
| D-1272 | AGGCAGAGACAAUAAAACAU{invAb} | 545 | AAUGUUUUAUUGUCUCUGCCUUU | 546 |
| D-1273 | GGCAGAGACAAUAAAACAUU{invAb} | 547 | AAAUGUUUUAUUGUCUCUGCCUU | 548 |
| D-1274 | AGACAAUAAAACAUUCCUGU{invAb} | 549 | AACAGGAAUGUUUUAUUGUCUUU | 550 |
| D-1275 | GACAAUAAAACAUUCCUGUG{invAb} | 551 | UCACAGGAAUGUUUUAUUGUCUU | 552 |
| D-1276 | ACAAUAAAACAUUCCUGUGA{invAb} | 553 | UUCACAGGAAUGUUUUAUUGUUU | 554 |
| D-1277 | CAAUAAAACAUUCCUGUGAA{invAb} | 555 | UUUCACAGGAAUGUUUUAUUGUU | 556 |
| D-1278 | AAUAAAACAUUCCUGUGAAA{invAb} | 557 | AUUUCACAGGAAUGUUUUAUUUU | 558 |
| D-1279 | AUAAAACAUUCCUGUGAAAG{invAb} | 559 | ACUUUCACAGGAAUGUUUUAUUU | 560 |
| D-1280 | UAAAACAUUCCUGUGAAAGG{invAb} | 561 | ACCUUUCACAGGAAUGUUUUAUU | 562 |
| D-1281 | AAAACAUUCCUGUGAAAGGC{invAb} | 563 | UGCCUUUCACAGGAAUGUUUUUU | 564 |
| D-1282 | AAACAUUCCUGUGAAAGGCA{invAb} | 565 | AUGCCUUUCACAGGAAUGUUUUU | 566 |

To improve the potency and in vivo stability of TTR siRNA sequences, chemical modifications were incorporated into TTR siRNA molecules. Specifically, 2′-O-methyl and 2′-fluoro modifications of the ribose sugar were incorporated at specific positions within the TTR siRNAs. Phosphorothioate internucleotide linkages were also incorporated at the terminal ends of the antisense and/or sense sequences. Table 2 below depicts the modifications in the sense and antisense sequences for each of the modified TTR siRNAs. The nucleotide sequences in Table 2 are listed according to the following notations: A, U, G, and C=corresponding ribonucleotide; dC and dG=corresponding deoxyribonucleotide; dT=deoxythymidine; a, u, g, and c=corresponding 2′-O-methyl ribonucleotide; Af, Uf, Gf, and Cf=corresponding 2′-deoxy-2′-fluoro (“2′-fluoro”) ribonucleotide; [InvAb] is an inverted abasic residue; [Ab] is an abasic residue; GNA is a glycol nucleic acid and bases with the GNA backbone are shown as AgN, UgN, CgN, and GgN. Insertion of an “s” in the sequence indicates that the two adjacent nucleotides are connected by a phosphorothiodiester group (e.g. a phosphorothioate internucleotide linkage). Unless indicated otherwise, all other nucleotides are connected by 3′-5′ phosphodiester groups. Each of the siRNA compounds in Table 2 comprises a 19-21 base pair duplex region with either a 2 nucleotide overhang at the 3′ end of both strands or bluntmer at one or both ends. In some embodiments, each 5′ end of the sense strand has been linked to the GalNAc (referred to as {sGalNAc3}) structure below:

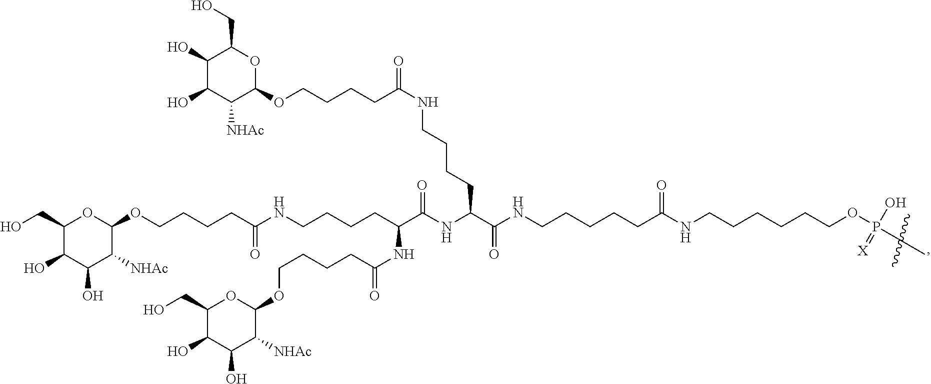

wherein X═O or S.

TABLE 2
siRNA Sequences Directed to TTR with Modifications

| | | | | SEQ ID |
|---|---|---|---|---|
| | | SEQ ID | | NO: |
| Duplex | | NO: | | (anti- |
| No. | Sense sequence (5′-3′) | (sense) | Antisense sequence (5′-3′) | sense) |
| D-2000 | ususcuugGfcAfGfGfAfuggcuucus{invAb} | 567 | asAfsgaagCfcauccuGfcCfaagaasusu | 568 |
| D-2001 | uscsuuggCfaGfGfAfUfggcuucucs{invAb} | 569 | usGfsagaaGfccauccUfgCfcaagasusu | 570 |
| D-2002 | csusuggcAfgGfAfUfGfgcuucucas{invAb} | 571 | asUfsgagaAfgccaucCfuGfccaagsusu | 572 |
| D-2003 | ususggcaGfgAfUfGfGfcuucucaus{invAb} | 573 | asAfsugagAfagccauCfcUfgccaasusu | 574 |
| D-2004 | usgsgcagGfaUfGfGfCfuucucaucs{invAb} | 575 | asGfsaugaGfaagccaUfcCfugccasusu | 576 |
| D-2005 | gsgscaggAfuGfGfCfUfucucaucgs{invAb} | 577 | asCfsgaugAfgaagccAfuCfcugccsusu | 578 |
| D-2006 | gscsaggaUfgGfCfUfUfcucaucgus{invAb} | 579 | asAfscgauGfagaagcCfaUfccugcsusu | 580 |
| D-2007 | csasggauGfgCfUfUfCfucaucgucs{invAb} | 581 | asGfsacgaUfgagaagCfcAfuccugsusu | 582 |
| D-2008 | asgsgaugGfcUfUfCfUfcaucgucus{invAb} | 583 | asAfsgacgAfugagaaGfcCfauccususu | 584 |
| D-2009 | gsgsauggCfuUfCfUfCfaucgucugs{invAb} | 585 | asCfsagacGfaugagaAfgCfcauccsusu | 586 |
| D-2010 | gsasuggcUfuCfUfCfAfucgucugcs{invAb} | 587 | asGfscagaCfgaugagAfaGfccaucsusu | 588 |
| D-2011 | asusggcuUfcUfCfAfUfcgucugcus{invAb} | 589 | asAfsgcagAfcgaugaGfaAfgccaususu | 590 |
| D-2012 | usgsgcuuCfuCfAfUfCfgucugcucs{invAb} | 591 | asGfsagcaGfacgaugAfgAfagccasusu | 592 |
| D-2013 | gscsuucuCfaUfCfGfUfcugcuccus{invAb} | 593 | asAfsggagCfagacgaUfgAfgaagcsusu | 594 |
| D-2014 | csusucucAfuCfGfUfCfugcuccucs{invAb} | 595 | asGfsaggaGfcagacgAfuGfagaagsusu | 596 |
| D-2015 | ususcucaUfcGfUfCfUfgcuccuccs{invAb} | 597 | asGfsgaggAfgcagacGfaUfgagaasusu | 598 |
| D-2016 | uscsucauCfgUfCfUfGfcuccuccus{invAb} | 599 | asAfsggagGfagcagaCfgAfugagasusu | 600 |
| D-2017 | csuscaucGfuCfUfGfCfuccuccucs{invAb} | 601 | asGfsaggaGfgagcagAfcGfaugagsusu | 602 |
| D-2018 | csasucguCfuGfCfUfCfcuccucugs{invAb} | 603 | asCfsagagGfaggagcAfgAfcgaugsusu | 604 |
| D-2019 | gscsuggaCfuGfGfUfAfuuugugucs{invAb} | 605 | asGfsacacAfaauaccAfgUfccagcsusu | 606 |
| D-2020 | csusggacUfgGfUfAfUfuugugucus{invAb} | 607 | asAfsgacaCfaaauacCfaGfuccagsusu | 608 |
| D-2021 | usgsgacuGfgUfAfUfUfugugucugs{invAb} | 609 | usCfsagacAfcaaauaCfcAfguccasusu | 610 |
| D-2022 | gsgsacugGfuAfUfUfUfgugucugas{invAb} | 611 | asUfscagaCfacaaauAfcCfaguccsusu | 612 |
| D-2023 | gsascuggUfaUfUfUfGfugucugags{invAb} | 613 | asCfsucagAfcacaaaUfaCfcagucsusu | 614 |
| D-2024 | ascsugguAfuUfUfGfUfgucugaggs{invAb} | 615 | asCfscucaGfacacaaAfuAfccagususu | 616 |
| D-2025 | csusgguaUfuUfGfUfGfucugaggcs{invAb} | 617 | asGfsccucAfgacacaAfaUfaccagsusu | 618 |
| D-2026 | usgsguauUfuGfUfGfUfcugaggcus{invAb} | 619 | asAfsgccuCfagacacAfaAfuaccasusu | 620 |
| D-2027 | gsgsuauuUfgUfGfUfCfugaggcugs{invAb} | 621 | asCfsagccUfcagacaCfaAfauaccsusu | 622 |
| D-2028 | usgsaaucCfaAfGfUfGfuccucugas{invAb} | 623 | asUfscagaGfgacacuUfgGfauucasusu | 624 |
| D-2029 | gsasauccAfaGfUfGfUfccucugaus{invAb} | 625 | asAfsucagAfggacacUfuGfgauucsusu | 626 |
| D-2030 | asasuccaAfgUfGfUfCfcucugaugs{invAb} | 627 | asCfsaucaGfaggacaCfuUfggauususu | 628 |
| D-2031 | asusccaaGfuGfUfCfCfucugauggs{invAb} | 629 | asCfscaucAfgaggacAfcUfuggaususu | 630 |
| D-2032 | uscscaagUfgUfCfCfUfcugauggus{invAb} | 631 | asAfsccauCfagaggaCfaCfuuggasusu | 632 |
| D-2033 | cscsaaguGfuCfCfUfCfugauggucs{invAb} | 633 | usGfsaccaUfcagaggAfcAfcuuggsusu | 634 |
| D-2034 | csasagugUfcCfUfCfUfgauggucas{invAb} | 635 | usUfsgaccAfucagagGfaCfacuugsusu | 636 |
| D-2035 | asasguguCfcUfCfUfGfauggucaas{invAb} | 637 | usUfsugacCfaucagaGfgAfcacuususu | 638 |
| D-2036 | asgsugucCfuCfUfGfAfuggucaaas{invAb} | 639 | asUfsuugaCfcaucagAfgGfacacususu | 640 |
| D-2037 | usgsuccuCfuGfAfUfGfgucaaagus{invAb} | 641 | asAfscuuuGfaccaucAfgAfggacasusu | 642 |
| D-2038 | gsusccucUfgAfUfGfGfucaaaguus{invAb} | 643 | asAfsacuuUfgaccauCfaGfaggacsusu | 644 |
| D-2039 | uscscucuGfaUfGfGfUfcaaaguucs{invAb} | 645 | asGfsaacuUfugaccaUfcAfgaggasusu | 646 |
| D-2040 | cscsucugAfuGfGfUfCfaaaguucus{invAb} | 647 | usAfsgaacUfuugaccAfuCfagaggsusu | 648 |
| D-2041 | csuscugaUfgGfUfCfAfaaguucuas{invAb} | 649 | asUfsagaaCfuuugacCfaUfcagagsusu | 650 |
| D-2042 | uscsugauGfgUfCfAfAfaguucuags{invAb} | 651 | usCfsuagaAfcuuugaCfcAfucagasusu | 652 |
| D-2043 | csusgaugGfuCfAfAfAfguucuagas{invAb} | 653 | asUfscuagAfacuuugAfcCfaucagsusu | 654 |
| D-2044 | usgsauggUfcAfAfAfGfuucuagaus{invAb} | 655 | asAfsucuaGfaacuuuGfaCfcaucasusu | 656 |
| D-2045 | gsasugguCfaAfAfGfUfucuagaugs{invAb} | 657 | asCfsaucuAfgaacuuUfgAfccaucsusu | 658 |
| D-2046 | gscscauuUfgCfCfUfCfugggaaaas{invAb} | 659 | asUfsuuucCfcagaggCfaAfauggcsusu | 660 |
| D-2047 | cscsauuuGfcCfUfCfUfgggaaaacs{invAb} | 661 | asGfsuuuuCfccagagGfcAfaauggsusu | 662 |
| D-2048 | csasuuugCfcUfCfUfGfggaaaaccs{invAb} | 663 | usGfsguuuUfcccagaGfgCfaaaugsusu | 664 |
| D-2049 | asusuugcCfuCfUfGfGfgaaaaccas{invAb} | 665 | asUfsgguuUfucccagAfgGfcaaaususu | 666 |
| D-2050 | ususugccUfcUfGfGfGfaaaaccags{invAb} | 667 | asCfsugguUfuucccaGfaGfgcaaasusu | 668 |
| D-2051 | ususgccuCfuGfGfGfAfaaaccagus{invAb} | 669 | asAfscuggUfuuucccAfgAfggcaasusu | 670 |
| D-2052 | usgsccucUfgGfGfAfAfaaccagugs{invAb} | 671 | usCfsacugGfuuuuccCfaGfaggcasusu | 672 |
| D-2053 | csuscuggGfaAfAfAfCfcagugagus{invAb} | 673 | asAfscucaCfugguuuUfcCfcagagsusu | 674 |
| D-2054 | uscsugggAfaAfAfCfCfagugagucs{invAb} | 675 | asGfsacucAfcugguuUfuCfccagasusu | 676 |
| D-2055 | csusgggaAfaAfCfCfAfgugagucus{invAb} | 677 | asAfsgacuCfacugguUfuUfcccagsusu | 678 |
| D-2056 | usgsggaaAfaCfCfAfGfugagucugs{invAb} | 679 | asCfsagacUfcacuggUfuUfucccasusu | 680 |
| D-2057 | gsgsgaaaAfcCfAfGfUfgagucuggs{invAb} | 681 | usCfscagaCfucacugGfuUfuucccsusu | 682 |
| D-2058 | gsgsaaaaCfcAfGfUfGfagucuggas{invAb} | 683 | asUfsccagAfcucacuGfgUfuuuccsusu | 684 |
| D-2059 | gsasaaacCfaGfUfGfAfgucuggags{invAb} | 685 | usCfsuccaGfacucacUfgGfuuuucsusu | 686 |
| D-2060 | asasaaccAfgUfGfAfGfucuggagas{invAb} | 687 | asUfscuccAfgacucaCfuGfguuuususu | 688 |
| D-2061 | asasaccaGfuGfAfGfUfcuggagags{invAb} | 689 | asCfsucucCfagacucAfcUfgguuususu | 690 |
| D-2062 | asasccagUfgAfGfUfCfuggagagcs{invAb} | 691 | asGfscucuCfcagacuCfaCfugguususu | 692 |
| D-2063 | gsusgaguCfuGfGfAfGfagcugcaus{invAb} | 693 | asAfsugcaGfcucuccAfgAfcucacsusu | 694 |
| D-2064 | usgsagucUfgGfAfGfAfgcugcaugs{invAb} | 695 | asCfsaugcAfgcucucCfaGfacucasusu | 696 |
| D-2065 | gsasgucuGfgAfGfAfGfcugcauggs{invAb} | 697 | asCfscaugCfagcucuCfcAfgacucsusu | 698 |
| D-2066 | gsuscuggAfgAfGfCfUfgcaugggcs{invAb} | 699 | asGfscccaUfgcagcuCfuCfcagacsusu | 700 |
| D-2067 | uscsuggaGfaGfCfUfGfcaugggcus{invAb} | 701 | asAfsgcccAfugcagcUfcUfccagasusu | 702 |
| D-2068 | csusggagAfgCfUfGfCfaugggcucs{invAb} | 703 | usGfsagccCfaugcagCfuCfuccagsusu | 704 |
| D-2069 | gsgsagagCfuGfCfAfUfgggcucacs{invAb} | 705 | usGfsugagCfccaugcAfgCfucuccsusu | 706 |
| D-2070 | gsasgagcUfgCfAfUfGfggcucacas{invAb} | 707 | usUfsgugaGfcccaugCfaGfcucucsusu | 708 |
| D-2071 | asgsagcuGfcAfUfGfGfgcucacaas{invAb} | 709 | asUfsugugAfgcccauGfcAfgcucususu | 710 |
| D-2072 | gsasgcugCfaUfGfGfGfcucacaacs{invAb} | 711 | asGfsuuguGfagcccaUfgCfagcucsusu | 712 |
| D-2073 | asgscugcAfuGfGfGfCfucacaacus{invAb} | 713 | asAfsguugUfgagcccAfuGfcagcususu | 714 |
| D-2074 | gscsugcaUfgGfGfCfUfcacaacugs{invAb} | 715 | usCfsaguuGfugagccCfaUfgcagcsusu | 716 |
| D-2075 | csusgcauGfgGfCfUfCfacaacugas{invAb} | 717 | asUfscaguUfgugagcCfcAfugcagsusu | 718 |
| D-2076 | usgscaugGfgCfUfCfAfcaacugags{invAb} | 719 | asCfsucagUfugugagCfcCfaugcasusu | 720 |
| D-2077 | gscsauggGfcUfCfAfCfaacugaggs{invAb} | 721 | usCfscucaGfuugugaGfcCfcaugcsusu | 722 |
| D-2078 | csasugggCfuCfAfCfAfacugaggas{invAb} | 723 | asUfsccucAfguugugAfgCfccaugsusu | 724 |
| D-2079 | gsgscucaCfaAfCfUfGfaggaggaas{invAb} | 725 | asUfsuccuCfcucaguUfgUfgagccsusu | 726 |
| D-2080 | gscsucacAfaCfUfGfAfggaggaaus{invAb} | 727 | asAfsuuccUfccucagUfuGfugagcsusu | 728 |
| D-2081 | csuscacaAfcUfGfAfGfgaggaauus{invAb} | 729 | asAfsauucCfuccucaGfuUfgugagsusu | 730 |
| D-2082 | csascaacUfgAfGfGfAfggaauuugs{invAb} | 731 | asCfsaaauUfccuccuCfaGfuugugsusu | 732 |
| D-2083 | ascsaacuGfaGfGfAfGfgaauuugus{invAb} | 733 | usAfscaaaUfuccuccUfcAfguugususu | 734 |
| D-2084 | csasacugAfgGfAfGfGfaauuuguas{invAb} | 735 | asUfsacaaAfuuccucCfuCfaguugsusu | 736 |
| D-2085 | asascugaGfgAfGfGfAfauuuguags{invAb} | 737 | usCfsuacaAfauuccuCfcUfcaguususu | 738 |
| D-2086 | ascsugagGfaGfGfAfAfuuuguagas{invAb} | 739 | usUfscuacAfaauuccUfcCfucagususu | 740 |
| D-2087 | csusgaggAfgGfAfAfUfuuguagaas{invAb} | 741 | asUfsucuaCfaaauucCfuCfcucagsusu | 742 |
| D-2088 | usgsaggaGfgAfAfUfUfuguagaags{invAb} | 743 | asCfsuucuAfcaaauuCfcUfccucasusu | 744 |
| D-2089 | gsasggagGfaAfUfUfUfguagaaggs{invAb} | 745 | asCfscuucUfacaaauUfcCfuccucsusu | 746 |
| D-2090 | asgsgaggAfaUfUfUfGfuagaagggs{invAb} | 747 | usCfsccuuCfuacaaaUfuCfcuccususu | 748 |
| D-2091 | gsgsaggaAfuUfUfGfUfagaagggas{invAb} | 749 | asUfscccuUfcuacaaAfuUfccuccsusu | 750 |
| D-2092 | gsasggaaUfuUfGfUfAfgaagggaus{invAb} | 751 | usAfsucccUfucuacaAfaUfuccucsusu | 752 |
| D-2093 | gsgsaauuUfgUfAfGfAfagggauaus{invAb} | 753 | usAfsuaucCfcuucuaCfaAfauuccsusu | 754 |
| D-2094 | gsasauuuGfuAfGfAfAfgggauauas{invAb} | 755 | asUfsauauCfccuucuAfcAfaauucsusu | 756 |
| D-2095 | asasuuugUfaGfAfAfGfggauauacs{invAb} | 757 | usGfsuauaUfcccuucUfaCfaaauususu | 758 |
| D-2096 | asasuuugUfaGfAfAfGfggauauacs{invAb} | 759 | usGfsuauaUfcccuucUfaCfaaauususu | 760 |
| D-2097 | asusuuguAfgAfAfGfGfgauauacas{invAb} | 761 | usUfsguauAfucccuuCfuAfcaaaususu | 762 |
| D-2098 | ususuguaGfaAfGfGfGfauauacaas{invAb} | 763 | usUfsuguaUfaucccuUfcUfacaaasusu | 764 |
| D-2099 | ususguagAfaGfGfGfAfuauacaaas{invAb} | 765 | asUfsuuguAfuaucccUfuCfuacaasusu | 766 |
| D-2100 | usgsuagaAfgGfGfAfUfauacaaags{invAb} | 767 | asCfsuuugUfauauccCfuUfcuacasusu | 768 |
| D-2101 | usgsuagaAfgGfGfAfUfauacaaags{invAb} | 769 | asCfsuuugUfauauccCfuUfcuacasusu | 770 |
| D-2102 | gsusagaaGfgGfAfUfAfuacaaagus{invAb} | 771 | asAfscuuuGfuauaucCfcUfucuacsusu | 772 |
| D-2103 | usasgaagGfgAfUfAfUfacaaagugs{invAb} | 773 | asCfsacuuUfguauauCfcCfuucuasusu | 774 |
| D-2104 | asgsaaggGfaUfAfUfAfcaaaguggs{invAb} | 775 | usCfscacuUfuguauaUfcCfcuucususu | 776 |
| D-2105 | gsasagggAfuAfUfAfCfaaaguggas{invAb} | 777 | usUfsccacUfuuguauAfuCfccuucsusu | 778 |
| D-2106 | gsasagggAfuAfUfAfCfaaaguggas{invAb} | 779 | usUfsccacUfuuguauAfuCfccuucsusu | 780 |
| D-2107 | asasgggaUfaUfAfCfAfaaguggaas{invAb} | 781 | usUfsuccaCfuuuguaUfaUfcccuususu | 782 |
| D-2108 | asgsggauAfuAfCfAfAfaguggaaas{invAb} | 783 | asUfsuuccAfcuuuguAfuAfucccususu | 784 |
| D-2109 | gsgsgauaUfaCfAfAfAfguggaaaus{invAb} | 785 | usAfsuuucCfacuuugUfaUfaucccsusu | 786 |
| D-2110 | gsgsauauAfcAfAfAfGfuggaaauas{invAb} | 787 | asUfsauuuCfcacuuuGfuAfuauccsusu | 788 |
| D-2111 | gsasuauaCfaAfAfGfUfggaaauags{invAb} | 789 | usCfsuauuUfccacuuUfgUfauaucsusu | 790 |
| D-2112 | asusauacAfaAfGfUfGfgaaauagas{invAb} | 791 | asUfscuauUfuccacuUfuGfuauaususu | 792 |
| D-2113 | usasuacaAfaGfUfGfGfaaauagacs{invAb} | 793 | usGfsucuaUfuuccacUfuUfguauasusu | 794 |
| D-2114 | asusacaaAfgUfGfGfAfaauagacas{invAb} | 795 | asUfsgucuAfuuuccaCfuUfuguaususu | 796 |
| D-2115 | asusacaaAfgUfGfGfAfaauagacas{invAb} | 797 | asUfsgucuAfuuuccaCfuUfuguaususu | 798 |
| D-2116 | usascaaaGfuGfGfAfAfauagacacs{invAb} | 799 | asGfsugucUfauuuccAfcUfuuguasusu | 800 |
| D-2117 | ascsaaagUfgGfAfAfAfuagacaccs{invAb} | 801 | usGfsguguCfuauuucCfaCfuuugususu | 802 |
| D-2118 | csasaaguGfgAfAfAfUfagacaccas{invAb} | 803 | usUfsggugUfcuauuuCfcAfcuuugsusu | 804 |
| D-2119 | asasguggAfaAfUfAfGfacaccaaas{invAb} | 805 | asUfsuuggUfgucuauUfuCfcacuususu | 806 |
| D-2120 | asasguggAfaAfUfAfGfacaccaaas{invAb} | 807 | asUfsuuggUfgucuauUfuCfcacuususu | 808 |
| D-2121 | asgsuggaAfaUfAfGfAfcaccaaaus{invAb} | 809 | asAfsuuugGfugucuaUfuUfccacususu | 810 |
| D-2122 | gsusggaaAfuAfGfAfCfaccaaaucs{invAb} | 811 | asGfsauuuGfgugucuAfuUfuccacsusu | 812 |
| D-2123 | usgsgaaaUfaGfAfCfAfccaaaucus{invAb} | 813 | asAfsgauuUfggugucUfaUfuuccasusu | 814 |
| D-2124 | gsgsaaauAfgAfCfAfCfcaaaucuus{invAb} | 815 | usAfsagauUfugguguCfuAfuuuccsusu | 816 |
| D-2125 | gsgsaaauAfgAfCfAfCfcaaaucuus{invAb} | 817 | usAfsagauUfugguguCfuAfuuuccsusu | 818 |
| D-2126 | gsasaauaGfaCfAfCfCfaaaucuuas{invAb} | 819 | asUfsaagaUfuuggugUfcUfauuucsusu | 820 |
| D-2127 | asasauagAfcAfCfCfAfaaucuuacs{invAb} | 821 | asGfsuaagAfuuugguGfuCfuauuususu | 822 |
| D-2128 | asasuagaCfaCfCfAfAfaucuuacus{invAb} | 823 | asAfsguaaGfauuuggUfgUfcuauususu | 824 |
| D-2129 | asusagacAfcCfAfAfAfucuuacugs{invAb} | 825 | asCfsaguaAfgauuugGfuGfucuaususu | 826 |
| D-2130 | asusagacAfcCfAfAfAfucuuacugs{invAb} | 827 | asCfsaguaAfgauuugGfuGfucuaususu | 828 |
| D-2131 | usasgacaCfcAfAfAfUfcuuacuggs{invAb} | 829 | usCfscaguAfagauuuGfgUfgucuasusu | 830 |
| D-2132 | asgsacacCfaAfAfUfCfuuacuggas{invAb} | 831 | usUfsccagUfaagauuUfgGfugucususu | 832 |
| D-2133 | csascuugGfcAfUfCfUfccccauucs{invAb} | 833 | asGfsaaugGfggagauGfcCfaagugsusu | 834 |
| D-2134 | ascsuuggCfaUfCfUfCfcccauuccs{invAb} | 835 | usGfsgaauGfgggagaUfgCfcaagususu | 836 |
| D-2135 | ascsuuggCfaUfCfUfCfcccauuccs{invAb} | 837 | usGfsgaauGfgggagaUfgCfcaagususu | 838 |
| D-2136 | csusuggcAfuCfUfCfCfccauuccas{invAb} | 839 | asUfsggaaUfggggagAfuGfccaagsusu | 840 |
| D-2137 | ususggcaUfcUfCfCfCfcauuccaus{invAb} | 841 | asAfsuggaAfuggggaGfaUfgccaasusu | 842 |
| D-2138 | usgsgcauCfuCfCfCfCfauuccaugs{invAb} | 843 | usCfsauggAfauggggAfgAfugccasusu | 844 |
| D-2139 | gsgscaucUfcCfCfCfAfuuccaugas{invAb} | 845 | asUfscaugGfaaugggGfaGfaugccsusu | 846 |
| D-2140 | gsgscaucUfcCfCfCfAfuuccaugas{invAb} | 847 | asUfscaugGfaaugggGfaGfaugccsusu | 848 |
| D-2141 | gscsaucuCfcCfCfAfUfuccaugags{invAb} | 849 | asCfsucauGfgaauggGfgAfgaugcsusu | 850 |
| D-2142 | csasucucCfcCfAfUfUfccaugagcs{invAb} | 851 | usGfscucaUfggaaugGfgGfagaugsusu | 852 |
| D-2143 | asuscuccCfcAfUfUfCfcaugagcas{invAb} | 853 | asUfsgcucAfuggaauGfgGfgagaususu | 854 |
| D-2144 | uscsucccCfaUfUfCfCfaugagcaus{invAb} | 855 | asAfsugcuCfauggaaUfgGfggagasusu | 856 |
| D-2145 | uscsucccCfaUfUfCfCfaugagcaus{invAb} | 857 | asAfsugcuCfauggaaUfgGfggagasusu | 858 |
| D-2146 | csusccccAfuUfCfCfAfugagcaugs{invAb} | 859 | asCfsaugcUfcauggaAfuGfgggagsusu | 860 |
| D-2147 | uscscccaUfuCfCfAfUfgagcaugcs{invAb} | 861 | usGfscaugCfucauggAfaUfggggasusu | 862 |
| D-2148 | cscsccauUfcCfAfUfGfagcaugcas{invAb} | 863 | asUfsgcauGfcucaugGfaAfuggggsusu | 864 |
| D-2149 | cscsauucCfaUfGfAfGfcaugcagas{invAb} | 865 | asUfscugcAfugcucaUfgGfaauggsusu | 866 |
| D-2150 | cscsauucCfaUfGfAfGfcaugcagas{invAb} | 867 | asUfscugcAfugcucaUfgGfaauggsusu | 868 |
| D-2151 | csasuuccAfuGfAfGfCfaugcagags{invAb} | 869 | asCfsucugCfaugcucAfuGfgaaugsusu | 870 |
| D-2152 | asusuccaUfgAfGfCfAfugcagaggs{invAb} | 871 | asCfscucuGfcaugcuCfaUfggaaususu | 872 |
| D-2153 | ususccauGfaGfCfAfUfgcagaggus{invAb} | 873 | asAfsccucUfgcaugcUfcAfuggaasusu | 874 |
| D-2154 | uscscaugAfgCfAfUfGfcagaggugs{invAb} | 875 | asCfsaccuCfugcaugCfuCfauggasusu | 876 |
| D-2155 | cscsaugaGfcAfUfGfCfagagguggs{invAb} | 877 | asCfscaccUfcugcauGfcUfcauggsusu | 878 |
| D-2156 | csasugagCfaUfGfCfAfgagguggus{invAb} | 879 | usAfsccacCfucugcaUfgCfucaugsusu | 880 |
| D-2157 | asusgagcAfuGfCfAfGfaggugguas{invAb} | 881 | asUfsaccaCfcucugcAfuGfcucaususu | 882 |
| D-2158 | usgsagcaUfgCfAfGfAfggugguaus{invAb} | 883 | asAfsuaccAfccucugCfaUfgcucasusu | 884 |
| D-2159 | gsasgcauGfcAfGfAfGfgugguauus{invAb} | 885 | asAfsauacCfaccucuGfcAfugcucsusu | 886 |
| D-2160 | asgscaugCfaGfAfGfGfugguauucs{invAb} | 887 | usGfsaauaCfcaccucUfgCfaugcususu | 888 |
| D-2161 | gscsaugcAfgAfGfGfUfgguauucas{invAb} | 889 | asUfsgaauAfccaccuCfuGfcaugcsusu | 890 |
| D-2162 | csasugcaGfaGfGfUfGfguauucacs{invAb} | 891 | usGfsugaaUfaccaccUfcUfgcaugsusu | 892 |
| D-2163 | asusgcagAfgGfUfGfGfuauucacas{invAb} | 893 | asUfsgugaAfuaccacCfuCfugcaususu | 894 |
| D-2164 | asgsagguGfgUfAfUfUfcacagccas{invAb} | 895 | usUfsggcuGfugaauaCfcAfccucususu | 896 |
| D-2165 | gsasggugGfuAfUfUfCfacagccaas{invAb} | 897 | asUfsuggcUfgugaauAfcCfaccucsusu | 898 |
| D-2166 | asgsguggUfaUfUfCfAfcagccaacs{invAb} | 899 | asGfsuuggCfugugaaUfaCfcaccususu | 900 |
| D-2167 | gsgsugguAfuUfCfAfCfagccaacgs{invAb} | 901 | usCfsguugGfcugugaAfuAfccaccsusu | 902 |
| D-2168 | gscscgccCfuGfCfUfGfagccccuas{invAb} | 903 | asUfsagggGfcucagcAfgGfgcggcsusu | 904 |
| D-2169 | cscsgcccUfgCfUfGfAfgccccuacs{invAb} | 905 | asGfsuaggGfgcucagCfaGfggcggsusu | 906 |
| D-2170 | csgscccuGfcUfGfAfGfccccuacus{invAb} | 907 | asAfsguagGfggcucaGfcAfgggcgsusu | 908 |
| D-2171 | gscsccugCfuGfAfGfCfcccuacucs{invAb} | 909 | asGfsaguaGfgggcucAfgCfagggcsusu | 910 |
| D-2172 | cscscugcUfgAfGfCfCfccuacuccs{invAb} | 911 | asGfsgaguAfggggcuCfaGfcagggsusu | 912 |
| D-2173 | cscsugcuGfaGfCfCfCfcuacuccus{invAb} | 913 | usAfsggagUfaggggcUfcAfgcaggsusu | 914 |
| D-2174 | csusgcugAfgCfCfCfCfuacuccuas{invAb} | 915 | asUfsaggaGfuaggggCfuCfagcagsusu | 916 |
| D-2175 | usgscugaGfcCfCfCfUfacuccuaus{invAb} | 917 | asAfsuaggAfguagggGfcUfcagcasusu | 918 |
| D-2176 | gscsugagCfcCfCfUfAfcuccuauus{invAb} | 919 | asAfsauagGfaguaggGfgCfucagcsusu | 920 |
| D-2177 | csusgagcCfcCfUfAfCfuccuauucs{invAb} | 921 | asGfsaauaGfgaguagGfgGfcucagsusu | 922 |
| D-2178 | usgsagccCfcUfAfCfUfccuauuccs{invAb} | 923 | usGfsgaauAfggaguaGfgGfgcucasusu | 924 |
| D-2179 | gsasgcccCfuAfCfUfCfcuauuccas{invAb} | 925 | asUfsggaaUfaggaguAfgGfggcucsusu | 926 |
| D-2180 | asgsccccUfaCfUfCfCfuauuccacs{invAb} | 927 | asGfsuggaAfuaggagUfaGfgggcususu | 928 |
| D-2181 | gscscccuAfcUfCfCfUfauuccaccs{invAb} | 929 | usGfsguggAfauaggaGfuAfggggcsusu | 930 |
| D-2182 | cscsccuaCfuCfCfUfAfuuccaccas{invAb} | 931 | asUfsggugGfaauaggAfgUfaggggsusu | 932 |
| D-2183 | cscscuacUfcCfUfAfUfuccaccacs{invAb} | 933 | asGfsugguGfgaauagGfaGfuagggsusu | 934 |
| D-2184 | cscsuacuCfcUfAfUfUfccaccacgs{invAb} | 935 | asCfsguggUfggaauaGfgAfguaggsusu | 936 |
| D-2185 | csusacucCfuAfUfUfCfcaccacggs{invAb} | 937 | asCfscgugGfuggaauAfgGfaguagsusu | 938 |
| D-2186 | usascuccUfaUfUfCfCfaccacggcs{invAb} | 939 | asGfsccguGfguggaaUfaGfgaguasusu | 940 |
| D-2187 | ascsuccuAfuUfCfCfAfccacggcus{invAb} | 941 | asAfsgccgUfgguggaAfuAfggagususu | 942 |
| D-2188 | csusccuaUfuCfCfAfCfcacggcugs{invAb} | 943 | asCfsagccGfugguggAfaUfaggagsusu | 944 |
| D-2189 | uscscuauUfcCfAfCfCfacggcugus{invAb} | 945 | asAfscagcCfguggugGfaAfuaggasusu | 946 |
| D-2190 | cscsuauuCfcAfCfCfAfcggcugucs{invAb} | 947 | asGfsacagCfcgugguGfgAfauaggsusu | 948 |
| D-2191 | csusauucCfaCfCfAfCfggcugucgs{invAb} | 949 | asCfsgacaGfccguggUfgGfaauagsusu | 950 |
| D-2192 | usasuuccAfcCfAfCfGfgcugucgus{invAb} | 951 | asAfscgacAfgccgugGfuGfgaauasusu | 952 |
| D-2193 | asusuccaCfcAfCfGfGfcugucgucs{invAb} | 953 | usGfsacgaCfagccguGfgUfggaaususu | 954 |
| D-2194 | ususccacCfaCfGfGfCfugucgucas{invAb} | 955 | asUfsgacgAfcagccgUfgGfuggaasusu | 956 |
| D-2195 | uscscaccAfcGfGfCfUfgucgucacs{invAb} | 957 | asGfsugacGfacagccGfuGfguggasusu | 958 |
| D-2196 | cscsaccaCfgGfCfUfGfucgucaccs{invAb} | 959 | usGfsgugaCfgacagcCfgUfgguggsusu | 960 |
| D-2197 | csasccacGfgCfUfGfUfcgucaccas{invAb} | 961 | usUfsggugAfcgacagCfcGfuggugsusu | 962 |
| D-2198 | ascscacgGfcUfGfUfCfgucaccaas{invAb} | 963 | asUfsugguGfacgacaGfcCfguggususu | 964 |
| D-2199 | cscsacggCfuGfUfCfGfucaccaaus{invAb} | 965 | asAfsuuggUfgacgacAfgCfcguggsusu | 966 |
| D-2200 | csascggcUfgUfCfGfUfcaccaaucs{invAb} | 967 | asGfsauugGfugacgaCfaGfccgugsusu | 968 |
| D-2201 | ascsggcuGfuCfGfUfCfaccaauccs{invAb} | 969 | asGfsgauuGfgugacgAfcAfgccgususu | 970 |
| D-2202 | csgsgcugUfcGfUfCfAfccaaucccs{invAb} | 971 | usGfsggauUfggugacGfaCfagccgsusu | 972 |
| D-2203 | gsgscuguCfgUfCfAfCfcaaucccas{invAb} | 973 | usUfsgggaUfuggugaCfgAfcagccsusu | 974 |
| D-2204 | gscsugucGfuCfAfCfCfaaucccaas{invAb} | 975 | asUfsugggAfuuggugAfcGfacagcsusu | 976 |
| D-2205 | usgsucguCfaCfCfAfAfucccaaggs{invAb} | 977 | usCfscuugGfgauuggUfgAfcgacasusu | 978 |
| D-2206 | gsuscgucAfcCfAfAfUfcccaaggas{invAb} | 979 | usUfsccuuGfggauugGfuGfacgacsusu | 980 |
| D-2207 | uscsgucaCfcAfAfUfCfccaaggaas{invAb} | 981 | asUfsuccuUfgggauuGfgUfgacgasusu | 982 |
| D-2208 | csgsucacCfaAfUfCfCfcaaggaaus{invAb} | 983 | asAfsuuccUfugggauUfgGfugacgsusu | 984 |
| D-2209 | csasccaaUfcCfCfAfAfggaaugags{invAb} | 985 | asCfsucauUfccuuggGfaUfuggugsusu | 986 |
| D-2210 | ascscaauCfcCfAfAfGfgaaugaggs{invAb} | 987 | asCfscucaUfuccuugGfgAfuuggususu | 988 |
| D-2211 | cscsaaucCfcAfAfGfGfaaugagggs{invAb} | 989 | usCfsccucAfuuccuuGfgGfauuggsusu | 990 |
| D-2212 | csasauccCfaAfGfGfAfaugagggas{invAb} | 991 | asUfscccuCfauuccuUfgGfgauugsusu | 992 |
| D-2213 | asasucccAfaGfGfAfAfugagggacs{invAb} | 993 | asGfsucccUfcauuccUfuGfggauususu | 994 |
| D-2214 | asuscccaAfgGfAfAfUfgagggacus{invAb} | 995 | asAfsguccCfucauucCfuUfgggaususu | 996 |
| D-2215 | uscsccaaGfgAfAfUfGfagggacuus{invAb} | 997 | asAfsagucCfcucauuCfcUfugggasusu | 998 |
| D-2216 | cscscaagGfaAfUfGfAfgggacuucs{invAb} | 999 | asGfsaaguCfccucauUfcCfuugggsusu | 1000 |
| D-2217 | cscsaaggAfaUfGfAfGfggacuucus{invAb} | 1001 | asAfsgaagUfcccucaUfuCfcuuggsusu | 1002 |
| D-2218 | csasaggaAfuGfAfGfGfgacuucucs{invAb} | 1003 | asGfsagaaGfucccucAfuUfccuugsusu | 1004 |
| D-2219 | csusgaagGfaCfGfAfGfggaugggas{invAb} | 1005 | asUfscccaUfcccucgUfcCfuucagsusu | 1006 |
| D-2220 | usgsaaggAfcGfAfGfGfgaugggaus{invAb} | 1007 | asAfsucccAfucccucGfuCfcuucasusu | 1008 |
| D-2221 | gsasaggaCfgAfGfGfGfaugggauus{invAb} | 1009 | asAfsauccCfaucccuCfgUfccuucsusu | 1010 |
| D-2222 | asasggacGfaGfGfGfAfugggauuus{invAb} | 1011 | asAfsaaucCfcaucccUfcGfuccuususu | 1012 |
| D-2223 | asgsgacgAfgGfGfAfUfgggauuucs{invAb} | 1013 | usGfsaaauCfccauccCfuCfguccususu | 1014 |
| D-2224 | gsgsacgaGfgGfAfUfGfggauuucas{invAb} | 1015 | asUfsgaaaUfcccaucCfcUfcguccsusu | 1016 |
| D-2225 | gsascgagGfgAfUfGfGfgauuucaus{invAb} | 1017 | asAfsugaaAfucccauCfcCfucgucsusu | 1018 |
| D-2226 | ascsgaggGfaUfGfGfGfauuucaugs{invAb} | 1019 | asCfsaugaAfaucccaUfcCfcucgususu | 1020 |
| D-2227 | csgsagggAfuGfGfGfAfuuucaugus{invAb} | 1021 | usAfscaugAfaaucccAfuCfccucgsusu | 1022 |
| D-2228 | gsasgggaUfgGfGfAfUfuucauguas{invAb} | 1023 | usUfsacauGfaaauccCfaUfcccucsusu | 1024 |
| D-2229 | asgsggauGfgGfAfUfUfucauguaas{invAb} | 1025 | asUfsuacaUfgaaaucCfcAfucccususu | 1026 |
| D-2230 | gsgsgaugGfgAfUfUfUfcauguaacs{invAb} | 1027 | asGfsuuacAfugaaauCfcCfaucccsusu | 1028 |
| D-2231 | gsgsauggGfaUfUfUfCfauguaaccs{invAb} | 1029 | usGfsguuaCfaugaaaUfcCfcauccsusu | 1030 |
| D-2232 | gsasugggAfuUfUfCfAfuguaaccas{invAb} | 1031 | usUfsgguuAfcaugaaAfuCfccaucsusu | 1032 |
| D-2233 | asusgggaUfuUfCfAfUfguaaccaas{invAb} | 1033 | asUfsugguUfacaugaAfaUfcccaususu | 1034 |
| D-2234 | usgsggauUfuCfAfUfGfuaaccaags{invAb} | 1035 | usCfsuuggUfuacaugAfaAfucccasusu | 1036 |
| D-2235 | gsgsgauuUfcAfUfGfUfaaccaagas{invAb} | 1037 | asUfscuugGfuuacauGfaAfaucccsusu | 1038 |
| D-2236 | gsgsauuuCfaUfGfUfAfaccaagags{invAb} | 1039 | asCfsucuuGfguuacaUfgAfaauccsusu | 1040 |
| D-2237 | gsasuuucAfuGfUfAfAfccaagagus{invAb} | 1041 | usAfscucuUfgguuacAfuGfaaaucsusu | 1042 |
| D-2238 | asusuucaUfgUfAfAfCfcaagaguas{invAb} | 1043 | asUfsacucUfugguuaCfaUfgaaaususu | 1044 |
| D-2239 | ususucauGfuAfAfCfCfaagaguaus{invAb} | 1045 | asAfsuacuCfuugguuAfcAfugaaasusu | 1046 |
| D-2240 | ususcaugUfaAfCfCfAfagaguauus{invAb} | 1047 | asAfsauacUfcuugguUfaCfaugaasusu | 1048 |
| D-2241 | uscsauguAfaCfCfAfAfgaguauucs{invAb} | 1049 | asGfsaauaCfucuuggUfuAfcaugasusu | 1050 |
| D-2242 | csasuguaAfcCfAfAfGfaguauuccs{invAb} | 1051 | usGfsgaauAfcucuugGfuUfacaugsusu | 1052 |
| D-2243 | asusguaaCfcAfAfGfAfguauuccas{invAb} | 1053 | asUfsggaaUfacucuuGfgUfuacaususu | 1054 |
| D-2244 | usgsuaacCfaAfGfAfGfuauuccaus{invAb} | 1055 | asAfsuggaAfuacucuUfgGfuuacasusu | 1056 |
| D-2245 | gsusaaccAfaGfAfGfUfauuccauus{invAb} | 1057 | asAfsauggAfauacucUfuGfguuacsusu | 1058 |
| D-2246 | usasaccaAfgAfGfUfAfuuccauuus{invAb} | 1059 | asAfsaaugGfaauacuCfuUfgguuasusu | 1060 |
| D-2247 | asasccaaGfaGfUfAfUfuccauuuus{invAb} | 1061 | asAfsaaauGfgaauacUfcUfugguususu | 1062 |
| D-2248 | ascscaagAfgUfAfUfUfccauuuuus{invAb} | 1063 | usAfsaaaaUfggaauaCfuCfuuggususu | 1064 |
| D-2249 | cscsaagaGfuAfUfUfCfcauuuuuas{invAb} | 1065 | asUfsaaaaAfuggaauAfcUfcuuggsusu | 1066 |
| D-2250 | csasagagUfaUfUfCfCfauuuuuacs{invAb} | 1067 | asGfsuaaaAfauggaaUfaCfucuugsusu | 1068 |
| D-2251 | asasgaguAfuUfCfCfAfuuuuuacus{invAb} | 1069 | usAfsguaaAfaauggaAfuAfcucuususu | 1070 |
| D-2252 | asgsaguaUfuCfCfAfUfuuuuacuas{invAb} | 1071 | usUfsaguaAfaaauggAfaUfacucususu | 1072 |
| D-2253 | gsasguauUfcCfAfUfUfuuuacuaas{invAb} | 1073 | usUfsuaguAfaaaaugGfaAfuacucsusu | 1074 |
| D-2254 | asgsuauuCfcAfUfUfUfuuacuaaas{invAb} | 1075 | asUfsuuagUfaaaaauGfgAfauacususu | 1076 |
| D-2255 | uscscauuUfuUfAfCfUfaaagcagus{invAb} | 1077 | asAfscugcUfuuaguaAfaAfauggasusu | 1078 |
| D-2256 | csasuuuuUfaCfUfAfAfagcagugus{invAb} | 1079 | asAfscacuGfcuuuagUfaAfaaaugsusu | 1080 |
| D-2257 | asusuuuuAfcUfAfAfAfgcaguguus{invAb} | 1081 | asAfsacacUfgcuuuaGfuAfaaaaususu | 1082 |
| D-2258 | ususuuuaCfuAfAfAfGfcaguguuus{invAb} | 1083 | asAfsaacaCfugcuuuAfgUfaaaaasusu | 1084 |
| D-2259 | ususuuacUfaAfAfGfCfaguguuuus{invAb} | 1085 | asAfsaaacAfcugcuuUfaGfuaaaasusu | 1086 |
| D-2260 | ususacuaAfaGfCfAfGfuguuuucas{invAb} | 1087 | asUfsgaaaAfcacugcUfuUfaguaasusu | 1088 |
| D-2261 | usascuaaAfgCfAfGfUfguuuucacs{invAb} | 1089 | asGfsugaaAfacacugCfuUfuaguasusu | 1090 |
| D-2262 | ascsuaaaGfcAfGfUfGfuuuucaccs{invAb} | 1091 | asGfsgugaAfaacacuGfcUfuuagususu | 1092 |
| D-2263 | csusaaagCfaGfUfGfUfuuucaccus{invAb} | 1093 | asAfsggugAfaaacacUfgCfuuuagsusu | 1094 |
| D-2264 | usasaagcAfgUfGfUfUfuucaccucs{invAb} | 1095 | usGfsagguGfaaaacaCfuGfcuuuasusu | 1096 |
| D-2265 | asasagcaGfuGfUfUfUfucaccucas{invAb} | 1097 | asUfsgaggUfgaaaacAfcUfgcuuususu | 1098 |
| D-2266 | asasgcagUfgUfUfUfUfcaccucaus{invAb} | 1099 | usAfsugagGfugaaaaCfaCfugcuususu | 1100 |
| D-2267 | asgsuccaGfgCfAfGfAfgacaauaas{invAb} | 1101 | usUfsuauuGfucucugCfcUfggacususu | 1102 |
| D-2268 | gsusccagGfcAfGfAfGfacaauaaas{invAb} | 1103 | usUfsuuauUfgucucuGfcCfuggacsusu | 1104 |
| D-2269 | uscscaggCfaGfAfGfAfcaauaaaas{invAb} | 1105 | asUfsuuuaUfugucucUfgCfcuggasusu | 1106 |
| D-2270 | cscsaggcAfgAfGfAfCfaauaaaacs{invAb} | 1107 | usGfsuuuuAfuugucuCfuGfccuggsusu | 1108 |
| D-2271 | csasggcaGfaGfAfCfAfauaaaacas{invAb} | 1109 | asUfsguuuUfauugucUfcUfgccugsusu | 1110 |
| D-2272 | asgsgcagAfgAfCfAfAfuaaaacaus{invAb} | 1111 | asAfsuguuUfuauuguCfuCfugccususu | 1112 |
| D-2273 | gsgscagaGfaCfAfAfUfaaaacauus{invAb} | 1113 | asAfsauguUfuuauugUfcUfcugccsusu | 1114 |
| D-2274 | asgsacaaUfaAfAfAfCfauuccugus{invAb} | 1115 | asAfscaggAfauguuuUfaUfugucususu | 1116 |
| D-2275 | gsascaauAfaAfAfCfAfuuccugugs{invAb} | 1117 | usCfsacagGfaauguuUfuAfuugucsusu | 1118 |
| D-2276 | ascsaauaAfaAfCfAfUfuccugugas{invAb} | 1119 | usUfscacaGfgaauguUfuUfauugususu | 1120 |
| D-2277 | csasauaaAfaCfAfUfUfccugugaas{invAb} | 1121 | usUfsucacAfggaaugUfuUfuauugsusu | 1122 |
| D-2278 | asasuaaaAfcAfUfUfCfcugugaaas{invAb} | 1123 | asUfsuucaCfaggaauGfuUfuuauususu | 1124 |
| D-2279 | asusaaaaCfaUfUfCfCfugugaaags{invAb} | 1125 | asCfsuuucAfcaggaaUfgUfuuuaususu | 1126 |
| D-2280 | usasaaacAfuUfCfCfUfgugaaaggs{invAb} | 1127 | asCfscuuuCfacaggaAfuGfuuuuasusu | 1128 |
| D-2281 | asasaacaUfuCfCfUfGfugaaaggcs{invAb} | 1129 | usGfsccuuUfcacaggAfaUfguuuususu | 1130 |
| D-2282 | asasacauUfcCfUfGfUfgaaaggcas{invAb} | 1131 | asUfsgccuUfucacagGfaAfuguuususu | 1132 |
| D-2283 | {sGalNAc3}ucuuggCfaGfGfAfUfggcuucuc | 1133 | usGfsagaaGfccauccUfgCfcaagasusu | 1134 |
| | s{invAb} | | | |
| D-2284 | {sGalNAc3}ucuuggCfaGfGfAfUfggcaucuc | 1135 | usGfsagauGfccauccUfgCfcaagasusu | 1136 |
| | s{invAb} | | | |
| D-2285 | {sGalNAc3}ucuuggCfaGfGfAfUfggguucuc | 1137 | usGfsagaaCfccauccUfgCfcaagasusu | 1138 |
| | s{invAb} | | | |
| D-2286 | {sGalNAc3}ucuuggCfaGfGfAfUfgccuucuc | 1139 | usGfsagaaGfgcauccUfgCfcaagasusu | 1140 |
| | s{invAb} | | | |
| D-2287 | {sGalNAc3}cuuggcAfgGfAfUfGfgcuucuca | 1141 | asUfsgagaAfgccaucCfuGfccaagsusu | 1142 |
| | s{invAb} | | | |
| D-2288 | {sGalNAc3}uggcagGfaUfGfGfCfuucucauc | 1143 | asGfsaugaGfaagccaUfcCfugccasusu | 1144 |
| | s{invAb} | | | |
| D-2289 | {sGalNAc3}gcaggaUfgGfCfUfUfcucaucgu | 1145 | asAfscgauGfagaagcCfaUfccugcsusu | 1146 |
| | s{invAb} | | | |
| D-2290 | {sGalNAc3}caggauGfgCfUfUfCfucaucguc | 1147 | asGfsacgaUfgagaagCfcAfuccugsusu | 1148 |
| | s{invAb} | | | |
| D-2291 | {sGalNAc3}caggauGfgCfUfUfCfucuucguc | 1149 | asGfsacgaAfgagaagCfcAfuccugsusu | 1150 |
| | sfinvAb} | | | |
| D-2292 | {sGalNAc3}aggaugGfcUfUfCfUfcaucgucu | 1151 | asAfsgacgAfugagaaGfcCfauccususu | 1152 |
| | s{invAb} | | | |
| D-2293 | {sGalNAc3}gauggcUfuCfUfCfAfucgucugc | 1153 | asGfscagaCfgaugagAfaGfccaucsusu | 1154 |
| | s{invAb} | | | |
| D-2294 | {sGalNAc3}ggcuucUfcAfUfCfGfucugcus | 1155 | gsAfsgcagAfcgaugaGfaAfgccsusu | 1156 |
| | {invAb} | | | |
| D-2295 | {sGalNAc3}ggcuUfcUfCfAfUfcgucugcus | 1157 | gsAfsgcagAfcgaugaGfaAfgccsusu | 1158 |
| | {invAb} | | | |
| D-2296 | {sGalNAc3}auggcuucUfcAfUfCfGfucugcu | 1159 | asAfsgcagAfcgaugaGfaAfgccaususu | 1160 |
| | s{invAb} | | | |
| D-2297 | {sGalNAc3}auggcuucUfcAfUfCfGfacugcu | 1161 | asAfsgcagUfcgaugaGfaAfgccaususu | 1162 |
| | s{invAb} | | | |
| D-2298 | {sGalNAc3}auggcuucUfcAfUfCfCfucugcu | 1163 | asAfsgcagAfggaugaGfaAfgccaususu | 1164 |
| | s{invAb} | | | |
| D-2299 | {sGalNAc3}auggcuUfcUfCfAfUfcgucugcu | 1165 | asAfsgcagacgauGfaGfaAfgccaususu | 1166 |
| | s{invAb} | | | |
| D-2300 | {sGalNAc3}auggcuUfcUfCfAfUfccucugcu | 1167 | asAfsgcagaggauGfaGfaAfgccaususu | 1168 |
| | s{invAb} | | | |
| D-2301 | {sGalNAc3}auggcuUfcUfcAfucgucugcus | 1169 | asAfsgcagAfcgauGfaGfaAfgccausus | 1170 |
| | {invAb} | | u | |
| D-2302 | {sGalNAc3}auggcuUfcUfcAfuccucugcus | 1171 | asAfsgcagAfggauGfaGfaAfgccausus | 1172 |
| | {invAb} | | u | |
| D-2303 | {sGalNAc3}auggcuucUfcAfucgucugcus | 1173 | asAfsgcagAfcgauGfaGfaagccaususu | 1174 |
| | {invAb} | | | |
| D-2304 | {sGalNAc3}ggcuUfcUfCfAfUfccucugcus | 1175 | asAfsgcagaggauGfaGfaAfgccsusu | 1176 |
| | {invAb} | | | |
| D-2305 | {sGalNAc3}auggcuucUfcAfUfCfCfucugc | 1177 | asAfsgcAfgAfggauGfaGfaAfgccausc | 1178 |
| | sus{invAb} | | sc | |
| D-2306 | {sGalNAc3}auggcuucUfcAfUfCfCfucugc | 1179 | asAfsgcaGfAfggauGfaGfaAfgccausc | 1180 |
| | sus{invAb} | | sc | |
| D-2307 | {sGalNAc3}auggcuUfcUfCfAfUfccucugc | 1181 | asAfsgcaGfAfggauGfaGfaAfgccausc | 1182 |
| | sus{invAb} | | sc | |
| D-2308 | {sGalNAc3}auggcuUfcUfCfAfUfccucugc | 1183 | asAfsgcAfgAfggauGfaGfaAfgccausc | 1184 |
| | sus{invAb} | | sc | |
| D-2309 | {sGalNAc3}auggcuUfcUfCfAfUfcgucugc | 1185 | asAfsgcagAfcgaugaGfaAfgccsasu | 1186 |
| | us{invAb} | | | |
| D-2310 | {sGalNAc3}auggcuUfcUfCfAfUfcgacugcu | 1187 | asAfsgcagUfcgaugaGfaAfgccsasu | 1188 |
| | s{invAb} | | | |
| D-2311 | {sGalNAc3}auggcuUfcUfCfAfUfccucugcu | 1189 | asAfsgcagAfggaugaGfaAfgccsasu | 1190 |
| | s{invAb} | | | |
| D-2312 | {sGalNAc3}ggcuucUfcAfUfCfGfucugcus | 1191 | asAfsgcagAfcgaugaGfaAfgccsusu | 1192 |
| | {invAb} | | | |
| D-2313 | {sGalNAc3}ggcuucUfcAfUfCfCfucugcus | 1193 | asAfsgcagAfggaugaGfaAfgccsusu | 1194 |
| | {invAb} | | | |
| D-2314 | {sGalNAc3}auggcuUfcUfcAfucgucugcus | 1195 | asAfsgcagacgauGfaGfaAfgccaususu | 1196 |
| | {invAb} | | | |
| D-2315 | {sGalNAc3}auggcuUfcUfcAfuccucugcus | 1197 | asAfsgcagaggauGfaGfaAfgccaususu | 1198 |
| | {invAb} | | | |
| D-2316 | {sGalNAc3}auggcuUfcUfCfAfUfcgucugcu | 1199 | asAfsgcagAfcgaugaGfaAfgccaususu | 1200 |
| | s{invAb} | | | |
| D-2317 | {sGalNAc3}auggcuUfcUfCfAfUfccucugcu | 1201 | asAfsgcagAfggaugaGfaAfgccaususu | 1202 |
| | s{invAb} | | | |
| D-2318 | {sGalNAc3}ggcuUfcUfcAfuccucugcus | 1203 | asAfsgcagaggauGfaGfaAfgccsusu | 1204 |
| | {invAb} | | | |
| D-2319 | {sGalNAc3}ggcuUfcUfcAfuccucugcus | 1205 | asAfsgcagAfggauGfaGfaAfgccsusu | 1206 |
| | {invAb} | | | |
| D-2320 | {sGalNAc3}ggcuUfcUfCfAfUfcgucugcus | 1207 | asAfsgcagAfcgaugaGfaAfgccsusu | 1208 |
| | {invAb} | | | |
| D-2321 | {sGalNAc3}ggcuUfcUfCfAfUfccucugcus | 1209 | asAfsgcagAfggaugaGfaAfgccsusu | 1210 |
| | {invAb} | | | |
| D-2322 | {sGalNAc3}gcuucuCfaUfCfGfUfcugcucc | 1211 | asAfsggagCfagacgaUfgAfgaagcsusu | 1212 |
| | us{invAb} | | | |
| D-2323 | {sGalNAc3}gcuucuCfaUfCfGfUfcuggucc | 1213 | asAfsggacCfagacgaUfgAfgaagcsusu | 1214 |
| | us{invAb} | | | |
| D-2324 | {sGalNAc3}cuucucAfuCfGfUfCfugcuccu | 1215 | asGfsaggaGfcagacgAfuGfagaagsusu | 1216 |
| | cs{invAb} | | | |
| D-2325 | {sGalNAc3}uucucaUfcGfUfCfUfgcuccuc | 121; | asGfsgaggAfgcagacGfaUfgagaasusu | 1218 |
| | cs{invAb} | | | |
| D-2326 | {sGalNAc3}ucucauCfgUfCfUfGfcuccucc | 1219 | asAfsggagGfagcagaCfgAfugagasusu | 1220 |
| | us{invAb} | | | |
| D-2327 | {sGalNAc3}ucucauCfgUfCfUfGfcucgucc | 1221 | asAfsggacGfagcagaCfgAfugagasusu | 1222 |
| | us{invAb} | | | |
| D-2328 | {sGalNAc3}cucaucGfuCfUfGfCfuccuccu | 1223 | asGfsaggaGfgagcagAfcGfaugagsusu | 1224 |
| | cs{invAb} | | | |
| D-2329 | {sGalNAc3}ucgucuGfcUfCfCfUfccucugs | 1225 | gsCfsagagGfaggagcAfgAfcgasusu | 1226 |
| | {invAb} | | | |
| D-2330 | {sGalNAc3}ucguCfuGfCfUfCfcuccucugs | 1227 | gsCfsagagGfaggagcAfgAfcgasusu | 1228 |
| | {invAb} | | | |
| D-2331 | {sGalNAc3}caucgucuGfcUfCfCfUfgcucu | 1229 | asCfsagagCfaggagcAfgAfcgaugsusu | 1230 |
| | gs{invAb} | | | |
| D-2332 | {sGalNAc3}caucguCfuGfCfUfCfcugcucu | 1231 | asCfsagagCfaggagcAfgAfcgasusg | 1232 |
| | gs{invAb} | | | |
| D-2333 | {sGalNAc3}caucguCfuGfCfUfCfcuccucu | 1233 | asCfsagagGfaggagcAfgAfcgaugsusu | 1234 |
| | gs{invAb} | | | |
| D-2334 | {sGalNAc3}caucguCfuGfCfUfCfcaccucu | 1235 | asCfsagagGfuggagcAfgAfcgaugsusu | 1236 |
| | gs{invAb} | | | |
| D-2335 | {sGalNAc3}ugccuuGfcUfGfGfAfcugguas | 1237 | asUfsaccaGfuccagcAfaGfgcasusu | 1238 |
| | {invAb} | | | |
| D-2336 | {sGalNAc3}ugccUfuGfCfUfGfgacugguas | 1239 | asUfsaccaGfuccagcAfaGfgcasusu | 1240 |
| | {invAb} | | | |
| D-2337 | {sGalNAc3}gcuggaCfuGfGfUfAfuuugugu | 1241 | asGfsacacAfaauaccAfgUfccagcsusu | 1242 |
| | cs{invAb} | | | |
| D-2338 | {sGalNAc3}gcuggaCfuGfGfUfAfuuucugu | 1243 | asGfsacagAfaauaccAfgUfccagcsusu | 1244 |
| | cs{invAb} | | | |
| D-2339 | {sGalNAc3}cuggacUfgGfUfAfUfuuguguc | 1245 | asAfsgacaCfaaauacCfaGfuccagsusu | 1246 |
| | us{invAb} | | | |
| D-2340 | {sGalNAc3}uggacuggUfaUfUfUfCfugucu | 1247 | usCfsagacAfgaaauaCfcAfguccasusu | 1248 |
| | gs{invAb} | | | |
| D-2341 | {sGalNAc3}uggacuGfgUfAfUfUfucugucu | 1249 | usCfsagacAfgaaauaCfcAfgucscsa | 1250 |
| | gs{invAb} | | | |
| D-2342 | {sGalNAc3}uggacuGfgUfAfUfUfugugucu | 1251 | usCfsagacAfcaaauaCfcAfguccasusu | 1252 |
| | gs{invAb} | | | |
| D-2343 | {sGalNAc3}ggacugGfuAfUfUfUfgugucug | 1253 | asUfscagaCfacaaauAfcCfaguccsusu | 1254 |
| | as{invAb} | | | |
| D-2344 | {sGalNAc3}acugguAfuUfUfGfUfgucugag | 1255 | asCfscucaGfacacaaAfuAfccagususu | 1256 |
| | gs{invAb} | | | |
| D-2345 | {sGalNAc3}cugguaUfuUfGfUfGfucugagg | 1257 | asGfsccucAfgacacaAfaUfaccagsusu | 1258 |
| | cs{invAb} | | | |
| D-2346 | {sGalNAc3}gguauuugUfgUfCfUfGfacgcu | 1259 | asCfsagCfgUfcagaCfaCfaAfauaccsa | 1260 |
| | sgs{invAb} | | sg | |
| D-2347 | {sGalNAc3}gguauuugUfgUfCfUfGfacgcus | 1261 | asCfsagcGfUfcagaCfaCfaAfauaccsa | 1262 |
| | gs{invAb} | | sg | |
| D-2348 | {sGalNAc3}gguauuUfgUfGfUfCfugacgcus | 1263 | asCfsagcGfUfcagaCfaCfaAfauaccsa | 1264 |
| | gs{invAb} | | sg | |
| D-2349 | {sGalNAc3}gguauuUfgUfGfUfCfugacgcus | 1265 | asCfsagCfgUfcagaCfaCfaAfauaccsa | 1266 |
| | gs{invAb} | | sg | |
| D-2350 | {sGalNAc3}gguauuUfgUfGfUfCfugaggcug | 1267 | asCfsagccUfcagacaCfaAfauaccsusu | 1268 |
| | s{invAb} | | | |
| D-2351 | {sGalNAc3}gguauuUfgUfGfUfCfugacgcug | 1269 | asCfsagcgUfcagacaCfaAfauaccsusu | 1270 |
| | s{invAb} | | | |
| D-2352 | {sGalNAc3}ugaaucCfaAfGfUfGfuccucuga | 1271 | asUfscagaGfgacacuUfgGfauucasusu | 1272 |
| | s{invAb} | | | |
| D-2353 | {sGalNAc3}ugaaucCfaAfGfUfGfugcucuga | 1273 | asUfscagaGfcacacuUfgGfauucasusu | 1274 |
| | s{invAb} | | | |
| D-2354 | {sGalNAc3}aauccaAfgUfGfUfCfcucugaug | 1275 | asCfsaucaGfaggacaCfuUfggauususu | 1276 |
| | s{invAb} | | | |
| D-2355 | {sGalNAc3}auccaaGfuGfUfCfCfucugaugg | 1277 | asCfscaucAfgaggacAfcUfuggaususu | 1278 |
| | s{invAb} | | | |
| D-2356 | {sGalNAc3}auccaaGfuGfUfCfCfucucaugg | 1279 | asCfscaugAfgaggacAfcUfuggaususu | 1280 |
| | s{invAb} | | | |
| D-2357 | {sGalNAc3}auccaaGfuGfUfCfCfucagaugg | 1281 | asCfscaucUfgaggacAfcUfuggaususu | 1282 |
| | s{invAb} | | | |
| D-2358 | {sGalNAc3}uccaagugUfcCfUfCfUfcauggu | 1283 | asAfsccauGfagaggaCfaCfuuggasusu | 1284 |
| | s{invAb} | | | |
| D-2359 | {sGalNAc3}uccaagUfgUfCfCfUfcucauggu | 1285 | asAfsccauGfagaggaCfaCfuugsgsa | 1286 |
| | s{invAb} | | | |
| D-2360 | {sGalNAc3}caagugUfcCfUfCfUfcauggus | 1287 | asAfsccauGfagaggaCfaCfuugsusu | 1288 |
| | {invAb} | | | |
| D-2361 | {sGalNAc3}uccaagUfgUfCfCfUfcugauggu | 1289 | asAfsccauCfagaggaCfaCfuuggasusu | 1290 |
| | s{invAb} | | | |
| D-2362 | {sGalNAc3}uccaagUfgUfCfCfUfcuguuggu | 1291 | asAfsccaaCfagaggaCfaCfuuggasusu | 1292 |
| | s{invAb} | | | |
| D-2363 | {sGalNAc3}uccaagUfgUfCfCfUfcucauggu | 1293 | asAfsccauGfagaggaCfaCfuuggasusu | 1294 |
| | s{invAb} | | | |
| D-2364 | {sGalNAc3}caagUfgUfCfCfUfcucauggus | 1295 | asAfsccauGfagaggaCfaCfuugsusu | 1296 |
| | {invAb} | | | |
| D-2365 | {sGalNAc3}ccaaguguCfcUfCfUfGfaugguc | 1297 | usGfsaccaUfcagaggAfcAfcuuggsusu | 1298 |
| | s{invAb} | | | |
| D-2366 | {sGalNAc3}ccaaguguCfcUfCfUfGfuugguc | 1299 | usGfsaccaAfcagaggAfcAfcuuggsusu | 1300 |
| | s{invAb} | | | |
| D-2367 | {sGalNAc3}ccaaguGfuCfCfUfCfugaugguc | 1301 | usGfsaccaucagaGfgAfcAfcuuggsusu | 1302 |
| | s{invAb} | | | |
| D-2368 | {sGalNAc3}ccaaguGfuCfcUfcugauggucs | 1303 | usGfsaccaUfcagaGfgAfcAfcuuggsusu | 1304 |
| | {invAb} | | | |
| D-2369 | {sGalNAc3}ccaaguGfuCfcUfcuguuggucs | 1305 | usGfsaccaacagaGfgAfcAfcuusgsg | 1306 |
| | {invAb} | | | |
| D-2370 | {sGalNAc3}ccaaguGfuCfcUfcuguuggucs | 1307 | usGfsaccaAfcagaGfgAfcAfcuusgsg | 1308 |
| | {invAb} | | | |
| D-2371 | {sGalNAc3}ccaaguGfuCfCfUfCfuguugguc | 1309 | usGfsaccaacagaGfgAfcAfcuusgsg | 1310 |
| | s{invAb} | | | |
| D-2372 | {sGalNAc3}ccaaguguCfcUfCfUfGfuuggus | 1311 | usGfsacCfaAfcagaGfgAfcAfcuuggsa | 1312 |
| | cs{invAb} | | su | |
| D-2373 | {sGalNAc3}ccaaguguCfcUfCfUfGfuuggus | 1313 | usGfsaccAfAfcagaGfgAfcAfcuuggsa | 1314 |
| | cs{invAb} | | su | |
| D-2374 | {sGalNAc3}ccaaguGfuCfCfUfCfuguuggus | 1315 | usGfsaccAfAfcagaGfgAfcAfcuuggsa | 1316 |
| | cs{invAb} | | su | |
| D-2375 | {sGalNAc3}ccaaguGfuCfCfUfCfuguuggus | 1317 | usGfsacCfaAfcagaGfgAfcAfcuuggsa | 1318 |
| | cs{invAb} | | su | |
| D-2376 | {sGalNAc3}ccaaguGfuCfCfUfCfugaugguc | 1319 | usGfsaccaUfcagaggAfcAfcuusgsg | 1320 |
| | s{invAb} | | | |
| D-2377 | {sGalNAc3}ccaaguGfuCfCfUfCfuguugguc | 1321 | usGfsaccaAfcagaggAfcAfcuusgsg | 1322 |
| | słinvAb} | | | |
| D-2378 | {sGalNAc3}aaguguCfcUfCfUfGfauggucs | 1323 | usGfsaccaUfcagaggAfcAfcuususu | 1324 |
| | {invAb} | | | |
| D-2379 | {sGalNAc3}aaguguCfcUfCfUfGfuuggucs | 1325 | usGfsaccaAfcagaggAfcAfcuususu | 1326 |
| | {invAb} | | | |
| D-2380 | {sGalNAc3}ccaaguGfuCfcUfcugauggucs | 1327 | usGfsaccaucagaGfgAfcAfcuuggsusu | 1328 |
| | {invAb} | | | |
| D-2381 | {sGalNAc3}ccaaguGfuCfCfUfCfugaugguc | 1329 | usGfsaccaUfcagaggAfcAfcuuggsusu | 1330 |
| | s{invAb} | | | |
| D-2382 | {sGalNAc3}ccaaguGfuCfCfUfCfugaagguc | 1331 | usGfsaccuUfcagaggAfcAfcuuggsusu | 1332 |
| | s{invAb} | | | |
| D-2383 | {sGalNAc3}ccaaguGfuCfCfUfCfuguugguc | 1333 | usGfsaccaAfcagaggAfcAfcuuggsusu | 1334 |
| | s{invAb} | | | |
| D-2384 | {sGalNAc3}ccaaguGfuCfCfUfCfucaugguc | 1335 | usGfsaccaUfgagaggAfcAfcuuggsusu | 1336 |
| | s{invAb} | | | |
| D-2385 | {sGalNAc3}aaguGfuCfCfUfCfugauggucs | 1337 | usGfsaccaUfcagaggAfcAfcuususu | 1338 |
| | {invAb} | | | |
| D-2386 | {sGalNAc3}aaguGfuCfCfUfCfuguuggucs | 1339 | usGfsaccaAfcagaggAfcAfcuususu | 1340 |
| | {invAb} | | | |
| D-2387 | {sGalNAc3}caagugUfcCfUfCfUfgaugguca | 1341 | usUfsgaccAfucagagGfaCfacuugsusu | 1342 |
| | s{invAb} | | | |
| D-2388 | {sGalNAc3}caagugUfcCfUfCfUfgaucguca | 1343 | usUfsgacgAfucagagGfaCfacuugsusu | 1344 |
| | s{invAb} | | | |
| D-2389 | {sGalNAc3}caagugUfcCfUfCfUfgaagguca | 1345 | usUfsgaccUfucagagGfaCfacuugsusu | 1346 |
| | s{invAb} | | | |
| D-2390 | {sGalNAc3}caagugUfcCfUfCfUfguugguca | 1347 | usUfsgaccAfacagagGfaCfacuugsusu | 1348 |
| | s{invAb} | | | |
| D-2391 | {sGalNAc3}uccucuGfaUfGfGfUfcaaaguuc | 1349 | asGfsaacuUfugaccaUfcAfgaggasusu | 1350 |
| | s{invAb} | | | |
| D-2392 | {sGalNAc3}uccucuGfaUfGfGfUfcauaguuc | 1351 | asGfsaacuAfugaccaUfcAfgaggasusu | 1352 |
| | s{invAb} | | | |
| D-2393 | {sGalNAc3}ccucugAfuGfGfUfCfaaaguucu | 1353 | usAfsgaacUfuugaccAfuCfagaggsusu | 1354 |
| | s{invAb} | | | |
| D-2394 | {sGalNAc3}ccucugAfuGfGfUfCfaaacuucu | 1355 | usAfsgaagUfuugaccAfuCfagaggsusu | 1356 |
| | s{invAb} | | | |
| D-2395 | {sGalNAc3}cucugaugGfuCfAfAfAfguucua | 1357 | asUfsagaaCfuuugacCfaUfcagagsusu | 1358 |
| | s{invAb} | | | |
| D-2396 | {sGalNAc3}cucugaUfgGfUfCfAfaaguucua | 1359 | asUfsagaaCfuuugacCfaUfcagsasg | 1360 |
| | s{invAb} | | | |
| D-2397 | {sGalNAc3}cugaugGfuCfAfAfAfguucuas | 1361 | asUfsagaaCfuuugacCfaUfcagsusu | 1362 |
| | {invAb | | | |
| D-2398 | {sGalNAc3}cucugaUfgGfUfCfAfaaguucua | 1363 | asUfsagaaCfuuugacCfaUfcagagsusu | 1364 |
| | s{invAb} | | | |
| D-2399 | {sGalNAc3}cugaUfgGfUfCfAfaaguucuas | 1365 | asUfsagaaCfuuugacCfaUfcagsusu | 1366 |
| | {invAb} | | | |
| D-2400 | {sGalNAc3}ucugauggUfcAfAfAfCfuucuag | 1367 | usCfsuagaAfguuugaCfcAfucagasusu | 1368 |
| | s{invAb} | | | |
| D-2401 | {sGalNAc3}ucugauGfgUfcAfaacuucuags | 1369 | usCfsuagaaguuuGfaCfcAfucasgsa | 1370 |
| | {invAb} | | | |
| D-2402 | {sGalNAc3}ucugauGfgUfcAfaacuucuags | 1371 | usCfsuagaAfguuuGfaCfcAfucasgsa | 1372 |
| | {invAb} | | | |
| D-2403 | {sGalNAc3}ucugauGfgUfCIAfAfacuucuag | 1373 | usCfsuagaaguuuGfaCfcAfucasgsa | 1374 |
| | s{invAb} | | | |
| D-2404 | {sGalNAc3}ucugauggUfcAfAfAfCfuucuas | 1375 | usCfsuaGfaAfguuuGfaCfcAfucagasg | 1376 |
| | gs{invAb} | | sg | |
| D-2405 | {sGalNAc3}ucugauggUfcAfAfAfCfuucuas | 1377 | usCfsuagAfAfguuuGfaCfcAfucagasg | 1378 |
| | gs{invAb} | | sg | |
| D-2406 | {sGalNAc3}ucugauGfgUfCfAfAfacuucuas | 1379 | usCfsuagAfAfguuuGfaCfcAfucagasg | 1380 |
| | gs{invAb} | | sg | |
| D-2407 | {sGalNAc3}ucugauGfgUfCfAfAfacuucuas | 1381 | usCfsuaGfaAfguuuGfaCfcAfucagasg | 1382 |
| | gs{invAb} | | sg | |
| D-2408 | {sGalNAc3}ucugauGfgUfCfAfAfacuucuag | 1383 | usCfsuagaAfguuugaCfcAfucasgsa | 1384 |
| | s{invAb} | | | |
| D-2409 | {sGalNAc3}ugauggUfcAfAfAfCfuucuags | 1385 | usCfsuagaAfguuugaCfcAfucasusu | 1386 |
| | {invAb; | | | |
| D-2410 | {sGalNAc3}ucugauGfgUfCfAfAfaguucuag | 1387 | usCfsuagaAfcuuugaCfcAfucagasusu | 1388 |
| | s{invAb} | | | |
| D-2411 | {sGalNAc3}ucugauGfgUfCfAfAfaguacuag | 1389 | usCfsuaguAfcuuugaCfcAfucagasusu | 1390 |
| | s{invAb | | | |
| D-2412 | {sGalNAc3}ucugauGfgUfCfAfAfacuucuag | 1391 | usCfsuagaAfguuugaCfcAfucagasusu | 1392 |
| | s{invAb} | | | |
| D-2413 | {sGalNAc3}ugauGfgUfCfAfAfacuucuags | 1393 | usCfsuagaAfguuugaCfcAfucasusu | 1394 |
| | {invAb} | | | |
| D-2414 | {sGalNAc3}cugaugguCfaAfAfGfUfucuaga | 1395 | asUfscuagAfacuuugAfcCfaucagsusu | 1396 |
| | s{invAb} | | | |
| D-2415 | {sGalNAc3}cugaugguCfaAfAfGfUfacuaga | 1397 | asUfscuagUfacuuugAfcCfaucagsusu | 1398 |
| | s{invAb} | | | |
| D-2416 | {sGalNAc3}cugaugguCfaAfAfGfAfucuaga | 1399 | asUfscuagAfucuuugAfcCfaucagsusu | 1400 |
| | s{invAb} | | | |
| D-2417 | {sGalNAc3}cugaugGfuCfAfAfAfguucuaga | 1401 | asUfscuagAfacuuugAfcCfaucsasg | 1402 |
| | s{invAb} | | | |
| D-2418 | {sGalNAc3}cugaugGfuCfAfAfAfguacuaga | 1403 | asUfscuagUfacuuugAfcCfaucsasg | 1404 |
| | s{invAb} | | | |
| D-2419 | {sGalNAc3}cugaugGfuCfAfAfAfgaucuaga | 1405 | asUfscuagAfucuuugAfcCfaucsasg | 1406 |
| | s{invAb} | | | |
| D-2420 | {sGalNAc3}gaugguCfaAfAfGfUfucuagas | 1407 | asUfscuagAfacuuugAfcCfaucsusu | 1408 |
| | {invAb} | | | |
| D-2421 | {sGalNAc3}gaugguCfaAfAfGfUfucuagas | 1409 | asUfscuagAfacuuugAfcCfaucsusu | 1410 |
| | {invAb} | | | |
| D-2422 | {sGalNAc3}gaugguCfaAfAfGfAfucuagas | 1411 | asUfscuagAfucuuugAfcCfaucsusu | 1412 |
| | {invAb} | | | |
| D-2423 | {sGalNAc3}cugaugGfuCfAfAfAfguucuaga | 1413 | asUfscuagAfacuuugAfcCfaucagsusu | 1414 |
| | s{invAb} | | | |
| D-2424 | {sGalNAc3}cugaugGfuCfAfAfAfgaucuaga | 1415 | asUfscuagAfucuuugAfcCfaucagsusu | 1416 |
| | s{invAb} | | | |
| D-2425 | {sGalNAc3}gaugGfuCfAfAfAfguucuagas | 1417 | asUfscuagAfacuuugAfcCfaucsusu | 1418 |
| | {invAb} | | | |
| D-2426 | {sGalNAc3}gaugGfuCfAfAfAfguucuagas | 1419 | asUfscuagAfacuuugAfcCfaucsusu | 1420 |
| | {invAb} | | | |
| D-2427 | {sGalNAc3}gaugGfuCfAfAfAfgaucuagas | 1421 | asUfscuagAfucuuugAfcCfaucsusu | 1422 |
| | {invAb} | | | |
| D-2428 | {sGalNAc3}gauggucaAfaGfUfUfCfuugaug | 1423 | asCfsaucaAfgaacuuUfgAfccaucsusu | 1424 |
| | s{invAb} | | | |
| D-2429 | {sGalNAc3}gauggucaAfaGfUfUfCfuugaus | 1425 | asCfsauCfaAfgaacUfuUfgAfccaucsa | 1426 |
| | gs{invAb} | | sg | |
| D-2430 | {sGalNAc3}gauggucaAfaGfUfUfGfuagaus | 1427 | asCfsauCfuAfcaacUfuUfgAfccaucsa | 1428 |
| | gs{invAb} | | sg | |
| D-2431 | {sGalNAc3}gauggucaAfaGfUfUfCfuugaus | 1429 | asCfsaucAfAfgaacUfuUfgAfccaucsa | 1430 |
| | gs{invAb} | | sg | |
| D-2432 | {sGalNAc3}gauggucaAfaGfUfUfGfuagaus | 1431 | asCfsaucUfAfcaacUfuUfgAfccaucsa | 1432 |
| | gs{invAb} | | sg | |
| D-2433 | {sGalNAc3}gaugguCfaAfAfGfUfucuugaus | 1433 | asCfsaucAfAfgaacUfuUfgAfccaucsa | 1434 |
| | gs{invAb} | | sg | |
| D-2434 | {sGalNAc3}gaugguCfaAfAfGfUfuguagaus | 1435 | asCfsaucUfAfcaacUfuUfgAfccaucsa | 1436 |
| | gs{invAb} | | sg | |
| D-2435 | {sGalNAc3}gaugguCfaAfAfGfUfucuugaus | 1437 | asCfsauClaAfgaacUfuUfgAfccaucsa | 1438 |
| | gs{invAb} | | sg | |
| D-2436 | {sGalNAc3}gaugguCfaAfAfGfUfuguagaus | 1439 | asCfsauCfuAfcaacUfuUfgAfccaucsa | 1440 |
| | gs{invAb} | | sg | |
| D-2437 | {sGalNAc3}gaugguCfaAfAfGfUfucuugaug | 1441 | asCfsaucaAfgaacuuUfgAfccasusc | 1442 |
| | s{invAb} | | | |
| D-2438 | {sGalNAc3}uggucaAfaGfUfUfCfuugaugs | 1443 | asCfsaucaAfgaacuuUfgAfccasusu | 1444 |
| | {invAb} | | | |
| D-2439 | {sGalNAc3}gaugguCfaAfAfGfUfucuagaug | 1445 | asCfsaucuAfgaacuuUfgAfccaucsusu | 1446 |
| | s{invAb} | | | |
| D-2440 | {sGalNAc3}gaugguCfaAfAfGfUfucuugaug | 1447 | asCfsaucaAfgaacuuUfgAfccaucsusu | 1448 |
| | s{invAb} | | | |
| D-2441 | {sGalNAc3}gaugguCfaAfAfGfUfuguagaug | 1449 | asCfsaucuAfcaacuuUfgAfccaucsusu | 1450 |
| | s{invAb} | | | |
| D-2442 | {sGalNAc3}ugguCfaAfAfGfUfucuugaugs | 1451 | asCfsaucaAfgaacuuUfgAfccasusu | 1452 |
| | {invAb} | | | |
| D-2443 | {sGalNAc3}ugggaaAfaCfCfAfGfugagucug | 1453 | asCfsagacUfcacuggUfuUfucccasusu | 1454 |
| | s{invAb} | | | |
| D-2444 | {sGalNAc3}aauuugUfaGfAfAfGfggauauac | 1455 | usGfsuauaUfcccuucUfaCfaaauususu | 1456 |
| | s{invAb} | | | |
| D-2445 | {sGalNAc3}aauuugUfaGfAfAfGfgcauauac | 1457 | usGfsuauaUfgccuucUfaCfaaauususu | 1458 |
| | s{invAb} | | | |
| D-2446 | {sGalNAc3}uuguagaaGfgGfAfUfAfuacaaa | 1459 | asUfsuuguAfuaucccUfuCfuacaasusu | 1460 |
| | s{invAb} | | | |
| D-2447 | {sGalNAc3}uuguagAfaGfGfGfAfuauacaaa | 1461 | asUfsuuguAfuaucccUfuCfuacsasa | 1462 |
| | ${invAb} | | | |
| D-2448 | {sGalNAc3}guagaaGfgGfAfUfAfuacaaas | 1463 | asUfsuuguAfuaucccUfuCfuacsusu | 1464 |
| | {invAb} | | | |
| D-2449 | {sGalNAc3}uuguagAfaGfGfGfAfuauacaaa | 1465 | asUfsuuguAfuaucccUfuCfuacaasusu | 1466 |
| | sfinvAb} | | | |
| D-2450 | {sGalNAc3}guagAfaGfGfGfAfuauacaaas | 1467 | asUfsuuguAfuaucccUfuCfuacsusu | 1468 |
| | {invAb} | | | |
| D-2451 | {sGalNAc3}gaagggAfuAfUfAfCfaaagugs | 1469 | csCfsacuuUfguauauCfcCfuucsusu | 1470 |
| | {invAb} | | | |
| D-2452 | {sGalNAc3}gaagGfgAfUfAfUfacaaagugs | 1471 | csCfsacuuUfguauauCfcCfuucsusu | 1472 |
| | {invAb} | | | |
| D-2453 | {sGalNAc3}uagaagggAfuAfUfAfCfaaagug | 1473 | asCfsacuuUfguauauCfcCfuucuasusu | 1474 |
| | s{invAb} | | | |
| D-2454 | {sGalNAc3}uagaagggAfuAfUfAfGfaaagug | 1475 | asCfsacuuUfcuauauCfcCfuucuasusu | 1476 |
| | s{invAb} | | | |
| D-2455 | {sGalNAc3}uagaagGIgAfUfAfUfagaaagug | 1477 | asCfsacuuucuauAfuCfcCfuucuasusu | 1478 |
| | s{invAb} | | | |
| D-2456 | {sGalNAc3}uagaagGfgAfuAfuagaaagugs | 1479 | asCfsacuuUfcuauAfuCfcCfuucuasus | 1480 |
| | {invAb} | | u | |
| D-2457 | {sGalNAc3}uagaagggAfuAfuagaaagugs | 1481 | asCfsacuuUfcuauAfuCfccuucuasusu | 1482 |
| | {invAb} | | | |
| D-2458 | {sGalNAc3}gaagGfgAfUfAfUfacaaagugs | 1483 | asCfsacuuuguauAfuCfcCfuucsusu | 1484 |
| | {invAb} | | | |
| D-2459 | {sGalNAc3}uagaagggAfuAfUfAfCfauagus | 1485 | asCfsacUfaUfguauAfuCfcCfuucuasc | 1486 |
| | gs{invAb} | | sa | |
| D-2460 | {sGalNAc3}uagaagggAfuAfUfAfCfuaagus | 1487 | asCfsacUfuAfguauAfuCfcCfuucuasc | 1488 |
| | gs{invAb} | | sa | |
| D-2461 | {sGalNAc3}uagaagggAfuAfUfAfGfaaagus | 1489 | asCfsacUfuUfcuauAfuCfcCfuucuasc | 1490 |
| | gs{invAb} | | sa | |
| D-2462 | {sGalNAc3}uagaagggAfuAfUfAfCfauagus | 1491 | asCfsacuAfUfguauAfuCfcCfuucuasc | 1492 |
| | gs{invAb} | | sa | |
| D-2463 | {sGalNAc3}uagaagggAfuAfUfAfCfuaagus | 1493 | asCfsacuUfAfguauAfuCfcCfuucuasc | 1494 |
| | gs{invAb} | | sa | |
| D-2464 | {sGalNAc3}uagaagggAfuAfUfAfGfaaagus | 1495 | asCfsacuUfUfcuauAfuCfcCfuucuasc | 1496 |
| | gs{invAb} | | sa | |
| D-2465 | {sGalNAc3}uagaagGfgAfUfAfUfacauagus | 1497 | asCfsacuAfUfguauAfuCfcCfuucuasc | 1498 |
| | gs{invAb} | | sa | |
| D-2466 | {sGalNAc3}uagaagGfgAfUfAfUfacuaagus | 1499 | asCfsacuUfAfguauAfuCfcCfuucuasc | 1500 |
| | gs{invAb} | | sa | |
| D-2467 | {sGalNAc3}uagaagGfgAfUfAfUfagaaagus | 1501 | asCfsacuUfUfcuauAfuCfcCfuucuasc | 1502 |
| | gs{invAb} | | sa | |
| D-2468 | {sGalNAc3}uagaagGfgAfUfAfUfacauagus | 1503 | asCfsacUfaUfguauAfuCfcCfuucuasc | 1504 |
| | gs{invAb} | | sa | |
| D-2469 | {sGalNAc3}uagaagGfgAfUfAfUfacuaagus | 1505 | asCfsacUfuAfguauAfuCfcCfuucuasc | 1506 |
| | gs{invAb} | | sa | |
| D-2470 | {sGalNAc3}uagaagGfgAfUfAfUfagaaagus | 1507 | asCfsacUfuUfcuauAfuCfcCfuucuasc | 1508 |
| | gs{invAb} | | sa | |
| D-2471 | {sGalNAc3}uagaagGfgAfUfAfUfacaaagug | 1509 | asCfsacuuUfguauauCfcCfuucsusa | 1510 |
| | sfinvAb} | | | |
| D-2472 | {sGalNAc3}uagaagGfgAfUfAfUfagaaagug | 1511 | asCfsacuuUfcuauauCfcCfuucsusa | 1512 |
| | s{invAb} | | | |
| D-2473 | {sGalNAc3}gaagggAfuAfUfAfGfaaagugs | 1513 | asCfsacuuUfcuauauCfcCfuucsusu | 1514 |
| | {invAb} | | | |
| D-2474 | {sGalNAc3}uagaagGfgAfuAfuagaaagugs | 1515 | asCfsacuuucuauAfuCfcCfuucuasusu | 1516 |
| | {invAb} | | | |
| D-2475 | {sGalNAc3}uagaagGfgAfUfAfUfacaaagug | 1517 | asCfsacuuUfguauauCfcCfuucuasusu | 1518 |
| | s{invAb} | | | |
| D-2476 | {sGalNAc3}uagaagGfgAfUfAfUfacauagug | 1519 | asCfsacuaUfguauauCfcCfuucuasusu | 1520 |
| | s{invAb} | | | |
| D-2477 | {sGalNAc3}uagaagGfgAfUfAfUfacuaagug | 1521 | asCfsacuuAfguauauCfcCfuucuasusu | 1522 |
| | s{invAb} | | | |
| D-2478 | {sGalNAc3}uagaagGfgAfUfAfUfagaaagug | 1523 | asCfsacuuUfcuauauCfcCfuucuasusu | 1524 |
| | s{invAb} | | | |
| D-2479 | {sGalNAc3}gaagGfgAfuAfuacaaagugs | 1525 | asCfsacuuuguauAfuCfcCfuucsusu | 1526 |
| | {invAb} | | | |
| D-2480 | {sGalNAc3}gaagGfgAfuAfuacaaagugs | 1527 | asCfsacuuUfguauAfuCfcCfuucsusu | 1528 |
| | {invAb} | | | |
| D-2481 | {sGalNAc3}gaagGfgAfUfAfUfagaaagugs | 1529 | asCfsacuuUfcuauauCfcCfuucsusu | 1530 |
| | {invAb} | | | |
| D-2482 | {sGalNAc3}uacaaaGfuGfGfAfAfauagacac | 1531 | asGfsugucUfauuuccAfcUfuuguasusu | 1532 |
| | s{invAb} | | | |
| D-2483 | {sGalNAc3}uacaaaGfuGfGfAfAfauugacac | 1533 | asGfsugucAfauuuccAfcUfuuguasusu | 1534 |
| | s{invAb} | | | |
| D-2484 | {sGalNAc3}caaaguGfgAfAfAfUfagacacca | 1535 | usUfsggugUfcuauuuCfcAfcuuugsusu | 1536 |
| | s{invAb} | | | |
| D-2485 | {sGalNAc3}guggaaAfuAfGfAfCfaccaaauc | 1537 | asGfsauuuGfgugucuAfuUfuccacsusu | 1538 |
| | s{invAb} | | | |
| D-2486 | {sGalNAc3}guggaaAfuAfGfAfCfagcaaauc | 1539 | asGfsauuuGfcugucuAfuUfuccacsusu | 1540 |
| | s{invAb} | | | |
| D-2487 | {sGalNAc3}ggaaauAfgAfCfAfCfcaaaucuu | 1541 | usAfsagauUfugguguCfuAfuuuccsusu | 1542 |
| | s{invAb} | | | |
| D-2488 | {sGalNAc3}ggaaauAfgAfCfAfCfcauaucuu | 1543 | usAfsagauAfugguguCfuAfuuuccsusu | 1544 |
| | s{invAb} | | | |
| D-2489 | {sGalNAc3}gaaauaGfaCfAfCfClaaaucuua | 1545 | asUfsaagaUfuuggug UfcUfauuucsusu | 1546 |
| | s{invAb} | | | |
| D-2490 | {sGalNAc3}gaaauaGfaCfAfCfCfaaaacuua | 1547 | asUfsaaguUfuuggugUfcUfauuucsusu | 1548 |
| | s{invAb} | | | |
| D-2491 | {sGalNAc3}gaaauaGfaCfAfCfCfaauucuua | 1549 | asUfsaagaAfuuggugUfcUfauuucsusu | 1550 |
| | s{invAb} | | | |
| D-2492 | {sGalNAc3}aaauagAfcAfCfCfAfaaucuuac | 1551 | asGfsuaagAfuuugguGfuCfuauuususu | 1552 |
| | s{invAb} | | | |
| D-2493 | {sGalNAc3}aauagaCfaCfCfAfAfaucuuacu | 1553 | asAfsguaaGfauuuggUfgUfcuauususu | 1554 |
| | s{invAb} | | | |
| D-2494 | {sGalNAc3}aauagaCfaCfCfAfAfaucauacu | 1555 | asAfsguauGfauuuggUfgUfcuauususu | 1556 |
| | s{invAb} | | | |
| D-2495 | {sGalNAc3}aauagaCfaCfCfAfAfaacuuacu | 1557 | asAfsguaaGfuuuuggUfgUfcuauususu | 1558 |
| | s{invAb} | | | |
| D-2496 | {sGalNAc3}auagacAfcCfAfAfAfucuuacug | 1559 | asCfsaguaAfgauuugGfuGfucuaususu | 1560 |
| | s{invAb} | | | |
| D-2497 | {sGalNAc3}auagacAfcCfAfAfAfucauacug | 1561 | asCfsaguaUfgauuugGfuGfucuaususu | 1562 |
| | s{invAb} | | | |
| D-2498 | {sGalNAc3}uagacaccAfaAfUfCfUfuacugg | 1563 | usCfscaguAfagauuuGfgUfgucuasusu | 1564 |
| | s{invAb} | | | |
| D-2499 | {sGalNAc3}uagacaCfcAfAfAfUfcuuacugg | 1565 | usCfscaguAfagauuuGfgUfgucsusa | 1566 |
| | s{invAb} | | | |
| D-2500 | {sGalNAc3}gacaccAfaAfUfCfUfuacuggs | 1567 | usCfscaguAfagauuuGfgUfgucsusu | 1568 |
| | {invAb} | | | |
| D-2501 | {sGalNAc3}uagacaCfcAfAfAfUfcuuacugg | 1569 | usCfscaguAfagauuuGfgUfgucuasusu | 1570 |
| | s{invAb} | | | |
| D-2502 | {sGalNAc3}uagacaCfcAfAfAfUfcuuucugg | 1571 | usCfscagaAfagauuuGfgUfgucuasusu | 1572 |
| | s{invAb} | | | |
| D-2503 | {sGalNAc3}gacaCfcAfAfAfUfcuuacuggs | 1573 | usCfscaguAfagauuuGfgUfgucsusu | 1574 |
| | {invAb} | | | |
| D-2504 | {sGalNAc3}agacacCfaAfAfUfCfuuacugga | 1575 | usUfsccagUfaagauuUfgGfugucususu | 1576 |
| | s{invAb} | | | |
| D-2505 | {sGalNAc3}agacacCfaAfAfUfCfuuucugga | 1577 | usUfsccagAfaagauuUfgGfugucususu | 1578 |
| | s{invAb} | | | |
| D-2506 | {sGalNAc3}agacacCfaAfAfUfCfuaacugga | 1579 | usUfsccagUfuagauuUfgGfugucususu | 1580 |
| | s{invAb} | | | |
| D-2507 | {sGalNAc3}acuuggCfaUfCfUfCfcccauucc | 1581 | usGfsgaauGfgggagaUfgCfcaagususu | 1582 |
| | s{invAb} | | | |
| D-2508 | {sGalNAc3}caucucCfcCfAfUfUfccaugagc | 1583 | usGfscucaUfggaaugGfgGfagaugsusu | 1584 |
| | s{invAb} | | | |
| D-2509 | {sGalNAc3}caucucCfcCfAfUfUfccuugagc | 1585 | usGfscucaAfggaaugGfgGfagaugsusu | 1586 |
| | s{invAb} | | | |
| D-2510 | {sGalNAc3}caucucCfcCfAfUfUfcgaugagc | 1587 | usGfscucaUfcgaaugGfgGfagaugsusu | 1588 |
| | s{invAb} | | | |
| D-2511 | {sGalNAc3}uccccauuCfcAfUfGfUfgcaugc | 1589 | usGfscaugCfacauggAfaUfggggasusu | 1590 |
| | s{invAb} | | | |
| D-2512 | {sGalNAc3}uccccauuCfcAfUfGfUfgcaugs | 1591 | usGfscaUfgCfacauGfgAfaUfggggasg | 1592 |
| | cs{invAb} | | sa | |
| D-2513 | {sGalNAc3}uccccauuCfcAfUfGfUfgcaugs | 1593 | usGfscauGfCfacauGfgAfaUfggggasg | 1594 |
| | cs{invAb} | | sa | |
| D-2514 | {sGalNAc3}uccccaUfuCfCfAfUfgugcaugs | 1595 | usGfscauGfCfacauGfgAfaUfggggasg | 1596 |
| | cs{invAb} | | sa | |
| D-2515 | {sGalNAc3}uccccaUfuCfCfAfUfgugcaugs | 1597 | usGfscaUfgCfacauGfgAfaUfggggasg | 1598 |
| | cs{invAb} | | sa | |
| D-2516 | {sGalNAc3}uccccaUfuCfCfAfUfgugcaugc | 1599 | usGfscaugCfacauggAfaUfgggsgsa | 1600 |
| | s{invAb} | | | |
| D-2517 | {sGalNAc3}cccauuCfcAfUfGfUfgcaugcs | 1601 | usGfscaugCfacauggAfaUfgggsusu | 1602 |
| | {invAb | | | |
| D-2518 | {sGalNAc3}uccccaUfuCfCfAfUfgagcaugc | 1603 | usGfscaugCfucauggAfaUfggggasusu | 1604 |
| | s{invAb} | | | |
| D-2519 | {sGalNAc3}uccccaUfuCfCfAfUfgugcaugc | 1605 | usGfscaugCfacauggAfaUfggggasusu | 1606 |
| | s{invAb} | | | |
| D-2520 | {sGalNAc3}cccaUfuCfCfAfUfgugcaugcs | 1607 | usGfscaugCfacauggAfaUfgggsusu | 1608 |
| | {invAb} | | | |
| D-2521 | {sGalNAc3}ccccauucCfaUfGfAfGfcaugca | 1609 | asUfsgcauGfcucaugGfaAfuggggsusu | 1610 |
| | s{invAb} | | | |
| D-2522 | {sGalNAc3}ccccauUfcCfAfUfGfagcaugca | 1611 | asUfsgcauGfcucaugGfaAfuggsgsg | 1612 |
| | s{invAb} | | | |
| D-2523 | {sGalNAc3}ccauucCfaUfGfAfGfcaugcas | 1613 | asUfsgcauGfcucaugGlaAfuggsusu | 1614 |
| | {invAb} | | | |
| D-2524 | {sGalNAc3}ccccauUfcCfAfUfGfagcaugca | 1615 | asUfsgcauGfcucaugGfaAfuggggsusu | 1616 |
| | s{invAb} | | | |
| D-2525 | {sGalNAc3}ccauUfcCfAfUfGfagcaugcas | 1617 | asUfsgcauGfcucaugGfaAfuggsusu | 1618 |
| | {invAb | | | |

### Example 2: Efficacy of Select TTR siRNA Molecules in RNA FISH Assay

siRNA molecules synthesized in Example 1 were screened in a fluorescent in situ hybridization assay targeting ribonucleic acid molecules (RNA FISH) to determine IC50 and maximum activity values.

RNA FISH (fluorescence in situ hybridization) assay was carried out to measure TTR mRNA knockdown by test siRNAs. HepG2 cells (purchased from ATCC) were cultured in Eagle's Minimum Essential Medium (EMEM) (ATCC® 30-2003™) supplemented with 10% fetal bovine serum (FBS, Sigma) and 1% penicillin-streptomycin (P-S, Corning). siRNAs were transfected into cells by reverse transfection using Lipofectamine RNAiMAX transfection reagent (Thermo Fisher Scientific). 1 μL of test siRNAs (in 10 data points dose with 1:3 dilution starting at 100 nM final concentration) or phosphate-buffered saline (PBS) vehicle and 4 μL of plain EMEM without supplements were added to PDL-coated CellCarrier-384 Ultra assay plates (PerkinElmer) by a Bravo automated liquid handling platform (Agilent). 5 μL of Lipofectamine RNAiMAX (Thermo Fisher Scientific), pre-diluted in plain EMEM without supplements (0.06 μL of RNAiMAX in 5 μL EMEM), was then dispensed into the assay plates by a Multidrop Combi reagent dispenser (Thermo Fisher Scientific). After 20-minute incubation of the siRNA/RNAiMAX mixture at room temperature (RT), 30 μL of HepG2 cells (2000 cells per well) in EMEM supplemented with 10% FBS and 1% P-S were added to the transfection complex using a Multidrop Combi reagent dispenser. The assay plates were incubated at RT for 20 mins prior to being placed in an incubator. Cells were incubated for 72 hrs at 37° C. and 5% CO2.

RNA FISH assay was performed 72 hours after siRNA transfection using the manufacturer's assay reagents and protocol (QuantiGene® ViewRNA HC Screening Assay from Thermo Fisher Scientific) on an in-house assembled automated FISH assay platform. In brief, cells were fixed in 4% formaldehyde (Thermo Fisher Scientific) for 15 mins at RT, permeabilized with detergent for 3 mins at RT and then treated with protease solution (1:4000 dilution in PBS) for 10 mins at RT. Target-specific probes (Thermo Fisher Scientific, Cat. #VX-02, Assay ID: VA6-17191-VC) or vehicle (target probe diluent without target probes as negative control) were incubated for 3 hours, whereas preamplifiers, amplifiers, and label probes were incubated for 1 hour each. All hybridization steps were carried out at 40° C. in a Cytomat 2 C-LIN automated incubator (Thermo Fisher Scientific). After hybridization reactions, cells were stained for 30 mins with Hoechst and CellMask Blue (Thermo Fisher Scientific) and then imaged on an Opera Phenix high-content screening system (PerkinElmer). The images were analyzed using a Columbus image data storage and analysis system (PerkinElmer) to obtain the mean spot count per cell. The mean spot count per cell was normalized using the high (PBS with target probes) and low (PBS without target probes) control wells. The high and low controls have normalized values of 100 and 0, respectively. The normalized values against the test siRNA concentrations were fitted to a 4-parameter sigmoidal model using Genedata Screener data analysis software (Genedata, Basel, Switzerland) to obtain IC50 values and maximum activity.

The results of the assay are shown in Table 3. TTR activity is expressed as a percentage of knockdown (% KD) at maximum and at 18 nM compared to control. Negative values indicate a decrease in TTR levels. The table also shows percent viability.

TABLE 3
RNA FISH assay on HepG2 cells

| | | Max activity | % KD at | % |
|---|---|---|---|---|
| Duplex No. | IC50 (M) | (% Max KD) | 18 nM | Viability |
| D-2000 | 8.93E-09 | −90.5 | −65.5 | 6.0 |
| D-2001 | 5.24E-09 | −97.4 | −83.6 | −18.1 |
| D-2002 | 4.04E-09 | −94.9 | −84.3 | −22.3 |
| D-2003 | 1.04E-08 | −94.6 | −75.2 | −10.3 |
| D-2004 | 4.84E-09 | −96.8 | −86.4 | −20.9 |
| D-2005 | 0.000000011 | −95.6 | −63.2 | −8.6 |
| D-2006 | 3.45E-09 | −92.2 | −86.4 | −12.1 |
| D-2007 | 1.05E-09 | −96.2 | −97.6 | 14.1 |
| D-2008 | 2.9E-09 | −94.2 | −93.2 | −14.7 |
| D-2009 | 3.65E-09 | −96.1 | −78.8 | −4.2 |
| D-2010 | 3.08E-09 | −96.8 | −92.5 | −27.2 |
| D-2011 | 1.85E-09 | −96.6 | −90.0 | −35.6 |
| D-2012 | 5.65E-10 | −81.7 | −76.0 | −26.9 |
| D-2013 | 5.17E-09 | −93.8 | −82.7 | −12.3 |
| D-2014 | 2.77E-09 | −95.8 | −95.8 | −1.0 |
| D-2015 | 6.13E-09 | −96.7 | −85.5 | −20.8 |
| D-2016 | 7.35E-10 | −96.2 | −91.9 | 7.0 |
| D-2017 | 2.59E-09 | −98.3 | −95.6 | −24.9 |
| D-2018 | 2.77E-09 | −93.0 | −91.4 | 0.3 |
| D-2019 | 2.29E-09 | −98.7 | −86.8 | −22.7 |
| D-2020 | 1.54E-09 | −95.8 | −89.6 | 32.0 |
| D-2021 | 2.6E-09 | −99.2 | −91.3 | −8.7 |
| D-2022 | 8.84E-10 | −98.6 | −82.1 | −4.1 |
| D-2023 | 2.65E-09 | −98.2 | −79.8 | −12.5 |
| D-2024 | 1.22E-09 | −97.8 | −93.7 | 1.4 |
| D-2025 | 2.92E-09 | −85.1 | −83.2 | −36.4 |
| D-2026 | 2.12E-09 | −92.6 | −79.4 | −23.4 |
| D-2027 | 1.6E-09 | −98.2 | −87.4 | −17.9 |
| D-2028 | 1.82E-09 | −97.3 | −82.1 | 11.4 |
| D-2029 | 1.12E-08 | −93.8 | −63.0 | −27.4 |
| D-2030 | 5.43E-09 | −97.6 | −80.6 | −13.3 |
| D-2031 | 3.14E-09 | −92.3 | −88.9 | −29.3 |
| D-2032 | 3.1E-09 | −97.6 | −93.1 | 18.7 |
| D-2033 | 1.16E-09 | −94.3 | −94.5 | −25.8 |
| D-2034 | 0.000000011 | −97.7 | −85.1 | −25.9 |
| D-2035 | 1.69E-08 | −97.7 | −48.0 | −24.4 |
| D-2036 | 4.89E-09 | −93.1 | −79.9 | −21.1 |
| D-2037 | 2.01E-08 | −83.7 | −50.4 | −8.1 |
| D-2038 | 2.19E-08 | −76.5 | −42.5 | −6.9 |
| D-2039 | 2.41E-09 | −85.4 | −89.9 | −24.6 |
| D-2040 | 3.3E-09 | −100.2 | −88.4 | −3.7 |
| D-2041 | 3.04E-09 | −94.1 | −83.5 | −26.3 |
| D-2042 | 2.85E-09 | −98.2 | −90.8 | −11.6 |
| D-2043 | 2.08E-09 | −95.6 | −94.7 | −11.0 |
| D-2044 | 3.53E-09 | −91.4 | −68.1 | 8.1 |
| D-2045 | 2.13E-09 | −93.6 | −94.9 | −26.3 |
| D-2046 | 1.32E-08 | −91.2 | −54.5 | −15.6 |
| D-2047 | 3.85E-08 | −95.5 | −75.6 | −45.9 |
| D-2048 | 3.12E-08 | −95.6 | −48.2 | −29.1 |
| D-2049 | 1.47E-08 | −94.3 | −58.9 | −21.2 |
| D-2050 | 0.000000162 | −79.8 | −14.4 | 2.2 |
| D-2051 | 5.56E-08 | −88.6 | −25.4 | −1.0 |
| D-2052 | Undefined | −38.3 | −20.3 | −9.9 |
| D-2053 | 2.79E-08 | −93.4 | −22.3 | −29.0 |
| D-2054 | 2.75E-08 | −92.4 | −45.0 | −8.4 |
| D-2055 | 0.000000101 | −81.4 | −29.8 | −11.1 |
| D-2056 | 3.06E-09 | −97.1 | −83.7 | −22.5 |
| D-2057 | 2.02E-08 | −95.7 | −45.1 | −21.7 |
| D-2058 | 2.22E-08 | −91.4 | −55.3 | 3.6 |
| D-2059 | >500E-9 | −19.7 | −1.4 | −5.6 |
| D-2060 | >500E-9 | −12.7 | −7.2 | 21.6 |
| D-2061 | 0.000000013 | −80.2 | −44.0 | 6.4 |
| D-2062 | 6.38E-08 | −85.9 | −11.0 | 12.4 |
| D-2063 | 3.42E-08 | −60.8 | −2.6 | −30.0 |
| D-2064 | 3.59E-08 | −62.0 | −24.8 | −8.9 |
| D-2065 | Undefined | −43.4 | −13.4 | 21.2 |
| D-2066 | 7.88E-08 | −74.1 | −33.7 | 2.9 |
| D-2067 | 0.000000152 | −78.8 | −10.5 | −19.1 |
| D-2068 | Undefined | −41.2 | 4.9 | 38.6 |
| D-2069 | 8.93E-08 | −64.0 | −10.2 | −8.2 |
| D-2070 | 6.24E-08 | −59.7 | −14.9 | −25.0 |
| D-2071 | Undefined | −45.3 | −15.5 | −6.6 |
| D-2072 | 6.82E-08 | −46.9 | −13.6 | −15.1 |
| D-2073 | 0.000000105 | −69.5 | −9.0 | −9.6 |
| D-2074 | 4.41E-08 | −78.6 | −33.3 | 3.2 |
| D-2075 | 1.84E-08 | −96.7 | −50.3 | −10.8 |
| D-2076 | 1.92E-08 | −97.6 | −50.3 | −16.3 |
| D-2077 | 7.51E-08 | −68.2 | −36.1 | −12.0 |
| D-2078 | 3.43E-08 | −96.3 | −39.7 | 3.0 |
| D-2079 | 0.000000152 | −84.6 | −16.3 | −0.3 |
| D-2080 | 9.15E-08 | −70.8 | −28.2 | −23.7 |
| D-2081 | >500E-9 | −22.7 | −11.3 | −46.2 |
| D-2082 | 2.06E-08 | −95.7 | −46.4 | 10.5 |
| D-2083 | 1.34E-08 | −96.9 | −65.8 | −24.0 |
| D-2084 | 6.49E-08 | −74.8 | −0.3 | −17.2 |
| D-2085 | 1.27E-08 | −95.3 | −63.9 | 0.2 |
| D-2086 | 2.88E-08 | −97.5 | −47.4 | −8.8 |
| D-2087 | 2.21E-08 | −92.9 | −46.9 | −17.8 |
| D-2088 | 2.89E-08 | −95.3 | −41.9 | −0.5 |
| D-2089 | 4.56E-08 | −90.9 | −41.8 | −3.8 |
| D-2090 | 8.57E-08 | −72.6 | −30.8 | −14.4 |
| D-2091 | 0.000000014 | −92.1 | −53.8 | −2.7 |
| D-2092 | 6.44E-08 | −79.1 | −1.7 | 11.6 |
| D-2093 | 6.46E-08 | −50.3 | −10.1 | −20.5 |
| D-2094 | 1.05E-08 | −97.7 | −70.3 | −27.4 |
| D-2095 | 1.27E-09 | −95.0 | −97.9 | −28.0 |
| D-2096 | 4.55E-09 | −94.5 | −94.9 | −2.4 |
| D-2097 | 4.08E-08 | −69.8 | −24.1 | 18.0 |
| D-2098 | 5.77E-08 | −61.8 | −16.0 | 12.0 |
| D-2099 | 2.79E-09 | −95.7 | −82.1 | 13.4 |
| D-2100 | 1.09E-08 | −94.3 | −63.4 | −15.6 |
| D-2101 | 4.98E-08 | −87.8 | −14.4 | −21.1 |
| D-2102 | 3.22E-08 | −90.5 | −62.2 | −3.8 |
| D-2103 | 3.39E-09 | −93.9 | −93.3 | −20.4 |
| D-2104 | 7.32E-09 | −92.6 | −78.5 | −18.7 |
| D-2105 | 1.08E-08 | −91.9 | −77.8 | −2.9 |
| D-2106 | 6.05E-09 | −91.3 | −51.3 | −21.5 |
| D-2107 | 4.65E-09 | −94.5 | −69.5 | −4.7 |
| D-2108 | 7.16E-09 | −96.3 | −77.8 | −10.3 |
| D-2109 | 1.21E-08 | −94.1 | −49.8 | −23.8 |
| D-2110 | 1.26E-08 | −97.8 | −66.0 | −0.6 |
| D-2111 | 5.71E-09 | −90.4 | −79.2 | 7.3 |
| D-2112 | 4.96E-09 | −95.3 | −72.6 | 3.3 |
| D-2113 | >500E-9 | 60.2 | −21.5 | −34.5 |
| D-2114 | 7.41E-09 | −94.8 | −73.9 | −26.3 |
| D-2115 | 2.34E-09 | −94.1 | −69.9 | −15.5 |
| D-2116 | 2.31E-09 | −97.0 | −90.7 | −27.7 |
| D-2117 | 9.66E-09 | −96.0 | −63.4 | −26.7 |
| D-2118 | 1.36E-09 | −96.6 | −88.8 | −20.4 |
| D-2119 | 5.08E-08 | −84.1 | −34.4 | −20.1 |
| D-2120 | 2.72E-08 | −81.8 | −32.4 | −22.9 |
| D-2121 | 1.96E-08 | −95.7 | −55.2 | −31.4 |
| D-2122 | 4.1E-09 | −93.5 | −83.2 | −13.8 |
| D-2123 | 1.27E-08 | −95.8 | −72.3 | −31.4 |
| D-2124 | 3.76E-09 | −99.1 | −85.7 | 3.1 |
| D-2125 | 3.49E-09 | −98.4 | −79.0 | 17.2 |
| D-2126 | 3.32E-09 | −95.5 | −83.8 | −20.8 |
| D-2127 | 1.42E-09 | −97.5 | −90.0 | −21.1 |
| D-2128 | 3.25E-09 | −98.0 | −81.4 | −8.7 |
| D-2129 | 3.25E-09 | −98.6 | 81.8 | −7.5 |
| D-2130 | 5.09E-09 | −97.4 | −81.4 | 0.7 |
| D-2131 | 6.66E-09 | −94.8 | −84.6 | −1.1 |
| D-2132 | 3.93E-09 | −94.7 | −93.6 | −8.7 |
| D-2133 | 3.23E-09 | −87.6 | −77.0 | −19.7 |
| D-2134 | 2.24E-09 | −94.8 | −87.9 | −13.2 |
| D-2135 | 1.03E-08 | −93.2 | −81.3 | −7.4 |
| D-2136 | 1.28E-08 | −94.3 | −52.5 | 5.8 |
| D-2137 | 5.04E-09 | −84.3 | −70.6 | 1.4 |
| D-2138 | 0.000000005 | −86.4 | −79.8 | −18.5 |
| D-2139 | 2.73E-08 | −67.2 | −37.9 | −11.0 |
| D-2140 | 1.47E-08 | −65.3 | −25.5 | −31.9 |
| D-2141 | 1.23E-08 | −92.3 | −53.2 | −18.7 |
| D-2142 | 3.11E-09 | −92.2 | −81.2 | −17.5 |
| D-2143 | 2.92E-08 | −75.5 | −26.3 | −6.4 |
| D-2144 | 0.000000033 | −62.8 | −38.9 | −20.3 |
| D-2145 | 8.61E-09 | −61.1 | −20.2 | −24.3 |
| D-2146 | 7.05E-09 | −86.8 | −57.8 | −15.1 |
| D-2147 | 3.47E-09 | −96.8 | −87.9 | −27.3 |
| D-2148 | 5.7E-09 | −92.0 | −82.9 | −20.7 |
| D-2149 | 1.84E-09 | −95.5 | −74.5 | −20.0 |
| D-2150 | 6.41E-09 | −94.9 | −72.8 | 3.0 |
| D-2151 | 0.00000001 | −91.7 | −69.9 | −13.5 |
| D-2152 | 0.000000109 | −78.2 | −10.4 | −19.5 |
| D-2153 | 8.35E-09 | −93.5 | −70.4 | −23.4 |
| D-2154 | 1.36E-08 | −89.7 | −65.0 | 19.7 |
| D-2155 | 4.07E-08 | −90.6 | −18.0 | 3.3 |
| D-2156 | 3.8E-09 | −97.3 | −73.0 | −16.0 |
| D-2157 | 1.48E-08 | −88.1 | −52.3 | −6.2 |
| D-2158 | 1.56E-08 | −89.4 | −60.6 | −9.2 |
| D-2159 | 0.000000127 | −53.0 | −6.4 | 7.2 |
| D-2160 | 1.94E-08 | −72.0 | −37.4 | −11.7 |
| D-2161 | 1.21E-08 | −90.2 | −61.9 | −7.7 |
| D-2162 | 6.33E-09 | −91.1 | −68.9 | −11.2 |
| D-2163 | 4.38E-08 | −68.8 | −22.3 | −28.9 |
| D-2164 | 0.000000013 | −98.6 | −67.5 | −5.6 |
| D-2165 | 2.36E-08 | −93.7 | −44.2 | −9.0 |
| D-2166 | 2.75E-08 | −48.2 | −18.8 | −3.3 |
| D-2167 | 2.97E-08 | −83.9 | −23.0 | 5.2 |
| D-2168 | 0.000000162 | −70.7 | −3.5 | −16.7 |
| D-2169 | 2.69E-08 | −94.5 | −24.5 | −9.1 |
| D-2170 | 6.92E-08 | −64.5 | 3.0 | −14.1 |
| D-2171 | 2.58E-08 | −80.8 | −29.6 | −19.7 |
| D-2172 | 0.000000021 | −93.9 | −49.2 | −15.9 |
| D-2173 | 2.85E-08 | −97.0 | −48.1 | −7.3 |
| D-2174 | 2.09E-08 | −90.3 | −56.2 | 11.6 |
| D-2175 | 3.13E-08 | −97.2 | −26.6 | 3.2 |
| D-2176 | 0.000000105 | −62.6 | 38.0 | −13.5 |
| D-2177 | 2.07E-08 | −93.4 | −47.0 | −22.9 |
| D-2178 | 1.39E-08 | −95.4 | −51.6 | −6.2 |
| D-2179 | 6.49E-08 | −77.9 | −24.2 | −3.9 |
| D-2180 | 1.21E-08 | −93.8 | −70.4 | −24.9 |
| D-2181 | 6.93E-08 | −92.1 | −27.7 | −30.9 |
| D-2182 | 5.78E-08 | −92.2 | −32.0 | −38.3 |
| D-2183 | 0.000000012 | −95.1 | −77.8 | −50.7 |
| D-2184 | 0.000000036 | −92.2 | −29.8 | −14.4 |
| D-2185 | 1.44E-08 | −92.6 | −58.3 | −32.4 |
| D-2186 | 1.08E-08 | −94.8 | −64.2 | −10.9 |
| D-2187 | 2.15E-08 | −91.0 | −49.5 | −13.8 |
| D-2188 | 3.47E-08 | −96.6 | −47.2 | −16.4 |
| D-2189 | 2.53E-08 | −94.3 | −42.2 | −47.9 |
| D-2190 | 8.19E-09 | −84.2 | −79.8 | −40.6 |
| D-2191 | 2.18E-08 | −90.4 | −74.1 | −57.4 |
| D-2192 | 0.000000123 | −82.0 | −13.8 | −25.4 |
| D-2193 | 2.59E-08 | −74.4 | −28.2 | −34.1 |
| D-2194 | 4.17E-08 | −95.5 | −19.5 | −38.3 |
| D-2195 | 4.18E-08 | −95.2 | −38.5 | −26.3 |
| D-2196 | 0.000000071 | −67.6 | −12.1 | −53.2 |
| D-2197 | 1.19E-08 | −92.7 | −79.8 | −51.8 |
| D-2198 | 0.000000035 | −81.4 | −19.7 | −20.1 |
| D-2199 | 5.19E-08 | −80.0 | −36.4 | −33.2 |
| D-2200 | 3.86E-08 | −76.5 | −18.8 | −24.9 |
| D-2201 | 1.38E-08 | −94.3 | −52.5 | −7.7 |
| D-2202 | 3.33E-08 | −91.1 | −33.2 | −16.5 |
| D-2203 | 0.000000038 | −79.7 | −36.8 | −12.7 |
| D-2204 | 1.43E-08 | −90.9 | −63.7 | −42.5 |
| D-2205 | 2.61E-08 | −88.9 | −53.5 | −32.7 |
| D-2206 | 0.000000091 | −77.5 | −33.9 | −21.8 |
| D-2207 | 5.62E-08 | −84.3 | −17.7 | −12.7 |
| D-2208 | 2.27E-08 | −85.2 | −39.7 | −40.3 |
| D-2209 | 4.73E-08 | −81.2 | −6.2 | −2.3 |
| D-2210 | 5.39E-08 | −77.8 | −14.4 | −6.3 |
| D-2211 | 0.000000135 | −59.4 | 9.3 | 5.6 |
| D-2212 | 9.24E-08 | −51.1 | −8.1 | 0.2 |
| D-2213 | 0.000000132 | −86.4 | 0.2 | −5.4 |
| D-2214 | Undefined | −29.6 | −2.1 | −5.9 |
| D-2215 | 9.19E-09 | −71.6 | −40.7 | −35.1 |
| D-2216 | 9.75E-08 | −98.0 | −10.3 | −27.3 |
| D-2217 | 8.76E-08 | −94.8 | −3.4 | 8.0 |
| D-2218 | 6.08E-08 | −93.3 | −17.2 | 0.7 |
| D-2219 | 0.000000067 | −100.8 | 9.6 | −17.6 |
| D-2220 | 0.000000102 | −92.1 | 8.1 | −15.2 |
| D-2221 | 8.43E-09 | −72.5 | −48.3 | −17.3 |
| D-2222 | 0.000000108 | −91.3 | −16.4 | −6.9 |
| D-2223 | 9.24E-08 | −91.7 | −12.2 | −9.5 |
| D-2224 | 0.000000085 | −92.1 | −6.8 | −12.9 |
| D-2225 | 8.24E-08 | −92.0 | −4.3 | 1.0 |
| D-2226 | 0.000000112 | −91.7 | −21.5 | −10.3 |
| D-2227 | 0.000000106 | −92.3 | −10.6 | −10.4 |
| D-2228 | 1.75E-09 | −96.2 | −86.1 | −12.0 |
| D-2229 | 7.81E-08 | −97.4 | −13.1 | −1.0 |
| D-2230 | 9.72E-09 | −95.9 | −16.5 | 11.1 |
| D-2231 | 7.96E-08 | −94.6 | −17.5 | −1.5 |
| D-2232 | 0.000000104 | −92.7 | −2.2 | −27.6 |
| D-2233 | 0.000000104 | −94.7 | −2.5 | −27.2 |
| D-2234 | 7.47E-08 | −101.0 | −17.4 | −34.3 |
| D-2235 | >500E-9 | 73.1 | 22.4 | −39.6 |
| D-2236 | 0.000000076 | −96.8 | −8.7 | −13.6 |
| D-2237 | 8.84E-08 | −93.5 | −6.5 | 9.4 |
| D-2238 | 0.000000113 | −96.8 | −17.0 | −7.6 |
| D-2239 | 4.23E-09 | −96.2 | −91.0 | −27.0 |
| D-2240 | 9.91E-08 | −96.5 | −6.1 | 5.6 |
| D-2241 | 0.00000011 | −88.8 | −20.2 | −38.2 |
| D-2242 | 0.000000063 | −98.5 | −16.4 | −27.5 |
| D-2243 | 1.47E-09 | −90.9 | −81.2 | −3.8 |
| D-2244 | 0.000000101 | −94.3 | −5.8 | −1.0 |
| D-2245 | 0.00000009 | −92.2 | −6.1 | −10.7 |
| D-2246 | 2.66E-09 | −98.9 | −92.4 | −32.3 |
| D-2247 | 8.66E-09 | −98.4 | −82.4 | 13.7 |
| D-2248 | 3.96E-09 | −94.5 | −87.9 | −21.3 |
| D-2249 | 7.13E-09 | −94.5 | −70.2 | 1.0 |
| D-2250 | 3.28E-09 | −96.6 | −89.7 | −23.3 |
| D-2251 | 4.09E-09 | −98.9 | −85.4 | −1.0 |
| D-2252 | 1.71E-09 | −96.9 | −81.3 | 1.5 |
| D-2253 | 6.32E-09 | −95.0 | −76.8 | −4.9 |
| D-2254 | 8.59E-09 | −96.4 | −84.9 | 3.3 |
| D-2255 | 2.95E-09 | −94.4 | −89.4 | −25.8 |
| D-2256 | 1.64E-09 | −99.5 | −96.6 | −5.7 |
| D-2257 | 1.06E-08 | −97.7 | −75.2 | −19.7 |
| D-2258 | 0.000000011 | −98.9 | −82.0 | 3.9 |
| D-2259 | 2.19E-08 | −95.9 | −42.5 | 18.2 |
| D-2260 | 3.35E-09 | −98.2 | −90.2 | −12.4 |
| D-2261 | 3.56E-09 | −95.3 | −88.8 | −2.7 |
| D-2262 | 1.16E-08 | −95.0 | −83.1 | 13.6 |
| D-2263 | 3.76E-09 | −99.7 | −90.4 | −18.5 |
| D-2264 | 1.18E-08 | −99.0 | −74.9 | −9.9 |
| D-2265 | 1.87E-08 | −97.4 | −53.7 | −20.3 |
| D-2266 | 8.67E-09 | −97.1 | −89.2 | −19.4 |
| D-2267 | 7.05E-09 | −87.7 | −75.1 | −5.1 |
| D-2268 | 1.18E-08 | −94.4 | −63.0 | 0.9 |
| D-2269 | 1.97E-08 | −96.3 | −52.4 | −16.4 |
| D-2270 | 5.4E-09 | −93.6 | −88.6 | −0.1 |
| D-2271 | 6.23E-09 | −91.8 | −80.7 | 15.4 |
| D-2272 | 9.1E-09 | −94.2 | −70.6 | 1.0 |
| D-2273 | 5.54E-09 | −90.4 | −73.4 | −1.6 |
| D-2274 | 5.84E-09 | −93.9 | −76.9 | 7.5 |
| D-2275 | 9.13E-09 | −92.9 | −68.3 | −19.5 |
| D-2276 | 1.54E-08 | −96.1 | −65.7 | −20.7 |
| D-2277 | 9.94E-09 | −95.6 | −83.3 | −11.5 |
| D-2278 | 1.47E-08 | −89.8 | −60.4 | |
| D-2279 | 7.06E-09 | −93.8 | −75.9 | |
| D-2280 | 9.35E-09 | −92.4 | −61.1 | |
| D-2281 | 8.87E-09 | −89.8 | −67.8 | |
| D-2282 | 7.27E-09 | −89.7 | −77.3 | |

### Example 3: Efficacy Screening TTR siRNA Molecules in a Humanized Mouse Model

Associated adenovirus (AAV; serotype DJ8; endotoxin-free, prepared internally by Amgen) diluted in phosphate buffered saline (Thermo Fisher Scientific, 14190-136) to 1e12 viral particles per animal, was injected intravenously into the tail vein of pathogen-free C57BL/6NCrl female mice (Charles River Laboratories Inc.) to drive expression of human TTR (hTTR) in the liver. Mice were generally 8-10 weeks of age at the beginning of the study and an n=4-6 animals were included per group. Every round of screening included at least two vehicle-treated control groups: AAV-empty vector and AAV-hTTR treated with vehicle. Fourteen days after administration of AAV, mice were treated with a single dose of siRNA (0.5 mM), via subcutaneous injection, at 0.5, 1.0, or 3.0 milligrams per kilogram of animal (as indicated in Table 4, below), diluted in phosphate buffered saline (Thermo Fisher Scientific, 14190-136). At 28 days post-siRNA injection, mice were anesthetized and then euthanized by a secondary physical method, following Association for Assessment and Accreditation of Laboratory Animal Care (AAALAC) guidelines. Livers were collected from the animals and immediately snap frozen in liquid nitrogen, processed for purified RNA using a QIACube instrument (Qiagen, 9001292) and RNeasy Mini QIAcube Kit (240) (Qiagen, 74116) according to manufacturer's instructions. RNA samples were analyzed using a NanoDrop™ 8000 Spectrophotometer (ThermoFisher, ND-8000GL). RNA was treated with RQ1 RNase-Free DNase (Promega, M6101) and prepared for Real-Time qPCR using the TaqMan™ RNA-to-CT™ 1-Step kit (Applied Biosystems, 4392653). Real-Time qPCR was run on a QuantStudio 7 Flex PCR machine. Results are based on gene expression of human TTR as normalized to mouse Thp (TaqMan™ assays from Invitrogen, Hs00174914 and Mm01277042, respectively), and presented as the relative knockdown of human TTR mRNA expression compared to vehicle-treated control animals. Endogenous mouse Ttr expression was also determined for comparison (Invitrogen, Mm00443267).

All animal procedures described herein were approved by the Institutional Animal Care and Use Committee (LACUC) of Charles River Accelerator and Development Lab (CRADL, AAALAC accredited) and cared for in accordance with the Guide for the Care and Use of Laboratory Animals, 8th Edition (National Research Council (U.S.). Mice were group-housed in a climate-controlled room at 22±2° C. with a twelve-hour light; twelve-hour darkness cycle (0600-1800 hours). Animals had ad libitum access to a regular chow diet (Envigo, 2920X) and were caged in irradiated Innovive Disposable IVC Rodent Caging Systems (San Diego, CA) with pre-filled sterile and acidified water bottles.

TABLE 4
In vivo transthyretin knockdown assay

| | Dose | Transthyretin | Transthyretin |
|---|---|---|---|
| Duplex | administered | hTTR | mTTR |
| number | (mg/kg) | knockdown (%) | knockdown (%) |
| D-2443 | 1 | −28.2 | −14.4 |
| D-2440 | 1 | 11.0 | −6.0 |
| D-2352 | 1 | −31.0 | −33.9 |
| D-2343 | 1 | −17.6 | −43.3 |
| D-2339 | 1 | −26.3 | 1.0 |
| D-2326 | 1 | −38.8 | 15.4 |
| D-2292 | 1 | −37.3 | 1.0 |
| D-2290 | 1 | −33.2 | −39.2 |
| D-2496 | 1 | −66.3 | 0.5 |
| D-2350 | 1 | −33.5 | −13.5 |
| D-2507 | 1 | −65.4 | −43.6 |
| D-2345 | 1 | 15.2 | −0.8 |
| D-2504 | 1 | −34.4 | −44.6 |
| D-2475 | 1 | −53.4 | −51.0 |
| D-2501 | 1 | −76.7 | −8.3 |
| D-2343 | 1 | −49.8 | −8.6 |
| D-2449 | 1 | −82.0 | −4.9 |
| D-2439 | 1 | −27.2 | −6.8 |
| D-2342 | 1 | −66.7 | −72.8 |
| D-2492 | 1 | −7.5 | 7.1 |
| D-2489 | 1 | −44.9 | −4.0 |
| D-2423 | 1 | −83.8 | −57.5 |
| D-2337 | 1 | −72.4 | −59.7 |
| D-2487 | 1 | −15.9 | −5.7 |
| D-2410 | 1 | −42.8 | −34.4 |
| D-2333 | 1 | −70.3 | −12.3 |
| D-2485 | 1 | −64.9 | −21.3 |
| D-2398 | 1 | −82.4 | −51.4 |
| D-2393 | 1 | −40.1 | −51.1 |
| D-2328 | 1 | −14.2 | 1.2 |
| D-2391 | 1 | −3.9 | −2.2 |
| D-2325 | 1 | −71.9 | −79.5 |
| D-2524 | 1 | −80.6 | −33.7 |
| D-2484 | 1 | −59.2 | 19.3 |
| D-2324 | 1 | −25.5 | 7.2 |
| D-2322 | 1 | −27.7 | 23.6 |
| D-2289 | 1 | −41.6 | −3.8 |
| D-2518 | 1 | −39.2 | −11.4 |
| D-2288 | 1 | −34.9 | −35.2 |
| D-2482 | 1 | −63.8 | 7.9 |
| D-2387 | 1 | −50.4 | −2.8 |
| D-2316 | 1 | −77.5 | 10.9 |
| D-2293 | 1 | −37.5 | 0.8 |
| D-2381 | 1 | −82.1 | −10.5 |
| D-2287 | 1 | −73.3 | −40.4 |
| D-2283 | 1 | −58.1 | −26.6 |
| D-2361 | 1 | 10.4 | −0.1 |
| D-2355 | 1 | 42.7 | −28.0 |
| D-2493 | 1 | −15.0 | 1.9 |
| D-2508 | 1 | −39.6 | 17.1 |
| D-2354 | 1 | −55.2 | 32.9 |
| D-2491 | 1 | −56.1 | 4.3 |
| D-2490 | 1 | −38.4 | 10.9 |
| D-2327 | 1 | −23.1 | 4.5 |
| D-2394 | 1 | −41.2 | 0.8 |
| D-2353 | 1 | −39.1 | 25.6 |
| D-2488 | 1 | −9.1 | 32.1 |
| D-2506 | 1 | −55.5 | 12.3 |
| D-2505 | 1 | −43.3 | −3.6 |
| D-2486 | 1 | −67.1 | 13.0 |
| D-2392 | 1 | −31.8 | −14.7 |
| D-2351 | 1 | −63.7 | −40.8 |
| D-2286 | 1 | −33.7 | 0.2 |
| D-2285 | 1 | −3.6 | 182.6 |
| D-2502 | 1 | −31.4 | 9.4 |
| D-2284 | 1 | −4.5 | 11.7 |
| D-2497 | 1 | 0.5 | 13.7 |
| D-2445 | 1 | −40.7 | 27.6 |
| D-2323 | 1 | −51.6 | 24.9 |
| D-2390 | 1 | −62.2 | 28.5 |
| D-2483 | 1 | −46.4 | 28.6 |
| D-2317 | 1 | −71.9 | 17.5 |
| D-2495 | 1 | −43.7 | 18.0 |
| D-2389 | 1 | −46.9 | 9.4 |
| D-2388 | 1 | −49.7 | 31.3 |
| D-2494 | 1 | 14.0 | 26.8 |
| D-2384 | 1 | −51.0 | 2.1 |
| D-2477 | 1 | −67.0 | −43.2 |
| D-2383 | 1 | −73.9 | 13.1 |
| D-2382 | 1 | −40.0 | −14.7 |
| D-2476 | 1 | −61.2 | −7.8 |
| D-2441 | 1 | −70.5 | −6.5 |
| D-2338 | 1 | −53.7 | −42.9 |
| D-2334 | 1 | −27.7 | −16.3 |
| D-2440 | 1 | −73.6 | −40.9 |
| D-2519 | 1 | −71.6 | −20.2 |
| D-2412 | 1 | −73.4 | −23.2 |
| D-2363 | 1 | 71.6 | −22.5 |
| D-2362 | 1 | −46.8 | −34.0 |
| S-2424 | 1 | −74.6 | −22.5 |
| D-2291 | 1 | −37.9 | −35.5 |
| D-2441 | 1 | −52.8 | −20.8 |
| D-2510 | 1 | −64.5 | −12.0 |
| D-2509 | 1 | −31.0 | −2.5 |
| D-2357 | 1 | −46.9 | −22.3 |
| D-2356 | 1 | −30.6 | −29.9 |
| D-2449 | 0.5 | −27.7 | −17.2 |
| D-2449 | 1 | −1.3 | 8.1 |
| D-2449 | 3 | −79.8 | 6.0 |
| D-2423 | 0.5 | −84.1 | −36.9 |
| D-2423 | 1 | −83.3 | −44.9 |
| D-2423 | 3 | −97.3 | −81.5 |
| D-2398 | 0.5 | 1.1 | 9.4 |
| D-2398 | 1 | 2.1 | 0.3 |
| D-2398 | 3 | −52.8 | −7.4 |
| D-2524 | 0.5 | −10.6 | −19.5 |
| D-2524 | 1 | −53.8 | −14.8 |
| D-2524 | 3 | −88.9 | −20.2 |
| D-2381 | 0.5 | 156.3 | 121.8 |
| D-2381 | 1 | −30.7 | −47.3 |
| D-2381 | 3 | −91.1 | −84.4 |
| D-2501 | 0.5 | 133.2 | 84.9 |
| D-2501 | 1 | 35.8 | 53.9 |
| D-2501 | 3 | −24.8 | 7.5 |
| D-2316 | 0.5 | −2.8 | 86.6 |
| D-2316 | 1 | −54.3 | 42.1 |
| D-2316 | 3 | −91.0 | 71.2 |
| D-2412 | 0.5 | 14.2 | 118.4 |
| D-2412 | 1 | −38.9 | 80.7 |
| D-2412 | 3 | −89.9 | 74.2 |
| D-2412 | 0.5 | −62.6 | −9.4 |
| D-2400 | 0.5 | −69.5 | −13.5 |
| D-2413 | 0.5 | −49.8 | −13.0 |
| D-2409 | 0.5 | −53.4 | −15.0 |
| D-2408 | 0.5 | −71.2 | −28.4 |
| D-2398 | 1 | −85.6 | −61.7 |
| D-2398 | 0.5 | −75.6 | −48.4 |
| D-2395 | 0.5 | −16.1 | −36.9 |
| D-2399 | 0.5 | −5.2 | −8.2 |
| D-2397 | 0.5 | 0.4 | −19.6 |
| D-2396 | 0.5 | −20.7 | −19.6 |
| D-2423 | 0.5 | −66.7 | −47.3 |
| D-2414 | 0.5 | −65.1 | −48.7 |
| D-2425 | 0.5 | −66.2 | −46.1 |
| D-2420 | 0.5 | −71.0 | −46.0 |
| D-2417 | 0.5 | −76.1 | −28.0 |
| D-2381 | 0.5 | −66.8 | −25.8 |
| D-2365 | 0.5 | −30.7 | −40.3 |
| D-2385 | 0.5 | −39.4 | −35.4 |
| D-2378 | 0.5 | −41.2 | −29.5 |
| D-2376 | 0.5 | −41.2 | −71.6 |
| D-2316 | 0.5 | −59.3 | −67.5 |
| D-2296 | 0.5 | −56.4 | −63.0 |
| D-2320 | 0.5 | −52.8 | −64.7 |
| D-2312 | 0.5 | −68.9 | −61.1 |
| D-2309 | 0.5 | −66.9 | −71.9 |
| D-2449 | 0.5 | −37.7 | −5.4 |
| D-2446 | 0.5 | −7.5 | 10.1 |
| D-2450 | 0.5 | 4.0 | −12.4 |
| D-2448 | 0.5 | 36.2 | −28.4 |
| D-2447 | 0.5 | 50.3 | 3.3 |
| D-2501 | 0.5 | −40.8 | −22.6 |
| D-2498 | 0.5 | 16.6 | −17.8 |
| D-2503 | 0.5 | −4.5 | −32.1 |
| D-2500 | 0.5 | −31.0 | −38.5 |
| D-2499 | 0.5 | −27.8 | −31.4 |
| D-2524 | 0.5 | −69.7 | −36.7 |
| D-2521 | 0.5 | −21.0 | −24.1 |
| D-2525 | 0.5 | −26.6 | −44.5 |
| D-2523 | 0.5 | −23.4 | −23.8 |
| D-2522 | 0.5 | −34.2 | −31.4 |
| D-2524 | 1 | −35.1 | 8.7 |
| D-2524 | 0.5 | −17.4 | −9.9 |
| D-2521 | 0.5 | −24.9 | −4.0 |
| D-2525 | 0.5 | −4.7 | −1.7 |
| D-2523 | 0.5 | −14.1 | 0.8 |
| D-2522 | 0.5 | −51.2 | 11.8 |
| D-2297 | 1 | −0.5 | −14.6 |
| D-2297 | 0.5 | −12.4 | −26.1 |
| D-2310 | 0.5 | −9.8 | −26.4 |
| D-2294 | 0.5 | −55.5 | −13.5 |
| D-2295 | 0.5 | −52.7 | −23.8 |
| D-2331 | 1 | −16.4 | −17.7 |
| D-2331 | 0.5 | 7.3 | −26.9 |
| D-2332 | 0.5 | −16.6 | −36.1 |
| D-2329 | 0.5 | −1.1 | −28.6 |
| D-2330 | 0.5 | −17.3 | −33.2 |
| D-2340 | 1 | −30.0 | −30.7 |
| D-2340 | 0.5 | −29.0 | −41.5 |
| D-2341 | 0.5 | −21.5 | −31.6 |
| D-2335 | 0.5 | −15.7 | −24.1 |
| D-2336 | 0.5 | −26.9 | −28.6 |
| D-2415 | 1 | −18.2 | −26.5 |
| D-2415 | 0.5 | −18.0 | −37.5 |
| D-2418 | 0.5 | 2.8 | −37.5 |
| D-2421 | 0.5 | −73.0 | −57.8 |
| D-2425 | 0.5 | −75.1 | −49.1 |
| D-2454 | 1 | 19.5 | −31.4 |
| D-2454 | 0.5 | −40.6 | −14.6 |
| D-2472 | 1 | −25.9 | −21.8 |
| D-2472 | 0.5 | −15.0 | −24.0 |
| D-2451 | 0.5 | −70.5 | −19.9 |
| D-2452 | 0.5 | −63.3 | −28.0 |
| D-2423 | 1 | −86.1 | −63.7 |
| D-2423 | 0.5 | −68.0 | −48.6 |
| D-2449 | 1 | −46.3 | −31.3 |
| D-2449 | 0.5 | −40.5 | −34.8 |
| D-2316 | 1 | −74.6 | −37.2 |
| D-2316 | 0.5 | −64.5 | −29.7 |
| D-2381 | 1 | −73.4 | −80.2 |
| D-2381 | 0.5 | −72.4 | −71.4 |
| D-2412 | 0.5 | −64.6 | −4.7 |
| D-2412 | 1 | −77.2 | −10.7 |
| D-2412 | 3 | −96.3 | −11.3 |
| D-2408 | 0.5 | −69.7 | 3.4 |
| D-2408 | 1 | −90.1 | 2.0 |
| D-2408 | 3 | −96.9 | −14.6 |
| D-2398 | 0.5 | −35.0 | −6.2 |
| D-2398 | 1 | −22.8 | −22.5 |
| D-2398 | 3 | −56.3 | −20.4 |
| D-2423 | 0.5 | −84.1 | −45.2 |
| D-2423 | 1 | −83.1 | −50.4 |
| D-2423 | 3 | −98.3 | −89.0 |
| D-2417 | 0.5 | −65.1 | −34.7 |
| D-2417 | 1 | −87.6 | −46.8 |
| D-2417 | 3 | −96.5 | −79.2 |
| D-2381 | 0.5 | −63.4 | −57.9 |
| D-2381 | 1 | −84.2 | −84.0 |
| D-2381 | 3 | −94.2 | −93.1 |
| D-2316 | 0.5 | −59.8 | −18.2 |
| D-2316 | 1 | −80.4 | −12.2 |
| D-2316 | 3 | −96.9 | −17.4 |
| D-2449 | 0.5 | −36.1 | −3.5 |
| D-2449 | 1 | −74.4 | −21.2 |
| D-2449 | 3 | 86.4 | −25.4 |
| D-2452 | 0.5 | −76.1 | −12.1 |
| D-2452 | 1 | 84.3 | −12.5 |
| D-2452 | 3 | −96.1 | −20.8 |
| D-2451 | 0.5 | −63.4 | −12.7 |
| D-2451 | 1 | −80.9 | −11.9 |
| D-2451 | 3 | −96.1 | −28.0 |
| D-2425 | 0.5 | −71.8 | −30.6 |
| D-2425 | 1 | −84.0 | −60.5 |
| D-2425 | 3 | −97.6 | −72.3 |
| D-2478 | 0.5 | −64.5 | −18.6 |
| D-2478 | 1 | −83.5 | −16.3 |
| D-2478 | 3 | −92.4 | −12.3 |
| ALNY #2 | 1 | −86.4 | −0.1 |
| (AD-87404) | | | |
| ALNY #2 | 0.5 | −72.9 | −3.3 |
| (AD-87404) | | | |
| D-2408 | 1 | −81.7 | −18.0 |
| D-2408 | 0.5 | −74.2 | −11.4 |
| D-2423 | 1 | −83.5 | −52.1 |
| D-2423 | 0.5 | −57.3 | −15.7 |
| D-2452 | 1 | −77.4 | −12.1 |
| D-2452 | 0.5 | −49.8 | 1.0 |
| D-2408 | 1 | −82.8 | −2.4 |
| D-2408 | 0.5 | −77.7 | 0.4 |
| D-2423 | 1 | −86.1 | −55.6 |
| D-2423 | 0.5 | −67.6 | −20.7 |
| D-2452 | 1 | −83.5 | −12.4 |
| D-2452 | 0.5 | −66.1 | 5.6 |
| D-2408 | 0.5 | −71.9 | −31.7 |
| D-2423 | 0.5 | −70.8 | −41.2 |
| D-2452 | 0.5 | −68.5 | −12.1 |
| D-2519 | 1 | −59.2 | 5.0 |
| D-2519 | 0.5 | −38.2 | −11.1 |
| D-2511 | 0.5 | −30.9 | −16.5 |
| D-2520 | 0.5 | −64.1 | −21.0 |
| D-2517 | 0.5 | −34.4 | −17.1 |
| D-2516 | 0.5 | −68.3 | −17.9 |
| D-2317 | 1 | −68.0 | −0.6 |
| D-2317 | 0.5 | −56.7 | −31.4 |
| D-2298 | 0.5 | −46.8 | 9.4 |
| D-2321 | 0.5 | −47.4 | −12.4 |
| D-2313 | 0.5 | −56.3 | −16.8 |
| D-2311 | 0.5 | −48.3 | −30.8 |
| D-2363 | 1 | −38.2 | 21.6 |
| D-2363 | 0.5 | −2.7 | 66.1 |
| D-2358 | 0.5 | 4.8 | 78.5 |
| D-2364 | 0.5 | −31.5 | 64.6 |
| D-2360 | 0.5 | −23.9 | 57.7 |
| D-2359 | 0.5 | −27.7 | 65.0 |
| D-2383 | 1 | −67.1 | 13.3 |
| D-2383 | 0.5 | −16.9 | 168.1 |
| D-2366 | 0.5 | −35.3 | 46.3 |
| D-2386 | 0.5 | 6.6 | 51.7 |
| D-2379 | 0.5 | −12.0 | 73.5 |
| D-2377 | 0.5 | −37.8 | 31.4 |
| D-2424 | 1 | −79.2 | 7.2 |
| D-2424 | 0.5 | 29.6 | −12.9 |
| D-2416 | 0.5 | −41.7 | −12.2 |
| D-2427 | 0.5 | −32.4 | 1.0 |
| D-2422 | 0.5 | −23.6 | 18.5 |
| D-2419 | 0.5 | −41.0 | 6.6 |
| D-2316 | 1 | −63.0 | 28.3 |
| D-2316 | 0.5 | −45.2 | 5.6 |
| D-2314 | 0.5 | −36.1 | −3.7 |
| D-2301 | 0.5 | −50.2 | 15.9 |
| D-2299 | 0.5 | −4.8 | 17.8 |
| D-2303 | 0.5 | −22.0 | −5.9 |
| D-2478 | 1 | −57.9 | 0.8 |
| D-2478 | 0.5 | −14.8 | −1.4 |
| D-2474 | 0.5 | −15.3 | 3.4 |
| D-2455 | 0.5 | 27.6 | 30.2 |
| D-2456 | 0.5 | −32.7 | 16.5 |
| D-2457 | 0.5 | 7.4 | −2.9 |
| D-2408 | 1 | −85.3 | 29.0 |
| D-2408 | 0.5 | −64.3 | 8.4 |
| D-2381 | 3 | −96.7 | 16.2 |
| D-2381 | 1 | −67.9 | −64.0 |
| D-2381 | 0.5 | −50.5 | −34.5 |
| D-2365 | 1 | −47.4 | −44.5 |
| D-2365 | 0.5 | −54.6 | −37.9 |
| D-2385 | 1 | −51.9 | 0.0 |
| D-2385 | 0.5 | −62.8 | −8.0 |
| D-2378 | 3 | −83.8 | −35.4 |
| D-2378 | 1 | −54.0 | −26.2 |
| D-2378 | 0.5 | −52.4 | −25.6 |
| D-2376 | 1 | −70.9 | −42.0 |
| D-2376 | 0.5 | −45.3 | −10.6 |
| D-2383 | 3 | −92.3 | −65.8 |
| D-2383 | 1 | −65.1 | −25.6 |
| D-2383 | 0.5 | −26.4 | 7.2 |
| D-2312 | 3 | −97.3 | 28.5 |
| D-2312 | 1 | −88.5 | 20.4 |
| D-2312 | 0.5 | −67.6 | 27.1 |
| D-2301 | 1 | −76.1 | 12.9 |
| D-2301 | 0.5 | −58.1 | 20.6 |
| D-2317 | 1 | −67.0 | 21.1 |
| D-2317 | 0.5 | −53.1 | −6.4 |
| D-2313 | 1 | −65.3 | 5.9 |
| D-2313 | 0.5 | −54.7 | 2.7 |
| D-2440 | 3 | −90.4 | −57.0 |
| D-2440 | 1 | −78.0 | −25.7 |
| D-2440 | 0.5 | −34.0 | −12.6 |
| D-2363 | 1 | −53.5 | −14.9 |
| D-2363 | 0.5 | −20.9 | 1.9 |
| D-2496 | 1 | −17.1 | 22.8 |
| D-2496 | 0.5 | −34.6 | 7.6 |
| D-2516 | 3 | −92.0 | 8.3 |
| D-2516 | 1 | −72.3 | −3.3 |
| D-2516 | 0.5 | −44.8 | −5.5 |
| D-2440 | 0.5 | −41.3 | 53.5 |
| D-2428 | 0.5 | −49.3 | 71.2 |
| D-2442 | 0.5 | −61.1 | 19.0 |
| D-2438 | 0.5 | −26.7 | 41.5 |
| D-2437 | 0.5 | −49.3 | 14.3 |
| D-2478 | 0.5 | −30.8 | 47.4 |
| D-2481 | 0.5 | −52.0 | 59.9 |
| D-2473 | 0.5 | −44.4 | 54.2 |
| D-2475 | 0.5 | −40.6 | 2.3 |
| D-2453 | 0.5 | −53.5 | 36.1 |
| D-2471 | 0.5 | −78.2 | 49.4 |
| D-2317 | 0.5 | −64.5 | 24.2 |
| D-2315 | 0.5 | −70.9 | −57.1 |
| D-2302 | 0.5 | −77.4 | −62.6 |
| D-2300 | 0.5 | −69.4 | −51.6 |
| D-2313 | 0.5 | −79.8 | −53.1 |
| D-2318 | 0.5 | −62.4 | −46.0 |
| D-2319 | 0.5 | −69.5 | −63.5 |
| D-2304 | 0.5 | −81.3 | −57.5 |
| D-2377 | 0.5 | −73.0 | −57.2 |
| D-2369 | 0.5 | −66.2 | −59.2 |
| D-2370 | 0.5 | −78.7 | −75.6 |
| D-2371 | 0.5 | −85.1 | −63.3 |
| D-2478 | 0.5 | −79.1 | −64.1 |
| D-2478 | 0.5 | −82.2 | −61.2 |
| D-2380 | 0.5 | −22.2 | −13.9 |
| D-2368 | 0.5 | −46.8 | −36.3 |
| D-2367 | 0.5 | −45.1 | −21.1 |
| D-2408 | 0.5 | −76.7 | −5.3 |
| D-2401 | 0.5 | −67.8 | 15.6 |
| D-2402 | 0.5 | −69.4 | −21.6 |
| D-2403 | 0.5 | −70.1 | −5.5 |
| D-2452 | 0.5 | −73.8 | −14.8 |
| D-2479 | 0.5 | −72.0 | 3.8 |
| D-2480 | 0.5 | −67.1 | −8.3 |
| D-2458 | 0.5 | −65.9 | −3.2 |
| D-2308 | 0.5 | −59.4 | 10.6 |
| D-2307 | 0.5 | −53.8 | 13.1 |
| D-2305 | 0.5 | −73.1 | 15.6 |
| D-2306 | 0.5 | −62.2 | 21.5 |
| D-2349 | 0.5 | −73.2 | −67.6 |
| D-2348 | 0.5 | −64.8 | −65.5 |
| D-2346 | 0.5 | −62.9 | −44.7 |
| D-2347 | 0.5 | −68.4 | −60.8 |
| D-2375 | 0.5 | −71.2 | −27.7 |
| D-2374 | 0.5 | −54.8 | −27.9 |
| D-2372 | 0.5 | −73.9 | −29.1 |
| D-2373 | 0.5 | −60.8 | −38.7 |
| D-2407 | 0.5 | −79.3 | −16.7 |
| D-2406 | 0.5 | −69.0 | −22.0 |
| D-2404 | 0.5 | −75.2 | −7.9 |
| D-2405 | 0.5 | −70.4 | −13.2 |
| D-2435 | 0.5 | −71.8 | −55.3 |
| D-2433 | 0.5 | −64.6 | −56.1 |
| D-2429 | 0.5 | −70.2 | −52.6 |
| D-2431 | 0.5 | −66.8 | −45.7 |
| D-2436 | 0.5 | −81.0 | −6.7 |
| D-2434 | 0.5 | −62.2 | −3.6 |
| D-2430 | 0.5 | −67.9 | 23.1 |
| D-2432 | 0.5 | −73.4 | −9.3 |
| D-2468 | 0.5 | −62.3 | −19.8 |
| D-2465 | 0.5 | 69.2 | −5.1 |
| D-2459 | 0.5 | −63.8 | −7.5 |
| D-2462 | 0.5 | −50.8 | −5.9 |
| D-2469 | 0.5 | −59.9 | 0.9 |
| D-2466 | 0.5 | −61.9 | −7.3 |
| D-2460 | 0.5 | 72.8 | −12.8 |
| D-2463 | 0.5 | −51.2 | −9.7 |
| D-2470 | 0.5 | −65.0 | −9.6 |
| D-2467 | 0.5 | −72.8 | 0.0 |
| D-2461 | 0.5 | −54.1 | −19.2 |
| D-2464 | 0.5 | −79.2 | −13.3 |
| D-2515 | 0.5 | −55.8 | −23.1 |
| D-2514 | 0.5 | −56.7 | −24.7 |
| D-2512 | 0.5 | −50.7 | 10.6 |
| D-2513 | 0.5 | −44.7 | −4.9 |
| D-2478 | 0.5 | −54.3 | −6.1 |
| D-2478 | 1 | −64.5 | −14.0 |
| D-2302 | 0.5 | −48.0 | 2.7 |
| D-2313 | 0.5 | −73.9 | −8.1 |
| D-2304 | 0.5 | −59.7 | −2.5 |
| D-2370 | 0.5 | −56.1 | −7.9 |
| D-2371 | 0.5 | −44.1 | 0.6 |
| D-2403 | 0.5 | −71.2 | −4.3 |

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

The foregoing written specification is considered to be sufficient to enable one skilled in the art to practice the invention. The foregoing description and examples detail certain embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

What is claimed:

1. An RNAi construct comprising a sense strand and an antisense strand, wherein the antisense strand comprises a region having at least 15 contiguous nucleotides differing by no more than 3 nucleotides from an antisense sequence of: SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, or 1132, and wherein the RNAi construct inhibits the expression of a Transthyretin (TTR) mRNA.

2. An RNAi construct comprising a sense strand and an antisense strand, wherein the antisense strand has a transcript binding start location at nucleotide 195 to 201, and comprises a region having at least 15 contiguous nucleotides differing by no more than 3 nucleotides from an antisense sequence of SEQ ID NO: 18, 20, 22, 24, 26, 28, 584, 586, 588, 590, 592, 594, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, or 1214.

3. An RNAi construct comprising a sense strand and an antisense strand, wherein the antisense strand has a transcript binding start location at nucleotide 195 to 201, and comprises a region having at least 15 contiguous nucleotides differing by no more than 3 nucleotides from an antisense sequence and wherein the sense strand comprises a region having at least 15 contiguous nucleotides differing by no more than 3 nucleotides from a sense sequence, wherein the sense and antisense sequences comprise the sequence of SEQ ID NO: 17 and 18; 19 and 20; 21 and 22; 23 and 24; 25 and 26; 27 and 28; 583 and 584; 585 and 586; 587 and 588; 589 and 590; 591 and 592; 593 and 594; 1151 and 1152; 1153 and 1154; 1155 and 1156; 1157 and 1158; 1159 and 1160; 1161 and 1162; 1163 and 1164; 1165 and 1166; 1167 and 1168; 1169 and 1170; 1171 and 1172; 1173 and 1174; 1175 and 1176; 1177 and 1178; 1179 and 1180; 1181 and 1182; 1183 and 1184; 1185 and 1186; 1187 and 1188; 1189 and 1190; 1191 and 1192; 1193 and 1194; 1195 and 1196; 1197 and 1198; 1199 and 1200; 1201 and 1202; 1203 and 1204; 1205 and 1206; 1207 and 1208; 1209 and 1210; 1211 and 1212; or 1213 and 1214.

4. An RNAi construct comprising a sense strand and an antisense strand, wherein the antisense strand has a transcript binding start location at nucleotide 236 to 239, and comprises a region having at least 15 contiguous nucleotides differing by no more than 3 nucleotides from an antisense sequence of SEQ ID NO: 50, 52, 54, 56, 616, 618, 620, 622, 1256, 1258, 1260, 1262, 1264, 1266, 1268, or 1270.

5. An RNAi construct comprising a sense strand and an antisense strand, wherein the antisense strand has a transcript binding start location at nucleotide 236 to 239, and comprises a region having at least 15 contiguous nucleotides differing by no more than 3 nucleotides from an antisense sequence and wherein the sense strand comprises a region having at least 15 contiguous nucleotides differing by no more than 3 nucleotides from a sense sequence, wherein the sense and antisense sequences comprise the sequence of SEQ ID NO: 49 and 50; 51 and 52; 53 and 54; 55 and 56; 615 and 616; 617 and 618; 619 and 620; 621 and 622; 1255 and 1256; 1257 and 1258; 1259 and 1260; 1261 and 1262; 1263 and 1264; 1265 and 1266; 1267 and 1268; or 1269 and 1270.

6. An RNAi construct comprising a sense strand and an antisense strand, wherein the antisense strand has a transcript binding start location at nucleotide 277 to 283, and comprises a region having at least 15 contiguous nucleotides differing by no more than 3 nucleotides from an antisense sequence of SEQ ID NO: 62, 64, 66, 68, 70, 72, 74, 628, 630, 632, 634, 636, 638, 640, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, or 1348.

7. An RNAi construct comprising a sense strand and an antisense strand, wherein the antisense strand has a transcript binding start location at nucleotide 277 to 283, and comprises a region having at least 15 contiguous nucleotides differing by no more than 3 nucleotides from an antisense sequence and wherein the sense strand comprises a region having at least 15 contiguous nucleotides differing by no more than 3 nucleotides from a sense sequence, wherein the sense and antisense sequences comprise the sequence of SEQ ID NO: 61 and 62; 63 and 64; 65 and 66; 67 and 68; 69 and 70; 71 and 72; 73 and 74; 627 and 628; 629 and 630; 631 and 632; 633 and 634; 635 and 636; 637 and 638; 639 and 640; 1275 and 1276; 1277 and 1278; 1279 and 1280; 1281 and 1282; 1283 and 1284; 1285 and 1286; 1287 and 1288; 1289 and 1290; 1291 and 1292; 1293 and 1294; 1295 and 1296; 1297 and 1298; 1299 and 1300; 1301 and 1302; 1303 and 1304; 1305 and 1306; 1307 and 1308; 1309 and 1310; 1311 and 1312:1313 and 1314; 1315 and 1316; 1317 and 1318; 1319 and 1320; 1321 and 1322; 1323 and 1324; 1325 and 1326; 1327 and 1328; 1329 and 1330; 1331 and 1332; 1333 and 1334; 1335 and 1336; 1337 and 1338; 1339 and 1340; 1341 and 1342; 1343 and 1344; 1345 and 1346; or 1347 and 1348.

8. The RNAi construct of any one of claims 1-7, wherein the sense strand comprises a sequence that is sufficiently complementary to the sequence of the antisense strand to form a duplex region of about 15 to about 30 base pairs in length.

9. The RNAi construct of any one of claims 1-8, wherein the duplex region is about 17 to about 24 base pairs in length.

10. The RNAi construct of any one of claims 1-9, wherein the duplex region is about 19 to about 21 base pairs in length.

11. The RNAi construct of claim 10, wherein the duplex region is 19 base pairs in length.

12. The RNAi construct of any one of claims 1-11, wherein the sense strand and the antisense strand are each about 15 to about 30 nucleotides in length.

13. The RNAi construct of claim 12, wherein the sense strand and the antisense strand are each about 19 to about 27 nucleotides in length.

14. The RNAi construct of claim 13, wherein the sense strand and the antisense strand are each about 21 to about 25 nucleotides in length.

15. The RNAi construct of claim 14, wherein the sense strand and the antisense strand are each about 21 to about 23 nucleotides in length.

16. The RNAi construct of any one of claims 1-15, which comprises at least one blunt end.

17. The RNAi construct of any one of claims 1-16, which comprises at least one nucleotide overhang of 1 to 4 unpaired nucleotides.

18. The RNAi construct of claim 17, wherein the nucleotide overhang has two unpaired nucleotides.

19. The RNAi construct of claim 17 or 18, wherein the RNAi construct comprises a nucleotide overhang at the 3′ end of the sense strand, the 3′ end of the antisense strand, or the 3′ end of both the sense strand and the antisense strand.

20. The RNAi construct of any one of claims 17-19, wherein the nucleotide overhang comprises a 5′-UU-3′ dinucleotide or a 5′-dTdT-3′ dinucleotide.

21. The RNAi construct of any one of claims 1-20, wherein the RNAi construct comprises at least one modified nucleotide.

22. The RNAi construct of claim 21, wherein the modified nucleotide is a 2′-modified nucleotide.

23. The RNAi construct of claim 21, wherein the modified nucleotide is a 2′-fluoro modified nucleotide, a 2′-O-methyl modified nucleotide, a 2′-O-methoxyethyl modified nucleotide, a 2′-O-allyl modified nucleotide, a bicyclic nucleic acid (BNA), a glycol nucleic acid, an inverted base, or combinations thereof.

24. The RNAi construct of claim 23, wherein the modified nucleotide is a 2′-O-methyl modified nucleotide, a 2′-O-methoxyethyl modified nucleotide, a 2′-fluoro modified nucleotide, or combinations thereof.

25. The RNAi construct of claim any one of claims 21-24, wherein all of the nucleotides in the sense and antisense strands are modified nucleotides.

26. The RNAi construct of claim 25, wherein the modified nucleotides are 2′-O-methylmodified nucleotides, 2′-fluoro modified nucleotides, or combinations thereof.

27. The RNAi construct of any one of claims 1-26, which comprises at least one phosphorothioate internucleotide linkage.

28. The RNAi construct of claim 27, wherein the RNAi construct comprises two consecutive phosphorothioate internucleotide linkages at the 3′ end of the antisense strand.

29. The RNAi construct of claim 27, wherein the RNAi construct comprises two consecutive phosphorothioate internucleotide linkages at both the 3′ and 5′ ends of the antisense strand and two consecutive phosphorothioate internucleotide linkages at the 5′ end of the sense strand.

30. The RNAi construct of any one of claims 1-29, wherein the sense strand comprises a sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, or 565.

31. The RNAi construct of any one of claims 1-29, wherein the sense strand comprises a sequence of SEQ ID NO: 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1471, 1473, 1475, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, or 1617.

32. The RNAi construct of any one of claims 1-31, wherein the RNAi construct is any one of the duplex compounds of: SEQ ID NO: 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; 27 and 28; 29 and 30; 31 and 32; 33 and 34; 35 and 36; 37 and 38; 39 and 40; 41 and 42; 43 and 44; 45 and 46; 47 and 48; 49 and 50; 51 and 52; 53 and 54; 55 and 56; 57 and 58; 59 and 60; 61 and 62; 63 and 64; 65 and 66; 67 and 68; 69 and 70; 71 and 72; 73 and 74; 75 and 76; 77 and 78; 79 and 80; 81 and 82; 83 and 84; 85 and 86; 87 and 88; 89 and 90; 91 and 92; 93 and 94; 95 and 96; 97 and 98; 99 and 100; 101 and 102; 103 and 104; 105 and 106; 107 and 108; 109 and 110; 111 and 112; 113 and 114; 115 and 116; 117 and 118; 119 and 120; 121 and 122; 123 and 124; 125 and 126; 127 and 128; 129 and 130; 131 and 132; 133 and 134; 135 and 136; 137 and 138; 139 and 140; 141 and 142; 143 and 144; 145 and 146; 147 and 148; 149 and 150; 151 and 152; 153 and 154; 155 and 156; 157 and 158; 159 and 160; 161 and 162; 163 and 164; 165 and 166; 167 and 168; 169 and 170; 171 and 172; 173 and 174; 175 and 176; 177 and 178; 179 and 180; 181 and 182; 183 and 184; 185 and 186; 187 and 188; 189 and 190; 191 and 192; 193 and 194; 195 and 196; 197 and 198; 199 and 200; 201 and 202; 203 and 204; 205 and 206; 207 and 208; 209 and 210; 211 and 212; 213 and 214; 215 and 216; 217 and 218; 219 and 220; 221 and 222; 223 and 224; 225 and 226; 227 and 228; 229 and 230; 231 and 232; 233 and 234; 235 and 236; 237 and 238; 239 and 240; 241 and 242; 243 and 244; 245 and 246; 247 and 248; 249 and 250; 251 and 252; 253 and 254; 255 and 256; 257 and 258; 259 and 260; 261 and 262; 263 and 264; 265 and 266; 267 and 268; 269 and 270; 271 and 272; 273 and 274; 275 and 276; 277 and 278; 279 and 280; 281 and 282; 283 and 284; 285 and 286; 287 and 288; 289 and 290; 291 and 292; 293 and 294; 295 and 296; 297 and 298; 299 and 300; 301 and 302; 303 and 304; 305 and 306; 307 and 308; 309 and 310; 311 and 312; 313 and 314; 315 and 316; 317 and 318; 319 and 320; 321 and 322; 323 and 324; 325 and 326; 327 and 328; 329 and 330; 331 and 332; 333 and 334; 335 and 336; 337 and 338; 339 and 340; 341 and 342; 343 and 344:345 and 346; 347 and 348; 349 and 350; 351 and 352; 353 and 354; 355 and 356; 357 and 358; 359 and 360; 361 and 362; 363 and 364; 365 and 366; 367 and 368; 369 and 370; 371 and 372; 373 and 374; 375 and 376; 377 and 378; 379 and 380; 381 and 382; 383 and 384; 385 and 386; 387 and 388; 389 and 390; 391 and 392; 393 and 394; 395 and 396; 397 and 398; 399 and 400; 401 and 402; 403 and 404; 405 and 406; 407 and 408; 409 and 410; 411 and 412; 413 and 414; 415 and 416; 417 and 418; 419 and 420; 421 and 422; 423 and 424; 425 and 426; 427 and 428; 429 and 430; 431 and 432; 433 and 434; 435 and 436; 437 and 438; 439 and 440; 441 and 442; 443 and 444; 445 and 446; 447 and 448; 449 and 450; 451 and 452; 453 and 454; 455 and 456; 457 and 458; 459 and 460; 461 and 462; 463 and 464; 465 and 466; 467 and 468; 469 and 470; 471 and 472; 473 and 474; 475 and 476; 477 and 478; 479 and 480; 481 and 482; 483 and 484; 485 and 486; 487 and 488; 489 and 490; 491 and 492; 493 and 494; 495 and 496; 497 and 498; 499 and 500; 501 and 502; 503 and 504; 505 and 506; 507 and 508; 509 and 510; 511 and 512; 513 and 514; 515 and 516; 517 and 518; 519 and 520; 521 and 522; 523 and 524; 525 and 526; 527 and 528; 529 and 530; 531 and 532; 533 and 534; 535 and 536; 537 and 538; 539 and 540; 541 and 542; 543 and 544; 545 and 546; 547 and 548; 549 and 550; 551 and 552; 553 and 554; 555 and 556; 557 and 558; 559 and 560; 561 and 562; 563 and 564; or 565 and 566.

33. The RNAi construct of any one of claims 1-31, wherein the RNAi construct is any one of the duplex compounds of: SEQ ID NO: 567 and 568; 569 and 570; 571 and 572; 573 and 574; 575 and 576; 577 and 578; 579 and 580; 581 and 582; 583 and 584; 585 and 586; 587 and 588; 589 and 590; 591 and 592; 593 and 594; 595 and 596; 597 and 598; 599 and 600; 601 and 602; 603 and 604; 605 and 606; 607 and 608; 609 and 610; 611 and 612; 613 and 614; 615 and 616; 617 and 618; 619 and 620; 621 and 622; 623 and 624; 625 and 626; 627 and 628; 629 and 630; 631 and 632; 633 and 634; 635 and 636; 637 and 638; 639 and 640; 641 and 642; 643 and 644; 645 and 646; 647 and 648; 649 and 650; 651 and 652; 653 and 654; 655 and 656; 657 and 658; 659 and 660; 661 and 662; 663 and 664; 665 and 666; 667 and 668; 669 and 670; 671 and 672; 673 and 674; 675 and 676; 677 and 678; 679 and 680; 681 and 682; 683 and 684; 685 and 686; 687 and 688; 689 and 690; 691 and 692; 693 and 694; 695 and 696; 697 and 698; 699 and 700; 701 and 702; 703 and 704; 705 and 706; 707 and 708; 709 and 710; 711 and 712; 713 and 714; 715 and 716; 717 and 718; 719 and 720; 721 and 722; 723 and 724; 725 and 726; 727 and 728; 729 and 730; 731 and 732; 733 and 734; 735 and 736; 737 and 738; 739 and 740; 741 and 742; 743 and 744; 745 and 746; 747 and 748; 749 and 750; 751 and 752; 753 and 754; 755 and 756; 757 and 758; 759 and 760; 761 and 762; 763 and 764; 765 and 766; 767 and 768; 769 and 770; 771 and 772; 773 and 774; 775 and 776; 777 and 778; 779 and 780; 781 and 782; 783 and 784; 785 and 786; 787 and 788; 789 and 790; 791 and 792; 793 and 794; 795 and 796; 797 and 798; 799 and 800; 801 and 802; 803 and 804; 805 and 806; 807 and 808; 809 and 810; 811 and 812; 813 and 814; 815 and 816; 817 and 818; 819 and 820; 821 and 822; 823 and 824; 825 and 826; 827 and 828; 829 and 830; 831 and 832; 833 and 834; 835 and 836; 837 and 838; 839 and 840; 841 and 842; 843 and 844; 845 and 846; 847 and 848; 849 and 850; 851 and 852; 853 and 854; 855 and 856; 857 and 858; 859 and 860; 861 and 862; 863 and 864; 865 and 866; 867 and 868; 869 and 870; 871 and 872; 873 and 874; 875 and 876; 877 and 878; 879 and 880; 881 and 882; 883 and 884; 885 and 886; 887 and 888; 889 and 890; 891 and 892; 893 and 894; 895 and 896; 897 and 898; 899 and 900; 901 and 902; 903 and 904; 905 and 906; 907 and 908; 909 and 910; 911 and 912; 913 and 914; 915 and 916; 917 and 918; 919 and 920; 921 and 922; 923 and 924; 925 and 926; 927 and 928; 929 and 930; 931 and 932; 933 and 934; 935 and 936; 937 and 938; 939 and 940; 941 and 942; 943 and 944; 945 and 946; 947 and 948; 949 and 950; 951 and 952; 953 and 954; 955 and 956; 957 and 958; 959 and 960; 961 and 962; 963 and 964; 965 and 966; 967 and 968; 969 and 970; 971 and 972; 973 and 974; 975 and 976; 977 and 978; 979 and 980; 981 and 982; 983 and 984; 985 and 986; 987 and 988; 989 and 990; 991 and 992; 993 and 994; 995 and 996; 997 and 998; 999 and 1000; 1001 and 1002; 1003 and 1004; 1005 and 1006; 1007 and 1008; 1009 and 1010; 1011 and 1012; 1013 and 1014; 1015 and 1016; 1017 and 1018; 1019 and 1020; 1021 and 1022; 1023 and 1024; 1025 and 1026; 1027 and 1028; 1029 and 1030; 1031 and 1032; 1033 and 1034; 1035 and 1036; 1037 and 1038; 1039 and 1040; 1041 and 1042; 1043 and 1044; 1045 and 1046; 1047 and 1048; 1049 and 1050; 1051 and 1052; 1053 and 1054; 1055 and 1056; 1057 and 1058; 1059 and 1060; 1061 and 1062; 1063 and 1064; 1065 and 1066; 1067 and 1068; 1069 and 1070; 1071 and 1072; 1073 and 1074; 1075 and 1076; 1077 and 1078; 1079 and 1080; 1081 and 1082; 1083 and 1084; 1085 and 1086; 1087 and 1088; 1089 and 1090; 1091 and 1092:1093 and 1094; 1095 and 1096; 1097 and 1098; 1099 and 1100; 1101 and 1102; 1103 and 1104; 1105 and 1106; 1107 and 1108; 1109 and 1110; 1111 and 1112; 1113 and 1114; 1115 and 1116; 1117 and 1118; 1119 and 1120; 1121 and 1122; 1123 and 1124; 1125 and 1126; 1127 and 1128; 1129 and 1130; 1131 and 1132; 1133 and 1134; 1135 and 1136; 1137 and 1138; 1139 and 1140; 1141 and 1142; 1143 and 1144; 1145 and 1146; 1147 and 1148; 1149 and 1150; 1151 and 1152; 1153 and 1154; 1155 and 1156; 1157 and 1158; 1159 and 1160; 1161 and 1162; 1163 and 1164; 1165 and 1166; 1167 and 1168; 1169 and 1170; 1171 and 1172; 1173 and 1174; 1175 and 1176; 1177 and 1178; 1179 and 1180; 1181 and 1182; 1183 and 1184; 1185 and 1186; 1187 and 1188; 1189 and 1190; 1191 and 1192; 1193 and 1194; 1195 and 1196; 1197 and 1198; 1199 and 1200; 1201 and 1202; 1203 and 1204; 1205 and 1206; 1207 and 1208; 1209 and 1210; 1211 and 1212; 1213 and 1214; 1215 and 1216; 1217 and 1218; 1219 and 1220; 1221 and 1222; 1223 and 1224; 1225 and 1226; 1227 and 1228; 1229 and 1230; 1231 and 1232; 1233 and 1234; 1235 and 1236; 1237 and 1238; 1239 and 1240; 1241 and 1242; 1243 and 1244; 1245 and 1246; 1247 and 1248; 1249 and 1250; 1251 and 1252; 1253 and 1254; 1255 and 1256; 1257 and 1258; 1259 and 1260; 1261 and 1262; 1263 and 1264; 1265 and 1266; 1267 and 1268; 1269 and 1270; 1271 and 1272; 1273 and 1274; 1275 and 1276; 1277 and 1278; 1279 and 1280; 1281 and 1282; 1283 and 1284; 1285 and 1286; 1287 and 1288; 1289 and 1290; 1291 and 1292; 1293 and 1294; 1295 and 1296; 1297 and 1298; 1299 and 1300; 1301 and 1302; 1303 and 1304; 1305 and 1306; 1307 and 1308; 1309 and 1310; 1311 and 1312; 1313 and 1314; 1315 and 1316; 1317 and 1318; 1319 and 1320; 1321 and 1322; 1323 and 1324; 1325 and 1326; 1327 and 1328; 1329 and 1330; 1331 and 1332; 1333 and 1334; 1335 and 1336; 1337 and 1338; 1339 and 1340; 1341 and 1342; 1343 and 1344; 1345 and 1346; 1347 and 1348; 1349 and 1350; 1351 and 1352; 1353 and 1354; 1355 and 1356; 1357 and 1358; 1359 and 1360; 1361 and 1362; 1363 and 1364; 1365 and 1366; 1367 and 1368; 1369 and 1370; 1371 and 1372; 1373 and 1374; 1375 and 1376; 1377 and 1378; 1379 and 1380; 1381 and 1382; 1383 and 1384; 1385 and 1386; 1387 and 1388; 1389 and 1390; 1391 and 1392; 1393 and 1394; 1395 and 1396; 1397 and 1398; 1399 and 1400; 1401 and 1402; 1403 and 1404; 1405 and 1406; 1407 and 1408; 1409 and 1410; 1411 and 1412; 1413 and 1414; 1415 and 1416; 1417 and 1418; 1419 and 1420; 1421 and 1422; 1423 and 1424; 1425 and 1426; 1427 and 1428; 1429 and 1430; 1431 and 1432; 1433 and 1434; 1435 and 1436; 1437 and 1438; 1439 and 1440; 1441 and 1442; 1443 and 1444; 1445 and 1446; 1447 and 1448; 1449 and 1450; 1451 and 1452; 1453 and 1454; 1455 and 1456; 1457 and 1458; 1459 and 1460; 1461 and 1462; 1463 and 1464; 1465 and 1466; 1467 and 1468; 1469 and 1470; 1471 and 1472; 1473 and 1474; 1475 and 1476; 1477 and 1478; 1479 and 1480; 1481 and 1482; 1483 and 1484; 1485 and 1486; 1487 and 1488; 1489 and 1490; 1491 and 1492; 1493 and 1494; 1495 and 1496; 1497 and 1498; 1499 and 1500; 1501 and 1502; 1503 and 1504; 1505 and 1506; 1507 and 1508; 1509 and 1510; 1511 and 1512; 1513 and 1514; 1515 and 1516; 1517 and 1518; 1519 and 1520; 1521 and 1522; 1523 and 1524; 1525 and 1526; 1527 and 1528; 1529 and 1530; 1531 and 1532; 1533 and 1534; 1535 and 1536; 1537 and 1538; 1539 and 1540; 1541 and 1542; 1543 and 1544; 1545 and 1546; 1547 and 1548; 1549 and 1550; 1551 and 1552; 1553 and 1554; 1555 and 1556; 1557 and 1558; 1559 and 1560; 1561 and 1562; 1563 and 1564; 1565 and 1566; 1567 and 1568; 1569 and 1570; 1571 and 1572; 1573 and 1574; 1575 and 1576; 1577 and 1578; 1579 and 1580; 1581 and 1582; 1583 and 1584; 1585 and 1586; 1587 and 1588; 1589 and 1590; 1591 and 1592; 1593 and 1594; 1595 and 1596; 1597 and 1598; 1599 and 1600; 1601 and 1602; 1603 and 1604; 1605 and 1606; 1607 and 1608; 1609 and 1610; 1611 and 1612; 1613 and 1614; 1615 and 1616; or 1617 and 1618.

34. The RNAi construct of any one of claims 1-33, wherein the RNAi construct is a small interfering RNA (siRNA).

35. The RNAi construct of any one of claims 1-35, wherein the RNAi construct reduces the expression level of TTR in liver cells following incubation with the RNAi construct as compared to the TTR expression level in liver cells that have been incubated with a control RNAi construct.

36. The RNAi construct of claim 35, wherein the liver cells are Hep3B or HepG2 cells.

37. The RNAi construct of any one of claims 1-36, wherein the RNAi construct inhibits at least 40% of TTR expression at 5 nM in Hep3B cells in vitro.

38. The RNAi construct of any one of claims 1-36, wherein the RNAi construct inhibits at least 40% of TTR expression at 5 nM in HepG2 cells in vitro.

39. The RNAi construct of any one of claims 1-38, wherein the RNAi construct inhibits TTR expression in Hep3B cells with an IC50 of less than about 5 nM.

40. The RNAi construct of any one of claims 1-38, wherein the RNAi construct inhibits TTR expression in HepG2 cells with an IC50 of less than about 5 nM.

41. The RNAi construct of any one of claims 1-40, further comprising a ligand that binds to one or more proteins expressed on the surface of liver cells.

42. A composition comprising the RNAi construct of any one of claims 1-41 and a pharmaceutically acceptable carrier, excipient, or diluent.

43. A method for reducing the expression of TTR in a patient in need thereof comprising administering to the patient the RNAi construct of any one of claims 1-41 or the composition of claim 44.

44. The method of claim 43, wherein the expression level of TTR in hepatocytes is reduced in the patient following administration of the RNAi construct as compared to the TTR expression level in a patient not receiving the RNAi construct.

45. The method of any one of claims 43-44, wherein the patient suffers from transthyretin-associated cardiomyopathy (ATTR-CM).

46. A method for reducing the expression of TTR in vivo comprising administering to the animal the RNAi construct of any one of claims 1-41 or the composition of claim 42.

47. The method of claim 46, wherein the TTR is human TTR (hTTR).

48. The method of any one of claim 46 or 47, wherein expression of TTR is knocked down by at least 45% in an animal expressing hTTR by associated adenovirus (AAV) injection, and wherein the animal is administered the RNAi construct or the composition at 0.5 milligrams per kilogram of animal.

49. The method of claim 48, wherein the expression of TTR is knocked down by at least 50%, by at least 60%, or by at least 70%.

50. The method of any one of claim 46 or 47, wherein expression of TTR is knocked down by at least 60% in an animal expressing hTTR by associated adenovirus (AAV) injection, and wherein the animal is administered the RNAi construct or the composition at 1.0 milligrams per kilogram of animal.

51. The method of claim 50, wherein the expression of TTR is knocked down by at least 80%.

52. The method of any one of claim 46 or 47, wherein expression of TTR is knocked down by at least 90% in an animal expressing hTTR by associated adenovirus (AAV) injection, and wherein the animal is administered the RNAi construct or the composition at 0.5 milligrams per kilogram of animal.

53. The method of claim 52, wherein the expression of TTR is knocked down by at least 95%.
